SEQUENCE LISTING (161 sequences)

SEQ ID NO: 1 - DNA, 21 bp, synthetic construct
  misc_feature 1..21: note = SNCA1 targeting guide sequence
  source 1..21: mol_type = other DNA
ggtgtttgcagtaaacctaac

SEQ ID NO: 2 - DNA, 21 bp, synthetic construct
  misc_feature 1..21: note = SNCA1 targeting guide sequence
  source 1..21: mol_type = other DNA
gtccacctttaactcattgtg

SEQ ID NO: 3 - DNA, 21 bp, synthetic construct
  misc_feature 1..21: note = SNCA1 targeting guide sequence
  source 1..21: mol_type = other DNA
tttgcagtaaacctaacaaga

SEQ ID NO: 4 - DNA, 21 bp, synthetic construct
  misc_feature 1..21: note = SNCA1 targeting guide sequence
  source 1..21: mol_type = other DNA
tatttccctctactcaccaca

SEQ ID NO: 5 - DNA, 21 bp, synthetic construct
  misc_feature 1..21: note = SNCA1 targeting guide sequence
  source 1..21: mol_type = other DNA
tctggtcattcttcccacaaa

SEQ ID NO: 6 - DNA, 21 bp, synthetic construct
  misc_feature 1..21: note = SNCA1 targeting guide sequence
  source 1..21: mol_type = other DNA
gaaggcgagttaagaatagct

SEQ ID NO: 7 - DNA, 21 bp, synthetic construct
  misc_feature 1..21: note = SNCA1 targeting guide sequence
  source 1..21: mol_type = other DNA
aaaagccctcggtgactgtct

SEQ ID NO: 8 - DNA, 21 bp, synthetic construct
  misc_feature 1..21: note = SNCA1 targeting guide sequence
  source 1..21: mol_type = other DNA
aatgagattattattgggggt

SEQ ID NO: 9 - DNA, 21 bp, synthetic construct
  misc_feature 1..21: note = SNCA1 targeting guide sequence
  source 1..21: mol_type = other DNA
gtcattcttcccacaaaaaga

SEQ ID NO: 10 - DNA, 21 bp, synthetic construct
  misc_feature 1..21: note = SNCA1 targeting guide sequence
  source 1..21: mol_type = other DNA
accagtttaacccaccacgtt

SEQ ID NO: 11 - DNA, 21 bp, synthetic construct
  misc_feature 1..21: note = SNCA1 targeting guide sequence
  source 1..21: mol_type = other DNA
ggatatcaaattcagaacgtg

SEQ ID NO: 12 - DNA, 21 bp, synthetic construct
  misc_feature 1..21: note = SNCA1 targeting guide sequence
  source 1..21: mol_type = other DNA
cagcaggcagccaggcaggct

SEQ ID NO: 13 - DNA, 21 bp, synthetic construct
  misc_feature 1..21: note = SNCA1 targeting guide sequence
  source 1..21: mol_type = other DNA
tcttgtttgtgagcaaggtaa

SEQ ID NO: 14 - DNA, 21 bp, synthetic construct
  misc_feature 1..21: note = SNCA1 targeting guide sequence
  source 1..21: mol_type = other DNA
ggccacctgattgtttcctgg

SEQ ID NO: 15 - DNA, 21 bp, synthetic construct
  misc_feature 1..21: note = SNCA1 targeting guide sequence
  source 1..21: mol_type = other DNA
ttcatattatagtgaacagct

SEQ ID NO: 16 - DNA, 21 bp, synthetic construct
  misc_feature 1..21: note = SNCA1 targeting guide sequence
  source 1..21: mol_type = other DNA
tcatgatattatatagcaata

SEQ ID NO: 17 - DNA, 21 bp, synthetic construct
  misc_feature 1..21: note = SNCA1 targeting guide sequence
  source 1..21: mol_type = other DNA
aactgttactttattcctaat

SEQ ID NO: 18 - DNA, 21 bp, synthetic construct
  misc_feature 1..21: note = SNCA1 targeting guide sequence
  source 1..21: mol_type = other DNA
tagataacaatgattcatatt

SEQ ID NO: 19 - DNA, 21 bp, synthetic construct
  misc_feature 1..21: note = SNCA1 targeting guide sequence
  source 1..21: mol_type = other DNA
gttgaggaagaaatcataaat

SEQ ID NO: 20 - DNA, 21 bp, synthetic construct
  misc_feature 1..21: note = SNCA1 targeting guide sequence
  source 1..21: mol_type = other DNA
gtaattacagatgaagatagt

SEQ ID NO: 21 - DNA, 21 bp, synthetic construct
  misc_feature 1..21: note = SNCA1 targeting guide sequence
  source 1..21: mol_type = other DNA
actggccattaggctttctct

SEQ ID NO: 22 - DNA, 21 bp, synthetic construct
  misc_feature 1..21: note = SNCA1 targeting guide sequence
  source 1..21: mol_type = other DNA
gcaaaacctgccatatacaac

SEQ ID NO: 23 - DNA, 21 bp, synthetic construct
  misc_feature 1..21: note = SNCA1 targeting guide sequence
  source 1..21: mol_type = other DNA
tgaacatcgagaaaggggaag

SEQ ID NO: 24 - DNA, 21 bp, synthetic construct
  misc_feature 1..21: note = SNCA1 targeting guide sequence
  source 1..21: mol_type = other DNA
gccactggaccacccctgtat

SEQ ID NO: 25 - DNA, 21 bp, synthetic construct
  misc_feature 1..21: note = SNCA1 targeting guide sequence
  source 1..21: mol_type = other DNA
atttatctaggactcatatac

SEQ ID NO: 26 - DNA, 21 bp, synthetic construct
  misc_feature 1..21: note = SNCA1 targeting guide sequence
  source 1..21: mol_type = other DNA
cgcacctgacagcaataattt

SEQ ID NO: 27 - DNA, 21 bp, synthetic construct
  misc_feature 1..21: note = SNCA1 targeting guide sequence
  source 1..21: mol_type = other DNA
ggaaatttgctccttattatt

SEQ ID NO: 28 - DNA, 21 bp, synthetic construct
  misc_feature 1..21: note = SNCA1 targeting guide sequence
  source 1..21: mol_type = other DNA
acagtggatgaagccctttgt

SEQ ID NO: 29 - DNA, 21 bp, synthetic construct
  misc_feature 1..21: note = SNCA1 targeting guide sequence
  source 1..21: mol_type = other DNA
gtattgtatgtctcccaggaa

SEQ ID NO: 30 - DNA, 21 bp, synthetic construct
  misc_feature 1..21: note = SNCA1 targeting guide sequence
  source 1..21: mol_type = other DNA
gaagccctttgtttggattaa

SEQ ID NO: 31 - DNA, 21 bp, synthetic construct
  misc_feature 1..21: note = SNCA1 targeting guide sequence
  source 1..21: mol_type = other DNA
aggtgcgtctgccatacaacc

SEQ ID NO: 32 - DNA, 21 bp, synthetic construct
  misc_feature 1..21: note = SNCA1 targeting guide sequence
  source 1..21: mol_type = other DNA
ccatacaaccatgagtcctga

SEQ ID NO: 33 - DNA, 1280 bp, Mus musculus
  source 1..1280: mol_type = genomic DNA
caaagtggacagaggggaggggaggggatgcgaggggaggggaggggaatgatcgcgaaaggcttccaaaccttgtccttgtttttcaccatttctgaaatatatgctgagtgcaactatgggaagaccattttcataatctataatactcctccttttaaaggactttcgttaaccgcttgcaaaggtgagtgccgggtagaggacattagctcgctaagtccctagaaatcacacttggagactaagcaggctttcccaggagaagtccaaagccaacataagcaggaggctgggggctggccgttaaccgcaaggcagtggttgagccctcgggatcatcccggcggggggcgcagcatctccgccaaggccgcaggctctcaccatcagctgcccgagccaccctgtacctcgcagtccactcgccctgcccacgccccgcgccgcccgctcaccttcagcccctgggagtccatggccgccggctacccggagggtgcccaccgctgcccgccgcagggttaggggttcagacccacttcccgggccctcaaaccctaagggacgcggcgtgcagcacgaggggcgtggcccgatctccattggttgtcggcgtgagggggcggagcttagttgtaggactaggaaggagggggccaccggagcaggcgaggagggaaccccgagggaggaccgcgagggcgactggggctggaatccgctgagcattgagtgctgccgagttgtggggctagaggagggaggtccagcctggaaacggcgcgaggaggagggattgggtggagcaagagatatgagattaaagaataaagatgatgaagcagcaaataggagggagagccatgccgcttttcatccctgcaaacaaaggccgactccattttctcagcattttttgtggaagccgatttgcgcaatgcggcttagtacttgaccagggaaaatgatttacctgacacgtgtagtaatcgtgtctgggccacaaggtggcgcagaaaaatcacagttcggcaaaaaccttgaagcctggcttgggcttgttctaaatcttttcaggcgctgctgtaattttgctattcgagtgcttattaaactgctccgccagatttccacccccaaagtcttatttaaaaatatgtggttacctcttttagatttctatttcttaagtgtttgctgtagtttggatctaaactgtccctcaaagacacacgtgctgaatgttccccagcccgtgtgctgttgggagtggtgga

SEQ ID NO: 34 - DNA, 618 bp, Mus musculus
  source 1..618: mol_type = genomic DNA
aatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttatttt

SEQ ID NO: 35 - DNA, 832 bp, Mus musculus
  source 1..832: mol_type = genomic DNA
ataaaattttattttcctaaaatttacatttaaaagccatccaacttaccaaagtgatttcaaaccacaattacattttatctcaactaccagcattttatttagccaggatctacatgagacacatatcatgatgtgctatgtacatcttgttatacagtgttatattgataacaaatgtcatcaatataacattgaattaatcttccataatttatggggaaaaaaggagcagccttactgaagggcaaagttatacaacagctttacagaagctgcatgcgagtgcagtaccgggacacgggcacggacggcggcactataaccattttccgtggtggtaatcttgctttcatctgacacagaaaagagaccgccgttttgaaaactcacagaactagcctcacggttttgtgagtccattgagcgctggctgcgaagaacggtgtttaactcgagaaatcattgaacaagttttagaaaataaagatgcttatgacaatttcaaacttgaaggtctccaaagaaggactgagatattggtgagaggagtaaagagaatcctggtgcatttatttcatgcttccttctgttcgaagatcattttgaggtttataaaaggtggggtgatccaaaaatctccaggctgagagtcctggctgaggctgtgaactgggctgcagagaaagggccacgcctccctcctctgctcgcattactcagcagcttttctgcatgtggctggctgcagacaatctaaacccttccgctgtcgctccccccttatactgttctgccaaaaggaaggcagagaggaaatcagctacggggc

SEQ ID NO: 36 - DNA, 261 bp, Mus musculus
  source 1..261: mol_type = genomic DNA
tctgacagagcaagtcttgacctgcttaacattatgttatgctagtcattttaaaatgagtctttatttcccatagaaggtcagtttttttacattattatataatcttttgacagaataacaaataacattctgaatgtctcatttctaaatacaaaacatcttagtataaaattatgcattgttttaaatgcttggaagtaggtccacatgtagaaaacaaagtacgtatgataaaaaatatcaaaattgtatattcag

SEQ ID NO: 37 - DNA, 663 bp, Mus musculus
  source 1..663: mol_type = genomic DNA
aatgttttgatatttaggagaaaattgcaaaacaaaatgatgacagtgtttgaaagtgtttgatcagtgccaagcatcactttatgtacttggcaaacatgacttgaggccttaagctgtgatttgcaaatgtagattggaatcaagatctttatagatgaggaagcaaaaatcagaagacaaaataacattatcaacttgatctcatgtgcagccagggctgaactgcaaatgctgatttgccccagtctgggctcctcaaatcgttccttggaatcctattagttggaactttatctctgctcgtggcagggtgcctgggaccatgtttataaatatctgctgaatgaagaataagtgagtcaatcgaaccagaactcactttggttagttaatttcattcgtggtatttatggagagcagaagaaagaattccagagacacgatttgtcaaaactctctaaagaaaatgatgacactatatattgatgaaaatgaatgttcttgttcttgctttatttgattttcttgtccccccactccccatctgctagggtctcattacagcatagttcttgaatatcccaggttgacctgaagttacaatatattcttgatttagatggcagacattgggaatattttgactcttaaaatttaata

SEQ ID NO: 38 - DNA, 606 bp, Mus musculus
  source 1..606: mol_type = genomic DNA
ttgtcactttgttactctacagtgttgcctggagttcgatacttcattactctatagtggggtgaagaagttcaccttcttattttcatttccttccctcaatgatttcttcagagctagctcttaccagctagaaattcttcaaacgacactcgtgccttccttcacacaggttgaactatttgtctctaatgccctaaagtactggtgttcaatcttccaggcacttccaatgatctgaaatctgacctgcttaggtcagctggctctgagattatggtattctagtcctcaaaccaacctgttggctcgttggttttgtaccaaacacactgacttacatagctcaaaataccactggccttttaaaaatggcatatcacattccaggggaggatcaaaactgctggctggtgatatttgtcaagtctctcaaagttgcactttccaggattttcaattcactgaattcttagacagacatgtttatgtgaaagaattctttatatattttttctcctctttgagtgggcaaatgaaaatcttgacctctgggttccttattttatttgactctctgtagtatttaaatcttaaaattttcct

SEQ ID NO: 39 - DNA, 2430 bp, Mus musculus
  source 1..2430: mol_type = genomic DNA
gatactgtataattaattaggcctccaatcatgccttcccagcctccacggatggagaaaccctctccgccatgccttaaagaggaattgctgtaataaatgagtctcctgatagcaaatttctcagcaagggggaatcgcgtaaatggagacatagtattgacagcaaagtccaatgtgttatttttaccagaacgaactctccggttcaagcctttgaaagagacatttgaaaaccaaaaacaaacaatgtaatggagcgaggaaaaaagccacagaagtgagtggcagggagtttaaaagagcagatgccactgccaggtctatgggacataaccagccacttgtgctgggtcttggcagtttataatgctacctcatcttctccgcgaaattgttttcccgtaaatctctgtggccatccattcctgtctacacattatgttcctaaaatagacaccatctaaaaatcacttcaaggagctttgtggaggaaggcctaaattgcaacactcctccagcgaagatagatgcagtgtttgatggcattaccagtcggtagccaggaaggggagtttgtgaggagtttttccaccacagttaatctgtttctggaaggaaagggaagtgtcagacttcccgaggaggcaaacgtgtgtggaagctctcatttgcatcacccccggcctgtcaggtattgcagcaaaagggagaggtgagctaccctggctctccttgggcaggagggacagaatcaggaagcatcaacctcagcatggaattttcctattcctgtttggcatcctcctcttgggatgatttacagcgcgggttggagaaacacgctctgccactccactagcgcaccagatagacagtgcagacctgcagatccatacccgaggagaagccacatttcctacgtgtgatagcaacagcgtttggcaatttgcgactttgctactgcagcttagaaaatatttagtcacatgcacatctgaacagaaagacacccaggcttgactcagtcatttccgtcagacacacgaaagaaaaagcgtctctgctcacaagcttatttggactgctttgttgaaaggaggggcggcagacactttgtagatgtggcaagagggctttatatccagacctcaaacaggtaggagagaaggaagccaggagaggtaaggaaggggcgtggaaaagcctcacagccacctcgaagaaaacagtttttttgccctgttcagaaagcaagaggttccacagtggttttgtgtcaatggagcacatctgcagtatcattgccgttggtgacctctgtctaattaaaagtaagtcagtccttcccacccggcattgtctgaaacccgggactctttatcactttgctaaagttcatttgcaagtgtagttaaggaagagtcaggggggaaacagcatctgtcccttctggtcctggggaggaggcactcctttccaagagtcaagcctctgcccaaagaagctgcctcccctgcaatgctaggatccaggagcagccccgctgccttcttgcttcctctgtgaggtctaatttttgcatcatctttaggagcgatatgacctctattcacagccatcgaatccagttccaaagcaccaatgacagagggggcttcaagacaagaccttgcctaggaggatgcaggcaagcaaaggcaagagctggcccgatgccaagttattttaggccaaagaatctcatccttctatcaaaatgctgaactgcaaaacgaacctgatttcagttcatggaaggttgagaggaggaggagggggaggggaggaggaggaagagaggaggaggggaggaggagggaggaggaggaggggaggaggagggggacagttggtccgaattcacatgcaaaaatagacttcctgttctgccccaactcttatttccgtgggctcttctccccaaggatttaccaggtaagaattcaccaccaaagaagatcacaatgagataatcagatggcttacctgataaaaaggaaaattatccatctgcagtgaggagcaacatctccccacgacgagtccgcaccttccgttgcaacgattcagattccttcttgcaaaaggtgaccaagtgcttcacaagggctgcagcctcataggggcagaacacacgtacacaaacacacgcacacacacacacacatgcaccagagacctctgcagtatcctctcggcttcatcctcgcctcactctatggtacctaatacaaatcagcaaatagcttgttttaaaaaaaaaagaaagaaaaaaaagcggagacagcacctaacgttacagtgccatctagtggctacatcgtaaataggttctcacagcctggatttctgtgttctttctcaaccgcttccttctggttccttttt

SEQ ID NO: 40 - DNA, 1644 bp, Mus musculus
  source 1..1644: mol_type = genomic DNA
attgatctccaactttttaaatccctctgtcattttaaatgaggtgcactcggttgtgtcatcatctcggttttaattgtgtgaaaatttcctgccaatctcacaccgctgcggcaacctcaccttgctacttgccctgcaagttctgcagtgtgccgttctgagtatgccgttttaactagttcttgcagcaggacacaaagcgagatagtctgatagaaccagttctcctctggttttacctttactcttagatgagttaagggtcacatcaaaccagggctcagcccgccagatctcctaagcacagcccctcctgacccaatgcagttaacccaacctcattcagcgctagtatcaaatgacactggagctgctgcagtatgcatcccgagactaagtaggcaggatttattatcagcagaagtcccctaactaccaggttattcaagctccgttcttgtcacaaacaggcgcggcggaagacacagtgcagcagactcagagctcatttacaagacaagcgaattctcagttagagacaagggcagcgcggcagcgaactgcagtaaatcttttcacgctcacagcaacatctaacaatgctctcctgcaacgcctcagatcaaacgaatcctacttggtttaaacatcaaatcaacaccataaaaaaggcttcattagcaaagttcaatttaggatgtttttaatcgtgtcttaattctagaaccagtgcgagactttccatgcttattcaagcatgctgacagaattggaacctcttagaattgcctacctgcacctatcagcctggctgacaggagcccgccaaaggattaaaaaaaaacaaaacccaaaacataaaatcatgcaaaaaaatatttacccccgaaagatgtatgtagttaaagctcagcttcctgcagcctcgatagcccctgaagtgttaatctgaagaaacagttccatgagtttccacaggccggtagtgagtctcctacacttgacctagacagacttacataatgaagcatcagtgctggggagcttgcacgatgtcatcaccagcaagagtaagaagtattggcagcagcaagcaggcgggcaggctgagatcttgcatggaaatcatgaaccaggtcttgcttttcgtttttgaaacgttttggaaggagagttatgaatagcccagaaataggtctcattttgtgggtaggaagaatgaccagaagcatgaaagctaaatctcctggcaagtgcaggggacctctcttggagtgtgcagtaaacccgaggggacgacttctcctgctgtcaactcctgaaccatcacatctggagtgaaggaaggggctggtgaagccttgtaataaatgcaaaggatgctgctgagagctttggtctgcctttaactcattgtggtgagtagaggggatgtggcagtatgcaatgagagttggttgtgtaggttgctttgcagagtaataaccaaaaaaaaaaaaatctgtgaagtgctcaatactttagacacattttaataaacaagatgatagtaaaattactcttctccatcaaattgagactgtgctgggttaaactgttttaatgcattttaactcctgatgttcatccaagtaataagag

SEQ ID NO: 41 - DNA, 521 bp, Mus musculus
  source 1..521: mol_type = genomic DNA
atctcaagtgtatgtaacatgagctacagtcttaaaacctacaaacagtacatccagtctcctaccatgattctgagtgtgatgatttcatatgagcacaagatgacatcatactatttagttatatgtaaaatcatggtcttacatgggttgtggacaaaaccatctagttttggaggtgacagaaatagagaggacgccatgcactacttaaaaataatcgcagccttcttttcttagctagggagtttgctgctatgagccacattaagaccagggtgaggagatgagacgatacaggggcatgaaagaacacggtgatctactttctcctgttaattaacgagtaaggaaatagacattaaaagaagttaaatgtgtctgagccaacgtaggtgaggtttcccccaaattcacctggtagttttgctactgcagtatagtaaatacttgttttcatttgtttttttttttttgttttttttgtttttttgtttttttgtctttttgttttttttttt

SEQ ID NO: 42 - DNA, 547 bp, Mus musculus
  source 1..547: mol_type = genomic DNA
tattgcaaaaggaaggaatgagacagtttatgcagagctaagggtttgtgcgttattatgattaatcacaaggacagctgccaagcttccatcatgacaatattctctgggagaattcatcaggttctactgtctattaatttctgttgatgtatcttatctggcatcttcaatgacagaggacacttgttagtttttttttttaagtgaaggttaaaagacaaagttcattaaagaaatgatttatatatgacatttaagaactagcaatgtcattgcttcaagaaaattatgagaatttagtcttggtaggagtttacaccatgtccttgaagtgtctaattatgtgacttgatagttttacttagtacatatcgattaggctgtatctattatttatcaagaaattatggaaggaggcaatgtggcataggcatacacattctgattttaaaataatcctgcttttaccattaactccttctcagataattctgaatacatatcttgtctatgaatctgtgtaatcatggaaaaagaaaaaatc

SEQ ID NO: 43 - DNA, 504 bp, Mus musculus
  source 1..504: mol_type = genomic DNA
actgtttgagcgggcagagcaagtgcgaaaagattaaaaagtgttctcctcatcctttctgctcatatgaccagcgctgcagtgtcgcgcgccaggcgcacgcccgctcggcctggctccgcgccagggggcgcccggctcagcgcacagctcagcagctcagtcccgccgcaccgccaagctgagcgggccacatgcccatggggtccgggcattcaggaggctcgcccaggaccccgccgccaggggccatctcctgatctaaagggccaccagcagagactggagcgttctgccgctgtcaagggcatacgggaggcagagaggctccgatctgggcatctcggacaatgtcctctcgcgtggggaaaaaatgatgctgaccagcctccaccccgatgtggcccctgggctatgcactctgtctctccacgcctccagtaccctcttgtcttctgtcctggatgcaaatttatgccgcgaaaaatctgaatgttaagaact

SEQ ID NO: 44 - DNA, 202 bp, Mus musculus
  source 1..202: mol_type = genomic DNA
acgcctccagtaccctcttgtcttctgtcctggatgcaaatttatgccgcgaaaaatctgaatgttaagaacttgaaacctgagccacaggacccagggaggaggttgttggactatcagagctcctctgtgcagaggctctctgtctgggctccatccctccctcacctgaccacattctgaaacttgatttggtttctgc

SEQ ID NO: 45 - DNA, 533 bp, Mus musculus
  source 1..533: mol_type = genomic DNA
tctctctggtcatgtgttgtgttaaagatgggaggatcactgtgtgggacctgaggcaggtgggcagcgtctggggtgatgatgatactggggaccaagggacagcttcccagctgcccagtagctcaagttgcctgacaacttgctgtcaacactgtctcccctagctgagccctggaaggctgcccttcctggaaaccttaggagtgcctggctccagctgccccctcctggtgagtcagcgtctttgggccacagcttgccagctggcttccaaggctcctaggctacagcccctgacttcatagctctgggcatgaggcccacttcagaccccccagctgtcctgcacccttcagctgctttagctcctatgtctcctgcctcagttgacttagtggcctcagcctctaccccttgttcccagctggactgccagttccagtgtgccaggcttctgtgttgacagttcaatgtctgggctctctttatactgtggccccagttacagcatctgtttctccaactggaga

SEQ ID NO: 46 - DNA, 496 bp, Mus musculus
  source 1..496: mol_type = genomic DNA
tcggttctctctgtagtctgcaaagaacccacttcttgcccagtgagagattcgggagatttccactgcactgcctgaggccactgtgctttaatgagaagaacacccgcttcctctcaaggccagcagcggtctgaatccccagcccccaaacacactgcagcttgcccagctagctgctcctgtgaggggtgaccttgacttgaagttagccccaacttgctggacaactccaccactttctgagccccatggtatatgtgagcattgttgggaggtgagaggtagtgttgtaatatttaacttattggactcgttagagaaattaaaatcactgtatgctgcaactagcttatagaagggatccaaagactcttagatttttctttttccgggaaacaaatttattaaagagtgttgtagaggccaggccgtagcgaattcctgcttgagatgtccttggtctatgttgcgttttgaagacacccctccccgc

SEQ ID NO: 47 - DNA, 318 bp, Mus musculus
  source 1..318: mol_type = genomic DNA
tggccccgggggtggggtttccctgtgcgctcgcccccacccctcgtgtgtgccctcccctcccccgcccgcccctatgtatgtgtcaccgcgcaccattcccgcccacctacctccccgcggtgccagaggggctcacagagctgaggacgcgtgcggaacggctcaaggtccctcactctctctccgcatcctcgccggctagcatctgacgcaggtgccgagggagttcgggcacctttctgtgtcccttcccttcatcgaccctgctccgaaacctagtcagccagccaccactgcggccaccgcccgagggga

SEQ ID NO: 48 - DNA, 502 bp, Mus musculus
  source 1..502: mol_type = genomic DNA
agtcacagaattggactcctgtgtcccaggaatataagagcacaattgatgtagaggttcctttaaggatgacgccctccttcctggagactaactgcctgttagaaaggtctggagaaaggtgggggcaagctggagagagagcgcaggcaagaagagaatcggaccagagaggaaatttgtagggaaactggtaaaacggtttcttttttcaaaaagttgaatccagggcaagaacggaaatggttgagtttactttgaaaaactcagagcctccttgtatgcagctgtgtgtgtgagtgtgtgtgtgtgtgtgtgtgtgtgtgtgtgtgtgtgtgtgtgtgtgtgtgtgttggaggttgggccctgtgggctggctagagtgtgtgaaggcagctctgctccctcaatggccttacagtgtatcctaaagaagctttttgttaaactcctgaataggtggattggggggtgtgaggctggacatcacgaaatagctatt

SEQ ID NO: 49 - DNA, 474 bp, Mus musculus
  source 1..474: mol_type = genomic DNA
acggcctcgctttccttcccattgagaaaggtctctctgtccacgagtgagcgtgggctggcaaagtcagtatcctcacataaaagcctcactgagtctctgagaggcttggaacccagacagaatggaattgctccctatttttctttacaactgagaaaacacacacaaacacaagagcatatttattgcgataattctatcccaaagtttttaagaagaagaaataaaattagtcttgctgcctactccgcaccagccccctccctttcattccttcacatttcctggtctaagccagggagaaaaacagagccagcccaaaccccacagagcaccattgcatcccagacaatgtccagcttgttcagagagcgagggaagaaacacaactccgaaacatacacaagacttccaagtgaagggtgttctgagggaggaacaggctagctttgtgtctgggtcttgggtaaa

SEQ ID NO: 50 - DNA, 674 bp, Mus musculus
  source 1..674: mol_type = genomic DNA
aagtggggagggcttctaaggttggttctggaagcccgtctgccaaatgtgttttcactttgtatggctgatgaaagctacagttcgtccccggtgtcttcctaacccagcctggtgtttactctgtaagatgtcgcgtgcgtgcgtgcgtgtgcaagcgggtgtgtgttcgcacctatgtgtagagacaggagatcaatcctcgctgtcttccgcagtccgtctccacggagggttttttgtttttttctgagacagagcttctcatctggcggggaagttccctcattaagttccttccagccctgccctgtgcattactgcctcctaagcgctgggaagccagcctgcttcacctgcctcgcttttctgttttcctgagtctgggattcaggtcctcctgcatacacgcgcatgcgtgagtctacccctgagccacctcccggctcctccacagcatgtttgacagaaacaaacaccttctcacccttgtttaaatttccactgtcttaaaaacagctatgccaccagggactttgactatacaagggtctctgcgcatgtttctggaaccttggaaatctgtccagtcatcggccaccaaccaagtgtgtgtttttgacggttagttctgaagctttgcaacacatcaggggttacagaaagcacagttg

SEQ ID NO: 51 - DNA, 339 bp, Mus musculus
  source 1..339: mol_type = genomic DNA
ccgctgttttacatactaattgcatttctgtaaaggaaaaaaaaatcattgagataaatacatagctgatagcatgtttaaaggtccttctgtatttcctcccccattatctgcaatggcacacttgggcaaacactggcctggggcccaggtacagatggagcaggcagatctagtgtcagctgtttcctccccgcgactggacctccagattgcatcctctgtcagaactttgaaggaaggctttcagtaaagatggtattctaatttcctttctgatgctgtgataaaaggcatctcaccaaaagcaacttagggaaggaaagagtttatttcagc

SEQ ID NO: 52 - DNA, 502 bp, Mus musculus
  source 1..502: mol_type = genomic DNA
tcggtggtgtcctggccccacacagggaaaatgaaacaggagtctgacgactgtccattgagagcagctaagcttgctgtgcctggactgattggacacatggaagacagtgttgggtcgtccaccaaagattggcctggagccaccgtacacagagccgagacacattacccccccacaccccgctacccccctggcctggaaggagactgtcaggcgtgacaaaacaggaatctgagcctaagaatattcacagattgcaggcccagaaggaagtaggaagttcatattctctttatagcctcgctcgctgggggaccggcatgtcttttattgttaatataaaaatcaatatagcaggaggcgagtctttgccataatgatgttggctccttgcagcaggcgcgtggaattagattgctcataggtgtgcctctgtcgcctccccgggctccagagagaagctgtgggctttgccaataagcacagtcacagagtatgc

SEQ ID NO: 53 - DNA, 341 bp, Mus musculus
  source 1..341: mol_type = genomic DNA
ccggcactgcctccggtgctccgaaagaatgtaaagggaaggttgtacccatcaagcccctcttgctctaccctgtgccccccatgctgtcagccaagtctggcccccaaggatacccctccttccctggggtgtcctggagcttggaattggcttcattttctccagaaggaggaatcagcttttcccagccgtctttgagattatctttattcttcaaggagctccagtggccagagcctctgtgcatcctgccaagtgccctgggatcttggaaatctgcccaggggctcagtggaggcctccactcctccagacagcacacagttatgaaagaaaac

SEQ ID NO: 54 - DNA, 443 bp, Mus musculus
  source 1..443: mol_type = genomic DNA
tcggggtttaaccgttcagagtaattttccattataattttgtgttctaaataaatagaaccagcttcactctagaaacgcttgtctgagtgctgtacaatgcatgtttaagtggctctggtgaaattactaatcatacccctgcagtggataaaatagaattgtttgtgagttttgctcattcgatgggaggacgctaaatttataaaagtagactcatgcccttgatagtgaatttaaataggtgcatatgtaaacagatacttactcatttagagacaccgtcatcagggagctttgtgtgagaatgttttctatgctccggctcctgctctgtggtaggggaactaagcagacatcccaggtccatttcctaacccagtgtctctagttgagtaatcacacaaggcagacatcccagctgggactctatatttaaaccc

SEQ ID NO: 55 - DNA, 545 bp, Mus musculus
  source 1..545: mol_type = genomic DNA
ctcttcccactggtctgggggcactcctcttcctgagggctatggttctcagaccacataaaattcaaatctcctggaacagcgcagctccctcctccccccacagcgagctccggatggtgtctgagtttcatctttacaaggggcctttaaagccgcctgggcccccttcaaaatgcccagccccctttgtccagatggggactggggtggccaggctgcccggctgattgtgtgccgagcagtcctcccagggtaggctgtgatttatacgcacgggcttgtcaccgggtgaaaggaagggccactttttcattttgatcaaatgctaggtttgaaagccacccactgccgtaaactcagctggatccgcggggcccgagatgaaacacattgcctgctttgttgctgagatggtgtttcggaaggctgtgtgaatgctcttcccttggctcagctctcacacacacaagaagtgtagtgcagagactgactccctggctaggacctcagtcagggggagagattggagaaggtgctgttca

SEQ ID NO: 56 - DNA, 557 bp, Mus musculus
  source 1..557: mol_type = genomic DNA
acgctgctgggctcagaggtggccaggagcacattggccttagggagttgtgaggagaggacttcagagctctgagttgggtttgtgctgtttttttctggcttcctgtttggaggaggctgtctgcacagcgttggctccgcagctgttaattggcaggaagcgataatcacgcaagaatggctgaaatcatcagttagggatcttgtttttttggaagtaagtgaaatcctgagatagcctgccctaattatttcctggcttaaagaaaccaggtgcattgaagacatttttttttcctcttctgatttatggaggtcttagggtctgagttttgaaagtgctgagggaatgaagttctgggtgtcgtcctaggaatacaaaacttttctatatatctcacgataaaaatctcttcgattagcttatctaaattctgacaagttaaaatgtgtcctagatttttttttgttgtttctgtaggtaccagtttaaatatttcattacgtctattggggggggggtgtatgtgtgttctcatgcatgcacacatgtgc

SEQ ID NO: 57 - DNA, 316 bp, Mus musculus
  source 1..316: mol_type = genomic DNA
tcggtattttgagaagtacagtgagtttcttaaacactcagctctgacacttccaaggactcttcctggaatgttgtaccttggcaaagcttccgagtgtgtctgaagctcgaccgggactgagtgggttaaactggagatgttctgccgtccctaatgagaaaggaatgaaggtgcccttgtcactggtgaccccggcgcagggcctctgggctagaaacagagtgttatttctttgaagagacatcttgacaacccaagttcatctggacgcagaaccaaaggcagctttgatctgcttgccagtgtccctgcc

SEQ ID NO: 58 - DNA, 858 bp, Mus musculus
  source 1..858: mol_type = genomic DNA
tcggaatagctggctccaccaatccccagcagtgccaggggatggggcagggtcaatgggggccagtaactgacagctgttccgtgggttattttgggccaggggctgagcttctcggttccagggtgttaggattaaaatagccacctgcagggcgatttgcctgtagcagtgtaggggcttttccaaaaagcaaacattcaaagcccggcagaggagtagaatacggggttggtttgaggaatggcagggaaggaattctatcactgtctaagaccaacctcacctctgaggagctgggctctaagcgttggcatctctctgaccagatcactttcctgggccatcaaaatgaagggttgatggggaaagaagggttggtaactcctaaacacagcttctggccttcttcagtcctcaaagggagcggtgagatgtccccacccttgccttttcacagatggttccacaggagttcagggggcgcttggaggctctggttgcaggtcaccaaactgccccagtacctcttgctttggccttgctcagagctacagaagattcgtgttgggaggataatcagttctatctccaagctcaggaccagcatggaaaacgctgactggccctggctatccttccagtcacagcgggaccttggcagccacccgcctagctcacaccctcctcctgcaggaggtggggcaaaagcaggtgggaggagggggtcgaaggggaagcccaaccatcctggagcctagccaaagctcaacttcgcagccccaggcggggacttgacagcctgcgggctttgttcaggacttcgagttagagtgacctgggaccctttggcagaga

SEQ ID NO: 59 - DNA, 955 bp, Mus musculus
  source 1..955: mol_type = genomic DNA
ccggaggtatttctaccagacagatttgtctaaggagatttgctaggtccatttaggagaggacctagaaaggtctaaactattgcgcaaactgtatttataagtttttccacctgccttccgttcgtttattgctgttgtctcagtgtgtacgcaggatttgccttgtaagcagaattaggagagcagaagcctatctttaacaacagaagtgcttaggagagcttgtggccaatcgcattgagcagctagctctcttgaggccagagcctgagaaaggccccctgggaggtctgcgcccagcctgttccttggcgaggggccgggccaggcttgagagattaatctcaatctaccatctgctgtgtcattgtctatttctaggatggcagtttgtcaccctcgccactcaggtgtcagctgtgggaagaagtcatcattcaggagatggatcctgggggtgtgcacgggaggaggaccccagccactcacgggagtgaggcacccatcccaggagagaagctgggccagaagattgggctggctggtgtgacgaatggatagaaggaagggccactacagggtggaaaccagaaggacacttaagctgcccaccggggcgtcaccccaaggcgaggtgatcttcacagacaccttttctatagccctccaggatgcagaagcctccaagctgggggtttggcaggaaatctgggcagccatagataatgtgtggttgcagggcaggaactgtgaggacagggacgaactcatctgtgatttccacttttcacaatccatcgcagggccactgagcacaggccagagaacccaaccctggctgagtagacaggaaggtatcaacggaaggatgccagctgatcctcagagctgaagaagcaggctcagaaaaggacagggctgcagctcagctgcaagagtgctccctcggcac

SEQ ID NO: 60 - DNA, 424 bp, Mus musculus
  source 1..424: mol_type = genomic DNA
cagtgatgtcatcagccatttcaacctgctgagaatttaaaggcacaggcacccacttcccatattgaaggtggcaagggctaagtggctacccgctaggaacacttcagctggctgctgatgctagaacatccaggggtaacataagcacccatcagcttggctctcctgcatgctgaaggagacccccacctgtctacctccagacagtaggcaggacccacccttgatccagggaggggcacaggcacactcacaggtaatacagggacaggtgggtcagaggtcagcagcctcctttccagggcctcagggacatctgccttgctctgtcacttttctaactttctgtttagctcaggaattgtcttggcccccacccttgccccatttttaagccatgggctagctcccccctccacct

SEQ ID NO: 61 - DNA, 628 bp, Mus musculus
  source 1..628: mol_type = genomic DNA
atagtggcgctatgtgggagatgtctgggtggcagagagaagcatcagggaagatgatggcaagggagcactccttctcggagggagcactccttctcggagggtcctgtcataggactggctcctcgctcccctttctgggacagatggatattaccttcattcctatgctgccactcaggaaatgggctggaggaggagtgacttttccaaggtcacacagctggggctgagctcagacatccctcctccctctcctctgctccccctcctcccaggcacccgatgtcagctgctctgaagtgagtggctatttttattctggtgcgagggacctgaaacagcagagccatgtaggttagaagactctaggacaccggcctcagcaggcaaggaaaagctcactgcaggcacaggaccctgctaagcttgcctgggggaactcttgttccttaaagactaccctgtcctgggcttctgggtctccaaacagttcttcattagaggtcagaatagagcacagaaccagaataagccgtgttcatagccctcatcctgtctccagctcgaacactaagcttgaaattgactaagacttgatcccatgccaaggcaaagcctctactct

SEQ ID NO: 62 - DNA, 766 bp, Mus musculus
  source 1..766: mol_type = genomic DNA
ccgttaacttcacttaagaaatcatgactgccaaagtggagtcttacaagattttgtcctagcatactccttcttagcacgctttctagagttggactgttaaatttgtgccagatacatcaatgaataccgcctgcctatctttttcttccaactgaatttacttatctaaaaaagaaatcgtctaaaaattcacctgctaataaatgtaaatgtccttaccttaaccagtcattaaggaataccagctaaatcagggtcacacatcaagggtctccacagacgtgagtgtccattttaaatggtacagtagcattgtgggttcccaatcgtttaagtgccatgaccatccagttttgacaccgagtctcttagagttacaagctttctaatttgggagcatgattaatccctcctatgtgataagttttaaccttctaatatttctttggattgaaaaaagcaaatgagctgcggcaaagaatggcaaataactacagtgcttaacacagtttaataacctgaaatgaagcaagtgtgtgctatgtttccattaaaaagtttccagccacaattaattgaacaaaaacttgtcttgttccaagattattcttggaaatgtaattttaaagcctgtgtgaaatgaggaaacttaactttttataccatatgaaagcaatttcattttttaggaatgattttggatagacttccgattggatattttccattggaactaacagtgtagaggcttggggtggggaga

SEQ ID NO: 63 - DNA, 223 bp, Mus musculus
  source 1..223: mol_type = genomic DNA
tcgtgaaatccttaaaagccacatgaggcaggctttgaagctccaacttctcattttaaagtaattccatagaagaataaaactgaacctaactcaaagtctctggtgtgaggaggtagacccagatttgggttgtctctcaggcccatattcagttttctttgatttcagtaatatggacaatcaaactgacagatgaaacgtatcacgtgaaaacttacac

SEQ ID NO: 64 - DNA, 288 bp, Mus musculus
  source 1..288: mol_type = genomic DNA
cagagtactgcccttagatccttcttgaacagaatgctacattaggcattccatgagttcccccttcccaggaggcctgtgcccagtctagttgccatagatggccatcagacatttgcaatgacatcaggagactatctgagcagccactgccctaaagaatctgctagcaggagactggggatatcagtgtcttcttggccgacaactgcattccaaaggtcaactggggtgaaaaggatgggcttggagcatctggtgcgttttcagccacaaggcagctaacaa

SEQ ID NO: 65 - DNA, 912 bp, Mus musculus
  source 1..912: mol_type = genomic DNA
tcacgtggcacacgcagcgcccccccttctaccctcaacacacacacacacacacacacacacacacacacacacacacacacacacactagcagcctgggagctctagaaactagccggggaaagagtccccaccccctcagctccgtctgccggagaaaagtgggtgggaggaagttgcgacccacggagaagagcagataggaggggagagccatgtcggaggggaggaccagcagtaattgggtgacgttgcagccgttgtgcctctccagtccgcccagccagcgggtcagctttcaacacaaatcagacagagggattgcttctgcctgcaatcaataactcagtcgagtgagctgctgaaaccagaggcgtcccttgtctgctctttatggggccctgcggactgaatcccagcccctctgcttcacaaacaaagaagtgggtaccaaatgccagaggaaaattttaaccccaaacttccatcgcatcccctggttacgatctgttaaaatgaaaagagcacggacctagtaaggtctctagaagaaagggttaaagggagaaatctacgccttaaatttcaggtctcctgtgatatgaaatggagtagaaccagtgtttcaataagcatttaacaatgggaagagaagatgtaaagtcccattaaatttagctgtccccatagatgctttcgagtcagccgcacacttataagcaaattaccaatcaagtcttatttgcacttactgatagaagggaagaagaagaaaaaatgtatcatactttgtagaggagaatttggaaggggggtgttagataatttattacagactttatttcgtgaagagtaaggttaagaggtcctaggagatatatcgtgatcccaacttaataatatacattaaa

SEQ ID NO: 66 - DNA, 190 bp, Mus musculus
  source 1..190: mol_type = genomic DNA
cctgcaagagtggaacccgagggtcagtcccggctctccatctcttcctcgcactgccccgctccctcttttggaaccccgtggcttcccaacagccactccaaagtcgagggcatctcacaagttaggaggaaaaaagaaaacaagaaaaagaaagcaaaaaagtgtctccgagcccctgagcacttac

SEQ ID NO: 67 - DNA, 517 bp, Mus musculus
  source 1..517: mol_type = genomic DNA
cacttttccacatccatttaaaccttggaaatgctctgctttctccaaacatgacctgaaaacccagctaggagatgcttttcagagcccaagatgatttgagatacaaaggtcttaggaaaatgggttctctaggtcccacacttgtgtaccctcattcccatggctagcaggaaatatctgaggagaaggaggaagggagggagggaaggagacagggtgggagggaaggacaaagggaggggaacagagaacatcatttgggctgctgctcactagaagccttttgctgagtcaagttcttggctcccttgagcagacagctctggacttgcatgtgaccacaaataggattcaaagacaccccctgttcaaaggacagtcacaccgtcatcacatctcttgccctgctgcagaaagttctagctgtggtccagttccatatgagctgcctagaaggtggcatccccgggtgagactgggaccttggagcatttacttctttatcccattgtcc

SEQ ID NO: 68 - DNA, 1767 bp, Homo sapiens
  source 1..1767: mol_type = genomic DNA
tctaatggacatacagtaacccttcataaatatttgctgaacgagtgattcagtgaacaaatgaatagagaagaccaacatccgaaaagttattttattttcaagcctcatgtctttaactgttttatatcagcctttcttaagttgaccgtcattaatatttgctgaatgaatgagtcagtgataaacagagaagaccatcaccctaaaataacgacccctccacttttaagtcttacgtctttaatgggtttcatataatctttctgcgctctttttactgtccagtgtgggagctgacactagtttgccttaagtccttaaaaatcgcacccggaggcgcagtgtcataggtaacccaagctttcctagtaaacatgatacaaaagtaaacacaaccaacagcatggggaccagcaattcagaaacaccgagcgggcgggctgcccagacctgggcttccccagcagggcccgcggagaccggccgtgagcagaggctgcaggcccaccccgcaacccgagcagccggggcaccgcagggaaacagcggcctagcgaagccacccgagctccctccgcgcccccgggccaaaaggccgcaaaggaactccgcccgcccgcccgctcacccgctcacccgctcacccgctcaccttcaattcctgcgagtccatggctgccccgaggccgggccgcggggctctggggattgtctcgccgcagcctaaaggaagacgcagaattcagctcccctagcctcccggagcgctctagcgccccgggccccagcgggaggggcggggtcgcgccgcgattggctgtcggagggagaggcgggcctgtgtggcggggatcgtgctgtaatggagcaggggcggcggggacccggaggtgagggctgcgagggccgcccgggagggtccgggctgggaaaagggcctccgccggagagtgcagctggaaaaggaggtcacactgggaaacggctgtctgaggacagtgggtgggcgggccgaggaaatggaattcaggaataaaggaaacggagtatgaagaaggggaagtctgtttcctgtcactggttgtaaaggaagacaccattttctgcacgtttgtctggaggcggattcccgcagtgcggctctcagcaaggctctgccggcgcgggaaaaagcggtcaactttcacgtgggcaagttgttttacggccacaaggtggcgcagaaaaaaaaaatcacacgttcttaacagaaatacggtgcgcttgggcccgtctttgcaggcgttgctgcaatctttgttagaatgtgtgttcaattagcccttttttaccagccccgataataagagggacaaataaattaaacttccagaaaattagtgtcttgttttcaatgatactactgattttaaactgagaataaaatgaatcccaatgcaaatttttatgtttgcaccccattaggcaactcaatcagtcacacatagatttcttaagtccaggaaattaaatggaaatataatagactaagattttctatttctgcttaaataaatatttaaaatagtgcataaggtctgagatttaagtgatctttgcagaatctttcacgtggattccaaattttgatcctagtgttaattatcttactttagttgacatgatacgtagttgccttttccagattttaagtttcttaaggagtttataaacattgactttttccccatgccaataggttatgtaaggacagtcttgagaaaa

SEQ ID NO: 69 - DNA, 850 bp, Homo sapiens
  source 1..850: mol_type = genomic DNA
agtgtgggcctcccagggctgtttagctagcaatgagagaggcactgcctatatccaagttgtatatggcaggttttgcacaaagtggattactcgaagagaaagcctaatggccagtctattcatcttcccctttctcgatgttcatcttttctctcccagctctcctttattctcaattttctttttttttttttttttgctcagcctccatctcacttccgttgctgtcctctccccaccccttcccactctggactgtgcctctcctttgtagacacttcaagtccattctatttcattcaaaaaccatggtctagaagtaacttaatgtaaacccacaaagatggagacagaatgaatgccattcttcttgctgctctctcagacaatgcaggtcatttttgcctatggtgctggtaaagccaggagttatgtagctataagtagcagccagaggaaatagtgcctgagtcagcaattgtctttttattgctgtggggcaataatgggagaaaaaatcaggcttggtacaattccctttgaaggaaaaagatgccaacactagcattttaacacaaaatgctggttgggggttgggaggaaggatgcttacattccttctttggaaatatctactttgataaccattttggtaaaataatgcagtgttttcagtgtgcaaatcctttcaggactcatggttgtatggcagacgcacctgacagcaataatttaagggtaccctgagaatgactctgtggtctaaaaagaatgtgtgtttggaagtctgaggtaagaaatctggctggaagtggccaacctggaaatttgctccttattattaagg

SEQ ID NO: 70 - DNA, 845 bp, Homo sapiens
  source 1..845: mol_type = genomic DNA
atagtgcaaagtttaaatttcattttcctaagatttgttttaaaataacacgatttacccaagtgatttcaaaccacaattacattctgtttaaattactaatattttattgcatcacaatctgcatgaaacagatgtcaggatataatgaactaacctgcattgtatttttatttttgtctcctgtggcataacgatttcataggaaagagaactacacagctgactgactgatggggaaagttacacaatggatagctttgcagcaacatactaatgcggtagggagatgctgcagagaggctagaaataaaatcatttctttccggagcagcactgcttgctgtcggctgagacaaaaaagagatttcctttttttcctttcttttttttgaaaactcacataacattaattctgttaagcactggatacacggaaaggtgtttaccttagaaaatcatttagcaatttttagaaactagacatatagcaattttaaatctttttaactatctaatgaccaaagcagagggtcctcacaagagggatttagatgctactgaattgaataaagaaaatatggatacatttattgtatgccttattcagtttgaggttcattttgagtttagaaatagggatataaaaacatcaggggttaaatagcatgggtaaaggacatgaaccaagctgcagagaagaggctgactgcctgctatatttgcaggcattactcagcacttttcttaaaccgatacatcttgctggctgcataagcaagacaagacccttttccctatggctcaggaaggcagagaagtcaacttcagccttgaaaaaggca

SEQ ID NO: 71 - DNA, 268 bp, Homo sapiens
  source 1..268: mol_type = genomic DNA
tgccagacagaacaagttttagtgtagttgatagtaagttgtgcccagaatattaaattgagtcaaatttattttccacataaagtcacagttttatatgtcattatataatctcttggcagaaataaggaataacattctgaatgttgcactccaaaattcaaagaatcttagtataaaaatatctagcattttagatgtttcaaagtagggccaaatgcagaaaataagttggatatgataaaaataccagaaagttctattcagt

SEQ ID NO: 72 - DNA, 895 bp, Homo sapiens
  source 1..895: mol_type = genomic DNA
catgtaaaattaatatgatcttttagtcacttagaaaaaataccataaagaacactaatagtgtttaaaagcatctacccagtgccaagaactgcattatgtattggtgaacataactttagactttaccatacaacgtgaaaatatatattattatcactattttacagatgaagcaataaaagtcagaaaaaatgtagctaattaaagtgatactgtgtatagctagagcagtgtatagctagagctgatttgtctgactctagccctagtttctttccattatatcaatttcctggaaatgtatctctgttcatggcatagtgcctgacactatgcttattaatatcttttgaataaaagaaccactgagtgatttgaaataaaactaaatttagttagttaattttattggtggtatatagagatagtaggaaaaataattgaaaagagacataaacagatttgccaatactttctaagaaaaattatggaactagagtttagtcaaaatgaatgctttcattgttagaattcaactttaatctttgcagaatacaaacaaagacccattttctagaagaagtaacagggaagagagagtaagaaagagataatgatgaacattgtctaatgttacagcataatctagtaaggtaagaacagaagagagttcattgacttaccaacatagttgtccctaatcacctctgtgaacctagagtgctacgatataataatgattgtggtggtttaaaaagtaaatggggctgggcatggtggctcacatctgtaatcccatcactttggaaggctgaggcaggtgtattgcttgagctcacaagctcgacaccagcctgggcaacatggcaaaaccccgtctctacaaaaaata

SEQ ID NO: 73 - DNA, 666 bp, Homo sapiens
  source 1..666: mol_type = genomic DNA
tccactttttgctattccacagagatttcaggaagaaaaatcacactcctattttctttttctttgcttactgatttctatttagtttcttttttttttttttttttttttttttttgagaaagcgtctcactctcttgcgcaggctggagtgcagtggctagattcttcttgagtatgctcaaacttcctttttggaatgtcttccaaaggcactcttgccttcatttgtacaagttgattgaccctttaaaggccttaaatattattgtgcgacctcacagactcctcaaatcacctgaaacctgaaatgctgaggcccaggtggcactgaaatgatggtattctagacctgacaccggactgttttctccttggttttgtcccaacacactgacatacatagcccaaaatactactggcctttttaagtggcatatcacattccagggtaatatcaaaactgctgcctggtagcatttgtgaagtctcaaagtaactctttccaggattttcaaatccactgaatttcttagattgaaatatgtatgtgacagaattctcttagctttctttcctctatgaatatgtaattggaaactctgagatccggtttctcatctttattggattttttctttaatcttaaaattatgaatatttgctt

SEQ ID NO: 74 - DNA, 5125 bp, Homo sapiens
  source 1..5125: mol_type = genomic DNA
tggcaaaaacgcaaaacgttgatggataacggtgatgacttacacaacaatgcgaatgcatttaatgccactgaacagtacacttaaaaatggttaagatgatgaattttgtgttatatatgtttcaccacaatacaaaatattctttaaaaaagacttttggaaatactgtatctacttaattacaggatgtcaaactaatacaggctgatagtatcatttgtccccttgacacacaatcttgggtccagagattttgttcaccacaccttttagcatcactaaaaagggcacaataagaatatggtttcagaaaaagacaattcaaatattggtcttgtcctttagctatgtgaattcaatcaaattactcaaattctttgagtccaatatacttattttcttaaaataggattataatattgactgtaggagtgctacagaaataaaggcatgaaaaatatttataaattacaaatgttattaataatatttatacttccaaaaatgttgacaagaaatagagtaactaccccataataaagccacagcatctggaagctatattggattaagcaagaactaaaggttaaaatttcggattaaattttttttgcatgatactgctagtattatcaacattgggaaggcaatttcttgaatatttcttatatactattgaaatgtattcattattagttcaagttataattaccagtgacagattaaattacattcacttgtctttggttaaccatgacatttgacagaaggcaaatttctgcacttaagaaatgtattaaaaactaaaatgtatattaccttctaaaaaacttagctggtccatctttattgatgaatagtaggaagatatcaaaatagttatagggtgatgagatgtggcaagcatgcagtgctatggtatggtattacaaagcacaggattcttaactttgcctggaggagttgggaaatttcacataggagttgacctttgagcagcctcaaggataggaggaagatcttactagacggacaaaggcattccaagtagcagaaggcatgcgccaagagggaagcagagaacagtgtggggagtgttggtaactttgatattattaaagcggaggaagaaggataagaaatataaatggccaaataatttgcggccatattattattaaaataatgctatgattttagactttatcctgaagcactaacttaaattttaagcaaagggtaggttttgatttttagaactgatatgctagtcctatgatgacctggagcagccagaacctagaagctggaagatgagttgggaatctgcactagttcagatgagaggtgataagggtcttcattagagcagtgggttaggatacgacagactggatgtgttagctagctatcaagcaaacagagctgaggagacatgttaaccaattagtatgaaggaaggggaaagctcaaggcgatctggagattctgagagagaaaaggggcaatctgtcgtgagagcagtaattagatctagaagaggaatttttcaactacttaaattaggtcaaatttgtatggtacatttctgaaataagctaaaatagagccttaatctaaagtacaagatgagttactgaggataaccaataatgtacacataaaatgaacggagatgcatgttttagagtaattccaacaaaatagatctgtggataagtatgtaaggtactagtaagaataaagcatacaacacaagattaaaaactcttaagattaaaaatatcacatagacaataaaaatttacttaaaattttgtggttgtttttgagaccaagtctcactctgtcaccgaggctggagtgcagtggtgtgatcttggctcacggcaacctccacctcccaggttcaagcgattctcctgctgtgtttttaattgacttggtgttttacagtcattcactgatccattcaaccaataaacatctatgttgccacatccatgtgtgtggcattttgtctgatattgggaatatggtgtgatccctgaactcgaggagtctacagtgtaataaagaacacaaatatgcacataaatatttttagtaaaatactataagtaataaagacgtatggacaaagtaaccgaggattagggttaccaactcatcccagtttgcctgtgactttctgttagcatgggaagtcctgcatccaggaaaaccctttgctccgaggcaaatctaggatggttggtcatgctacccagcaccacataagacattttttaatgtaggtggtggtagtgggatgcagatttacttttttattttcccccaagagggatatattttagattatgtgtttggaaagagaaaagggataaggaagctaaagttcattttaggcaaaaggaaaaaacccaagcaaagacttggaagcatggttgtatgtcatttggtgttgctggagtacaagatccttatattgcatttataaaaaattgcttttatatttgtttacaaacagtccaaagcagccagtctactaagccaatttttttgggaaaaaggctgctgccaagcaacagaaacttacgtgaaacaaaacccacaagacacatgaagacttcttcaaatcttagaaaactataatgtgtgagattcttcaaatcttagaaaactataatacttttaatgacttaaaatattcacagtggaagaagtctgttttttaaagaaataaagttagatcattgtctcaaagggaaagactgtgaaatgggaacagcttgagatagaatgaatatattatgtatattacttttaaatggtagtttagagaagaggaataagagaaaacagtgtggaacacaaggtagaaatggcagggaaaaaaacgatacaggcctgaagaaaataaaagtaggtttgggcaatgtgggtggcaagatgagcccatattttggacccagagtgagtggaagaggtgagatggtcaagtagttcacagcattcttttaaataacatctgagtatactctggaatagacagggcaaaaaacaaatgaaattgcctgtggtagtccccatattaaataaatttaatttatttaattaaaaaactcaaagagtaaaaataataaagagaaagtgttgatattactgtaaaaaacagaccatattttctcctttcaatttttgtgctcgctggggatattttatttttaaatacaaactgatgttctctaaattcaaacatctttttattaaaagcgttactagaggtagcctgcagagcatatctagttctttgagttgccctgcttgaaggattgccacctctgtgtctctgaggctctgagcacagatgcctagggcatcagcctgagatgaaggtggtggggtttagaagaactgaaacacagctctaggacttcctctgccatttcaaactctttttagtaacacaggtagtaacatacagtcatgtattagtcaaacagttcccacctctttctttttctctttcctctcctccctccatccctgcttccctttcttccttttctccctcactcttctttccatctagtatttgttgagtactaactaggccagatattcttctaaattctggaaacacagcagtgaaggacaacgtttctggtcacttgatgcttccattctatgatttgtagttttttgcttttgtttcacaagggctacacaaaacccaaaaatcctaaagccaaaccccaaaaactcaactgaacagaaaacaggataaagggacagagagcaagaagtggtgctacttgatgtaggccagtgagggaggggcattctaaggaagaagcagcaccagagcaaaggcacaaaagaaaatgaggcagcagccagatagacctgtgggagcagcatattccgggaagtagaaacagcaaacaaaaaggctcaaggctgtaattggcttgggcttttgcctacctagtttctgttgtccctttcttctttactatcagaattctgattttattctacagggtattagattcagctaaaagataacatttcccaccttcttttgcagccacggaggtgatactactaagggtttttttgtttgtttgtttgttttgtttttaataaaacgatgatttgctggttgggaagcccttttgcccagctctgcacccaccttccccacctgggccctggaacatcaatgcccagcactgcggtggtcatcttgaaccatgaggtgatgctcaggcaagtcagggtaacaacgtagaacaaaaagagagaaggctctggtttcctgatgatactgtggaaccgccacaccagtccctacacagtgtacttttcatttcttttacagagagaaaactaaaaaccgtgatttttagcctagaattttcaggggtatctctgccattttcaatgaaagtatttctaaattcttcataggctagggatggaaacacagatgagttatgacgacgctgcaataatctatgtggaagatggcaatgccttagaccagggtggcactaacagaggtggtaaaaagtgatggcattctaggtatactttgaatgtagcactaacaaggatttgctgatagactggaggtgatatatgagagaaagatgagacaaaggttaactgtgaggtctgggccggcacaacagtgagcagtgatgccagtcactgaggtgagaggtgggggtggagcaaacttagaggcgagggaaagttcaggtgttctattttggacatgttaagcttgagttactcctagacatctgagtgggaatgcaaagaggcagaggtgtacatgagtaagggctgcagataaatgtttaggacacatctgcacatacatgggatataaagccacgcacctggacaaagtcacctagggggtgaatatataaaaagaaagggagttcaggaactgaagctacgagtgttctaatatttaaacgtcatacagagaagagaactccaaaaaaggaaactcaaacagcaggcaatggggcaaaagaagaactgagtgagtttaggatctcagagacaaatgaagaaagtctttcattgtggaagggaataa

SEQ ID NO: 75 - DNA, 1637 bp, Homo sapiens
  source 1..1637: mol_type = genomic DNA
tttgtcttcaactttttaaatatccatctattttttagattagatgccatctgttgtattatcatatctggttaaatcttattaaaattcctggccaatatatcactctgctgcggcaatcttaccctgctacctctctaggcttttctgcagcattcaataaagagcctgctattttaactaattgctgaggcaggacacagtgtgagagtctaaaacagaatcagcgctatattgtttctacttttacccatagagtgaacaactgttcatatcaaacctggaatcatcccttcaagtctcctaaacacagcacagtttgagccaatgcagttaatccaccctccttcagtgctagtgtcgaatggcgcttttgctgcagtattcaccttgaaactaagtaggcaggattcattatttgttgaagtcacctaactgccagtttattcttatacgatcacaaacaatcacaacagaagacaaatacaggcatatatataactcacttacaagggaagcaaatttgcagccagagacaagggcaacgtaacagccaagaactgcagtaaatctctttgagctaatagccacctctaataatgctctcctacaacacctccaatcaaataaattgtattcagagtttaaatatcaaatcaacattcattccttgatggttacagatgatgtccgataagcaaatttgaatttatgatttatttactccttaagtgtctcaaagccaggattactggaagacttactatgcttattcaagcatgctgacagaactgtaatctcagtaatttctccctgcacctctcagcatgactgacaggcatctgccaagactgtagtacataaactgctgaaacatgcaaaaatatttacccccaaaagatgtagtaaaagctcagcttcctccagcttccataacccctgaagtgttaatctggaggaacagttccatgagtttccacaggccagcagtgtgtctcctacacttgacctagacagccttacataacgaagcaccagtgctggggagctctctgaatgtcatcaccagcaagagcaagaagtattggcagcagcaggcagccaggcaggctgggagtttgcatggaaatcatgaagtctttcttgtcttcctcttttgaaatattttggaaggcgagttaagaatagctcagaaactggtctcattctttttgtgggaagaatgaccagaagcataaaagctaagtcttccagcaagtgcaagacacctcttttggtgtttgcagtaaacctaacaagaatgaattgctatcagtaaagtcctgtaccatgacacctaaaaggaaggaaatggtaaagcaaagtaataactcaaagacagtcaccgagggcttttgtccacctttaactcattgtggtgagtagagggaaatatgtatatatgtaatgagattattattgggggtgtgagttgttttgcaggatggtaactaaaggatttgtaaagagtgtttatgttccttaaagatcttgtttgtgagcaaggtaataggatatcaaattcagaacgtggtgggttaaactggttgtatttaatgtatttcaacttctgaaaatcttatgcaactaataagaaa

SEQ ID NO: 76 - DNA, 637 bp, Homo sapiens
  source 1..637: mol_type = genomic DNA
tcccaaatgtgtgcaatgcaagttatgcttttaaaagctagaaataataaaaccagtcttctattctgattttgagtatggtgatatagtattattaataaaagatgacattatattgtttaattatatataaaattgtggtttgatatgagttgttggctaatatgtataattcctgaggtaacagaaatagagaaggaaccacacatcatttaaaaataatcttaatgttctgctcttagctgggaaacctatctgctaatgcatcacactaagtagagtgaggaaataagagaatttagatctatgagggaacacagtgatctaattccaacccattacttaactcataaggaaactgaggtagaaagaagttagatgatatgcctgacatagaggaagaggtgagtgaaaaatggttttcctgacactaacttgttattttgtcagctatactgcaatgaataattgtcttttgatactggagtaaaggcttgatgtacagtgatttttttatatcatacaaatgacagaaaaaaaaagtggagtagtactaaatatctgcttttagcagtagtctgattttggaaaaacaagttctgtactgattggaatgagaaactttcttcagttattt

SEQ ID NO: 77 - DNA, 587 bp, Homo sapiens
  source 1..587: mol_type = genomic DNA
tatcccagaaagaaggaaatggtcagtttatctggagttaagcatttgtgtattatcatgattaatcacaggaacagttgccaagctttcattataaaaatattctccaggagaattcatcaagttccattgcctattaatttctgtccatgcattttatttggcatcttcaatgacagaggacacttttaaaaaaaagaaatgaagacaaaagaaaaagttcattagagaaataatgtatgtgtgatatttaaaaattaagccacatcatcatcctaagaaaactacgagactttagttttagtataaacttgcagtgtgttcttgagatttctaaatataaggcttaacattttttccttaatacacatcgatttggcatctcatatgtattatttatcaggaaattataaaacaaagtaaaatgatgttttactaaaatgcacatcatttttagatatgggattttaaaacttgatttataatactacttttaccatgaaatactcttttgttgtatgaccttgagtacatttcccatctgtgaatctgtgtaatcatgtacaaaaataaatgagacaaaacct

SEQ ID NO: 78 - DNA, 629 bp, Homo sapiens
  source 1..629: mol_type = genomic DNA
tcagcaagtctgtcatcgacatcctgcaactgtttgagcgggcagagcaagtgcgaaaagattaaaaagtgcttttctcatcatttctgctcatatgaccagcgctgcagtgctgcgcgccgggcgcacgcccgccgggcctggcatggcgccaggggcccggactctgagcgcagcgggagcggctcagtccagccgcgccgctgagcagcgccggccgccggcaagaaggcgcgcggacctgctaccactcctgcaccgccaggccaggggtccgcgggatcccaggggctgcggccagggcacgagggaaggggccacctctgggatttagggggcactggcgtcaccagctgggtctggaaagtccacctgccgtcaaggacacgcaggaggtgcgccgtctcagatctgggaaccttggcggatgtcctgccgcgtgggggaagatcctgaaccttcagcggccagcctgcacctcaggacctcctaggccctgctccctttctctctccactcctacctcagcctctgctctggtctgtcctggatgcaaatttatgctgcaaaatctgagcgctgaggtcctgaaacctgacccacccgacgcagggaggaggtggcaggga

SEQ ID NO: 79 - DNA, 237 bp, Homo sapiens
  source 1..237: mol_type = genomic DNA
tccctttctctctccactcctacctcagcctctgctctggtctgtcctggatgcaaatttatgctgcaaaatctgagcgctgaggtcctgaaacctgacccacccgacgcagggaggaggtggcagggacagggacagggacaggcaggagctgctggggcccacttcgggtgccccatcccacatctggccagggatgcatattctaaaacctgatttgatgttttacttttattt

SEQ ID NO: 80 - DNA, 594 bp, Homo sapiens
  source 1..594: mol_type = genomic DNA
cctgggctgcactaagtcccagtgtgaccttgggttgtgaccttctctgggcttctgtctccttctgatgtgttgatgacgtcagtggtcccatgtagtgggacctggggactgcaacttaaggtattggcaggtaggcagggccttgggctgtggtggccctgggtggtggggaccagggagagcagctgtccagctgcccagtaactcaagttccctgacatcgctgtcaacattgtctcctgcagctcagccctggatggctgcccttcctggaaaccttaggatacctctgctggctccagctgccccctccctgtgagtcagctccttcaagccacagcccgccagatggcttccaaggcaccaaggatgcagctcctgacctgatgcctctcagctccaggacttcccaggacccctcagctgccctggaccctgctgctactgccgtcacctctgcaccttgtccccagctgggctgctgactcagatatgccaggctcctatgctatcatttcaactcccaggctcagctcactccaggagcctagttggagaatggatttccccagctgaaggacgcttcagcta

SEQ ID NO: 81 - DNA, 638 bp, Homo sapiens
  source 1..638: mol_type = genomic DNA
gcacttcagttccttttcatcaaggaactgattaaagtgtggtctattatctctgtagtggggagtcccgaaagattccacttcttcctcttcttgcccaatgagagggtcaggaagcttccaccaccatcctgactgtggccaccacatcctggtgtgaagcaccaccagcttcctccacaagaactctgaaggtcaccagccagcttgagtcctccgaaggtgctgtggctcaaccagcagcctgtgtagcagagagcacaagacctgggactcgaactcagtcccaccttcaagagggatctccaccactttctgagcctcagttttcacatctttggttaggggcaggtgggaagttgccatatttaccttgttgggctctttggaaaaattaaatgaaatgttaatgtatgttacacaacgggctcataggagggattcaaaagctgctagttctttttctccttttcctgaaaacggtattaaagagtactgtagagacactggagaattcctgcttcatatgagatgtccttggtctctcccggggaatttgaagacccagagactcgcagctccccgtgagcctccccgctgacgccttacctccccctctccaccatcccctgccatcc

SEQ ID NO: 82 - DNA, 389 bp, Homo sapiens
  source 1..389: mol_type = genomic DNA
tgccttcccctccccctgtcggccgcccctcggtccctgggggtggggtttccctttgcgctcgccccctcccgcccccacccctcacgggccctcccctcccccgcccgtccctatgtatgtgtcacagcgcgccatgcccgcccgcccgcccacctacctccccgccgctccagagggggctcgcagagctgaggacgcgcgcagcgctgctcaaggtctctctctctcagcaccctcgccggccggcgtctgacgcgggtgccagggtctccgggcacctttcagtgtccattccctcagccagccaggactccgcaacccagcagttgccgctgcggccacagcccgaggggacctgcggacaggacgccggcaggaggaggggt

SEQ ID NO: 83 - DNA, 673 bp, Homo sapiens
  source 1..673: mol_type = genomic DNA
gccctgggaaaggggatcaggagcacatcttgggaacaggagccttcctctcctgctgtcaacggcctggcctcgtggccatgcttcgtgtcctgatggtagccgcactgccgccctgataacttaaaggaagccccttcaatgggatgaagggcccgtttctgtgacagctactttgggagtggcagctccccttcccccagatccaacaggacacagccatccctgcgagggagtctgggcccctggacgcagttgaagaagggccactgggaggccggcagaacaaggggagggctggagaaggagcgggagtgcaggcgagaggaggaccagagagggggaatttgtagagaaaacggtaaaacggtttcttttttcaaaagttgaatccagggcaagaacggaaacggtggagtttacttttaaaaactcagagcctccttataagtggtggagtgtgtgtgtgtgtgtgtgtgtgtgtgtgtgtgtgtgtgcatgctggcagggcgggccctgcgggctggccaggcctgtgagaggtgacttctctccctctcaatggccttacagtgtatcctaaagaagctttttgttaaactcatgaataggtggatttggggtgtgtgatgctgggcatcacgatttggatatttggttacctttgaggtta

SEQ ID NO: 84 - DNA, 595 bp, Homo sapiens
  source 1..595: mol_type = genomic DNA
ttcatttcaggctccccgtccccttcctctgcctcacctatgctgtcgccctgtcagcacctaattcagcttcccatggagaaaggcctccctgttgacagggccgtgctgggactcagggctgccaaagtcagtcttctcgcataaaaggctcagtgagtcctggagacacttggaagccagacagaatggaatttctccctattttctttacagctgagaaaacacacacaaacacaagagcatatttattgcgatatttctatcccaaagtttgtctttaaaaaaaaaaaagaaataaaattagtttctctgcccactccacccctatccccctacccccactctcctccccacctcttttcattccttcccatttcctggtttaggccagggagagaaatcaagccgtccaagccccacagagcactcctacacccccggacaatgtccagcttgttcagagagtgagggaagaaacacagctccgaaaacatacacacaacctcccaatgaagggtgttctgagggaagaacaggcgggccttgtgtctgaacacgaatccctaaggctctgggaagagaggagccggaa

SEQ ID NO: 85 - DNA, 1031 bp, Homo sapiens
  source 1..1031: mol_type = genomic DNA
ggtcttaaagtaggaaaacacatggtgtgatttgggttggtgaatagtcgacttgtagatctgcggactcgtcaacattgctctgacatcagattttctgaagagcagtgtgggctccttccccagggctggccgagttttgaggggaactgcaggttctgatgtttccaactctgtatctctgccctcgtcatttccatggacaagttatttgtgctggtgtgagatccagaatcggtcctgctacgtgacaatgcctggagcacggagccaggaacccaagctgcctcaccagcgtgttagggttgttactgtgcccgttttagaagatcacttggaggtgcaaaaatagagcagtttttttttgtttttttttttttgacatggagtctcgctctgtcgcccaggctggagtgcagtggcgcaatctcggctcactgcaagctccgcctccccggttcacgccattctcctgccttagcctcccgagtagctgggactacaggagcctgccaccatgcccggctaatttttttgtattttttagtagagacggggtttgaccatgttagccaggatggtctcgatctcctgacttcatgatccgcccgcctcggcctcccaaagtgctgggattacaggcgtgagccaccacgcctggccaagagcagatttttttaaaaaaattaagtacctctattcatttgcaccttcactacccagtgaggagatcaaaatttcctagagcaaatgcattcgatgccactcacagatttcgacaggagagcacaatttcaggaacgcctacatcaaagcactaattggcacttttacagtgtctttctccgcacgtgagccttgctggtggaaggagctgtcatagtaatgcgtattcctccatgcccagtgagtagggtgacggtcaattcacagttcactaggcacaaaagatgacggggctctcctctgctcgggacagcaagaaggttgaggtgatacggtttgtcggtgtccccacccaaatctcatct

SEQ ID NO: 86 - DNA, 477 bp, Homo sapiens
  source 1..477: mol_type = genomic DNA
catgaaagtcactgttttacatactaattgcattttccccaaaacatcaagctaaatacataactgatagcatgtttaaaggtcctatgtttcacctcaaattatctcatatttcacatgaggcaaaccctgtcctgaggccctgatgaagatgggcaggcagatctgatatcagctgcttttctttccttgatggaacctctggattgcgtgccctatcctataatgtgaaaaaagggcttccagaaaaggtggaggaattactttctgaaattctgaaaggctggatccaaaggtgcagaaaggaacattatttcctaccatataaaacccagtagggcgtgtgatgctgggacactgtatgagtccattctcatactgccataaagaagtacctgagactgggtattttataaaggaaagaggtttggttgactcacagttctgcaggcttaatagaaagcataactgggaggc

SEQ ID NO: 87 - DNA, 566 bp, Homo sapiens
  source 1..566: mol_type = genomic DNA
aaaaatgaagaatttatatgccaactccaggagaaatatttcagtgaggtcctggcttggtgcaaggatatggaaccagagccaagaattctatcagttaaaagcagcttagtttcctgagcctggactgatgggggacgtggaagacaaagtgtcaggtccatcagtggaagattggccttgagccactgtacacagaatggagagcccactggcctaaaaggagagattgtcaggcgtgacgaagcaggaattttagccgaagaatattcacaaattacaggccaagagggaagtggggacgttcgtcttctcttcatagccttgctcgttgggggaccagctgtcctttattgttaatagaaaaatcaatatagcaagaggcgaatctttgctgtgataacattggctcctttcaccaggcgtgtggaattagattactgatagatgcacctctgtcgcctccccaggctccagatagaatctatgggctttgccaataagcacggtaacagagtgtggatcaggaaccagcgggtggccaatggcagtggagaaaatgtaat

SEQ ID NO: 88 - DNA, 472 bp, Homo sapiens
  source 1..472: mol_type = genomic DNA
atagaccatataattctcacatgtcaaggttttaagccaaagccctcaggcaccacttctgattttcttgaaggatcaaaaataaaaggttgcaaccctcacagccgtaggctcctgcagcaactctttggtgcacctgtcaccctgatacctgggaggaggctctgagtccatgctgtgggaaggtgctggccttcatggatgggcctcccctggtgtgtccactgtggacctgagtgggtgtcccagagccctggctctcccttcttttcctctagaaagggaatcggcatgttcccaatcatctctgagattatctttattcttcaaggagttgcagtggctcttgccaagtgccctggggtcttggacatctgcctagtggccctgtagagacctccaccctccagacagctcagaatttgctaaagaaaatgtgaattttggagtccagggctagaaaatatgacat

SEQ ID NO: 89 - DNA, 489 bp, Homo sapiens
  source 1..489: mol_type = genomic DNA
aagaaaagttttattttgcctctgtagtattggggtttaagtgatcacggtaattttccattatcattttgtgttttaaataaatacaacaggctttattgtgaaaatatttgtctaatattgggcagtaaatgtttaagtgattttggtttaattactattacagtcatactattacagtgcataaaatagaattcttcttgagtttgttcattagatgggaagaggctgcatttttaaaaaatatatgcatgcctataatactacatttaaatatgtgcgtatataaagagatgctttcttatttatatacatggtcattatagagctttgtgagaaatagaattttctctgtgcaatctgtactctgggaggggttatttgctgacactgtatgcccatttcctaacagaatgtctctagttaagtaatcatatgatgaagacatcccagctgggactctatatttaagccaagttactatttcta

SEQ ID NO: 90 - DNA, 579 bp, Homo sapiens
  source 1..579: mol_type = genomic DNA
ctaaaaatgcctcctcgcctctgattttagccgtggttgttggagtaccggttccagcaggagctgtgatttccattgagctctcaaaccaaataaaatgcaaatctccgaggatggctcctctccctgcccccacagttgtgctccgaatagtgtctgagtttcatttttacaaggggcctttaaaaactcctgggccccttgaaaactcccagccccctttgtccagatggggatggaggtggccaggctgccccgttgattgtgtgccgaggagccctccccgggaaggctgtgatttatacgcgcaggcttgtcacggggtgaaaggaagggccactttttcattttgatccaatgttaggtttgaaagccacccactgctgtaaactcagctggatccgcgggccgtgattaaacacattgcccgctttgttgccgagatggtgtttcggaaggcgctgtgaatgcacttccctttgcggggctcacacagacaagatgtgtgttgcaaggatgaggcgcctgctcggcctccagcccagggccgggaagggagaaggtgctgtgcgtcgctgc

SEQ ID NO: 91 - DNA, 754 bp, Homo sapiens
  source 1..754: mol_type = genomic DNA
ttgaggcaagagcgagggtggcatatccagggtggccactgggtctggagtgtcagtagcagggcagatttagaaggtgactttgcatacctaggcaaggccagctcatgcgggatgtcggagcccatgggaagcaccttgcgtttgaggctgcctgcggtgggaagcttcagagtttcaagcggggctttgctatgggtttgttctgctttcccgttttcccctttggaggaggcttacagagatagtgatgactttgcagctgttaatcatcaggaagctgtaatcactaagaatgtttgaaatcatcagttaaggatttttagaaggaagtaaaccaaagaaatactgcagtagcctgccctaattatttcctgggcttaaagtaaccaggtgcattggagagattatttttcttcttctgatttatgaaggtctcagggtccaaattttgaaactgctgatcgaatttgttcttggatgttgtcatagaaatctgaaactttcctacttgtctgagagtgaaatttctttgattattcactcaagggtttgataggtttaaaaaaaggccttcgggacatctcttgttataaagtgtcaactttagatatcaagagaatcatgatatatttattactacaaaagagaaaataagcaactgaaaaactcatgaacttgaagcatgaagcaaaccccttaagttctaggggtttcaagatgtggatgccaacatgtgatgacatttaaaaga

SEQ ID NO: 92 - DNA, 378 bp, Homo sapiens
  source 1..378: mol_type = genomic DNA
ggcttttggtttttacaaaatattacaagttgcctaaatagtccgtgtttaaggacatagagccagagctctttctggaatgtcatacctcggcagggccttttgtgcatgttttaagctgattctgaaattagggggttaaaatggaagcgccgagccatccctaaagagagggaggcgaatgtgcccttgttgctggtgaccccagaacaaggcctctgggctgagaacaggagagaatgttatttctttgaaaagccatcttgacaatccaagtccgtttggctgcagcaccaaaggcagctttgatctgctcgccagtgtccctgccgggaaaaggattagggtccttccagaggacagcagagccaggctgcc

SEQ ID NO: 93 - DNA, 1490 bp, Homo sapiens
  source 1..1490: mol_type = genomic DNA
gcccaggctggagtgtggtggcaaaatctcagataactgaaacctctgcttcccaggctcaagccatcctcccacctctgtctgcagagtagctgagactataggcatgtgccacaatgctcagataattacttaacattctagtagagtctagtagacatgggctatcactatgttgccctggctggtctggaactcctgggctcaagtgattgttctgccttggcttcccaaagtgttgggattacggctgtaagccgccatgcttggcttcgctttacaatttttttttttttttttttgagacagagtcttactctgccacccaggctggagtgtagtggctagattttggctcactgcaaactctggcccttgggttaagagattctcctgcctcagcttcccaagtagctgggattacaggcatggacaaccatacctggctaatattttgtattagcagagacggtatttcaccgtgtcggccgggctggtctcgaactcccgacctcatgatccgcctacctcgggctcccaaagtgctgggattacaggcatgagccaccgtgcttggccaagaagacattttgttttctcaaaaaagtggagatctgagcttcaaagatccttggtaacacttcccagtgctatcagtgtagtggtgcagtggctaataattcatggaccctataggagggatcttgcctgctctttagaggttgggacacactcttcttggtaccagaagggcagaactatgcctctgtggccacttattgcagaatggaattggagtaaactgagggccctttcacacatgctagagaactgactttggccctaggagaagtgggggttgcaggggattggcctgagaaacttgccttttcactggattgtcctctagagtttttcactggagatttgtcagaatgagcctccagtccccatccagactcctggagctggcaggccagagcctgctgaggaaccagttcttgaccatcttcatcctggacgagctgcccagggaggtcttccctctgatgttcatggaggcctccagcatgagacattttgaggccctgaagctgatggtgcaggcctggcccttcctccgcctccctctgggatccctgatgaagacacctcatctggagaccttgcaagctgtgctgaagggacttgatacactgctggcccagaagcttcgccccaggtgaggtgactcaggtggcctggtgggaagggtccaggcatccagggaagggacagctggctcaggaggagtggtggggttggggagctagggtggctcagaggcttctgatggtgcccatgagagaccttgaccattgcccagatcctctggaaaaggactgctcaccatacagggtccactgaggaaacaggaacctgcttcctcccagtggaaggtaaaggttctagaagtgagaaccaggcagaatccaagggggagcgggatg

SEQ ID NO: 94 - DNA, 1739 bp, Homo sapiens
  source 1..1739: mol_type = genomic DNA
ttgggttccaatggaactacagagagcaatgactactggtcctgaatgtgggtataaagttttaccctaaaagtaccttgtatttttttccaaggccaaaatataacaactcatttgcaccctggaaaggtatgtttatttaaaaaaaaagattgcatttgcaaacagtagagaacactgctcttttttatttaaaaaaatctttaccatggaaaaacaataaagtttgcgtgtgtgtttattggtctggggacttaaagaacaaagataccttgtggattgcagacaaatgaaacccacagaggtttgctttgggtaggtttcaccatacaggtgtttcaatagaccactttgcaaataataaattacttaacactcaaggccgctagaggccactaaaaaggagtttatggccaaggcacagggctggtggctggctcagtgagcggtggcaggatattaatgagactcagagcctggacgtgctctggatccagttaaatgtaatagagttggaaaaccacctgcccccagccactgacggcacctaggattcatgcctgtaactttgaccatctgagcctgtagggacattggggaggagggggagggtgagaggaggcagtggcaacagcagcatggatgttccatgcaaacccttctctgtcaccagggaaagcagtctgagcacatgaatttcttagcctctcttccaggatgaagcctagtttgaaccagcactgccaggttgaagtgttactgcatcctgcagccagagccagggcatgtggccacccccttggtccctgctgtggtacccagagtcacttggaacatgtgtgaagccaggatgagggtgcatataccctccagatgctgatatctaaatatttacaagtcacaaatacagagaaactgttttttttttgttattgttgttgttgttttgagaaggagtctcgctctgtcgcccaggctggagtgcagtagcgtgatctcggctcaaagttctgcctcccgggttcacgccattctcctgcctcagcctcccaagtagctgggattataggcatgcaccaccacgcctagcaaattttgtatttttagtagagatggggtttctccatgttggtcaggctggtctcaactcctgatctcaggtgatctgcccacctcagcctcccaaagtgctgggattacaggtgtgagccaccgcacccggccaacaaaagtaccttcttaatgacttcgaagactaggtttaaatggtaaattattaaattcttggaaatctgccacagaatatggcattgtggggacagctgagctgattgaaacctgctccctttctcttcccactcccagctccatcctgcaccttaggggtctatgcacacctgtgtggacatcccaccctcacatccaacctctattcacattccccaccaccatcctgtgtggccactcagcctgctctaaagcagggatgctgggaagatgcccacatccaagcttggaatcgtttttgccagaaattgggggccctaagtacccaaaaaatgttctagaaggggacatgttctggatggccatggactccttgctccctggggaagagcacagctggaggaggactggagcaaggccccctaaagcactggacccaagataatgcccctcttgcccaggtccaagggctgtactagtggtacccgctgtcatcacagcattcattactg

SEQ ID NO: 95 - DNA, 736 bp, Homo sapiens
  source 1..736: mol_type = genomic DNA
cctcggaaccaaggttggctctggcacctgtagggccacgggcagctatgtcagcttcctcggaaggaccgaggctggctctggcatctccccgaccaatcctggctccactgtgtacccctgaagggcagaaaacagctactgcccaccgcagctccagcctggccccaacatctgtgggccagctggtgatgtctgcctcagctggaccaaagcctcccccagcgaccacaggctcagttctggctccgacgtccctggggctggtgatgcctgcctcagcagggccaagatctcccccagtcaccctggggcccaatctggccccaacctccagagaccagaagcaggagccacctgcctccgtgggacccaagccaacactggcagcctctggcctgagcctggccctggcttctgaggagcagcccccagaactcccctccaccccttccccggtgcccagtccagttctgtctccaactcaggaacaggccctggctccagcatccacggcatcaggcgcagcctctgtgggacagacatcagctagaaagagggatgccccagcccctagacctctccctgcttctgaggggcatctccagcctccagctcagacatctggtcctacaggctccccaccctgcatccaaacctccccagaccctcggctctccccctccttccgagcccggcctgaggccctccacagcagccctgaggatcctgtttt

SEQ ID NO: 96 - DNA, 1288 bp, Homo sapiens
  source 1..1288: mol_type = genomic DNA
agcctggccaacatggtgaaacgctgcctctactaaaaatacaaaaattagccaggcgtgatggcggtacctgtggtctcagctactggggaggctgagacaggagaatcacttgaacccgggaggtggaggttgcagtgagccgagattgcaccactgcactccagcctgggtgacagagcgagactccatctcagaaaaagaaaaaaaaaaaaaagagtctctgagtttacagatgagggccctggcattcagagaggctgaggaactcacccagcctgtcaacggcagaaccagagccaaatccaggatttgctagcttcaaagctatgttctcactcactccctaaggaggctgtgggcagaaggaaccctgggctgggaggcagcacagggcttggtatttatactagacctgttctgcctcagtttcccagtctgtaaagtggccctttgtctcaggcaatttgtgctaagacccaagagccttaagtgtgtgggatactagagggtctcccctgatgtggccccctgcccctgccttgcctggacagtttgccttcagggacgacatgccactggtgcggctggaggtggcagatgagtgggtgcggcccgagcaggcggtggtgaggtaccgcatggaaacagtgttcgcccgcagctcctgggactggatcggcttataccgggtgagaggggcagtggtggtcagcgactcagggaagaaaggggcctggaggagcagctgaacagcatggtggggtcactggcttgtccagatcttgatgccacactgggagactgctgggatcagacattatagggtcacaacactgattccacaacactgatcccccaggtgggtttccgccattgcaaggactatgtggcttatgtctgggccaaacatgaagatgtggatgggaatacctaccaggtacttaaaaggagtgggagagtcagggcaagtccttgttgcctttgggacctcagaactcaccttgggggctctcaggtggcctccctgacccccaacttaggcttatacccctgggcctaccaggtaacattcagtgaggaatcactgcccaagggccatggagacttcatcctgggctactatagtcacaaccacagcatcctcatcggcatcactgaacccttccaggtaagtaggccagactgctgggctgggggtgcctaaagacttttgtcaaatgccacagcctctacattctgctccttgagttcagacaaataacctgacctcccaagatctgccaac

SEQ ID NO: 97 - DNA, 1647 bp, Homo sapiens
  source 1..1647: mol_type = genomic DNA
acatcttaaacagtcttttaatgttatgaatttgatttttcaagaaatacatgtcatttattttcaaaacgaaatgatagctatactttctagagtctatcaatagtatttaaaataagatactcataactttcaaatactgcttttactagtcatcactcgtcattaaatgtaactgtatattcaagagctttctaataatagcctttaattaaacgaaggactgttagagggtttctgttgccctttgaagttcttaattattacttgtatccagcattttatggtacacttaaggttaaattaaatcatttaaatataccttgaagagaaatatgaagacttttgcccattttaattaaatctctgaatttcagtatttgaaaataataacatatgttttgatttttttttcatggccgaatggcaaaatgctcactatattaaacaacaaaaaaagaaatggtagctttttatgggactaatcgctaagcagatgcatgtaaatgagctattttctatgcatggcttccaaaagtgctaattaaatagttggtattcaaggctatgctcgctcattgtttagtgacacacaaatccagcgatgtgtgccagcagacattttaagttgaatgttttctcctctacggtctttgtcatgaaatggtggcaccatgatgagaacactagtgtaagcaaaacattgaaatatgctttaataatgttttaaccatgtagtgacactagcctagttttctaatgaatttttaatttctgttttcttataagggtgatatgagttatcgctgatgcatattaaatcatatacatgagtcattttctctaaatttgcataaaatggctaaatgctaatgcaccaaatggagcttactatatgtggtacagcaaatattcccttgaagattttctgcaatcaatctcctgtatttcattagcaaccagataaggtgtggtctgcagaataaaaaaagaaaagtgtgtagctcatgaacttatgaggcttcagatgatttctacgtggtgattagagtggattctgcaattagaatttatgtaggtaaaacacacatgtgcttcctttaaaggcacagtgcaacaaaagttctgaatacagccttgcaattgttaaacaatgaaaaggcaccattcaattattgtgatttttttacatctataattaaatgaaggaaagccatactttaaatttagtatcatttgattggcataacccttactgaaattttacaatttccctactatgtttataaaagaacttttaaaaataaccatgtgtgaaatattttgtttgctaactgttcccattttccttgtcaaataatggtgaagaattttctggactaatgtttaacatttaaaaatgttttttctatcatcaaatactcttactgaactgacattaggatcatatgctttataaaaaattgcattagggtaacagtattattgggcaaaccagagatgtttacttgaaggataaacttgctgcttactcactccactcatcaacccttttctcgtctcctacagttccaccatctggaatattttttaacccagtaaagaaaaaattggggaaggggatggctatttaaaaataaatgcttt

SEQ ID NO: 98 - DNA, 315 bp, Homo sapiens
  source 1..315: mol_type = genomic DNA
tattatcctagtaaatccttaaaaaactttaagaggtgggtgattttataattcccattttacagatcctggtactggggctttctggtcattaaaacacctgcctaaaaccactaatcagtaaatgggaggctggcttttgaacccagttttggtcgttgttcttaatcattattctttattgtttatggacatgtttgtctaatagcataatatgtagaatcaaagaaatgatattaagtgtggaaatggagtctccaaactctttatgcttgtttaaacgatcttctctctcgagagtgtatcttcatcctt

SEQ ID NO: 99 - DNA, 403 bp, Homo sapiens
  source 1..403: mol_type = genomic DNA
tttcccttactcagctaacaaacatttaccaagtatctgctgtgtgctaacgcttaggtgttaaactgggcatacaaactgaatgagaaagagtttgcctccacagagctgagcgtcctagagagatgtgcccagatgttgcaatcataatgcaatgagaaatgtaatgttggtacaggctactatgtaagcacaggaaagaggtgcataacttgtctgttagagtcaggaaaggcttttctcaaatggctgaactgaattctgtgggatgacaaagagtgctcaatagcatgaagcagaagaaggaaaggcatgctaggattgcataggtaagagtaagcggccgtgacattgccaagtggcggcacagtgtagcaattaagagcacacactgaggccgggt

SEQ ID NO: 100 - DNA, 1572 bp, Homo sapiens
  source 1..1572: mol_type = genomic DNA
ataaaatatcaggtaaatataatctctcatctctcatcttctctccatctccctatgtccctttccttctctctctctctctctctctcacacacacacacacacacacacacacacacacacacacacacagagagagagacacacatgttcttcctctaaaaagaaaaaccaataatcctctactgagacagttgtgaatcaaaggtttcttctgcaggagttacatccatctctgaatttcctagagagcagcaaagggccttgtgttttattccccttccacacttaatcactggactgtgggcccagactgaatgagtagctcattagaatcactgagttcactgaggggatgagagattccttcctggctgggtgctaagtgatactcccataaggattttgtggttacaaaacgtgctggatatggaggtaacctgtctgggagtcctgtcactccaaggatcacttggaatgctctggaaaaacacatgacctggctgaatgagttctgttgaattgtttagcctacaccttcatttcagcagcttatactgcattaatgaggttattgttcctttgccgtccaattgttcccaagctgattttttgcatatatgttttacatccttaacaagaatgcctgtctcctgctgtttcagagtctcttccacagtgctgagcatgagtggagcttgctaaatcattgctaaatgaagcaatgggctgtaagcatgtcctgtgggatctgcatcttcagatcatcctgaagtactcaacaaccacatcttcttccaggaacagagcccaacataaactggtagggtttgctgtcttagacagctaagagaacgaggagtggagctagtgaacaagcagtgaagggggcagttccttaatgccatccgaactgaatttcaacagtctgacaagctagcgttttgggtaaatatcccagtatacttgtcacagagttaagtaaaatggacttccttcaaaggaagtgcttttaatacaataactgtttttgtttttttaaccaatggattaaaaatttaacacatttactaaatctggcatatttatatattgtatctaaacagatattcaagctgcattataatataatcataaaaaaactgatctcagtctgtctgttaagcctttgtgagtctttgtgccattgttggagtagtgctaattatcaagcaaagacatgataattacgcagagcttttttgctaaaagaaggaatctttttcaacacccacgcactgcacaatttcctatgaccctgtagcactactctggtatggcccagaaatttgtatttctgtgtaaaggctggaaattatattatttctatctctcccgataccttttcttcttgtgagtaaactgtttttagagggttaaggaagaggggtaatggtccaaatgggaaatataaaactaatgacccttcatgaaacatattatgctcctcattaaacttattcagttaaatgtattccattaaattaaaataaataggatttaaaattttacccaggagcagtaaacaatctt

SEQ ID NO: 101 - DNA, 219 bp, Homo sapiens
  source 1..219: mol_type = genomic DNA
tgctgaaccagtctccgctgcatcgtctccgctcgcgctcgggacctgcaacagaagggaatgggacccgagtgtcagtctggactctccatctccccgcactactccgctccccctttttagcccgctctcaaaaagcctcttcaacatcaagggcatctcccaagttgaaaagaaaaaaaatttctctggagcctctcagcacttacttatttagca

SEQ ID NO: 102 - DNA, 668 bp, Homo sapiens
  source 1..668: mol_type = genomic DNA
caagctgatgggatcctcccatgggaaaagtgggcctcacacccttcctcacccttccctacttcctgggcaatgttctgcttcccccaaactgaagcaggaggcccagagaggaggcggtttcctgggaggaacccaaaccaatgtgagatgagaaggtctttaggaaatgggggtctctgagaaccggttcttaaaggtcaagcacttgagcacctcgcaaactcctgacaattgaaacatatctgaagagtcttcttcagatatgtctctgtgtgtgtgtgtgtgtgtgtgtgtgtgtgtgtgtgagagagagagagagagagagagagagagaatatgaatgtgcagtgtcccagtcctgatctcctggactggtgccagccagccagatgcctgcccttggctggccaagtttttggctcctgaaagtaggcagctctggacttgtacgaggccacagagagagttccaagccccacctggctcaggcgacaacctctcaacctgaagtcaatctccggtggcatcacagggccctcctggcagcagcccagttccccacatgaaccgaatggtcctttcttaaattttgagccgggggctgcctaaaaggggctgcccccgcaagcattttacctccctaacaccattctctgcccgtgcca

SEQ ID NO: 103 - DNA, 2817 bp, synthetic construct
  source 1..2817: mol_type = other DNA
ccaaagaagaagcggaaggtcggtatccacggagtcccagcagccaagaggaactacatcctgggcctggccatcggcatcaccagcgtgggctacggcatcatcgactacgagaccagggacgtgatcgacgccggcgtgaggctgttcaaggaggccaacgtggagaacaacgagggcaggaggagcaagaggggcgccaggaggctgaagaggaggaggaggcacaggatccagagggtgaagaagctgctgttcgactacaacctgctgaccgaccacagcgagctgagcggcatcaacccctacgaggccagggtgaagggcctgagccagaagctgagcgaggaggagttcagcgccgccctgctgcacctggccaagaggaggggcgtgcacaacgtgaacgaggtggaggaggacaccggcaacgagctgagcaccaaggagcagatcagcaggaacagcaaggccctggaggagaagtacgtggccgagctgcagctggagaggctgaagaaggacggcgaggtgaggggcagcatcaacaggttcaagaccagcgactacgtgaaggaggccaagcagctgctgaaggtgcagaaggcctaccaccagctggaccagagcttcatcgacacctacatcgacctgctggagaccaggaggacctactacgagggccccggcgagggcagccccttcggctggaaggacatcaaggagtggtacgagatgctgaagaggaggaggaggcacaggatcctgagccccgtggtgaagaggagcttcatccagagcatcaaggtgatcaacgccatcatcaagaagtacggcctgcccaacgacatcatcatcgagctggccagggagaagaacagcaaggacgcccagaagatgatcaacgagatgcagaagaggaacaggcagaccaacgagaggatcgaggagatcatcaggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccaggagcgtgagcttcgacaacagcttcaacaacaaggtgctggtgaagcaggaggaggccagcaagaagggcaacaggacccccttccagtacctgagcagcagcgacagcaagatcagctacgagaccttcaagaagcacatcctgaacctggccaagggcaagggcaggatcagcaagaccaagaaggagtacctgctggaggagagggacatcaacaggttcagcgtgcagaaggacttcatcaacaggaacctggtggacaccaggtacgccaccaggggcctgatgaacctgctgaggagctacttcagggtgaacaacctggacgtgaaggtgaagagcatcaacggcggcttcaccagcttcctgaggaggaagtggaagttcaagaaggagaggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccacagggtggacaagaagcccaacaggaagctgatcaacgacaccctgtacagcaccaggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagcaggaacaaggtggtgaagctgagcctgaagccctacaggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtacagggtgatcggcgtgaacaacgacctgctgaacaggatcgaggtgaacatgatcgacatcacctacagggagtacctggagaacatgaacgacaagaggcccccccacatcatcaagaccatcgccagcaagacccagagcatcaagaagtacagcaccgacatcctgggcaacctgtacgaggtgaagagcaagaagcacccccagatcatcaagaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctg

SEQ ID NO: 104 - DNA, 2817 bp, synthetic construct; identical to SEQ ID NO: 103
  source 1..2817: mol_type = other DNA

SEQ ID NO: 105 - DNA, 2817 bp, synthetic construct
  source 1..2817: mol_type = other DNA
ccaaagaagaagagaaaggttggcattcacggggtgccagccgccaaaaggaattatatcctcggattggcaataggcataaccagtgtaggttatggaatcatagactatgaaacccgggacgttatagacgcgggagtacggttgttcaaagaggcaaatgttgaaaacaatgagggccggcggtccaaacggggtgcacggaggcttaaacgccgcaggagacatcggattcaaagggtcaagaaacttctttttgattataacctcctgaccgaccacagtgagctctccgggataaatccttacgaagccagagtcaagggacttagccaaaagctgtcagaggaagagttttcagcggcactgcttcatcttgcaaaaaggcggggcgtccataatgttaatgaggtagaagaagatactgggaacgagctcagtacgaaggaacaaatctcaagaaatagcaaggctcttgaggaaaaatacgtagcagaacttcagttggaaagacttaagaaagacggagaggttcggggaagtattaaccgctttaagacttcagattatgttaaagaggcaaagcaattgttgaaagttcagaaagcttatcatcaattggatcaatcatttatagatacatacatagatctccttgaaaccagacggacgtactacgaaggtccaggagaagggtcacccttcggatggaaggacatcaaggaatggtacgaaatgcttaaaaggaggcgaagacatagaattctcagtcctgtcgtgaagcgaagcttcatacagagcataaaggtaataaacgctataatcaagaagtacggattgcctaacgatattataatagaattggcacgagagaaaaacagtaaggatgcacagaagatgattaacgagatgcagaagcgaaatcgccaaacgaatgagcgcatagaagagattataagaactacaggcaaggagaacgccaagtatcttatagaaaagataaaactccatgacatgcaggaaggtaaatgtctttattctctcgaggctatcccgttggaggatctgctgaataaccctttcaattacgaagtagaccatatcatacctcgatccgtttcattcgataattcctttaacaataaagtgctcgttaaacaggaagaggcatccaagaaagggaatagaactccgtttcaatacctgtctagttctgactctaaaataagttatgaaacctttaaaaagcatatactcaaccttgcgaagggcaaggggcggatatcaaagaccaaaaaggaatacctcctcgaggagcgggacattaatagattttctgtgcagaaagattttataaaccgcaacttggtagacacgcgatatgctacacgaggtctgatgaacttgctccgatcttattttcgcgtcaataatctggatgtcaaggtgaagtctattaacggtggtttcacatccttcctgcgacgaaagtggaagtttaagaaagagcggaataagggctacaagcatcatgccgaggacgctctgattatcgctaacgcggattttatctttaaagagtggaagaaactcgacaaagctaagaaagtgatggagaaccagatgtttgaggaaaagcaggccgaatcaatgccggaaatcgagacggagcaggaatataaggagattttcatcactcctcatcagataaagcatattaaagacttcaaagactacaagtatagccacagggtggacaagaagcccaacaggaaactgattaacgatacactctattccacccggaaagatgataaaggaaatacgctgatcgtaaataacctcaacggactttacgataaggataatgataaactcaaaaagcttatcaataagtctcccgagaagctgctcatgtaccaccatgatccccagacataccaaaagctgaagctcatcatggaacagtatggagatgagaagaacccgttgtacaaatactatgaagagacgggcaactaccttactaagtacagcaagaaggataacgggccggtgatcaaaaagattaagtattacgggaataaacttaatgcgcatcttgacatcactgacgactaccccaattctcggaataaggttgtaaaactgtcccttaagccatatcggttcgacgtctatctcgacaacggtgtttacaagttcgtaacagtcaagaatcttgatgttataaaaaaagagaattattacgaagttaactcaaagtgctatgaggaagcgaaaaaacttaaaaaaatatccaaccaggccgagtttatagcttccttttataaaaacgatctgataaaaatcaacggcgaattgtacagagtaatcggtgttaataatgatttgctgaataggatagaagttaatatgatagatattacttacagggaatatttggaaaacatgaatgacaagcggcccccgcacatcatcaagacaatcgcatccaaaactcagtccatcaagaagtattctacggacattcttggaaatctttatgaggtaaagagcaaaaaacatccgcaaatcattaaaaagggaaagagacccgcagccactaagaaggccgggcaggcgaagaagaaaaagggttctgaggctagcgggtctggccgagcggatgctttggatgattttgacttggacatgcttggttctgacgctcttgatgattttgatctggatatgttgggaagcgacgcgctggatgattttgatctggatatgttgggctcagacgcacttgatgacttcgaccttgatatgctg

SEQ ID NO: 106 - DNA, 4104 bp, synthetic construct
  source 1..4104: mol_type = other DNA
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagac

SEQ ID NO: 107 - DNA, 39 bp, synthetic construct
  misc_feature 1..39: note = VP16(1) transactivator
  source 1..39: mol_type = other DNA
gacgcgctggacgatttcgatctcgacatgctgggttct

SEQ ID NO: 108 - DNA, 78 bp, synthetic construct
  misc_feature 1..78: note = VP16(2) transactivator
  source 1..78: mol_type = other DNA
gacgcgctggacgatttcgatctcgacatgctgggttctgatgccctcgatgactttgacctggatatgttgggaagc

SEQ ID NO: 109 - DNA, 117 bp, synthetic construct
  misc_feature 1..117: note = VP16(3) transactivator
  source 1..117: mol_type = other DNA
gacgcgctggacgatttcgatctcgacatgctgggttctgatgccctcgatgactttgacctggatatgttgggaagcgacgcattggatgactttgatctggacatgctcggctcc

SEQ ID NO: 110 - DNA, 150 bp, synthetic construct
  misc_feature 1..150: note = VP64 transactivator
  source 1..150: mol_type = other DNA
gacgcgctggacgatttcgatctcgacatgctgggttctgatgccctcgatgactttgacctggatatgttgggaagcgacgcattggatgactttgatctggacatgctcggctccgatgctctggacgatttcgatctcgatatgtta

SEQ ID NO: 111 - DNA, 1569 bp, synthetic construct
  misc_feature 1..1569: note = VPR transactivator
  source 1..1569: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 112 - DNA, 357 bp, synthetic construct
  misc_feature 1..357: note = p65 transactivator
  source 1..357: mol_type = other DNA
cctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctg

SEQ ID NO: 113 - DNA, 570 bp, synthetic construct
  misc_feature 1..570: note = Rta transactivator
  source 1..570: mol_type = other DNA
cgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 114 - protein, 1145 aa, synthetic construct
  REGION 1..1145: note = NLS-SadCas9-NLS-VP64
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSEASGSGRADALDDFDLDMLGSDALDDFDLDMLGSDALDDFDLDMLGSDALDDFDLDML

SEQ ID NO: 115 - DNA, 3429 bp, synthetic construct
  misc_feature 1..3429: note = NLS-SadCas9-NLS-VP64
  source 1..3429: mol_type = other DNA
ccaaagaagaagcgaaaagttggaatccatggggtcccggccgctaaaagaaactatatcctggggttggcaataggaatcaccagcgtcggttacggtataatagattatgagacacgagatgtaatcgatgcgggagtacgcttgttcaaagaagccaacgtagaaaataatgagggacggcggtctaaacgaggagctagacggcttaagcgacgccgccggcaccggatccaacgcgttaaaaagttgctttttgactataatctgcttacggatcactccgagctctctgggattaatccgtacgaagcccgcgtcaaggggttgagccaaaaattgagtgaagaggaatttagcgctgcgctcttgcacctggcgaaaaggcggggtgtacataacgtaaatgaggttgaggaggatactggtaacgaactcagcaccaaggagcaaataagtcggaacagcaaagcattggaggaaaaatacgtggcggagctgcaacttgagcggctcaagaaggatggggaagtgcgcgggagcataaaccgatttaagaccagcgattatgtgaaagaagctaagcaattgttgaaggttcagaaggcttaccatcagctcgaccaatcattcattgatacgtatatagacctgttggaaacacgacgcacctattatgaaggtccgggtgaaggaagtcccttcgggtggaaggacatcaaagaatggtacgaaatgcttatgggacattgtacttacttccctgaggaactgaggagcgtaaagtatgcctataatgcagatttgtacaacgcgctgaacgacttgaataatctcgtgataactcgggacgagaacgagaaacttgagtattacgagaagttccagatcatcgagaatgtctttaagcagaagaaaaaaccgaccttgaaacaaattgccaaggagatactggtaaatgaagaagacattaagggttatcgagtaacgtcaactgggaaaccagagttcacaaacttgaaagtctaccacgatataaaagatataacggctcgaaaggagatcatcgaaaatgcagagttgttggatcagatagcgaagatattgaccatctatcaaagcagcgaggatatacaggaagagctgaccaatttgaattccgagctcacacaggaggaaatagagcagatcagtaacttgaaaggttacacggggactcacaacctgagtttgaaggcgatcaacttgattctggatgagttgtggcataccaatgacaatcaaatcgccatatttaacaggcttaagcttgtccccaaaaaagtggacctctcacagcagaaagaaattccaaccaccctcgtggacgacttcatattgagtcccgtggtgaagcgatcatttatccaatcaatcaaagtaatcaatgccatcattaaaaaatacggcttgccaaatgacataatcattgagttggcacgcgaaaaaaatagtaaggatgctcaaaagatgataaacgagatgcagaaacggaatcgccaaactaatgaacgcattgaggaaataattcgcactaccgggaaggaaaacgcaaagtatttgattgagaaaatcaaactgcacgacatgcaagagggcaaatgtctttacagcctggaggcgatccctctcgaagacttgcttaacaacccgtttaactatgaggtcgatcatattatacctcgatctgtgagttttgataacagtttcaataacaaagttcttgtcaagcaagaggaggcctccaagaaaggtaatcgcaccccgttccagtatttgtctagtagcgattccaaaatatcatatgaaacttttaagaagcatatcttgaaccttgctaaggggaaggggcgcatctccaagacaaagaaagagtatcttctggaagaacgcgatatcaacaggttttcagttcaaaaagacttcatcaacagaaacctcgtggatacgcggtatgccacacgaggactcatgaatcttctcaggtcatactttcgggttaacaacctggacgtaaaagttaaaagcatcaacggggggtttacgtccttcttgcgccgcaaatggaaattcaaaaaggagagaaacaagggctacaaacatcacgcggaagacgcactgattatcgcgaacgctgattttatttttaaagaatggaagaaacttgataaggcgaaaaaagtaatggaaaatcagatgtttgaggagaagcaggcggagtctatgccagaaattgaaacggagcaagaatacaaagaaatctttataacgccacatcagataaagcatattaaggactttaaagattacaaatactcacaccgggtcgacaaaaaacccaatagggagctgataaacgacactctgtactccacacggaaagacgacaaaggcaacacactcattgttaacaacttgaatggcttgtacgataaagacaatgacaagctgaaaaaacttataaataaatcaccggagaaactgctgatgtatcatcatgacccgcaaacgtatcaaaagctcaagctgataatggaacagtatggagatgagaaaaatccattgtacaaatattacgaagaaactggtaactatcttacaaaatactcaaaaaaggataatgggccagttataaagaagataaagtattacggcaacaagcttaatgctcatctggacatcacagatgactatcccaactcccggaataaagtcgtaaagctgagccttaaaccgtacagattcgatgtttatctggacaatggagtctataaattcgtaactgtgaaaaacttggatgtgataaagaaagagaactactatgaggtaaattctaaatgttacgaagaagcgaaaaaattgaaaaaaatctcaaatcaagccgagtttatcgcaagcttctacaacaatgatctcattaaaattaacggagaactctaccgggttataggagtaaataatgacctcctgaaccgaatagaggtgaatatgatagatattacttatagagagtatttggagaacatgaacgataagaggcctcctcggattattaagaccatcgcctcaaagactcagtcaatcaaaaaatattcaacggacattcttgggaatttgtacgaggttaaaagtaaaaaacatcctcaaataatcaaaaaagggaagaggccggcggctacgaaaaaggctggacaggcgaagaagaaaaagggttcagaagcgagtgggagcggtcgcgcggacgctctcgatgatttcgacctggacatgcttgggtccgatgctctcgacgattttgatctggacatgctcggttctgacgcgcttgacgacttcgacctcgacatgttggggtcagacgcgctcgacgatttcgacctcgatatgctg

SEQ ID NO: 116 - DNA, 3429 bp, synthetic construct; identical to SEQ ID NO: 115
  misc_feature 1..3429: note = NLS-SadCas9-NLS-VP64 (optimized 1)
  source 1..3429: mol_type = other DNA

SEQ ID NO: 117 - DNA, 3429 bp, synthetic construct
  misc_feature 1..3429: note = NLS-SadCas9-NLS-VP64 (optimized 2)
  source 1..3429: mol_type = other DNA
cccaagaagaagagaaaagtcggcatccacggcgtgcccgccgccaagagaaactacatcctgggcctggccatcggcatcacaagcgtgggctacggcatcatcgactacgagacaagagacgtgatcgacgccggcgtgagactgttcaaggaggccaacgtggagaacaacgagggcagaagaagcaagagaggcgctagaagactgaagaggaggagaagacatagaattcagagagtgaagaagctgctgttcgactacaacctgctgaccgaccacagcgagctcagcggcatcaacccctacgaggctagagtgaagggcctgtctcagaagctgagcgaggaggagttcagcgccgccctgctgcacctggccaagagaagaggcgtgcacaacgtgaacgaggtggaggaggacaccggcaacgagctgagcaccaaggagcagatctcgagaaatagcaaggccctggaggagaagtacgtggccgagctgcagctggagagactgaagaaggacggcgaggtgagaggcagcattaacagattcaagacaagcgactacgtgaaggaggccaagcagctgctgaaggtgcagaaggcctaccatcagctggatcagagcttcatcgacacctacatcgacctgctggagacaagaagaacctactacgagggccccggcgagggcagccccttcggctggaaggatatcaaggagtggtacgagatgctgatgggccactgcacctacttccccgaggagctgagaagcgtgaagtacgcctacaacgccgacctgtacaacgccctgaacgacctgaacaatctggtgatcacaagagacgagaacgagaagctggagtactacgagaagttccaaatcatcgagaacgtgtttaagcagaagaagaagcccaccctgaagcagatcgcgaaggagatactggtgaacgaggaggacatcaaaggctatagagtgacaagcaccggcaagcccgagttcaccaacctgaaggtgtaccacgacatcaaggacatcaccgctagaaaggaaattatcgagaatgcggaactgctggatcagatcgcgaaaatactgaccatctatcagagcagcgaggacatccaagaggagctgaccaacctgaactcagagctgacccaagaggagatcgagcaaatttctaacctgaagggctacaccggcacccacaacctgagcctgaaggccattaacctgatcctggacgagctgtggcacaccaacgacaatcagatagccatttttaacagactgaagctggtgcccaagaaggtggacctgtctcagcagaaggagatccccaccaccctggtggacgacttcatcctgagccccgtggtgaagagaagcttcatccaaagcatcaaggtcattaacgccattattaaaaagtacggcctgcccaacgacatcatcatcgagctggctagagagaagaacagcaaggacgctcagaagatgatcaacgagatgcagaagagaaacagacagaccaacgagagaatcgaggagatcatcagaaccaccggcaaggagaacgccaagtacctgatcgagaagatcaagctgcacgacatgcaagagggcaagtgcctgtacagcctggaggccatccccctggaggacttgctcaacaaccccttcaactacgaggtggaccacatcatccctagaagcgtgagcttcgacaacagcttcaacaacaaggtgctggtgaagcaagaggaggctagcaaaaagggtaacagaaccccctttcagtacctgagcagcagcgacagcaagatcagctacgagaccttcaagaagcacatcctgaacctggccaagggcaagggcagaatcagcaagaccaagaaggagtacctgctggaggagagagacattaaccggttcagtgtgcagaaggacttcatcaacagaaacctggtggacacaagatacgccacaagaggcctgatgaacctgctgagaagctacttcagagtgaacaacctggacgtgaaggtaaaatctattaatggcggcttcacaagcttcctgagaagaaagtggaagttcaagaaggaacgcaacaagggctacaagcaccacgccgaggacgccctgatcatcgccaacgccgacttcatcttcaaggagtggaagaagctggacaaggccaagaaggtgatggagaatcagatgttcgaggagaagcaagccgagagcatgcccgagatcgagaccgagcaagagtacaaggagatcttcatcaccccccatcagatcaagcacatcaaggacttcaaggactacaagtacagccacagagtggacaagaagcccaacagagagctgatcaacgacaccctgtacagcacaagaaaggacgacaagggcaacaccctgatcgttaacaacctgaatggactgtacgacaaggacaacgacaagctgaaaaaactgatcaacaagagccccgagaagctgctgatgtaccaccacgaccctcagacctatcagaagctgaagctgatcatggagcagtacggcgacgagaagaaccccctgtacaagtactacgaggagaccggcaactacctgaccaagtacagcaagaaggacaacggccccgtgattaagaagatcaaatactacggcaacaagctgaacgcccacctggacatcaccgacgactaccccaacagcagaaacaaggtggtgaagctgtccctgaagccttacagattcgacgtgtacctggacaacggcgtgtacaagttcgtgaccgtgaagaacctggatgtaataaaaaaggagaactattacgaggtgaacagcaagtgctacgaggaggccaagaagttaaagaaaatcagcaaccaagccgagttcatcgctagcttctacaacaacgatctgatcaagatcaacggcgagctgtacagagtgatcggcgtgaataatgacctgctgaatcggatcgaggtgaacatgatcgacatcacctacagagagtacctggagaacatgaacgacaagagaccccctagaatcatcaagaccatcgctagcaagacccaaagtatcaaaaaatacagcaccgacatcctgggcaacctgtacgaggtgaagagcaagaaacaccctcagatcatcaagaagggaaaaagacccgccgccaccaagaaggccggccaagccaagaagaaaaagggcagcgaggctagcggcagcggcagagccgatgccctggacgacttcgatctggacatgctgggaagcgacgccctagatgacttcgaccttgacatgctggggtctgatgcgctagatgatttcgatcttgatatgctcgggagcgacgctctagacgactttgatttggatatgctg

SEQ ID NO: 118 - protein, 941 aa, synthetic construct
  REGION 1..941: note = NLS-miniSadCas9v2-NLS-VP64
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLKRRRRHRILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKGKRPAATKKAGQAKKKKGSEASGSGRADALDDFDLDMLGSDALDDFDLDMLGSDALDDFDLDMLGSDALDDFDLDML

SEQ ID NO: 119 - DNA, 2817 bp, synthetic construct; identical to SEQ ID NO: 103
  misc_feature 1..2817: note = NLS-miniSadCas9v2-NLS-VP64
  source 1..2817: mol_type = other DNA

SEQ ID NO: 120 - DNA, 2817 bp, synthetic construct; identical to SEQ ID NO: 105
  misc_feature 1..2817: note = NLS-miniSadCas9v2-NLS-VP64 (optimized 1)
  source 1..2817: mol_type = other DNA

SEQ ID NO: 121 - DNA, 2817 bp, synthetic construct
  misc_feature 1..2817: note = NLS-miniSadCas9v2-NLS-VP64 (optimized 2)
  source 1..2817: mol_type = other DNA
ccaaagaaaaagagaaaagtgggcatccacggcgtgcccgccgccaagagaaactacatcctgggcctggccatcggcatcacaagcgtgggctacggcatcatcgactacgagacaagagacgtgatcgacgccggcgtgagactgttcaaggaggccaacgtggagaacaacgagggcagaagaagcaagagaggcgctagaagattgaaacggagaagaagacaccggattcagagagtgaagaagctgctgttcgactacaacctgctgaccgaccacagcgagctgagcggcatcaacccctacgaggctagagtgaagggcctgtctcagaagctgagcgaggaggagttcagcgccgccctgctgcacctggccaagagaagaggcgtgcacaacgtgaacgaggtggaggaggacaccggcaacgagctgagcaccaaggagcagatcagcagaaacagcaaggccctggaggagaagtacgtggccgagctgcagctggagagactgaagaaggacggcgaggtgagaggcagcattaaccgcttcaagacaagcgactacgtgaaggaggccaagcagctgctgaaggtgcagaaggcctaccatcagctggatcagagcttcatcgacacctacatcgacctgctggagacaagaagaacctactacgagggccccggcgagggcagccccttcggctggaaggacatcaaggagtggtacgagatgcttaaacgacgtcgtagacacagaatcttaagccccgtggtgaagagaagcttcatccaaagcatcaaggtgatcaacgccatcattaaaaagtacggcctacccaacgacatcatcatcgagctggctagagagaagaacagcaaggacgctcagaagatgatcaacgagatgcagaagagaaacagacagaccaacgagagaatcgaggagatcatcagaaccaccggcaaggagaacgccaagtacctgatcgagaagatcaagctgcacgacatgcaagagggcaagtgcctgtacagcctggaggccatccccctggaggacctgctgaacaacccattcaactacgaggtggaccacatcatccctagaagcgtgagcttcgacaacagcttcaacaacaaggtgctggtgaagcaagaggaggctagcaagaagggcaacagaaccccctttcagtacctgagcagcagcgacagcaagatcagctacgagaccttcaagaagcacatcctgaacctggccaagggcaagggcagaatcagcaagaccaagaaggagtacctgctggaggagagagacatcaatagatttagcgtgcagaaggacttcatcaacagaaacctggtggacacaagatacgccacaagaggcctgatgaacctgctgagaagctacttcagagtgaacaacctcgatgtcaaggtaaaatccatcaatggcggcttcacaagcttcctgagaagaaagtggaagttcaaaaaggagcgtaacaagggctacaagcaccacgccgaggacgccctgatcatcgccaacgccgacttcatcttcaaggagtggaagaagctggacaaggccaagaaggtgatggagaatcagatgttcgaggagaagcaagccgagagcatgcccgagatcgagaccgagcaagagtacaaggagatcttcatcaccccccatcagatcaagcacatcaaggacttcaaggactacaagtacagccacagagtggacaagaagcccaacagaaagctgataaacgatactctgtacagcacaagaaaggacgacaagggcaacaccctgatcgtgaacaatctgaacggcctgtacgacaaggacaacgacaaactgaagaaattaataaacaagagccccgagaagctgctgatgtaccaccacgaccctcagacctatcagaagctgaagctgatcatggagcagtacggcgacgagaagaaccccctgtacaagtactacgaggagaccggcaactacctgactaagtacagcaagaaggacaacggccccgtaataaagaagatcaaatactacggcaacaagctgaacgcccacctggacatcaccgacgactaccccaacagcagaaacaaggtggtgaagctgagcctgaagccctacagattcgacgtgtacctggacaacggcgtgtacaagttcgtgaccgtgaagaacctggacgtgatcaagaaggaaaactattacgaggtgaacagcaagtgctacgaggaggccaagaagctgaagaagatctcaaaccaagccgagttcatcgctagcttctacaagaacgacctgatcaagatcaacggcgagctgtacagagtgatcggcgtgaacaacgatctgctgaacagaattgaggtgaacatgatcgacatcacctacagagagtacctggagaacatgaacgacaagagacccccccacatcatcaagaccatcgctagcaagacacagagcatcaagaaatacagcacggacatcctgggcaacctgtacgaggtgaaaagcaagaaacaccctcagatcataaaaaagggcaaaagacccgccgccaccaagaaggccggccaagccaagaagaagaagggcagcgaggctagcggcagcggcagagccgatgccctggacgacttcgatctcgacatgctgggcagcgacgccctagatgacttcgaccttgatatgctgggtagcgatgcgctagatgatttcgatcttgatatgctcgggagcgacgctctagacgatttcgacctggacatgctg

SEQ ID NO: 122 - protein, 977 aa, synthetic construct
  REGION 1..977: note = NLS-miniSadCas9v4-NLS-VP64
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELGSKRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKKGSEASGSGRADALDDFDLDMLGSDALDDFDLDMLGSDALDDFDLDMLGSDALDDFDLDML

SEQ ID NO: 123 - DNA, 2925 bp, synthetic construct
  misc_feature 1..2925: note = NLS-miniSadCas9v4-NLS-VP64
  source 1..2925: mol_type = other DNA
ccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctgggcagcaagagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctg

SEQ ID NO: 124 - DNA, 2925 bp, synthetic construct
  misc_feature 1..2925: note = NLS-miniSadCas9v4-NLS-VP64 (optimized 1)
  source 1..2925: mol_type = other DNA
ccaaagaaaaaaaggaaagtaggtattcacggggtacctgccgctaaacgaaactatattctcggactggcaatagggatcacaagtgttggatacggcattatagattatgaaactcgagacgtgattgatgcaggtgtacggttgtttaaagaggccaacgttgaaaacaacgaggggcgccggagtaaacgcggagcgcgaaggctcaagaggcggagaagacaccgcattcagagagtaaaaaaactgcttttcgactataatctcttgacagatcacagcgaactgtcaggtatcaacccctacgaggcgagggttaagggactcagtcaaaagctttcagaagaagaattttccgctgcccttcttcatttggcaaagcgaagaggtgtgcataatgtgaatgaagttgaggaagatacgggcaacgagctgtccactaaggagcagatctcacggaatagtaaggccctcgaggaaaagtacgtcgctgaactgcagcttgaacggctgaaaaaggatggggaggtccgcgggtctatcaaccgcttcaaaacaagtgactacgttaaggaggccaagcaattgcttaaggttcaaaaagcgtaccatcaattggatcaaagctttattgatacgtatattgacctcctggaaacaaggcgaacttactacgaggggcccggcgaaggctccccctttggatggaaagacatcaaggaatggtacgaaatgttgatgggacattgcacctatttccccgaagagctcagatctgtaaaatacgcatacaatgctgatctgtacaacgctctcaacgatttgaacaatcttgtcattacgagggacgaaaatgaaaagcttgagtactatgaaaaattccagattattgagaacgttttcaagcagaagaagaaaccaacgttgaaacaaattgcaaaagaaatacttgtaaatgaagaggatataaaagggtatcgagttacaagcacggggaaaccagagtttaccaacttgaaagtataccacgatataaaggatattacagcacggaaggagatcattgagaacgccgaactccttgaccaaatcgcgaagattctgactatttatcaatccagtgaggacatccaggaggagttgacaaaccttaattccgagcttacccaagaggaaattgagcaaatatcaaaccttaaaggctataccggtacgcataacttgagtctcaaggccataaatcttattcttgatgaactctggcataccaatgacaatcagatcgcgatctttaaccggctcaagcttgtccctaaaaaagtggacctttcacaacagaaagaaattcctaccacgctcgtcgatgactttattttgagcccagtggttaaacgaagttttattcaaagtatcaaggtaattaacgctataatcaaaaagtatggattgccaaacgatataatcattgaactgggttcaaaacggtacgcgacccgaggtttgatgaacctcctgcggagctatttccgggttaacaatttggacgtgaaggtgaagagcatcaatggcggttttacaagtttcctgagacgcaaatggaaattcaagaaggagcggaataaaggttataagcaccatgctgaagacgccttgataattgcgaatgccgacttcatttttaaagaatggaagaaacttgacaaagctaagaaggtgatggaaaatcagatgtttgaagagaagcaagcggaatctatgcccgaaatcgagaccgaacaagaatataaagagatattcattacacctcatcagatcaagcacataaaggatttcaaggattacaaatatagccatcgggtggataaaaaacccaatcgcgaattgataaatgacacgttgtactcaactaggaaagatgacaagggaaacactcttatagtaaacaacctgaatggactctacgataaagataatgacaagcttaaaaaactcattaataaatctccagagaagttgcttatgtatcaccacgacccccagacgtaccagaaactgaaattgattatggaacaatatggcgatgagaaaaacccgctgtacaagtactatgaggaaaccgggaactatctcactaaatattccaaaaaagacaacggaccggtaatcaaaaaaatcaaatactacggtaacaaactcaacgcacatttggacatcacggacgactacccgaactccagaaacaaagtagttaagctgtctctgaagccgtatagatttgacgtttacctggacaacggcgtttataagttcgtcacagtcaagaatttggacgtcatcaagaaagaaaactactacgaagtgaattccaagtgttacgaggaagcaaaaaagctcaagaagatttctaatcaagctgaatttatcgccagtttctacaataatgatctgataaaaattaacggcgaactctacagagtgatcggggttaacaacgacctcctgaatagaatagaggtgaacatgatagatattacttatagggagtacctcgaaaatatgaatgacaaacgacctccgagaataatcaagacgattgctagtaaaacccagagcattaaaaagtactccacggatatactgggtaatctctacgaggttaagtcaaaaaaacaccctcaaataattaagaagggtaaaagacccgctgccacgaagaaagcggggcaggcgaaaaaaaaaaaaggcagtgaagcctcaggtagcggcagagctgacgcacttgatgatttcgatcttgacatgctgggatcagatgcgttggatgatttcgatcttgacatgcttggttctgatgccctggatgactttgacttggacatgcttggctcagacgcccttgatgacttcgatctggatatgctg

SEQ ID NO: 125 - DNA, 2925 bp, synthetic construct
  misc_feature 1..2925: note = NLS-miniSadCas9v4-NLS-VP64 (optimized 2)
  source 1..2925: mol_type = other DNA
cccaaaaaaaaaagaaaggtgggcatccacggcgtgcccgccgccaagagaaactacatcctgggcctggccatcggcatcacaagcgtgggctacggcatcatcgactacgagacaagagacgtgatcgacgccggcgtgagactgttcaaggaggccaacgtggagaacaacgagggcagaagaagcaagagaggcgctagaagactgaagcggagacgtagacatcgtattcagagagtgaagaagctgctgttcgactacaacctgctgaccgaccactcggagctgagcggaatcaacccctacgaggctagagtgaagggcctgtctcagaagctgagcgaggaggagttcagcgccgccctgctgcacctggccaagagaagaggcgtgcacaacgtgaacgaggtggaggaggacaccggcaacgagctgagcaccaaggaacagatcagcagaaacagcaaggccctggaggagaagtacgtggccgagctgcagctggagagactgaagaaggacggcgaggtgagaggcagcatcaacagattcaagacaagcgactacgtgaaggaggccaagcagctgctgaaggtgcagaaggcctaccatcagctggatcagagcttcatcgacacctacatcgacctgctggagacaagaagaacctactacgagggccccggcgagggcagccccttcggctggaaggacatcaaggagtggtacgagatgctgatgggccactgcacctacttccccgaggagctgagaagcgtgaagtacgcctacaacgccgacctgtacaacgccctgaacgacctgaacaacctcgtgatcacaagagacgagaacgagaagctggagtattatgagaagtttcagatcatcgagaacgtgtttaagcagaaaaagaaacccaccctgaagcagattgccaaggagatcctggtgaacgaggaggacatcaaaggctacagagtgacaagcaccggcaagcccgagttcaccaacctgaaggtgtaccacgacatcaaggacatcaccgctagaaaggaaattatcgagaatgcggaactgctggatcagatagccaaaatccttaccatctatcagagcagcgaggacatccaagaggagctgaccaacctgaactcagaactgacccaagaggagatcgaacagatttccaatctgaaaggctacaccggcacccacaacctgagccttaaagcaataaacctgatcctggacgagctgtggcacaccaacgacaatcagatcgcaatcttcaacagactgaagctggtgcccaagaaggtggacctgtctcagcagaaggagatccccaccaccctggtggacgacttcatcctgagccccgtggtgaagagaagcttcattcagagcatcaaagtgataaacgccattatcaaaaagtacggcctgcccaacgacatcatcatcgagctgggcagcaagagatacgccacaagaggcctgatgaacctgctgagaagctacttcagagtgaataacctagacgttaaggtaaagagcatcaatggcggcttcacaagcttcctgagaagaaagtggaagtttaagaaagagagaaataagggctacaagcaccacgccgaggacgccctgatcatcgccaacgccgacttcatcttcaaggagtggaagaagctggacaaggccaagaaggtgatggagaatcagatgttcgaggagaagcaagccgagagcatgcccgagatcgagaccgagcaagagtacaaggagatcttcatcaccccccatcagatcaagcacatcaaggacttcaaggactacaagtacagccacagagtggacaagaagcccaacagagagctgatcaacgacaccctgtacagcacaagaaaggacgacaagggcaacaccctgatcgtgaataacctgaatggcctgtacgacaaggacaacgacaagctgaagaagttgatcaacaagagccccgagaagctgctgatgtaccaccacgaccctcagacctatcagaagctgaagctgatcatggagcagtacggcgacgagaagaaccccctgtacaagtactacgaggagaccggcaactacctgaccaagtacagcaagaaggacaacggccccgtgattaaaaagatcaagtactacggcaacaagctgaacgcccacctggacatcaccgacgactaccccaacagcagaaacaaggtggtgaagctgagcttaaagccttatagattcgacgtgtacctggacaacggcgtgtacaagttcgtgaccgtgaagaacttggacgtgataaaaaaagaaaactactacgaggtgaacagcaagtgctacgaggaggccaagaagctgaaaaaaataagcaaccaagccgagttcatcgctagcttctacaacaatgacctgatcaagatcaacggcgagctgtacagagtgatcggcgtgaacaacgacttactgaacagaatcgaggtgaacatgatcgacatcacctacagagagtacctggagaacatgaacgacaagagaccccctagaatcatcaagaccatcgctagcaagacacagagcattaagaagtatagcaccgacatcctgggcaacctgtacgaggtgaaatcgaagaagcaccctcagattatcaagaagggtaagagacccgccgccaccaagaaggccggccaagccaagaaaaaaaagggcagcgaggctagcggcagcggcagagccgatgccctggatgacttcgatttggacatgttgggttctgacgccctagatgacttcgacctagatatgctggggagcgatgcgctagatgatttcgatcttgatatgctcgggagcgacgctctagacgacttcgatttggatatgctg

SEQ ID NO: 126 - protein, 773 aa, synthetic construct
  REGION 1..773: note = NLS-miniSadCas9v5-NLS-VP64
MAPKKKRKVGIHGVPAAKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLKRRRRHRILSPVVKRSFIQSIKVINAIIKKYGLPNDIIIELGSKRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKGKRPAATKKAGQAKKKKGSEASGSGRADALDDFDLDMLGSDALDDFDLDMLGSDALDDFDLDMLGSDALDDFDLDML

SEQ ID NO: 127 - DNA, 2313 bp, synthetic construct
  misc_feature 1..2313: note = NLS-miniSadCas9v5-NLS-VP64
  source 1..2313: mol_type = other DNA
ccaaagaagaagcggaaggtcggtatccacggagtcccagcagccaagaggaactacatcctgggcctggccatcggcatcaccagcgtgggctacggcatcatcgactacgagaccagggacgtgatcgacgccggcgtgaggctgttcaaggaggccaacgtggagaacaacgagggcaggaggagcaagaggggcgccaggaggctgaagaggaggaggaggcacaggatccagagggtgaagaagctgctgttcgactacaacctgctgaccgaccacagcgagctgagcggcatcaacccctacgaggccagggtgaagggcctgagccagaagctgagcgaggaggagttcagcgccgccctgctgcacctggccaagaggaggggcgtgcacaacgtgaacgaggtggaggaggacaccggcaacgagctgagcaccaaggagcagatcagcaggaacagcaaggccctggaggagaagtacgtggccgagctgcagctggagaggctgaagaaggacggcgaggtgaggggcagcatcaacaggttcaagaccagcgactacgtgaaggaggccaagcagctgctgaaggtgcagaaggcctaccaccagctggaccagagcttcatcgacacctacatcgacctgctggagaccaggaggacctactacgagggccccggcgagggcagccccttcggctggaaggacatcaaggagtggtacgagatgctgaagaggaggaggaggcacaggatcctgagccccgtggtgaagaggagcttcatccagagcatcaaggtgatcaacgccatcatcaagaagtacggcctgcccaacgacatcatcatcgagctgggcagcaagaggtacgccaccaggggcctgatgaacctgctgaggagctacttcagggtgaacaacctggacgtgaaggtgaagagcatcaacggcggcttcaccagcttcctgaggaggaagtggaagttcaagaaggagaggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccacagggtggacaagaagcccaacaggaagctgatcaacgacaccctgtacagcaccaggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagcaggaacaaggtggtgaagctgagcctgaagccctacaggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtacagggtgatcggcgtgaacaacgacctgctgaacaggatcgaggtgaacatgatcgacatcacctacagggagtacctggagaacatgaacgacaagaggcccccccacatcatcaagaccatcgccagcaagacccagagcatcaagaagtacagcaccgacatcctgggcaacctgtacgaggtgaagagcaagaagcacccccagatcatcaagaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctg

SEQ ID NO: 128 - DNA, 2313 bp, synthetic construct
  misc_feature 1..2313: note = NLS-miniSadCas9v5-NLS-VP64 (optimized 1)
  source 1..2313: mol_type = other DNA
ccaaagaagaagcgcaaagtgggaatacacggggtaccagccgcgaaaagaaattacatcctggggcttgccatagggataacctctgtgggatacgggattattgactatgaaacacgcgatgtcatagatgctggtgtacggcttttcaaagaagcaaatgtagaaaataatgagggccgaagaagtaaacggggagccagacggttgaagagaagacggaggcatcgcatacaaagagttaagaagctcttgtttgattacaatttgcttacggatcactcagaattgtccggaataaatccgtatgaggcgcgggttaagggactgtcccaaaaactgtctgaagaggagtttagtgcagccctccttcatctcgctaaacgccgcggcgtccataatgtcaatgaggtagaagaggacacaggtaacgagctcagtacgaaagagcaaatctctcggaactctaaggccttggaggagaaatatgttgccgaacttcaattggaaaggctgaaaaaagatggagaggtgcggggatctattaatcggttcaaaacgagcgattacgttaaggaggctaaacagttgctgaaggtccagaaagcttaccatcagttggatcaaagtttcatcgatacctacattgatctgttggagacccgaaggacatactacgaaggaccgggagaaggctctcccttcggatggaaagatattaaggagtggtatgagatgttgaagaggcggcggcgccataggatcttgtcaccggtcgttaaaaggtctttcatccaatcaatcaaagttatcaacgctatcataaaaaagtacggattgcctaatgacattataatagaactcggttcaaaacggtacgccaccagaggtctgatgaacttgctcaggtcatacttccgggttaacaatctggacgtcaaagtcaaaagcatcaacggaggattcacatcctttttgcgaagaaaatggaaattcaaaaaggagcgaaacaagggctataagcatcatgctgaagacgcccttataatcgcgaatgcagactttatcttcaaagaatggaagaagttggataaagcaaagaaggttatggagaatcaaatgtttgaggaaaaacaagctgagagcatgccagaaatcgaaacagagcaagaatataaagagatcttcatcaccccgcaccaaataaagcacataaaggatttcaaggattataagtactcccacagggtagataaaaagccgaaccgaaaattgataaacgatacattgtactctacgcgcaaggacgataaagggaacacacttatagtaaacaatctcaacggtctttacgacaaggacaacgataagttgaaaaagctcatcaataaatctcccgagaagcttctgatgtaccaccacgacccgcagacctatcaaaagttgaaattgattatggagcaatacggggacgagaaaaaccccctgtacaagtactacgaggaaacagggaactaccttacgaagtacagtaagaaagataatgggcctgtgattaaaaaaataaaatactatggaaacaaactcaatgcgcatttggacataacagacgactaccccaattcaaggaacaaagtcgtcaaattgtctcttaagccatatcggttcgacgtgtacctggacaacggagtctacaaattcgttaccgttaagaaccttgacgtaatcaaaaaagaaaactattacgaggttaactcaaagtgttatgaagaggccaagaagcttaaaaagatatcaaatcaagcagaatttatcgcaagcttctataagaatgacttgataaagatcaatggggagttgtatcgggttattggagtgaacaatgatctgcttaatagaatagaggtcaacatgattgatattacctatcgggaatacttggagaacatgaatgacaaaagacccccacacatcataaagacaattgcatcaaagacgcagtcaattaaaaaatattccactgacattctcggcaatctgtatgaagtcaaatctaaaaagcacccacaaatcatcaaaaagggcaagcggcctgccgcgaccaaaaaagctggccaagccaaaaagaaaaagggctccgaagcttctggtagcggccgagcggacgccttggatgacttcgatttggatatgctgggtagtgacgcgctggacgactttgacctcgatatgcttggcagtgacgccctcgacgacttcgacctggacatgctgggttcagatgctctggacgattttgacctcgacatgctg

SEQ ID NO: 129 - DNA, 2313 bp, synthetic construct
  misc_feature 1..2313: note = NLS-miniSadCas9v5-NLS-VP64 (optimized 2)
  source 1..2313: mol_type = other DNA
cccaagaaaaaaagaaaagtgggcatccacggcgtgcccgccgccaagagaaactacatcctgggcctggccatcggcatcacaagcgtgggctacggcatcatcgactacgagacaagagacgtgatcgacgccggcgtgagactgttcaaggaggccaacgtggagaacaacgagggcagaagaagcaagagaggcgctagaagattgaaacgccggaggagacatcggattcagagagtgaagaagctgctgttcgactacaacctgctgaccgaccacagcgagctgagcggcatcaacccctacgaggctagagtgaagggcctgtctcagaagctgagcgaggaggagttcagcgccgccctgctgcacctggccaagagaagaggcgtgcacaacgtgaacgaggtggaggaggacaccggcaacgagctgagcaccaaggagcagatcagcagaaacagcaaggccctggaggagaagtacgtggccgagctgcagctggagagactgaagaaggacggcgaggtgagaggcagcatcaacagattcaagacaagcgactacgtgaaggaggccaagcagctgctgaaggtgcagaaggcctaccatcagctggatcagagcttcatcgacacctacatcgacctgctggagacaagaagaacctactacgagggccccggcgagggcagccccttcggctggaaggacatcaaggagtggtacgagatgcttaagcgacgccgtcggcacagaatcttaagccccgtggtgaagagaagcttcatccaaagcataaaggtgatcaacgccattataaagaagtacgggctgcccaacgacatcatcatcgagctgggcagcaagagatacgccacaagaggcctgatgaacctgctgagaagctacttcagagttaacaacctagacgtgaaagttaagagcataaacggcggcttcacaagcttcctgagaagaaagtggaagttcaagaaggagagaaataagggctacaagcaccacgccgaggacgccctgatcatcgccaacgccgacttcatcttcaaggagtggaagaagctggacaaggccaagaaggtgatggagaatcagatgttcgaggagaagcaagccgagagcatgcccgagatcgagaccgagcaagagtacaaggagatcttcatcaccccccatcagatcaagcacatcaaggacttcaaggactacaagtacagccacagagtggacaagaagcccaacagaaagctcatcaatgacaccctgtacagcacaagaaaggacgacaagggcaacaccctgatcgtgaacaacctgaacggcctgtacgacaaggacaacgacaagctcaagaaactgatcaacaagagccccgagaagctgctgatgtaccaccacgaccctcagacctatcagaagctgaagctgatcatggagcagtacggcgacgagaagaaccccctgtacaagtactacgaggagaccggcaactacctgaccaagtacagcaagaaggacaacggccccgtgatcaagaagatcaaatactacggcaacaagctgaacgcccacctggacatcaccgacgactaccccaacagcagaaacaaggtggtgaagctgagcctgaagccctacagattcgacgtgtacctggacaacggcgtgtacaagttcgtgaccgtgaagaacctggatgttatcaaaaaggagaattactacgaggtgaacagcaagtgctacgaggaggccaagaaactgaagaaaatctcgaaccaagccgagttcatcgctagcttctacaagaacgacctgatcaagatcaacggcgagctgtacagagtgatcggcgtgaacaacgacctgctgaacagaatcgaggtgaacatgatcgacatcacctacagagagtacctggagaacatgaacgacaagagacccccccacatcatcaagaccatcgctagcaagacacagtcgatcaaaaagtacagcaccgacatcctgggcaacctgtacgaggtaaaaagcaagaagcaccctcagatcatcaagaaaggcaaaagacccgccgccaccaagaaggccggccaagccaagaagaagaagggcagcgaggctagcggcagcggcagagccgatgccctggacgacttcgatttggacatgctgggtagcgacgccctagatgacttcgacctggatatgctggggagcgatgcgctagatgatttcgatcttgatatgctcgggagcgacgctctagacgatttcgacttagatatgctg

SEQ ID NO: 130-131 - skipped (2 entries)

SEQ ID NO: 132 - RNA, 13 nt, synthetic construct
  misc_feature 1..13: note = Kozak sequence
  source 1..13: mol_type = other RNA
gccgccaccatgg

SEQ ID NO: 133-134 - skipped (2 entries)

SEQ ID NO: 135 - DNA, 251 bp, synthetic construct
  misc_feature 1..251: note = woodchuck hepatitis virus post-transcriptional element 3
  source 1..251: mol_type = other DNA
gataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtt

SEQ ID NO: 136 - DNA, 82 bp, synthetic construct
  misc_feature 1..82: note = SV40 polyA signal
  source 1..82: mol_type = other DNA
aacttgtttattgcagcttataatggttacaaataaagcaatagcatcacaaatttcacaaataaagcatttttttcactgc

SEQ ID NO: 137 - DNA, 5377 bp, synthetic construct
  misc_feature 1..5377: note = pAAV-SCN1A_SE1 expression cassette
  source 1..5377: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctagcctgcaagtcgaggagcgcagccttccagaagcagagcgcggcgccttaagctgcagaagttggtcgtgaggcactgggcacgtaagtatcaaggttacaagacaggtttaaggagaccaatagaaactgggcttgtcgagacagagaagactcttgcgtttctgataggcacctattggtcttactgacatccactttgcctttctctccacagcgagctcgctagcgcgatcgctgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttctggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 138 - DNA, 4765 bp, synthetic construct
  misc_feature 1..4765: note = pAAV-SCN1A_SE1_mini-SadCas9-v2 expression cassette
  source 1..4765: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctagcctgcaagtcgaggagcgcagccttccagaagcagagcgcggcgccttaagctgcagaagttggtcgtgaggcactgggcacgtaagtatcaaggttacaagacaggtttaaggagaccaatagaaactgggcttgtcgagacagagaagactcttgcgtttctgataggcacctattggtcttactgacatccactttgcctttctctccacagcgagctcgctagcgcgatcgctgccaccatggccccaaagaagaagcggaaggtcggtatccacggagtcccagcagccaagaggaactacatcctgggcctggccatcggcatcaccagcgtgggctacggcatcatcgactacgagaccagggacgtgatcgacgccggcgtgaggctgttcaaggaggccaacgtggagaacaacgagggcaggaggagcaagaggggcgccaggaggctgaagaggaggaggaggcacaggatccagagggtgaagaagctgctgttcgactacaacctgctgaccgaccacagcgagctgagcggcatcaacccctacgaggccagggtgaagggcctgagccagaagctgagcgaggaggagttcagcgccgccctgctgcacctggccaagaggaggggcgtgcacaacgtgaacgaggtggaggaggacaccggcaacgagctgagcaccaaggagcagatcagcaggaacagcaaggccctggaggagaagtacgtggccgagctgcagctggagaggctgaagaaggacggcgaggtgaggggcagcatcaacaggttcaagaccagcgactacgtgaaggaggccaagcagctgctgaaggtgcagaaggcctaccaccagctggaccagagcttcatcgacacctacatcgacctgctggagaccaggaggacctactacgagggccccggcgagggcagccccttcggctggaaggacatcaaggagtggtacgagatgctgaagaggaggaggaggcacaggatcctgagccccgtggtgaagaggagcttcatccagagcatcaaggtgatcaacgccatcatcaagaagtacggcctgcccaacgacatcatcatcgagctggccagggagaagaacagcaaggacgcccagaagatgatcaacgagatgcagaagaggaacaggcagaccaacgagaggatcgaggagatcatcaggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccaggagcgtgagcttcgacaacagcttcaacaacaaggtgctggtgaagcaggaggaggccagcaagaagggcaacaggacccccttccagtacctgagcagcagcgacagcaagatcagctacgagaccttcaagaagcacatcctgaacctggccaagggcaagggcaggatcagcaagaccaagaaggagtacctgctggaggagagggacatcaacaggttcagcgtgcagaaggacttcatcaacaggaacctggtggacaccaggtacgccaccaggggcctgatgaacctgctgaggagctacttcagggtgaacaacctggacgtgaaggtgaagagcatcaacggcggcttcaccagcttcctgaggaggaagtggaagttcaagaaggagaggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccacagggtggacaagaagcccaacaggaagctgatcaacgacaccctgtacagcaccaggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagcaggaacaaggtggtgaagctgagcctgaagccctacaggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtacagggtgatcggcgtgaacaacgacctgctgaacaggatcgaggtgaacatgatcgacatcacctacagggagtacctggagaacatgaacgacaagaggcccccccacatcatcaagaccatcgccagcaagacccagagcatcaagaagtacagcaccgacatcctgggcaacctgtacgaggtgaagagcaagaagcacccccagatcatcaagaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttctggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 139 - DNA, 4873 bp, synthetic construct
  misc_feature 1..4873: note = pAAV-SCN1A_SE1_mini-SadCas9-v4 expression cassette
  source 1..4873: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctagcctgcaagtcgaggagcgcagccttccagaagcagagcgcggcgccttaagctgcagaagttggtcgtgaggcactgggcacgtaagtatcaaggttacaagacaggtttaaggagaccaatagaaactgggcttgtcgagacagagaagactcttgcgtttctgataggcacctattggtcttactgacatccactttgcctttctctccacagcgagctcgctagcgcgatcgctgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctgggcagcaagagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttctggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 140 - DNA, 4261 bp, synthetic construct
  misc_feature 1..4261: note = pAAV-SCN1A_SE1_mini-SadCas9-v5 expression cassette
  source 1..4261: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctagcctgcaagtcgaggagcgcagccttccagaagcagagcgcggcgccttaagctgcagaagttggtcgtgaggcactgggcacgtaagtatcaaggttacaagacaggtttaaggagaccaatagaaactgggcttgtcgagacagagaagactcttgcgtttctgataggcacctattggtcttactgacatccactttgcctttctctccacagcgagctcgctagcgcgatcgctgccaccatggccccaaagaagaagcggaaggtcggtatccacggagtcccagcagccaagaggaactacatcctgggcctggccatcggcatcaccagcgtgggctacggcatcatcgactacgagaccagggacgtgatcgacgccggcgtgaggctgttcaaggaggccaacgtggagaacaacgagggcaggaggagcaagaggggcgccaggaggctgaagaggaggaggaggcacaggatccagagggtgaagaagctgctgttcgactacaacctgctgaccgaccacagcgagctgagcggcatcaacccctacgaggccagggtgaagggcctgagccagaagctgagcgaggaggagttcagcgccgccctgctgcacctggccaagaggaggggcgtgcacaacgtgaacgaggtggaggaggacaccggcaacgagctgagcaccaaggagcagatcagcaggaacagcaaggccctggaggagaagtacgtggccgagctgcagctggagaggctgaagaaggacggcgaggtgaggggcagcatcaacaggttcaagaccagcgactacgtgaaggaggccaagcagctgctgaaggtgcagaaggcctaccaccagctggaccagagcttcatcgacacctacatcgacctgctggagaccaggaggacctactacgagggccccggcgagggcagccccttcggctggaaggacatcaaggagtggtacgagatgctgaagaggaggaggaggcacaggatcctgagccccgtggtgaagaggagcttcatccagagcatcaaggtgatcaacgccatcatcaagaagtacggcctgcccaacgacatcatcatcgagctgggcagcaagaggtacgccaccaggggcctgatgaacctgctgaggagctacttcagggtgaacaacctggacgtgaaggtgaagagcatcaacggcggcttcaccagcttcctgaggaggaagtggaagttcaagaaggagaggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccacagggtggacaagaagcccaacaggaagctgatcaacgacaccctgtacagcaccaggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagcaggaacaaggtggtgaagctgagcctgaagccctacaggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtacagggtgatcggcgtgaacaacgacctgctgaacaggatcgaggtgaacatgatcgacatcacctacagggagtacctggagaacatgaacgacaagaggcccccccacatcatcaagaccatcgccagcaagacccagagcatcaagaagtacagcaccgacatcctgggcaacctgtacgaggtgaagagcaagaagcacccccagatcatcaagaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttctggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 141 - DNA, 5126 bp, synthetic construct
  misc_feature 1..5126: note = pAAV-SCN1A_SE2 expression cassette
  source 1..5126: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctagcctgcaagtcgaggagcgcagccttccagaagcagagcgcggcgccttaagctgcagaagttggtcgtgaggcactgggcacgtaagtatcaaggttacaagacaggtttaaggagaccaatagaaactgggcttgtcgagacagagaagactcttgcgtttctgataggcacctattggtcttactgacatccactttgcctttctctccacagcgagctcgctagcgcgatcgctgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagtggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 142 - DNA, 4514 bp, synthetic construct
  misc_feature 1..4514: note = pAAV-SCN1A_SE2_mini-SadCas9-v2 expression cassette
  source 1..4514: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctagcctgcaagtcgaggagcgcagccttccagaagcagagcgcggcgccttaagctgcagaagttggtcgtgaggcactgggcacgtaagtatcaaggttacaagacaggtttaaggagaccaatagaaactgggcttgtcgagacagagaagactcttgcgtttctgataggcacctattggtcttactgacatccactttgcctttctctccacagcgagctcgctagcgcgatcgctgccaccatggccccaaagaagaagcggaaggtcggtatccacggagtcccagcagccaagaggaactacatcctgggcctggccatcggcatcaccagcgtgggctacggcatcatcgactacgagaccagggacgtgatcgacgccggcgtgaggctgttcaaggaggccaacgtggagaacaacgagggcaggaggagcaagaggggcgccaggaggctgaagaggaggaggaggcacaggatccagagggtgaagaagctgctgttcgactacaacctgctgaccgaccacagcgagctgagcggcatcaacccctacgaggccagggtgaagggcctgagccagaagctgagcgaggaggagttcagcgccgccctgctgcacctggccaagaggaggggcgtgcacaacgtgaacgaggtggaggaggacaccggcaacgagctgagcaccaaggagcagatcagcaggaacagcaaggccctggaggagaagtacgtggccgagctgcagctggagaggctgaagaaggacggcgaggtgaggggcagcatcaacaggttcaagaccagcgactacgtgaaggaggccaagcagctgctgaaggtgcagaaggcctaccaccagctggaccagagcttcatcgacacctacatcgacctgctggagaccaggaggacctactacgagggccccggcgagggcagccccttcggctggaaggacatcaaggagtggtacgagatgctgaagaggaggaggaggcacaggatcctgagccccgtggtgaagaggagcttcatccagagcatcaaggtgatcaacgccatcatcaagaagtacggcctgcccaacgacatcatcatcgagctggccagggagaagaacagcaaggacgcccagaagatgatcaacgagatgcagaagaggaacaggcagaccaacgagaggatcgaggagatcatcaggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccaggagcgtgagcttcgacaacagcttcaacaacaaggtgctggtgaagcaggaggaggccagcaagaagggcaacaggacccccttccagtacctgagcagcagcgacagcaagatcagctacgagaccttcaagaagcacatcctgaacctggccaagggcaagggcaggatcagcaagaccaagaaggagtacctgctggaggagagggacatcaacaggttcagcgtgcagaaggacttcatcaacaggaacctggtggacaccaggtacgccaccaggggcctgatgaacctgctgaggagctacttcagggtgaacaacctggacgtgaaggtgaagagcatcaacggcggcttcaccagcttcctgaggaggaagtggaagttcaagaaggagaggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccacagggtggacaagaagcccaacaggaagctgatcaacgacaccctgtacagcaccaggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagcaggaacaaggtggtgaagctgagcctgaagccctacaggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtacagggtgatcggcgtgaacaacgacctgctgaacaggatcgaggtgaacatgatcgacatcacctacagggagtacctggagaacatgaacgacaagaggcccccccacatcatcaagaccatcgccagcaagacccagagcatcaagaagtacagcaccgacatcctgggcaacctgtacgaggtgaagagcaagaagcacccccagatcatcaagaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagtggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 143 - DNA, 4622 bp, synthetic construct
  misc_feature 1..4622: note = pAAV-SCN1A_SE2_mini-SadCas9-v4 expression cassette
  source 1..4622: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctagcctgcaagtcgaggagcgcagccttccagaagcagagcgcggcgccttaagctgcagaagttggtcgtgaggcactgggcacgtaagtatcaaggttacaagacaggtttaaggagaccaatagaaactgggcttgtcgagacagagaagactcttgcgtttctgataggcacctattggtcttactgacatccactttgcctttctctccacagcgagctcgctagcgcgatcgctgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctgggcagcaagagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagtggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 144 - DNA, 4010 bp, synthetic construct
  misc_feature 1..4010: note = pAAV-SCN1A_SE2_mini-SadCas9-v5 expression cassette
  source 1..4010: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctagcctgcaagtcgaggagcgcagccttccagaagcagagcgcggcgccttaagctgcagaagttggtcgtgaggcactgggcacgtaagtatcaaggttacaagacaggtttaaggagaccaatagaaactgggcttgtcgagacagagaagactcttgcgtttctgataggcacctattggtcttactgacatccactttgcctttctctccacagcgagctcgctagcgcgatcgctgccaccatggccccaaagaagaagcggaaggtcggtatccacggagtcccagcagccaagaggaactacatcctgggcctggccatcggcatcaccagcgtgggctacggcatcatcgactacgagaccagggacgtgatcgacgccggcgtgaggctgttcaaggaggccaacgtggagaacaacgagggcaggaggagcaagaggggcgccaggaggctgaagaggaggaggaggcacaggatccagagggtgaagaagctgctgttcgactacaacctgctgaccgaccacagcgagctgagcggcatcaacccctacgaggccagggtgaagggcctgagccagaagctgagcgaggaggagttcagcgccgccctgctgcacctggccaagaggaggggcgtgcacaacgtgaacgaggtggaggaggacaccggcaacgagctgagcaccaaggagcagatcagcaggaacagcaaggccctggaggagaagtacgtggccgagctgcagctggagaggctgaagaaggacggcgaggtgaggggcagcatcaacaggttcaagaccagcgactacgtgaaggaggccaagcagctgctgaaggtgcagaaggcctaccaccagctggaccagagcttcatcgacacctacatcgacctgctggagaccaggaggacctactacgagggccccggcgagggcagccccttcggctggaaggacatcaaggagtggtacgagatgctgaagaggaggaggaggcacaggatcctgagccccgtggtgaagaggagcttcatccagagcatcaaggtgatcaacgccatcatcaagaagtacggcctgcccaacgacatcatcatcgagctgggcagcaagaggtacgccaccaggggcctgatgaacctgctgaggagctacttcagggtgaacaacctggacgtgaaggtgaagagcatcaacggcggcttcaccagcttcctgaggaggaagtggaagttcaagaaggagaggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccacagggtggacaagaagcccaacaggaagctgatcaacgacaccctgtacagcaccaggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagcaggaacaaggtggtgaagctgagcctgaagccctacaggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtacagggtgatcggcgtgaacaacgacctgctgaacaggatcgaggtgaacatgatcgacatcacctacagggagtacctggagaacatgaacgacaagaggcccccccacatcatcaagaccatcgccagcaagacccagagcatcaagaagtacagcaccgacatcctgggcaacctgtacgaggtgaagagcaagaagcacccccagatcatcaagaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagtggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 145 - DNA, 5159 bp, synthetic construct
  misc_feature 1..5159: note = pAAV-SCN1A_SE3 expression cassette
  source 1..5159: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctgagctcgctagcgcgatcgctgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttctggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 146 - DNA, 4547 bp, synthetic construct
  misc_feature 1..4547: note = pAAV-SCN1A_SE3_mini-SadCas9-v2 expression cassette
  source 1..4547: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctgagctcgctagcgcgatcgctgccaccatggccccaaagaagaagcggaaggtcggtatccacggagtcccagcagccaagaggaactacatcctgggcctggccatcggcatcaccagcgtgggctacggcatcatcgactacgagaccagggacgtgatcgacgccggcgtgaggctgttcaaggaggccaacgtggagaacaacgagggcaggaggagcaagaggggcgccaggaggctgaagaggaggaggaggcacaggatccagagggtgaagaagctgctgttcgactacaacctgctgaccgaccacagcgagctgagcggcatcaacccctacgaggccagggtgaagggcctgagccagaagctgagcgaggaggagttcagcgccgccctgctgcacctggccaagaggaggggcgtgcacaacgtgaacgaggtggaggaggacaccggcaacgagctgagcaccaaggagcagatcagcaggaacagcaaggccctggaggagaagtacgtggccgagctgcagctggagaggctgaagaaggacggcgaggtgaggggcagcatcaacaggttcaagaccagcgactacgtgaaggaggccaagcagctgctgaaggtgcagaaggcctaccaccagctggaccagagcttcatcgacacctacatcgacctgctggagaccaggaggacctactacgagggccccggcgagggcagccccttcggctggaaggacatcaaggagtggtacgagatgctgaagaggaggaggaggcacaggatcctgagccccgtggtgaagaggagcttcatccagagcatcaaggtgatcaacgccatcatcaagaagtacggcctgcccaacgacatcatcatcgagctggccagggagaagaacagcaaggacgcccagaagatgatcaacgagatgcagaagaggaacaggcagaccaacgagaggatcgaggagatcatcaggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccaggagcgtgagcttcgacaacagcttcaacaacaaggtgctggtgaagcaggaggaggccagcaagaagggcaacaggacccccttccagtacctgagcagcagcgacagcaagatcagctacgagaccttcaagaagcacatcctgaacctggccaagggcaagggcaggatcagcaagaccaagaaggagtacctgctggaggagagggacatcaacaggttcagcgtgcagaaggacttcatcaacaggaacctggtggacaccaggtacgccaccaggggcctgatgaacctgctgaggagctacttcagggtgaacaacctggacgtgaaggtgaagagcatcaacggcggcttcaccagcttcctgaggaggaagtggaagttcaagaaggagaggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccacagggtggacaagaagcccaacaggaagctgatcaacgacaccctgtacagcaccaggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagcaggaacaaggtggtgaagctgagcctgaagccctacaggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtacagggtgatcggcgtgaacaacgacctgctgaacaggatcgaggtgaacatgatcgacatcacctacagggagtacctggagaacatgaacgacaagaggcccccccacatcatcaagaccatcgccagcaagacccagagcatcaagaagtacagcaccgacatcctgggcaacctgtacgaggtgaagagcaagaagcacccccagatcatcaagaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttctggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 147 - DNA, 4655 bp, synthetic construct
  misc_feature 1..4655: note = pAAV-SCN1A_SE3_mini-SadCas9-v4 expression cassette
  source 1..4655: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctgagctcgctagcgcgatcgctgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctgggcagcaagagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttctggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 148 - DNA, 4043 bp, synthetic construct
  misc_feature 1..4043: note = pAAV-SCN1A_SE3_mini-SadCas9-v5 expression cassette
  source 1..4043: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctgagctcgctagcgcgatcgctgccaccatggccccaaagaagaagcggaaggtcggtatccacggagtcccagcagccaagaggaactacatcctgggcctggccatcggcatcaccagcgtgggctacggcatcatcgactacgagaccagggacgtgatcgacgccggcgtgaggctgttcaaggaggccaacgtggagaacaacgagggcaggaggagcaagaggggcgccaggaggctgaagaggaggaggaggcacaggatccagagggtgaagaagctgctgttcgactacaacctgctgaccgaccacagcgagctgagcggcatcaacccctacgaggccagggtgaagggcctgagccagaagctgagcgaggaggagttcagcgccgccctgctgcacctggccaagaggaggggcgtgcacaacgtgaacgaggtggaggaggacaccggcaacgagctgagcaccaaggagcagatcagcaggaacagcaaggccctggaggagaagtacgtggccgagctgcagctggagaggctgaagaaggacggcgaggtgaggggcagcatcaacaggttcaagaccagcgactacgtgaaggaggccaagcagctgctgaaggtgcagaaggcctaccaccagctggaccagagcttcatcgacacctacatcgacctgctggagaccaggaggacctactacgagggccccggcgagggcagccccttcggctggaaggacatcaaggagtggtacgagatgctgaagaggaggaggaggcacaggatcctgagccccgtggtgaagaggagcttcatccagagcatcaaggtgatcaacgccatcatcaagaagtacggcctgcccaacgacatcatcatcgagctgggcagcaagaggtacgccaccaggggcctgatgaacctgctgaggagctacttcagggtgaacaacctggacgtgaaggtgaagagcatcaacggcggcttcaccagcttcctgaggaggaagtggaagttcaagaaggagaggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccacagggtggacaagaagcccaacaggaagctgatcaacgacaccctgtacagcaccaggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagcaggaacaaggtggtgaagctgagcctgaagccctacaggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtacagggtgatcggcgtgaacaacgacctgctgaacaggatcgaggtgaacatgatcgacatcacctacagggagtacctggagaacatgaacgacaagaggcccccccacatcatcaagaccatcgccagcaagacccagagcatcaagaagtacagcaccgacatcctgggcaacctgtacgaggtgaagagcaagaagcacccccagatcatcaagaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgttctggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 149 - DNA, 4908 bp, synthetic construct
  misc_feature 1..4908: note = pAAV-SCN1A_SE4 expression cassette
  source 1..4908: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctgagctcgctagcgcgatcgctgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagtggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 150 - DNA, 4296 bp, synthetic construct
  misc_feature 1..4296: note = pAAV-SCN1A_SE4_mini-SadCas9-v2 expression cassette
  source 1..4296: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctgagctcgctagcgcgatcgctgccaccatggccccaaagaagaagcggaaggtcggtatccacggagtcccagcagccaagaggaactacatcctgggcctggccatcggcatcaccagcgtgggctacggcatcatcgactacgagaccagggacgtgatcgacgccggcgtgaggctgttcaaggaggccaacgtggagaacaacgagggcaggaggagcaagaggggcgccaggaggctgaagaggaggaggaggcacaggatccagagggtgaagaagctgctgttcgactacaacctgctgaccgaccacagcgagctgagcggcatcaacccctacgaggccagggtgaagggcctgagccagaagctgagcgaggaggagttcagcgccgccctgctgcacctggccaagaggaggggcgtgcacaacgtgaacgaggtggaggaggacaccggcaacgagctgagcaccaaggagcagatcagcaggaacagcaaggccctggaggagaagtacgtggccgagctgcagctggagaggctgaagaaggacggcgaggtgaggggcagcatcaacaggttcaagaccagcgactacgtgaaggaggccaagcagctgctgaaggtgcagaaggcctaccaccagctggaccagagcttcatcgacacctacatcgacctgctggagaccaggaggacctactacgagggccccggcgagggcagccccttcggctggaaggacatcaaggagtggtacgagatgctgaagaggaggaggaggcacaggatcctgagccccgtggtgaagaggagcttcatccagagcatcaaggtgatcaacgccatcatcaagaagtacggcctgcccaacgacatcatcatcgagctggccagggagaagaacagcaaggacgcccagaagatgatcaacgagatgcagaagaggaacaggcagaccaacgagaggatcgaggagatcatcaggaccaccggcaaggagaacgccaagtacctgatcgagaagatcaagctgcacgacatgcaggagggcaagtgcctgtacagcctggaggccatccccctggaggacctgctgaacaaccccttcaactacgaggtggaccacatcatccccaggagcgtgagcttcgacaacagcttcaacaacaaggtgctggtgaagcaggaggaggccagcaagaagggcaacaggacccccttccagtacctgagcagcagcgacagcaagatcagctacgagaccttcaagaagcacatcctgaacctggccaagggcaagggcaggatcagcaagaccaagaaggagtacctgctggaggagagggacatcaacaggttcagcgtgcagaaggacttcatcaacaggaacctggtggacaccaggtacgccaccaggggcctgatgaacctgctgaggagctacttcagggtgaacaacctggacgtgaaggtgaagagcatcaacggcggcttcaccagcttcctgaggaggaagtggaagttcaagaaggagaggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccacagggtggacaagaagcccaacaggaagctgatcaacgacaccctgtacagcaccaggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagcaggaacaaggtggtgaagctgagcctgaagccctacaggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtacagggtgatcggcgtgaacaacgacctgctgaacaggatcgaggtgaacatgatcgacatcacctacagggagtacctggagaacatgaacgacaagaggcccccccacatcatcaagaccatcgccagcaagacccagagcatcaagaagtacagcaccgacatcctgggcaacctgtacgaggtgaagagcaagaagcacccccagatcatcaagaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagtggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 151 - DNA, 4404 bp, synthetic construct
  misc_feature 1..4404: note = pAAV-SCN1A_SE4_mini-SadCas9-v4 expression cassette
  source 1..4404: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctgagctcgctagcgcgatcgctgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctgggcagcaagagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagtggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 152 - DNA, 3792 bp, synthetic construct
  misc_feature 1..3792: note = pAAV-SCN1A_SE4_mini-SadCas9-v5 expression cassette
  source 1..3792: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactagaatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctgagctcgctagcgcgatcgctgccaccatggccccaaagaagaagcggaaggtcggtatccacggagtcccagcagccaagaggaactacatcctgggcctggccatcggcatcaccagcgtgggctacggcatcatcgactacgagaccagggacgtgatcgacgccggcgtgaggctgttcaaggaggccaacgtggagaacaacgagggcaggaggagcaagaggggcgccaggaggctgaagaggaggaggaggcacaggatccagagggtgaagaagctgctgttcgactacaacctgctgaccgaccacagcgagctgagcggcatcaacccctacgaggccagggtgaagggcctgagccagaagctgagcgaggaggagttcagcgccgccctgctgcacctggccaagaggaggggcgtgcacaacgtgaacgaggtggaggaggacaccggcaacgagctgagcaccaaggagcagatcagcaggaacagcaaggccctggaggagaagtacgtggccgagctgcagctggagaggctgaagaaggacggcgaggtgaggggcagcatcaacaggttcaagaccagcgactacgtgaaggaggccaagcagctgctgaaggtgcagaaggcctaccaccagctggaccagagcttcatcgacacctacatcgacctgctggagaccaggaggacctactacgagggccccggcgagggcagccccttcggctggaaggacatcaaggagtggtacgagatgctgaagaggaggaggaggcacaggatcctgagccccgtggtgaagaggagcttcatccagagcatcaaggtgatcaacgccatcatcaagaagtacggcctgcccaacgacatcatcatcgagctgggcagcaagaggtacgccaccaggggcctgatgaacctgctgaggagctacttcagggtgaacaacctggacgtgaaggtgaagagcatcaacggcggcttcaccagcttcctgaggaggaagtggaagttcaagaaggagaggaacaagggctacaagcaccacgccgaggacgccctgatcatcgccaacgccgacttcatcttcaaggagtggaagaagctggacaaggccaagaaggtgatggagaaccagatgttcgaggagaagcaggccgagagcatgcccgagatcgagaccgagcaggagtacaaggagatcttcatcaccccccaccagatcaagcacatcaaggacttcaaggactacaagtacagccacagggtggacaagaagcccaacaggaagctgatcaacgacaccctgtacagcaccaggaaggacgacaagggcaacaccctgatcgtgaacaacctgaacggcctgtacgacaaggacaacgacaagctgaagaagctgatcaacaagagccccgagaagctgctgatgtaccaccacgacccccagacctaccagaagctgaagctgatcatggagcagtacggcgacgagaagaaccccctgtacaagtactacgaggagaccggcaactacctgaccaagtacagcaagaaggacaacggccccgtgatcaagaagatcaagtactacggcaacaagctgaacgcccacctggacatcaccgacgactaccccaacagcaggaacaaggtggtgaagctgagcctgaagccctacaggttcgacgtgtacctggacaacggcgtgtacaagttcgtgaccgtgaagaacctggacgtgatcaagaaggagaactactacgaggtgaacagcaagtgctacgaggaggccaagaagctgaagaagatcagcaaccaggccgagttcatcgccagcttctacaagaacgacctgatcaagatcaacggcgagctgtacagggtgatcggcgtgaacaacgacctgctgaacaggatcgaggtgaacatgatcgacatcacctacagggagtacctggagaacatgaacgacaagaggcccccccacatcatcaagaccatcgccagcaagacccagagcatcaagaagtacagcaccgacatcctgggcaacctgtacgaggtgaagagcaagaagcacccccagatcatcaagaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagtggatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 153 - DNA, 4807 bp, synthetic construct
  misc_feature 1..4807: note = pAAV-SCN1A_SE5 expression cassette
  source 1..4807: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcgtcgactagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgagcgctcgcaggcaggctgggagtttgcagttatagtactctggaaacagaatctactataacaaggcaaaatgccgtgtttatctcgtcaacttgttggcgagattttttttctagatactaggacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctcgctcgctagcgcgatcgctgccaccatggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgtgaatcgaattccgctcgagctgatcaacttgtttattgcagcttataatggttacaaataaagcaatagcatcacaaatttcacaaataaagcatttttttcactgcgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 154 - protein, 2009 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 155 - DNA, 6030 bp, Homo sapiens
  source 1..6030: mol_type = genomic DNA
atggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataa

SEQ ID NO: 156 - protein, 1052 aa, synthetic construct
  REGION 1..1052: note = Sa dCas9
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 157 - protein, 848 aa, synthetic construct
  REGION 1..848: note = mini-Sadcas9-v2-del234-444
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLKRRRRHRILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 158 - protein, 884 aa, synthetic construct
  REGION 1..884: note = mini-Sadcas9-vp64-v4-del479-649
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELGSKRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 159 - protein, 680 aa, synthetic construct
  REGION 1..680: note = mini-Sadcas9-v5-del234-444,479-649
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLKRRRRHRILSPVVKRSFIQSIKVINAIIKKYGLPNDIIIELGSKRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 160 - DNA, 21 bp, synthetic construct
  source 1..21: mol_type = other DNA
caactcctgaaccatcacatc

SEQ ID NO: 161 - DNA, 21 bp, synthetic construct
  source 1..21: mol_type = other DNA
aaaggaaccagaaggaagcgg

(19) United States
(12) Patent Application Publication
(10) Pub. No.: US 2025/0082780 A1
(43) Pub. Date: Mar. 13, 2025

(54) COMPOSITIONS FOR MODULATING EXPRESSION OF SODIUM VOLTAGE-GATED CHANNEL ALPHA SUBUNIT 1 AND USES THEREOF

(71) Applicant: Regel Therapeutics, Inc., Cambridge, MA (US)

(72) Inventors: Jordane DIMIDSCHSTEIN, Boston, MA (US); Navneet MATHARU, San Francisco, CA (US)

(21) Appl. No.: 18/725,214

(22) PCT Filed: Dec. 28, 2022
(86) PCT No.: PCT/US2022/082447

§ 371 Date: Jun. 28, 2024

Related U.S. Application Data
(60) Provisional application No. 63/294,967, filed on Dec. 30, 2021.

(51) Int. Cl. A61K 48/00; C12N 9/22; C12N 15/11; C12N 15/86

(52) U.S. Cl. CPC A61K 48/0058; C12N 9/22; C12N 15/11; C12N 15/86; C12N 2310/20; C12N 2750/14143

(57) ABSTRACT

Provided herein are compositions and methods for increasing expression of the sodium voltage-gated channel alpha subunit 1 (SCN1A)gene in SCN1A-expressing cells. Further provided herein are uses of such compositions to treat disorders associated with SCN1A expression deficiency, such as Dravet syndrome.

### CROSS REFERENCE TO RELATED APPLICATIONS

This application claims the benefit of U.S. Provisional Patent Application No. 63/294,967, filed Dec. 30, 2021, the disclosure of which is hereby incorporated by reference in its entirety.

### REFERENCE TO AN ELECTRONIC SEQUENCE LISTING

The contents of the electronic sequence listing (REGT_001_01WO_SeqList_ST26.xml; Size: 366,590 bytes; and Date of Creation: Dec. 22, 2022) are herein incorporated by reference in their entirety.

### TECHNICAL FIELD

The disclosure relates to gene expression modulation and methods of using the same.

### BACKGROUND

Dravet syndrome (DS) is a pharmaco-resistant infantile epilepsy that causes cognitive impairment and is lethal in about 20% of patients by age 25 years. Heterozygous loss-of-function mutations in the sodium voltage-gated channel alpha subunit 1 (SCN1A) gene, which encodes the alpha subunit of sodium channel Nav1.1, cause about 85% of DS cases. Mouse models of DS reveal that parvalbumin-expressing GABAergic interneurons are the primary cell type impacted by SCN1A haploinsufficiency and underlie the severe and frequent seizures. There is no treatment to eliminate seizures in DS patients or to treat the underlying cause of the disease.

### SUMMARY

Provided herein is a vector comprising: (a) a transgene polynucleotide sequence encoding a sequence-specific DNA-targeting module (DTM) fused to a transactivator; (b) an enhancer polynucleotide sequence that specifically restricts expression of the transgene to sodium voltage-gated channel alpha subunit 1 (SCN1A)-expressing cells in the brain; and (c) a promoter polynucleotide sequence. In some embodiments, the SCN1A-expressing cells are GABAergic interneuron cells. In some embodiments, the SCN1A-expressing cells are parvalbumin (PV)-expressing interneurons.

In some embodiments of the vectors provided herein, the DTM comprises a nuclease-deficient CRISPR-associated protein. In some embodiments, the vector further comprises a polynucleotide sequence encoding a guide RNA (gRNA). In some embodiments, the gRNA targets the SCN1A gene. In some embodiments, the gRNA specifically hybridizes to a regulatory region of the SCN1A gene. In some embodiments, the regulatory region of the SCN1A gene is a promoter or an enhancer. In some embodiments, the gRNA is encoded by or specifically hybridizes to the nucleotide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 160 or 161. In some embodiments, the gRNA is encoded by or specifically hybridizes to the nucleotide sequence of SEQ ID NO: 11, 19, 21 or 27. In some embodiments, the gRNA is operatively linked to a promoter recognized by RNA polymerase III. In some embodiments, the gRNA is operatively linked to a human U6 promoter. In some embodiments, the vector further comprises a polynucleotide sequence encoding a second gRNA targeting the SCN1A gene.

In some embodiments of the vectors provided herein, the nuclease-deficient CRISPR-associated protein is a nuclease-deficient Cas1, Cas1B, Cas2, Cas3, Cas4, Cas5, Cas6, Cas7, Cas8, Cas9, Cas10, Cas11, Cas12, Cas13, CasX, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, or another Cas ortholog. In some embodiments, the nuclease-deficient CRISPR-associated protein is dCas9. In some embodiments, the dCas9 is Staphylococcus aureus dCas9, Streptococcus pyogenes dCas9 or Campylobacter jejuni dCas9 or a dCas9 from an orthologous bacterial species. In some embodiments, the dCas9 comprises the amino acid sequence of SEQ ID NO: 156, 157, 158 or 159. In some embodiments, the dCas9 comprises an amino acid sequence at least about 90%, about 91%, about 92%, about 93%, about 94%, about 95%, about 96%, about 97%, about 98% or about 99% identical to the amino acid sequence of SEQ ID NO: 156, 157, 158 or 159. In some embodiments, the dCas9 is encoded by the nucleotide sequence of SEQ ID NO: 103, 104, 105 or 106. In some embodiments, the dCas9 is encoded by a nucleotide sequence at least about 90%, about 91%, about 92%, about 93%, about 94%, about 95%, about 96%, about 97%, about 98% or about 99% identical to the nucleotide sequence of SEQ ID NO: 103, 104, 105 or 106. In some embodiments, the dCas9 comprises a fragment of the amino acid sequence of SEQ ID NO: 156, 157, 158 or 159, wherein the fragment is a protein that is capable of forming a complex with the gRNA and targeting the SCN1A gene.

In some embodiments of the vectors provided herein, the promoter is a minimal promoter. In some embodiments, the promoter is recognized by RNA polymerase II. In some embodiments, the promoter is a human U6 promoter, a mouse U6 promoter or a human H1 promoter.

In some embodiments of the vectors provided herein, the enhancer polynucleotide sequence comprises the nucleotide sequence of any one of SEQ ID NOs: 33-102. In some embodiments, the enhancer polynucleotide sequence comprises the nucleotide sequence of SEQ ID NO: 69 or 34.

In some embodiments of the vectors provided herein, the transactivator is VP16, VP32, VP48, VP64, VPR, a MS2-SAM system, p65, Rta, the CITE-D domains of p300 or a SunTag. In some embodiments, the transactivator is encoded by the nucleotide sequence of SEQ ID NO: 107, 108, 109, 110, 111, 112 or 113 or a nucleotide sequence at least about 90%, about 91%, about 92%, about 93%, about 94%, about 95%, about 96%, about 97%, about 98% or about 99% identical to the nucleotide sequence of SEQ ID NO: 107, 108, 109, 110, 111, 112 or 113.

In some embodiments of the vectors provided herein, the vector comprises, in 5′-3′ order: (a) the promoter polynucleotide sequence; (b) the enhancer polynucleotide sequence; and (c) the transgene polynucleotide sequence. In some embodiments of the vectors provided herein, the vector comprises, in 5′-3′ order: (a) the enhancer polynucleotide sequence; (b) the promoter polynucleotide sequence; and (c) the transgene polynucleotide sequence.

Provided herein is a vector, wherein (a) the transgene polynucleotide sequence encodes the amino acid sequence of SEQ ID NO: 114; or an amino acid sequence at least about 90%, about 91%, about 92%, about 93%, about 94%, about 95%, about 96%, about 97%, about 98% or about 99% identical to the amino acid sequence of SEQ ID NO: 114; or (b) the transgene polynucleotide sequence comprises or consists of the nucleotide sequence of SEQ ID NO: 115, 116 or 117, or a nucleotide sequence at least about 90%, about 91%, about 92%, about 93%, about 94%, about 95%, about 96%, about 97%, about 98% or about 99% identical to the nucleotide sequence of SEQ ID NO: 115, 116 or 117.

In some embodiments of the vectors provided herein, the vector further comprises an artificial intron.

In some embodiments of the vectors provided herein, the vector further comprises a Woodchuck Hepatitis Virus Posttranscriptional Regulatory Element (WPRE), a hepatitis B virus posttranscriptional regulatory element (HBVPRE), a RNA transport element (RTE), a WPRE3 or a ws13 regulatory element.

In some embodiments of the vectors provided herein, the vector further comprises a polyadenylation signal sequence. In some embodiments, the polyadenylation signal sequence is a SV40 polyadenylation signal sequence.

In some embodiments of the vectors provided herein, the vector is a viral vector. In some embodiments, viral vector is an adeno-associated virus (AAV) vector. In some embodiments, the AAV vector comprises a first AAV inverted terminal repeat (ITR) located upstream of the promoter polynucleotide sequence and a second AAV ITR located downstream of the transgene polynucleotide sequence. In some embodiments, the first AAV ITR is an AAV2 ITR and the second AAV ITR is an AAV2 ITR.

Provided herein is a vector, comprising in 5′-3′ order: (a) a 5′ ITR; (b) a RNA polymerase III promoter; (c) a polynucleotide sequence encoding a gRNA; (d) the enhancer polynucleotide sequence; (e) a minimal promoter; (f) an artificial intron; (g) the transgene polynucleotide sequence; (h) a WPRE; (i) a polyadenylation signal sequence; and (j) a 3′ ITR.

Provided herein is a vector, comprising in 5′-3′ order: (a) a 5′ ITR; (b) a RNA polymerase III promoter; (c) a polynucleotide sequence encoding a gRNA; (d) a minimal promoter; (e) the enhancer polynucleotide sequence; (f) an artificial intron; (g) the transgene polynucleotide sequence; (h) a WPRE; (i) a polyadenylation signal sequence; and (j) a 3′ ITR.

Provided herein is a vector comprising the nucleotide sequence of SEQ ID NO: 137, 141, 145, 149 or 153, or a nucleotide sequence at least about 90%, about 91%, about 92%, about 93%, about 94%, about 95%, about 96%, about 97%, about 98% or about 99% identical to the nucleotide sequence of SEQ ID NO: 137, 141, 145, 149 or 153.

In some embodiments of the vectors provided herein, the vector is suitable for delivery via a non-viral delivery system. In some embodiments, the non-viral delivery system is a lipid nanoparticle or an exosome.

Provided herein is a viral particle comprising a vector disclosed herein. In some embodiments, the viral particle is a recombinant AAV (rAAV) particle. In some embodiments, the rAAV particle is an AAV9, AAV-PHP.eB, AAV-DJ or AAV2 serotype particle.

Provided herein is a population of viral particles comprising a plurality of viral particles disclosed herein.

Provided herein is a pharmaceutical composition comprising a vector, a viral particle or the population of viral particles disclosed herein, and a pharmaceutically acceptable carrier, vehicle or diluent.

Provided herein is a cell comprising a vector or a viral particle disclosed herein. In some embodiments, the cell is a mammalian cell or an insect cell.

Provided herein is a method of producing a rAAV particle, the method comprising: (i) culturing a cell disclosed herein under conditions allowing for packaging the rAAV particle; and (ii) harvesting the cultured host cell or culture medium for collection of the rAAV particle. In some embodiments, the rAAV particle comprises an AAV9, AAV-PHP.eB, AAV-DJ or AAV2 capsid protein.

Provided herein is a method for treating Dravet syndrome (DS) in a subject having or suspected of having DS, the method comprising administering to the subject a therapeutically effective amount of a vector, a viral particle, a population of viral particles or a pharmaceutical composition disclosed herein.

Provided herein is a method for treating or reducing the risk, severity, frequency or length of epilepsy and/or seizures in a subject who has or is at risk of having epilepsy, seizures, or Dravet syndrome (DS), the method comprising administering to the subject a therapeutically effective amount of a vector, a viral particle, a population of viral particles or a pharmaceutical composition disclosed herein.

Provided herein is a method for preventing or reducing the risk of Sudden Unexpected Death in Epilepsy (SUDEP) in a subject who has or is at risk of having epilepsy, seizures, or Dravet syndrome (DS), the method comprising administering to the subject a therapeutically effective amount of a vector, a viral particle, a population of viral particles or a pharmaceutical composition disclosed herein.

In some embodiments of the methods disclosed herein, the subject is between about 2 years old and about 18 years old. In some embodiments, the subject is older than 18 years.

In some embodiments of the methods disclosed herein, the vector, viral particle, population or pharmaceutical composition is administered to the subject via intracerebroventricular injection, intrathecal injection, intracarotid artery injection, or intraparenchymal injection.

In some embodiments of the methods disclosed herein, the vector, viral particle, population or pharmaceutical composition is administered to the subject in a single dose. In some embodiments, the single dose comprises from about 10E+9 to about 10E+14 viral particles.

Provided herein is a method for increasing levels of SCN1A expression in SCN1A-expressing cells in the brain, the method comprising contacting the cells with a vector, a viral particle, a population of viral particles or a pharmaceutical composition disclosed herein. In some embodiments, the SCN1A-expressing cells comprise a loss-of-function mutation in one copy of the SCN1A gene. In some embodiments, the SCN1A-expressing cells are GABAergic interneuron cells. In some embodiments, the GABAergic interneuron cells express parvalbumin (PV).

Provided herein is a vector, a viral particle, a population of viral particles or a pharmaceutical composition disclosed herein, for use as a medicament.

### BRIEF DESCRIPTION OF THE DRAWINGS

FIG. 1 depicts a plasmid map of a vector provided herein incorporated into an AAV expression cassette. The vector encodes a fusion protein comprising dCas9 and a VP64 transactivator. “sa-dCas9” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 2 depicts a plasmid map of a vector provided herein incorporated into an AAV expression cassette. The vector encodes a fusion protein comprising dCas9 and a VP64 transactivator. “minisadcas9-v2-de1234-444” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 3 depicts a plasmid map of a vector provided herein incorporated into an AAV expression cassette. The vector encodes a fusion protein comprising dCas9 and a VP64 transactivator. “minisadcas9-vp64-v4-de1479-649” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 4 depicts a plasmid map of a vector provided herein incorporated into an AAV expression cassette. The vector encodes a fusion protein comprising dCas9 and a VP64 transactivator. “minisadcas9-v5-de1234-444,479-649” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 5 depicts a plasmid map of a vector provided herein incorporated into an AAV expression cassette. The vector encodes a fusion protein comprising dCas9 and a VP64 transactivator. “sa-dCas9” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 6 depicts a plasmid map of a vector provided herein incorporated into an AAV expression cassette. The vector encodes a fusion protein comprising dCas9 and a VP64 transactivator. “minisadcas9-v2-de1234-444” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 7 depicts a plasmid map of a vector provided herein incorporated into an AAV expression cassette. The vector encodes a fusion protein comprising dCas9 and a VP64 transactivator. “minisadcas9-vp64-v4-de1479-649” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 8 depicts a plasmid map of a vector provided herein incorporated into an AAV expression cassette. The vector encodes a fusion protein comprising dCas9 and a VP64 transactivator. “minisadcas9-v5-de1234-444,479-649” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 9 depicts a plasmid map of a vector provided herein incorporated into an AAV expression cassette. The vector encodes a fusion protein comprising dCas9 and a VP64 transactivator. “sa-dCas9” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 10 depicts a plasmid map of a vector provided herein incorporated into an AAV expression cassette. The vector encodes a fusion protein comprising dCas9 and a VP64 transactivator. “minisadcas9-v2-de1234-444” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 11 depicts a plasmid map of a vector provided herein incorporated into an AAV expression cassette. The vector encodes a fusion protein comprising dCas9 and a VP64 transactivator. “minisadcas9-vp64-v4-de1479-649” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 12 depicts a plasmid map of a vector provided herein incorporated into an AAV expression cassette. The vector encodes a fusion protein comprising dCas9 and a VP64 transactivator. “minisadcas9-v5-de1234-444,479-649” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 13 depicts a plasmid map of a vector provided herein incorporated into an AAV expression cassette. The vector encodes a fusion protein comprising dCas9 and a VP64 transactivator. “sa-dCas9” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 14 depicts a plasmid map of a vector provided herein incorporated into an AAV expression cassette. The vector encodes a fusion protein comprising dCas9 and a VP64 transactivator. “minisadcas9-v2-de1234-444” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 15 depicts a plasmid map of a vector provided herein incorporated into an AAV expression cassette. The vector encodes a fusion protein comprising dCas9 and a VP64 transactivator. “minisadcas9-vp64-v4-de1479-649” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 16 depicts a plasmid map of a vector provided herein incorporated into an AAV expression cassette. The vector encodes a fusion protein comprising dCas9 and a VP64 transactivator. “minisadcas9-v5-de1234-444,479-649” refers to the polynucleotide sequence encoding a dCas9. “S5_Scn1a_E2” refers to the enhancer polynucleotide sequence. “NLS” refers to a nuclear localization signal. “bp” refers to base pairs.

FIG. 17 depicts a plasmid map of a vector provided herein incorporated into an AAV expression cassette. The vector encodes a fusion protein comprising dCas9 and a VP64 transactivator. “sa-dCas9” refers to the polynucleotide sequence encoding a dCas9. “NLS” refers to a nuclear localization signal. “bp” refers to base pairs.

FIG. 18A-FIG. 18B depict results from experiments testing effects of a vector provided herein on modulation of SCN1A expression in vitro in human (FIG. 18A) and mouse (FIG. 18B) cell lines. The vector contained a dCas9-VP64 transgene driven by the E2 enhancer. All RNA level values were normalized to the level of SCN1A RNA obtained in control conditions (i.e., with non-targeting control gRNA). The human gRNAs used in FIG. 18A were hG-pA11 (SEQ ID NO: 11), hG-pB6 (SEQ ID NO: 19), hG-E1 (SEQ ID NO: 21), and hG-E7 (SEQ ID NO: 27). “C” refers to a non-targeting gRNA control.

FIG. 18C-FIG. 18G depict results from experiments testing effects of a vector provided herein in vivo in wild-type and Dravet model mice. The vector was administered via recombinant AAV. The vector contained a dCas9-VP64 transgene driven by the E2 enhancer. Modulation of SCNA1 expression was measured after vector administration in wild-type mice (FIG. 18C) and Dravet mice (FIG. 18D). All RNA level values were normalized to the level of SCN1A RNA obtained in control conditions (i.e., with non-targeting control gRNA). Effects of vector administration on seizure latency (FIG. 18E), time to first seizure (FIG. 18F), and survival (FIG. 18G) in Dravet model mice are shown. “C” refers to a non-targeting gRNA control. “WT” refers to mice wild-type for SCN1A. “HET” refers to mice heterozygous for SCN1A. “PV INs” refers to parvalbumin-expressing interneurons. “EEG” refers to electroencephalogram. “d” refers to days.

### DETAILED DESCRIPTION

The disclosure provides compositions and methods for increasing expression of the sodium voltage-gated channel alpha subunit 1 (SCN1A) gene specifically in SCN1A-expressing cells (e.g., SCN1A-expressing cells in the brain). Such cells include parvalbumin (PV)-expressing interneurons. Heterozygous loss-of-function mutations in the SCN1A gene, which encodes the alpha subunit of sodium channel Nav1.1, cause a haploinsufficiency that leads to aberrant function of parvalbumin (PV)-expressing GABAergic interneurons. SCN1A mutations are linked to Dravet syndrome (DS) a drug-resistant infantile epilepsy associated with seizures, developmental disabilities and increased mortality. The disclosure provides vectors comprising elements that restrict expression of a transgene encoding a fusion protein to specific subtypes of neurons affected by DS. The fusion protein specifically targets the SCN1A gene and modulates expression of said gene, thus increasing SCN1A expression in these neurons. The compositions and methods provided herein are useful for normalizing brain function and reducing symptoms associated with DS, including seizures. Therapeutic effects are achieved by selectively restoring normal cell activity in the brain.

### Vectors

Provided herein are vectors comprising regulatory and transgene elements that increase SCN1A expression in SCN1A-expressing cells. The disclosure provides a vector comprising: (a) a transgene polynucleotide sequence encoding a sequence-specific DNA-targeting module (DTM) fused to a transactivator; (b) an enhancer polynucleotide sequence that specifically restricts expression of the transgene to SCN1A-expressing cells in the brain; and (c) a promoter polynucleotide sequence. The transgene sequence encodes a fusion protein comprising a SCN1A-sequence-specific DTM fused to a transactivator.

In some embodiments, the SCN1A-expressing cells are GABAergic interneuron cells. In some embodiments, the SCN1A-expressing cells are PV-expressing interneurons.

Vectors provided herein comprise a transgene polynucleotide sequence encoding a programmable DTM targeted to a genomic sequence (or sequences) that regulates the SCN1A gene. In some embodiments, a transgene polynucleotide sequence is codon-optimized (e.g., optimized for expression in human cells).

An exemplary amino acid sequence of the alpha subunit of human Nav1.1 (UniProtKB Identifier No. P35498) is provided as SEQ ID NO: 154. An exemplary nucleotide sequence of the human SCN1A gene is provided as SEQ ID NO: 155.

In some embodiments, a DTM comprises components from a CRISPR/Cas system or derived from a from a CRISPR/Cas system. In some embodiments, a DTM comprises a nuclease-deficient CRISPR-associated protein. In some embodiments (for example, when a DTM comprises a nuclease-deficient CRISPR-associated protein), the vector further comprises a polynucleotide sequence encoding a guide RNA (gRNA). In some embodiments, a gRNA targets the SCN1A gene or a sequence that regulates the SCN1A gene. In some embodiments, a gRNA specifically hybridizes to a regulatory region of the SCNA1 gene (e.g., specifically hybridizes under conditions present in a nucleus of the cell). In some embodiments, a gRNA specifically hybridizes to a control region, promoter, enhancer, intron, exon, transcription start site, coding region, or non-coding region of the SCNA1 gene. In some embodiments, a gRNA specifically hybridizes to promoter 1a, promoter 1b or promoter 1c of the SCNA1 gene. In some embodiments, the gRNA is encoded by or specifically hybridizes to the nucleotide sequence of any sequence in Table 1. In some embodiments, the gRNA is encoded by or specifically hybridizes to the nucleotide sequence SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 160 or 161. In some embodiments, the gRNA is encoded by or specifically hybridizes to the nucleotide sequence of SEQ ID NO: 11, 19, 21 or 27. In some embodiments, the polynucleotide sequence encoding the gRNA is operatively linked to a promoter (e.g., a RNA polymerase III promoter). In such embodiments, the vector comprises two promoters: (1) the promoter regulating the expression of the transgene polynucleotide sequence; and (2) the promoter regulating the expression of the polynucleotide sequence encoding the gRNA. In some embodiments, the promoter regulating the expression of the polynucleotide sequence encoding the gRNA is recognized by RNA polymerase III. In some embodiments, the promoter regulating the expression of the polynucleotide sequence encoding the gRNA is a human U6 promoter.

In some embodiments, a vector comprises a first polynucleotide sequence encoding a first gRNA and a second polynucleotide sequence encoding a second gRNA. In such embodiments, both the first gRNA and the second gRNA target the SCN1A gene or a sequence that regulates the SCN1A gene.

In some embodiments, a DTM comprises a nuclease-deficient CRISPR-associated protein (also known as a catalytically inactive CRISPR nuclease). Such modified proteins can be referred to as “dead Cas” or “dCas” proteins. For example, a Cas9 protein can be rendered catalytically inactive by introducing point mutations into each of its two nucleolytic domains. Examples of such mutations include D10A and H840A. These mutations block the nucleolytic activity of Cas9 but do not impact its binding to its target. Thus, dCas proteins can be used to deliver cargo to specific genomic locations even though they lack the ability to cleave or nick target nucleic acid sequences.

In some embodiments, a DTM comprises a nuclease-deficient CRISPR-associated protein that is a nuclease-deficient Cas1, Cas1B, Cas2, Cas3, Cas4, Cas5, Cas6, Cas7, Cas8, Cas9, Cas10, Cas11, Cas12, Cas13, CasX, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, or another Cas ortholog. In some embodiments, a DTM comprises dCas9. In some embodiments, a dCas9 is Staphylococcus aureus (Sa) dCas9, Streptococcus pyogenes dCas9 or Campylobacter jejuni dCas9. In some embodiments, a dCas9 is from an orthologous bacterial species.

In some embodiments, a dCas9 comprises any of the amino acid sequences in Table 9. In some embodiments, a dCas9 comprises the amino acid sequence of SEQ ID NO: 156, 157, 158 or 159. In some embodiments, a dCas9 comprises an amino acid sequence at least about 90%, about 91%, about 92%, about 93%, about 94%, about 95%, about 96%, about 97%, about 98% or about 99% identical to the amino acid sequence of SEQ ID NO: 156, 157, 158 or 159. In some embodiments, a dCas9 comprises a fragment of the amino acid sequence of SEQ ID NO: 156, 157, 158 or 159, wherein the fragment is a protein that is capable of forming a complex with the gRNA and targeting the SCN1A gene.

In some embodiments, a dCas9 is encoded by a codon-optimized nucleotide sequence (e.g., optimized for expression in human cells). In some embodiments, a dCas9 is encoded by any of the nucleotide sequences in Table 4. In some embodiments, a dCas9 is encoded by the nucleotide sequence of SEQ ID NO: 103, 104, 105 or 106.

In some embodiments, a dCas9 is encoded by a nucleotide sequence at least about 90%, about 91%, about 92%, about 93%, about 94%, about 95%, about 96%, about 97%, about 98% or about 99% identical to the nucleotide sequence of SEQ ID NO: 103, 104, 105 or 106. In some embodiments, a dCas9 is encoded by a fragment of the nucleotide sequence of SEQ ID NO: 103, 104, 105 or 106, wherein the fragment encodes a protein that is capable of forming a complex with the gRNA and targeting the SCN1A gene.

In some embodiments, a DTM comprises a zinc finger transcription factor or a portion of a zinc finger transcription factor. Zinc finger transcription factors comprise a DNA binding domain comprising zinc finger motifs. In some embodiments, a DTM comprises a DNA binding domain of a zinc finger transcription factor. In some embodiments, the zinc finger transcription factor is a C2H2 zinc finger transcription factor. A C2H2 zinc finger transcription factor comprises Cys₂His₂ zinc finger motifs. In some embodiments, the C2H2 zinc finger transcription factor comprises the DNA binding domain of a Zif268 zinc finger transcription factor, or a sequence derived from said DNA binding domain. In some embodiments, the C2H2 zinc finger transcription factor comprises the DNA binding domain of a humanized C2H2 zinc finger transcription factor, or a sequence derived from said DNA binding domain. In some embodiments, a zinc finger transcription factor is derived from a vertebrate animal with low immunogenicity. In some embodiments, a DTM comprises a DNA binding domain of a zinc finger transcription factor (e.g., Zif268), wherein the DNA binding domain targets the SCN1A gene. In some embodiments, a DNA binding domain of a zinc finger transcription factor is genetically engineered to target the SCN1A gene. In some embodiments, a DNA binding domain of a zinc finger transcription factor is genetically engineered to target the coordinates of the SCN1A gene provided in Table 2 or Table 3. Examples of zinc finger transcription factors and their uses for modulation of gene expression are provided in U.S. Pat. No. 9,234,016 and US 2016/0039893.

In some embodiments, a DTM comprises a transcription activator-like protein effector (TALE) protein, or a portion of a TALE protein. TALE proteins comprise a central domain responsible for DNA binding, a nuclear localization signal, and a domain that activates the target gene transcription. In some embodiments, a DTM comprises a DNA binding domain of a TALE protein. In some embodiments, a DTM comprises a DNA binding domain of a TALE protein that targets the SCN1A gene. In some embodiments, a DNA binding domain of a TALE protein is genetically engineered to target the SCN1A gene. In some embodiments, a DNA binding domain of a TALE protein is genetically engineered to target the coordinates of the SCN1A gene provided in Table 2 or Table 3. Examples of TALE proteins and their uses for modulation of gene expression are provided in U.S. Pat. Nos. 8,586,526, 9,394,545 and 9,522,936.

The vectors provided herein encode a fusion protein wherein the DTM described above is functionally fused to a transactivator. In some embodiments, a transactivator is VP16, VP32, VP48, VP64, VPR, a MS2-SAM system, p65, Rta, the CITE-D domains of p300 or a SunTag. Exemplary SunTag constructs are provided in US 2017/0219596. In some embodiments, a transactivator is encoded by any of the nucleotide sequences in Table 5. In some embodiments, a transactivator domain is encoded by the nucleotide sequence of SEQ ID NO: 107, 108, 109, 110, 111, 112 or 113. In some embodiments, a transactivator is encoded by a nucleotide sequence at least about 90%, about 91%, about 92%, about 93%, about 94%, about 95%, about 96%, about 97%, about 98% or about 99% identical to the nucleotide sequence of SEQ ID NO: 107, 108, 109, 110, 111, 112 or 113.

In some embodiments, the vectors provided herein encode nuclear localization signals (NLSs) that flank the DTM in the fusion protein. In some embodiments, a NLS is a simian virus 40 (SV40) NLS. In some embodiments, a NLS is a nucleoplasmin NLS. In some embodiments, a vector encodes a SV40 NLS and a nucleoplasmin NLS flanking the DTM in coding frame with the fusion protein.

Vectors provided herein comprise a promoter sequence that regulates expression of the transgene. In some embodiments, a promoter is a minimal promoter. In some embodiments, a promoter is recognized by RNA polymerase II. In some embodiments, a promoter is a human U6 (hU6) promoter, a mouse U6 promoter or a human H1 promoter. In some embodiments, a promoter is an E2 promoter.

In some embodiments, a promoter is a constitutive promoter. In some embodiments, a promoter is an inducible promoter. In some embodiments, a promoter is a tissue-specific promoter. In some embodiments, a promoter is the chicken beta-actin (CBA) promoter, the GUSB240 promoter, the GUSB379 promoter, the HSVTK promoter, the CMV promoter, the SV40 early promoter, the SV40 late promoter, the metallothionein promoter, the murine mammary tumor virus (MMTV) promoter, the Rous sarcoma virus (RSV) promoter, the polyhedrin promoter, the EF-1 alpha promoter, the dihydrofolate reductase (DHFR) promoter or the phosphoglycerol kinase (PGK) promoter.

Vectors provided herein comprise an enhancer polynucleotide sequence that specifically restricts expression of the transgene to SCN1A-expressing cells (for example, PV-expressing interneuron cells) in the brain. In some embodiments, an enhancer polynucleotide sequence comprises any of the nucleotide sequences in Table 2 or Table 3. In some embodiments, an enhancer polynucleotide sequence comprises the nucleotide sequence of any one of SEQ ID NOs: 33-102. In some embodiments, an enhancer polynucleotide sequence comprises a nucleotide sequence at least about 90%, about 91%, about 92%, about 93%, about 94%, about 95%, about 96%, about 97%, about 98% or about 99% identical to the nucleotide sequence of any one of SEQ ID NOs: 33-102. In some embodiments, an enhancer polynucleotide sequence comprises the nucleotide sequence of SEQ ID NO: 69 or 34. In some embodiments, an enhancer polynucleotide sequence comprises a nucleotide sequence at least about 90%, 95%, 98% or 99% identical to the nucleotide sequence of SEQ ID NO: 69 or 34. Exemplary enhancer sequences that restrict expression of a transgene to SCN1A-expressing cells are disclosed in WO 2020/163102, the contents of which are hereby incorporated by reference in their entirety for all purposes, including the enhancer sequences disclosed therein.

In some embodiments, a vector provided herein comprises, in 5′-3′ order: (a) the promoter polynucleotide sequence; (b) the enhancer polynucleotide sequence; and (c) the transgene polynucleotide sequence. In some embodiments, a vector provided herein comprises, in 5′-3′ order: (a) the enhancer polynucleotide sequence; (b) the promoter polynucleotide sequence; and (c) the transgene polynucleotide sequence. The location and orientation of the enhancer polynucleotide sequence can be varied.

Provided herein is a vector encoding one of the transgenes in Table 6 (NLS-SadCas9-NLS-VP64, NLS-miniSadCas9v2-NLS-VP64, NLS-miniSadCas9v4-NLS-VP64 or NLS-miniSadCas9v5-NLS-VP64).

Provided herein is a vector comprising a transgene polynucleotide sequence that encodes the amino acid sequence of SEQ ID NO: 114. Further provided herein is a vector comprising a transgene polynucleotide sequence that encodes an amino acid sequence at least about 90%, about 91%, about 92%, about 93%, about 94%, about 95%, about 96%, about 97%, about 98% or about 99% identical to the amino acid sequence of SEQ ID NO: 114.

Provided herein is a vector comprising a transgene polynucleotide sequence that encodes the amino acid sequence of SEQ ID NO: 114, 118, 122 or 126. Further provided herein is a vector comprising a transgene polynucleotide sequence that encodes an amino acid sequence at least about 90%, about 91%, about 92%, about 93%, about 94%, about 95%, about 96%, about 97%, about 98% or about 99% identical to the amino acid sequence of SEQ ID NO: 114, 118, 122 or 126.

Provided herein is a vector comprising a transgene polynucleotide sequence comprising or consisting of the nucleotide sequence of SEQ ID NO: 115, 116 or 117. Further provided herein is a vector comprising a transgene polynucleotide sequence comprising or consisting of a nucleotide sequence at least about 90%, about 91%, about 92%, about 93%, about 94%, about 95%, about 96%, about 97%, about 98% or about 99% identical to the nucleotide sequence of SEQ ID NO: 115, 116 or 117.

Provided herein is a vector comprising a transgene polynucleotide sequence comprising or consisting of the nucleotide sequence of SEQ ID NO: 115, 116, 117, 119, 120, 121, 123, 124, 125, 127, 128 or 129. Further provided herein is a vector comprising a transgene polynucleotide sequence comprising or consisting of a nucleotide sequence at least about 90%, about 91%, about 92%, about 93%, about 94%, about 95%, about 96%, about 97%, about 98% or about 99% identical to the nucleotide sequence of SEQ ID NO: 115, 116, 117, 119, 120, 121, 123, 124, 125, 127, 128 or 129.

In some embodiments, a vector provided herein further comprises an artificial intron. In some embodiments, a vector provided herein further comprises a chimeric intron.

In some embodiments, a vector provided herein further comprises or encodes a woodchuck hepatitis virus post-transcriptional element (WPRE). See, e.g., Wang and Verma, Proc. Natl. Acad. Sci., USA, 96: 3906-3910 (1999). In some embodiments, a vector comprises or encodes a hepatitis B virus posttranscriptional regulatory element (HBVPRE) and/or a RNA transport element (RTE). In some embodiments, the WPRE or HBVPRE sequence is any of the WPRE or HBVPRE sequences disclosed in U.S. Pat. No. 6,136,597 or U.S. Pat. No. 6,287,814. In some embodiments, a vector provided herein further comprises or encodes a WPRE3 or a ws13 regulatory element. In some embodiments, a WPRE3 comprises or consists of

(SEQ ID NO: 135)
GATAATCAACCTCTGGATTACAAAATTTGTGAAAGATTGACTGGTATTC
TTAACTATGTTGCTCCTTTTACGCTATGTGGATACGCTGCTTTAATGCC
TTTGTATCATGCTATTGCTTCCCGTATGGCTTTCATTTTCTCCTCCTTG
TATAAATCCTGGTTAGTTCTTGCCACGGCGGAACTCATCGCCGCCTGCC
TTGCCCGCTGCTGGACAGGGGCTCGGCTGTTGGGCACTGACAATTCCGT
GGTGTT.

In some embodiments, a vector provided herein further comprises or encodes a polyadenylation (polyA) signal sequence. As used herein, a “polyadenylation signal sequence” refers to a DNA sequence that when transcribed regulates the addition of a polyA tail to the mRNA transcript. In some embodiments, a polyA signal sequence is a SV40, human, bovine or rabbit polyA signal sequence. In some embodiments, a polyA signal sequence is a SV40 polyA signal sequence. In some embodiments, a polyA signal sequence is a β-globin polyA signal sequence. In some embodiments, a polyA signal sequence is a human growth hormone polyA signal sequence or a bovine growth hormone polyA signal sequence. In some embodiments, a SV40 polyA signal sequence comprises or consists of

(SEQ ID NO: 136)
AACTTGTTTATTGCAGCTTATAATGGTTACAAATAAAGCAATAGCATCA
CAAATTTCACAAATAAAGCATTTTTTTCACTGC

In some embodiments, a vector provided herein further comprises or encodes a Kozak sequence (for example, a DNA sequence transcribed to an RNA Kozak sequence). In some embodiments, a vector comprises a Kozak sequence upstream of the transgene. In some embodiments, the Kozak sequence is encoded by GCCACC (SEQ ID NO: 130). In some embodiments, the Kozak sequence (e.g., RNA Kozak sequence) comprises or consists of ACCAUGG (SEQ ID NO: 131), GCCGCCACCAUGG (SEQ ID NO: 132), CCACCAUG (SEQ ID NO: 133) or CCACCAUGG (SEQ ID NO: 134).

In some embodiments, a vector provided herein further comprises a TATA transcriptional regulatory activation site (see, e.g., Francois et al., (2005) J. Virol. 79(17):11082-11094).

In some embodiments, a vector provided herein comprises (a) the promoter polynucleotide sequence; (b) the enhancer polynucleotide sequence; and (c) the transgene polynucleotide sequence; (d) an artificial intron; and (e) a WPRE. In some embodiments, a vector provided herein comprises (a) the promoter polynucleotide sequence; (b) the enhancer polynucleotide sequence; and (c) the transgene polynucleotide sequence; (d) a WPRE; and (e) a polyA signal sequence. In some embodiments, a vector provided herein comprises (a) the promoter polynucleotide sequence; (b) the enhancer polynucleotide sequence; and (c) the transgene polynucleotide sequence; (d) an artificial intron; and (e) a polyA signal sequence. In some embodiments, a vector provided herein comprises (a) the promoter polynucleotide sequence; (b) the enhancer polynucleotide sequence; and (c) the transgene polynucleotide sequence; (d) an artificial intron; (e) a WPRE; and (f) a polyA signal sequence.

In some embodiments, the vectors provided herein are plasmids or viral expression cassettes that comprise additional nucleic acid sequences. In some embodiments, the vectors provided herein may be used to generate recombinant virus particles to serve as viral vectors for gene delivery. In some embodiments, the vectors provided herein are formulated for use with via non-viral delivery systems. Further provided herein are plasmids comprising any of the vector nucleic acid sequences disclosed herein.

In some embodiments, a vector provided herein is non-integrating. In some embodiments, a vector provided herein is non-replicating.

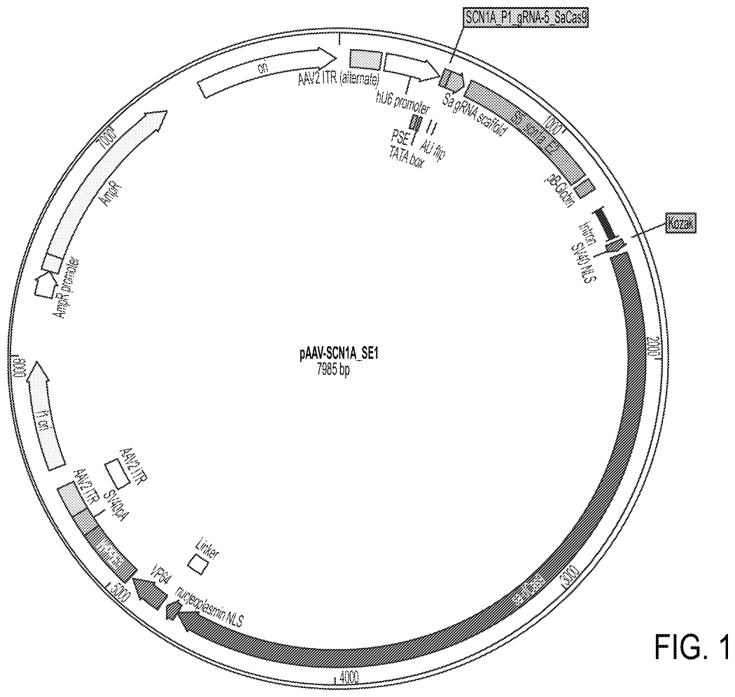

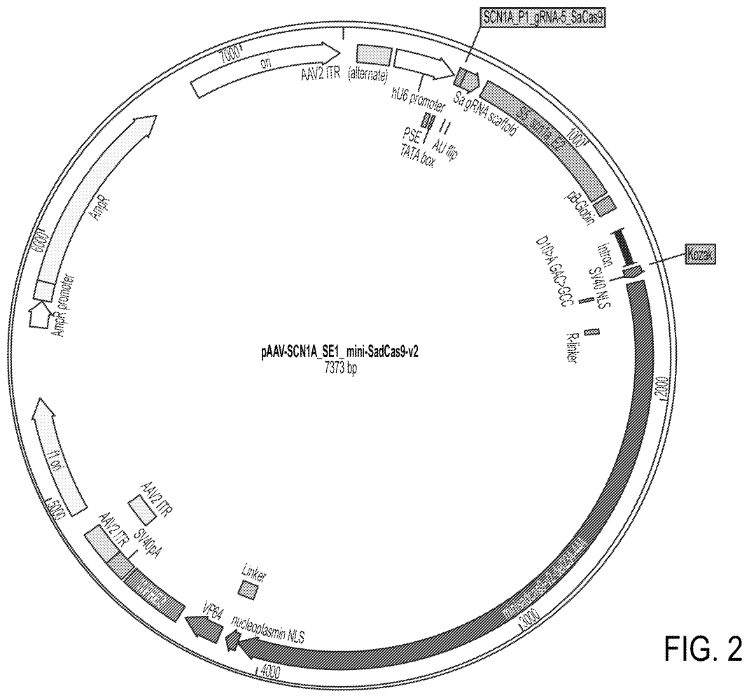

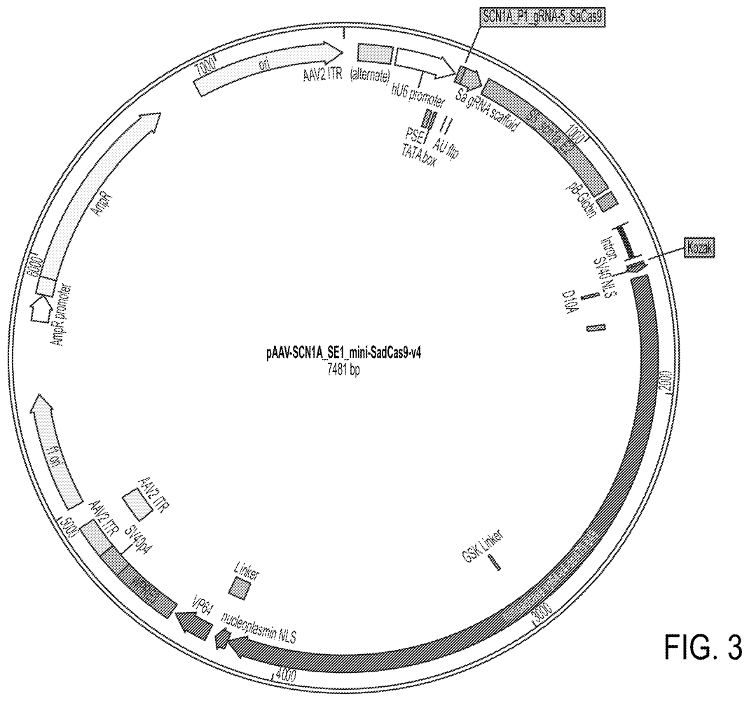

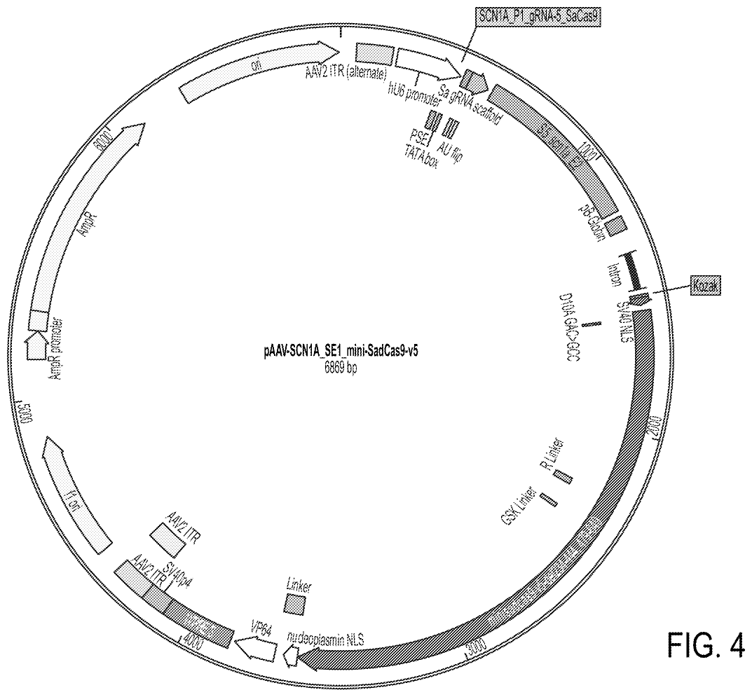

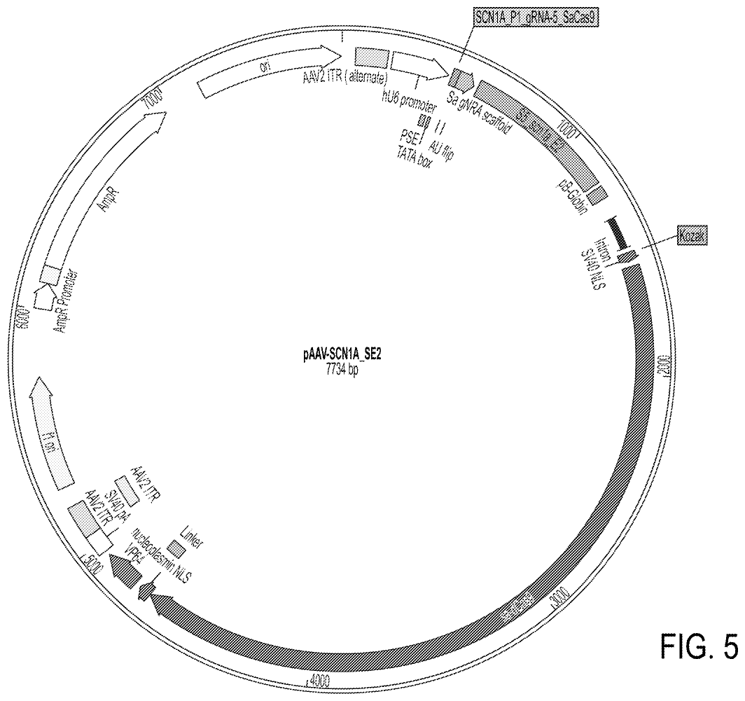

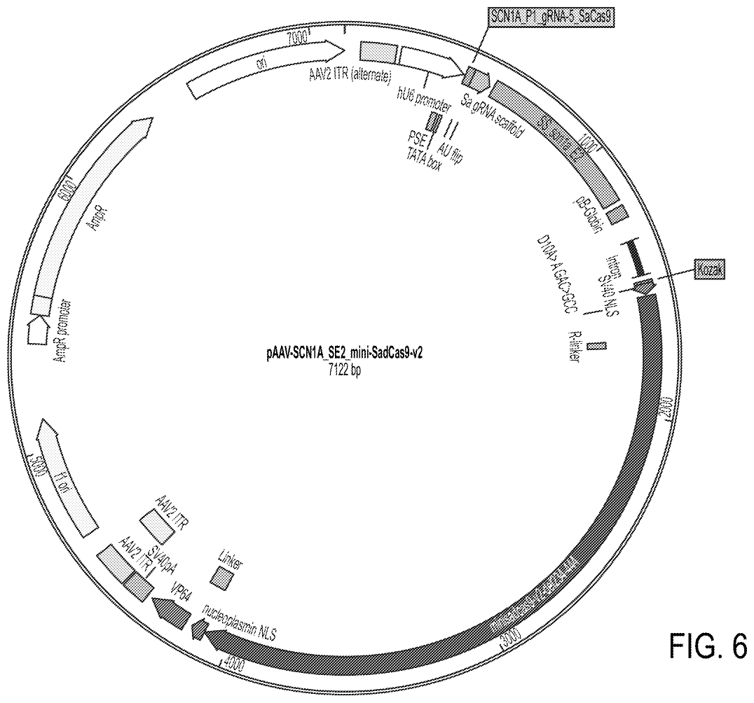

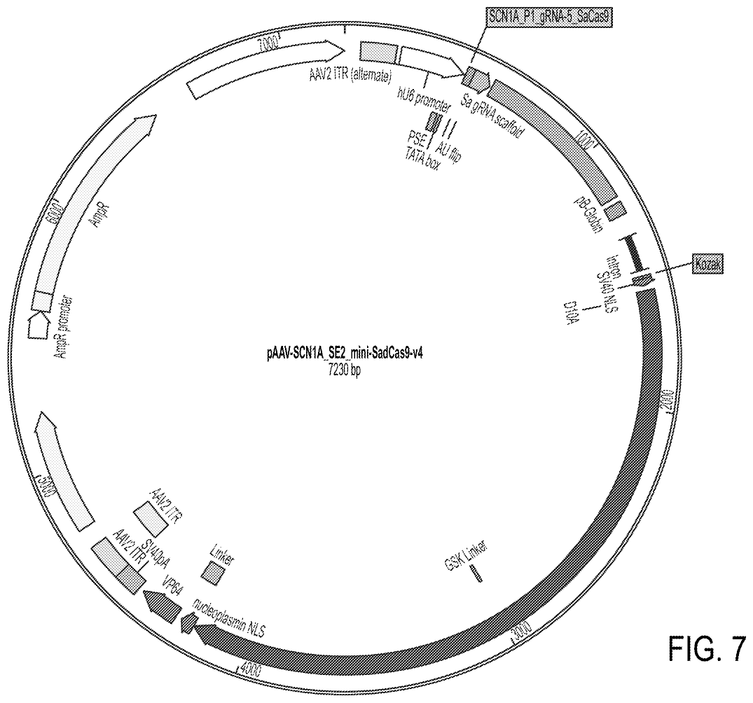

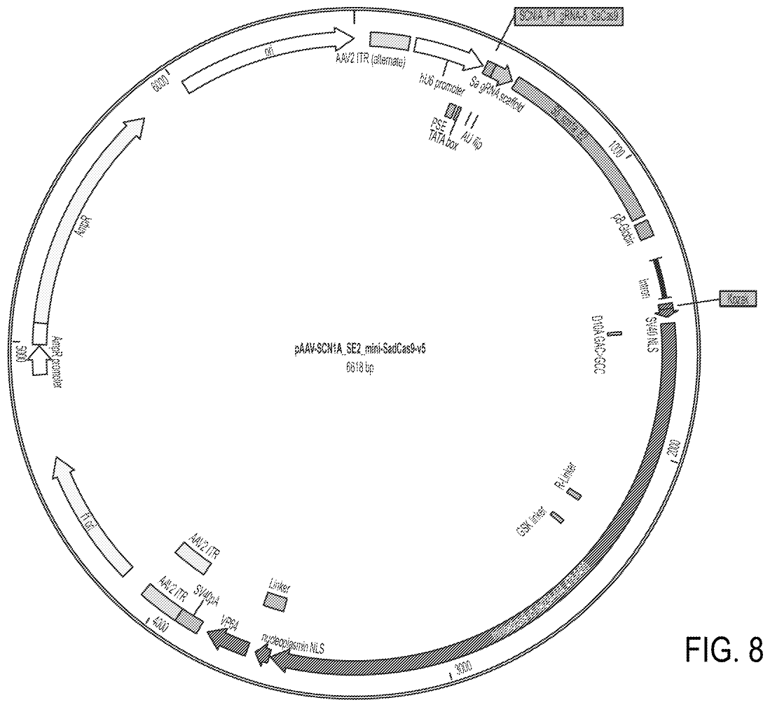

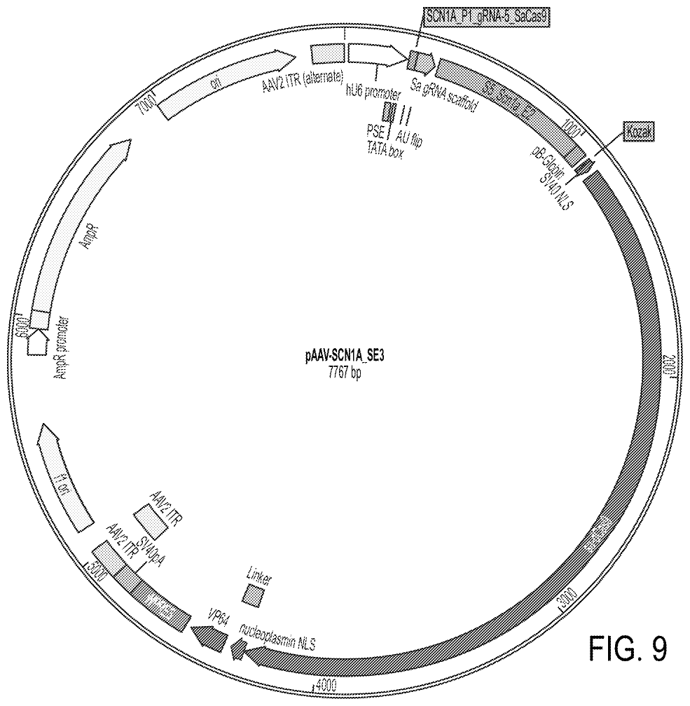

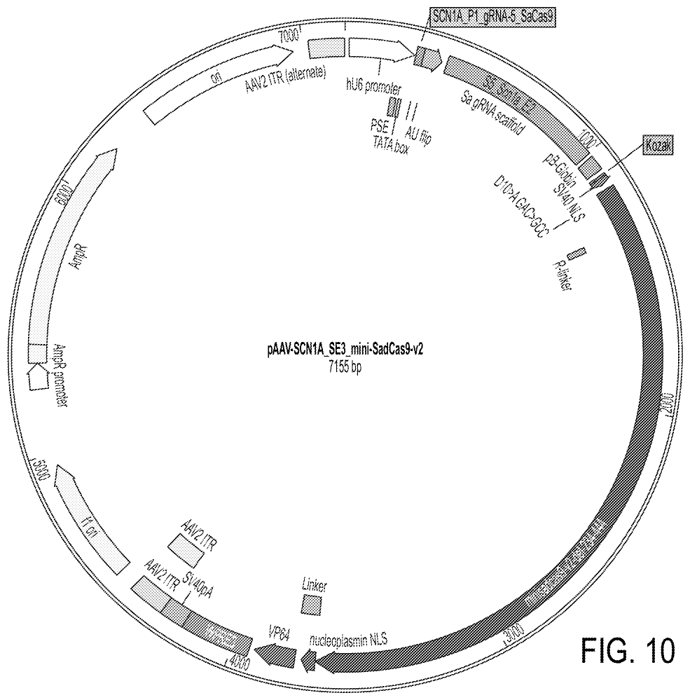

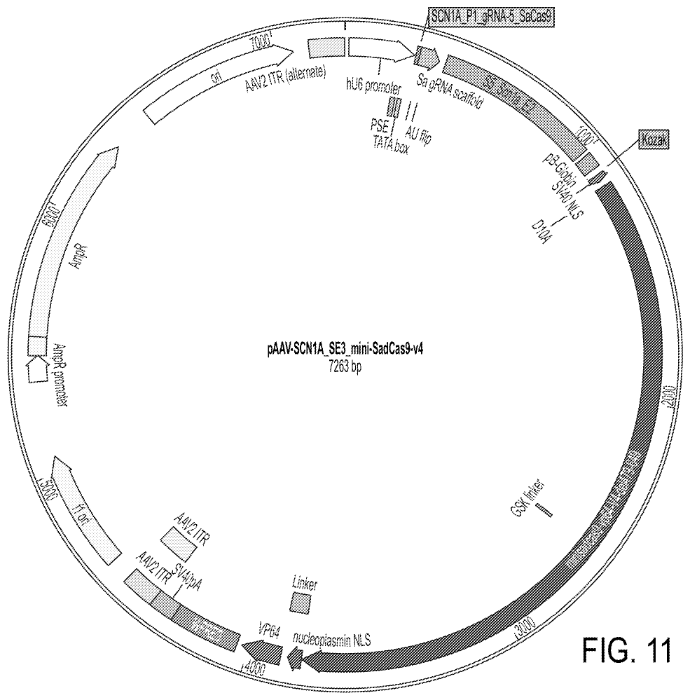

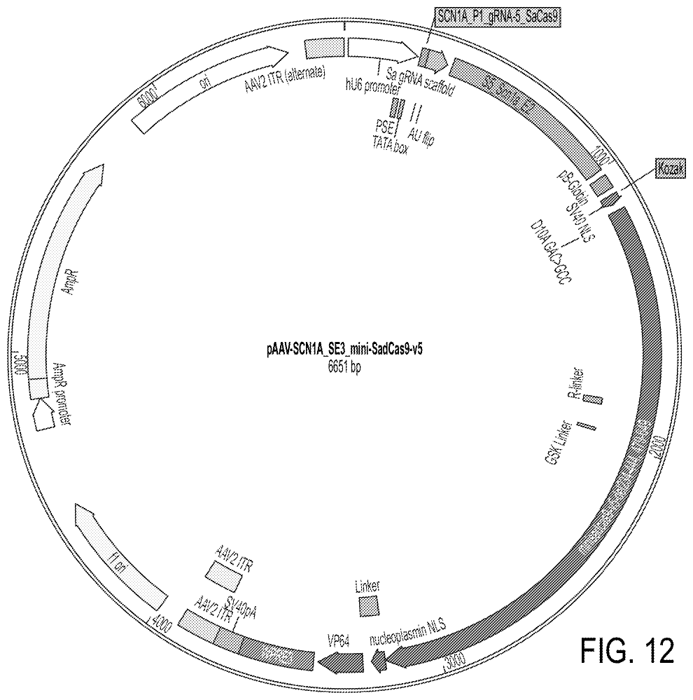

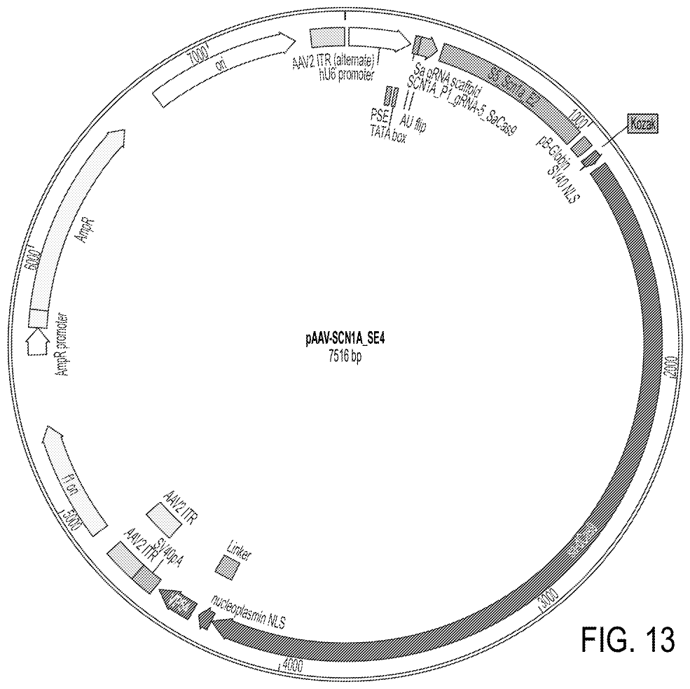

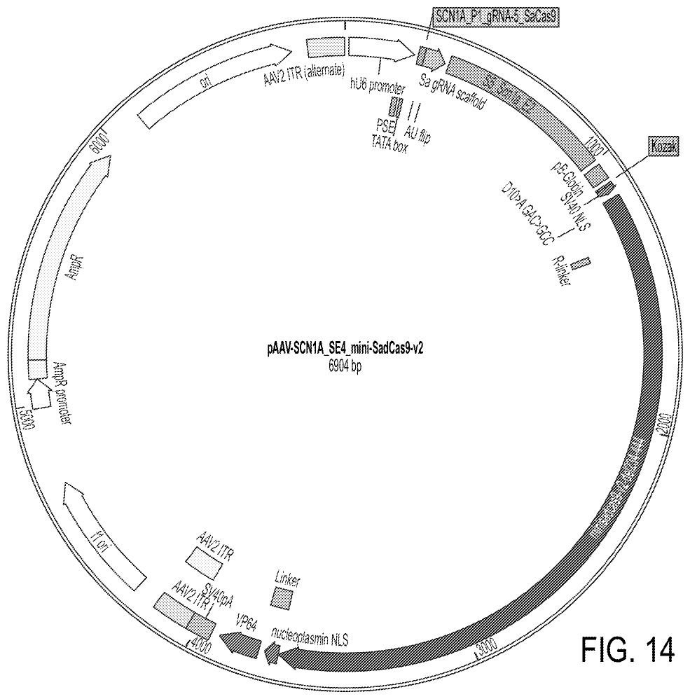

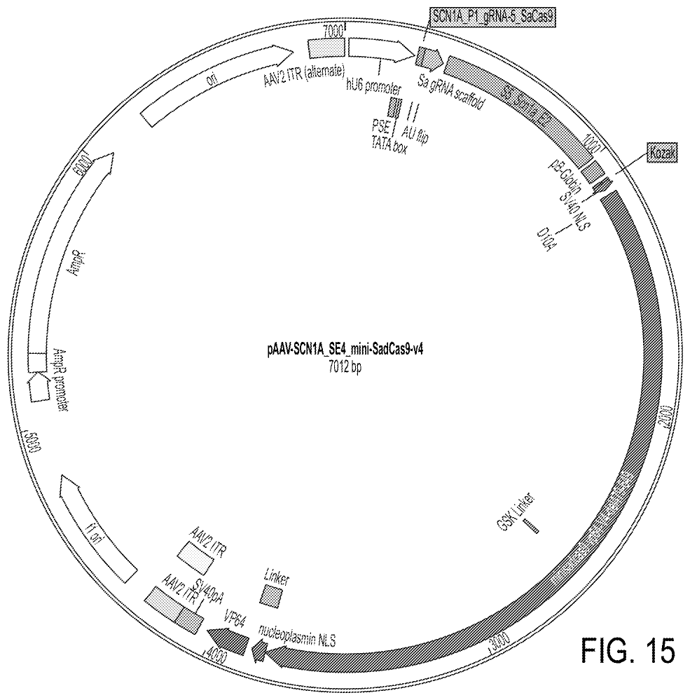

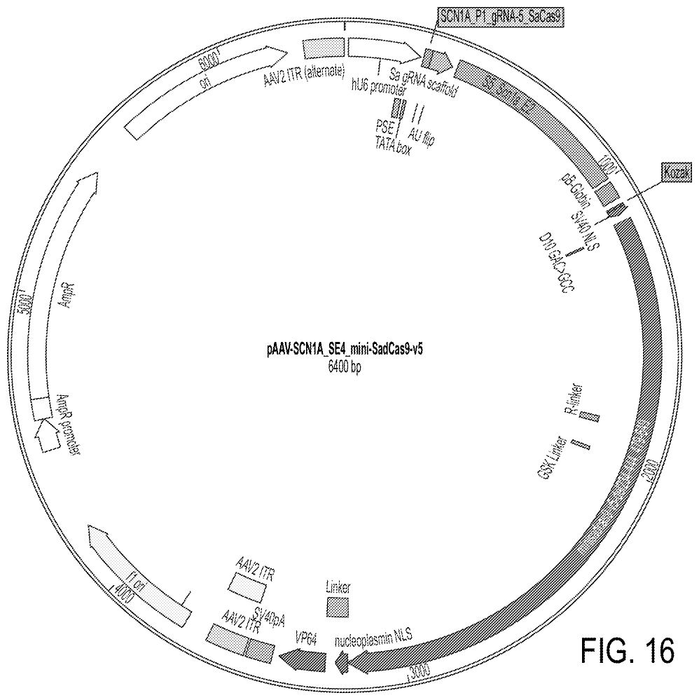

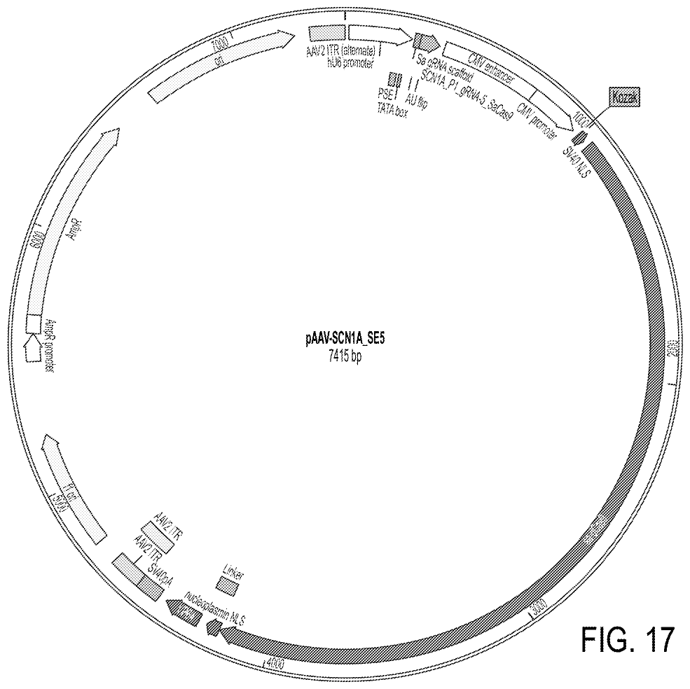

In some embodiments, a vector provided herein is a viral vector. In some embodiments, the viral vector is an adeno-associated virus (AAV) vector. In some embodiments, an AAV vector comprises a first AAV inverted terminal repeat (ITR) located upstream of the promoter polynucleotide sequence and a second AAV ITR located downstream of the transgene polynucleotide sequence. ITRs are sequences that mediate AAV proviral integration and packaging of AAV DNA into virions. In some embodiments, an AAV vector comprises a first AAV ITR and a second AAV ITR flanking the polynucleotide sequences to be packaged into a recombinant AAV (rAAV) particle. In some embodiments, the first AAV ITR is an AAV2 ITR and the second AAV ITR is an AAV2 ITR. Maps of exemplary AAV expression cassettes comprising vectors provided herein incorporated into plasmids are depicted in FIG. 1-FIG. 17. AAV expression cassettes and related plasmids provided herein can be used in production of rAAV particles.

In some embodiments, an AAV vector provided herein is self-complementary. In some embodiments, an AAV vector provided herein is single-stranded.

In some embodiments, a vector provided herein, comprises, in 5′-3′ order: (a) a 5′ ITR; (b) a RNA polymerase III promoter; (c) a polynucleotide sequence encoding a gRNA; (d) the enhancer polynucleotide sequence; (e) a minimal promoter; (f) an artificial intron; (g) the transgene polynucleotide sequence; (h) a WPRE; (i) a polyadenylation signal sequence; and (j) a 3′ ITR. In some embodiments, a vector provided herein, comprises, in 5′-3′ order: (a) a 5′ ITR; (b) a RNA polymerase III promoter; (c) a polynucleotide sequence encoding a gRNA; (d) a minimal promoter; (e) the enhancer polynucleotide sequence; (f) an artificial intron; (g) the transgene polynucleotide sequence; (h) a WPRE; (i) a polyadenylation signal sequence; and (j) a 3′ ITR. The location and orientation of the enhancer polynucleotide sequence can be varied.

Examples of vectors that are AAV expression cassettes and their sequences are provided in Table 7. In some embodiments, these sequences comprise, in 5′-3′ order: (a) a 5′ AAV2 ITR; (b) a hU6 promoter; (c) a polynucleotide sequence encoding a gRNA; (d) the enhancer polynucleotide sequence; (e) a beta-globin promoter; (f) the transgene polynucleotide sequence; and (h) a 3′ AAV2 ITR. In some embodiments, these sequences further comprise any combination of (1) an artificial intron; (2) a WPRE; and (3) a polyadenylation signal sequence (e.g., a SV40 polyadenylation signal sequence). In some embodiments, these sequences comprise, in 5′-3′ order: (a) a 5′ AAV2 ITR; (b) a hU6 promoter; (c) a polynucleotide sequence encoding a gRNA; (d) the enhancer polynucleotide sequence; (e) a beta-globin promoter; (f) an artificial intron; (g) the transgene polynucleotide sequence; (h) a WPRE (WPRE3); (i) a SV40 polyadenylation signal sequence; and (j) a 3′ AAV2 ITR.

In some embodiments, a vector comprises the nucleotide sequence of SEQ ID NO: 137, 141, 145, 149 or 153. In some embodiments, a vector comprises a nucleotide sequence at least about 90%, about 91%, about 92%, about 93%, about 94%, about 95%, about 96%, about 97%, about 98% or about 99% identical to the nucleotide sequence of SEQ ID NO: 137, 141, 145, 149 or 153.

In some embodiments, a vector comprises the nucleotide sequence of SEQ ID NO: 137, 138, 139, 140, 141, 142, 143, 144, 145, 146, 147, 148, 149, 150, 151, 152 or 153. In some embodiments, a vector comprises a nucleotide sequence at least about 90%, about 91%, about 92%, about 93%, about 94%, about 95%, about 96%, about 97%, about 98% or about 99% identical to the nucleotide sequence of SEQ ID NO: 137, 138, 139, 140, 141, 142, 143, 144, 145, 146, 147, 148, 149, 150, 151, 152 or 153.

Further provided herein is a viral particle (also referred to as a virion) comprising any of the vectors, expression cassettes or nucleic acid molecules provided herein. In some embodiments the viral particle is a rAAV particle. In some embodiments, the rAAV particle is an AAV9 serotype particle. In some embodiments, the rAAV particle is an AAV-PHP.eB, AAV-DJ or AAV2 serotype particle. In some embodiments, the rAAV is an AAV1, AAV2, AAV3 (including types 3A and 3B), AAV4, AAV5, AAV6, AAV7, AAV8, AAV9, AAV10, AAV11, AAV12, AAV13, AAVrh32.33, AAVrh.8, AAVrh.10, AAVrh32.33, AAVrh.74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, TM-AAV6, AAV-PHP.A, AAV-PHP.B, AAV-PHP.S, AAV-PHPeB, AAV-CAP.B10, AAV2-r3.45, AAV2-LSS, AAV2PFG, AAV2-PPS, AAV2-TLH or AAV2-GMN serotype particle.

Provided herein is a population of viral particles comprising a plurality of viral particles disclosed herein. Provided herein is a population of rAAV particles comprising a plurality of rAAV particles disclosed herein.

In some embodiments, a vector provided herein is suitable for delivery via a non-viral delivery system. In some embodiments, a vector provided herein is formulated for delivery via a non-viral delivery system. In some embodiments, a non-viral delivery system is a lipid nanoparticle or an exosome. In some embodiments, non-viral systems for gene delivery may be lipid-based, polymer-based or other nanomaterial-based. Cationic lipids or cationic polymers can be complexed with nucleic acid molecules to produce synthetic vehicles for gene delivery.

Provided herein is a cell comprising any of the vectors or viral particles disclosed herein. In some embodiments, the cell is a mammalian cell. In some embodiments, a mammalian cell is a HEK293 cell. In some embodiments, the cell is an insect cell. In some embodiments, the insect cell is a Spodoptera frugiperda cell (for example, the Sf9 or ExpiSf9™ cell lines). The Sf9 insect cell line (Thermo Fisher Scientific, Waltham, MA) is a clonal isolate derived from the parental S. frugiperda cell line IPLB-Sf-21-AE. ExpiSf9™ cells (Thermo Fisher Scientific, Waltham, MA) are a non-engineered derivative of Sf9 insect cells that have been adapted for high-density suspension growth.

### Pharmaceutical Compositions

Provided herein are pharmaceutical compositions comprising any of the vectors, viral particles, nucleic acid molecules, populations of viral particles disclosed herein, and a pharmaceutically acceptable carrier, vehicle or diluent. “Pharmaceutically acceptable” refers to a material that is not toxic or otherwise undesirable, i.e., the material may be administered to a subject without causing any undesirable biological effects. In general, a pharmaceutically acceptable material has one or more benefits that outweigh any undesirable biological effect that the material may have. Undesirable biological effects may include, for example, excessive toxicity, irritation, allergic response, and other problems and complications.

For injection, the carrier will typically be a liquid. For other methods of administration, the carrier may be either solid or liquid.

In some embodiments, a pharmaceutical composition may comprise other medicinal agents, pharmaceutical agents, stabilizing agents, buffers, adjuvants and/or diluents.

In some embodiments, a pharmaceutical composition comprises at least one pharmaceutically acceptable carrier, excipient, and/or vehicle, for example, solvents, buffers, solutions, dispersion media, coatings, antibacterial agents, antifungal agents, isotonic agents, and absorption delaying agents. In some embodiments, the pharmaceutically acceptable carrier, excipient, and/or vehicle comprises saline, buffered saline, dextrose, water, glycerol, sterile isotonic aqueous buffer, or a combination thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like), or a suitable mixture thereof. In some embodiments, the compositions disclosed herein further comprise emulsifying or wetting agents, or pH buffering agents. Such species may be present in small amounts (e.g., less than 10% by weight of the composition, such as less than 5% by weight of the composition, 2% by weight of the composition, 1% by weight of the composition, or less).

In some embodiments, a pharmaceutical composition further comprises one or more other pharmaceutical ingredients, such as one or more preservatives or chemical stabilizers. Examples of preservatives and chemical stabilizers include, but are not limited to, chlorobutanol, potassium sorbate, sorbic acid, sulfur dioxide, propyl gallate, parabens, ethyl vanillin, glycerin, phenol, parachlorophenol, and albumin. In some embodiments, the compositions disclosed herein further comprise antibacterial agents and/or antifungal agents, such as parabens, chlorobutanol, phenol, sorbic acid, and thimerosal; isotonic agents, such as sugars and sodium chloride; and/or agents delaying absorption, such as aluminum monostearate and gelatin.

In some embodiments, a pharmaceutical composition is in a form of an injectable solution or dispersion, such as an aqueous solution or dispersion. In some embodiments, a pharmaceutical composition is a sterile powder for the extemporaneous preparation of sterile injectable solutions or dispersions. Dispersions may be prepared in water, glycerol, liquid polyethylene glycols, oils, or any combination thereof. Delivery vehicles such as liposomes, nanocapsules, microparticles, microspheres, lipid particles, vesicles, and the like, may be used for the introduction of the pharmaceutical compositions provided herein.

In some embodiments, a pharmaceutical composition is suitable or formulated for intracerebroventricular injection, intrathecal injection, intracarotid artery injection, or intraparenchymal injection.

Methods of Producing rAAV Particles

Provided herein are methods of producing rAAV particles by using any of the vectors, AAV expression cassettes, nucleic acid molecules and cells disclosed herein.

Provided herein is a method of producing a rAAV particle, the method comprising: (i) culturing a cell comprising a vector or an AAV expression cassette disclosed herein under conditions allowing for packaging the rAAV particle; and (ii) harvesting the cultured host cell or culture medium for collection of the rAAV particle.

In some embodiments, a method of producing a rAAV particle comprises providing to a cell: (a) a vector (i.e., a nucleic acid template comprising an AAV expression cassette) comprising two AAV ITRs located 5′ and 3′ to the polynucleotide sequences desired to be packaged into the rAAV particle, and (b) AAV sequences sufficient for replication of the nucleic acid template and encapsidation into AAV protein capsids (e.g., AAV rep sequences and AAV cap sequences encoding the AAV capsid subunits, also referred to as “helper functions”). Typically, the AAV rep and cap sequences will not be flanked by AAV ITRs, to prevent rescue and/or packaging of these sequences.

The vector (nucleic acid template), rep sequences, cap sequences, and any other helper functions required for producing the rAAV particles disclosed herein may be delivered to the packaging host cell using any appropriate genetic element. Further details on methods of preparing rAAV particles are provided in Sambrook et al., Molecular Cloning: A Laboratory Manual, Cold Spring Harbor Press, Cold Spring Harbor, NY; Fisher et al, J. Virol., 70:520-532 (1993) and U.S. Pat. No. 5,478,745.

The nucleic acid template and AAV rep and cap sequences are provided under conditions such that virus vector comprising the nucleic acid template packaged within the AAV protein capsid is produced in the cell. The method can further comprise the step of collecting the virus vector from the cell. The virus vector can be collected from the medium and/or by lysing the cells.

The cell can be a cell that is permissive for AAV viral replication. Any suitable cell known in the art may be employed. In some embodiments, the cell is a mammalian cell (e.g., a HEK293 cell). In some embodiments, the cell can be a trans-complementing packaging cell line that provides functions deleted from a replication-defective helper virus, e.g., HEK293 cells or other Ela trans-complementing cells. The helper sequences may be embedded in a chromosome or maintained as a stable extrachromosomal element.

In some embodiments, rAAV particles are produced using the triple transfection method, as described in U.S. Pat. No. 6,001,650. In some embodiments, the rAAVs are produced by transfecting a host cell with an AAV vector (i.e., AAV expression cassette) to be packaged into rAAV particles, an AAV helper function vector, and an accessory function vector. An AAV helper function vector encodes the “AAV helper function” sequences (i.e., rep and cap), which function in trans for productive AAV replication and encapsidation. Non-limiting examples of AAV helper function vectors include pHLP19 and pRep6cap6 vector, described in U.S. Pat. Nos. 6,001,650 and 6,156,303, respectively. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (e.g., other than herpes simplex virus type-1) and vaccinia virus.

In some embodiments, rAAVs are produced using recombinant baculovirus vectors. Production of rAAVs using baculovirus vectors is described in, for example, Urabe et al. (2002) Hum Gene Ther 13(16):1935-1943; Smith et al. (2009) Mol Ther 17(11):1888-1896; U.S. Pat. Nos. 8,945,918; 9,879,282; and US 2018/0371495. In some embodiments, a baculovirus vector genome is derived from Autographa californica multicapsid nucleopolyhedrovirus (AcMNPV), Bombyx mori nuclear polyhedrosis virus (BmNPV), Helicoverpa armigera (HearNPV) or Spodoptera exigua MNPV. Baculovirus vectors are used to produce recombinant AAVs in insect cells (e.g., Spodoptera frugiperda cells). In some embodiments, the Sf9 or ExpiSf9™ Spodoptera frugiperda cell lines are used to produce rAAVs. In some embodiments, methods of the disclosure comprise co-infecting insect cells with populations of recombinant baculoviruses (rBVs) to produce rAAV disclosed herein. At least two populations of rBVs may be used in the methods of the disclosure. Methods for generating recombinant baculovirus are known in the art (see, e.g., the Bac-to-Bac© Baculovirus Expression System (Thermo Fisher Scientific, Waltham, MA)).

In some embodiments, a rAAV particle produced by the methods provided herein comprises an AAV9 capsid protein. In some embodiments, a rAAV particle produced by the methods provided herein comprises an AAV-PHP.eB, AAV-DJ or AAV2 capsid protein. In some embodiments, a rAAV particle produced by the methods provided herein comprises an AAV1, AAV2, AAV3 (including types 3A and 3B), AAV4, AAV5, AAV6, AAV7, AAV8, AAV9, AAV10, AAV11, AAV12, AAV13, AAVrh32.33, AAVrh.8, AAVrh.10, AAVrh32.33, AAVrh.74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, TM-AAV6, AAV-PHP.A, AAV-PHP.B, AAV-PHP.S, AAV-PHPeB, AAV-CAP.B10, AAV2-r3.45, AAV2-LSS, AAV2PFG, AAV2-PPS, AAV2-TLH or AAV2-GMN capsid protein.

### Methods of Using Vectors

Provided herein are methods for increasing the expression of wild-type (or normally functioning) SCN1A in a subject in need thereof, the method comprising administering to the subject a therapeutically effective amount of a vector, a viral particle, a population of viral particles or a pharmaceutical composition disclosed herein. The subject has a heterozygous loss-of-function mutation in the SCN1A gene. A loss-of-function mutation may be a nonsense or missense mutation or a deletion.

Provided herein is a method for increasing levels of SCN1A expression in SCN1A-expressing cells in the brain, the method comprising contacting the cells with a vector, a viral particle, a population of viral particles or a pharmaceutical composition disclosed herein. In some embodiments, the SCN1A-expressing cells comprise a loss-of-function mutation in one copy of the SCN1A gene. In some embodiments, the SCN1A-expressing cells are GABAergic interneuron cells. In some embodiments, the GABAergic interneuron cells express parvalbumin (PV).

In some embodiments, a vector provided herein increases SCN1A mRNA expression. Levels of mRNA expression may be measured by a Northern blot, a nuclease protection assay (NPA), in situ hybridization or reverse transcription-polymerase chain reaction (RT-PCR).

In some embodiments, a vector provided herein increases SCN1A protein expression (i.e., expression of the alpha subunit of sodium channel Nav1.1). Levels of protein expression may be measured by a Western blot or immunohistochemistry.

Provided herein is a method for treating Dravet syndrome (DS) in a subject having or suspected of having DS, the method comprising administering to the subject a therapeutically effective amount of a vector, a viral particle, a population of viral particles or a pharmaceutical composition disclosed herein.

Provided herein is a method for treating or reducing the risk, severity, frequency or length of a symptom of DS in a subject who has or is at risk of having DS, the method comprising administering to the subject a therapeutically effective amount of a vector, a viral particle, a population of viral particles or a pharmaceutical composition disclosed herein.

Provided herein is a method for treating or reducing the risk, severity, frequency or length of epilepsy and/or seizures in a subject who has or is at risk of having epilepsy, seizures, or DS, the method comprising administering to the subject a therapeutically effective amount of a vector, a viral particle, a population of viral particles or a pharmaceutical composition disclosed herein.

Provided herein is a method preventing or reducing the risk of Sudden Unexpected Death in Epilepsy (SUDEP) in a subject who has or is at risk of having epilepsy, seizures, or DS, the method comprising administering to the subject a therapeutically effective amount of a vector, a viral particle, a population of viral particles or a pharmaceutical composition disclosed herein. SUDEP may be defined as death in a patient with epilepsy that is not due to trauma, drowning, status epilepticus, or other known causes, but for which there is often evidence of an associated seizure.

In any of the methods provided herein, the vector may be a rAAV particle comprising the nucleotide sequence of SEQ ID NO: 137, 141, 145, 149 or 153, or a nucleotide sequence at least about 90%, about 91%, about 92%, about 93%, about 94%, about 95%, about 96%, about 97%, about 98% or about 99% identical to the nucleotide sequence of SEQ ID NO: 137, 141, 145, 149 or 153.

In any of the methods provided herein, the vector may be a rAAV particle comprising the nucleotide sequence of SEQ ID NO: 137, 138, 139, 140, 141, 142, 143, 144, 145, 146, 147, 148, 149, 150, 151, 152 or 153, or a nucleotide sequence at least about 90%, about 91%, about 92%, about 93%, about 94%, about 95%, about 96%, about 97%, about 98% or about 99% identical to the nucleotide sequence of SEQ ID NO: 137, 138, 139, 140, 141, 142, 143, 144, 145, 146, 147, 148, 149, 150, 151, 152 or 153.

In some embodiments, the rAAV comprises an AAV9 capsid protein. In some embodiments, the rAAV particle comprises an AAV-PHP.eB, AAV-DJ or AAV2 capsid protein. In some embodiments, the rAAV particle comprises an AAV1, AAV2, AAV3 (including types 3A and 3B), AAV4, AAV5, AAV6, AAV7, AAV8, AAV9, AAV10, AAV11, AAV12, AAV13, AAVrh32.33, AAVrh.8, AAVrh.10, AAVrh32.33, AAVrh.74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, TM-AAV6, AAV-PHP.A, AAV-PHP.B, AAV-PHP.S, AAV-PHPeB, AAV-CAP.B10, AAV2-r3.45, AAV2-LSS, AAV2PFG, AAV2-PPS, AAV2-TLH or AAV2-GMN capsid protein.

In some embodiments, the subject is human. In some embodiments, the subject is less than 2 years old. In some embodiments, the subject is between about 2 years old and about 18 years old. In some embodiments, the subject is older than 18 years.

In some embodiments, the vector, viral particle, population of viral particles or pharmaceutical composition is administered to the subject via intracerebroventricular injection, intrathecal injection, intracarotid artery injection, or intraparenchymal injection.

In some embodiments, the vector, viral particle, population of viral particles or pharmaceutical composition is administered to the subject in a single dose. In some embodiments, the single dose comprises from about 10E+9 to about 10E+14 viral particles.

Further provided herein is a vector, a viral particle, a population of viral particles or a pharmaceutical composition disclosed herein, for use as a medicament.

In some embodiments, efficacy of a vector, a viral particle, a population of viral particles or a pharmaceutical composition disclosed herein may be tested in an animal model of DS (e.g., a mouse model). In some embodiments, a mouse model is Scn1a-KO: 129S-Scn1atm1Kea/Mmjax; Scn1a-R1407X:Scn1aKIdneo; Scn1a-R613X:129S1/SvImJ-Scn1aem1Dsf/J; or Scn1a-A1783V: B6(Cg)-Scn1atm1.1Dsf/J.

A vector, a viral particle, a population of viral particles or a pharmaceutical composition disclosed herein may be tested in the following experiments:
In-vitro demonstration of the level of expression of a DTM at the RNA and protein levels;
In-vitro demonstration of upregulation of SCN1A gene for specific gRNA sequences at the RNA and protein levels;
In-vivo demonstration of specificity of expression of the transgene in the target cellular population in wild-type mouse brain using immunofluorescence;
In-vivo demonstration of upregulation of SCN1A in the brain of wild-type mice at the RNA and protein levels;
In-vivo demonstration of upregulation of SCN1A in the brain of Dravet mice at the RNA and protein levels;
Demonstration of increased survival of treated versus untreated Dravet mice; and
Demonstration of decreased seizure in treated versus untreated Dravet mice.

### Definitions

Unless otherwise noted, the terms used herein have definitions as ordinarily used in the art. Some terms are defined below, and additional definitions can be found within the rest of the detailed description.

The term “a” or “an” refers to one or more of that entity, i.e., can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein, the term “gRNA molecule” or “gRNA” refers to a guide RNA that is capable of targeting a CRISPR nuclease or a nuclease-deficient CRISPR-associated protein to a target nucleic acid. Depending on context, the term “gRNA molecule” refers to a guide ribonucleic acid or to a nucleic acid encoding a gRNA.

As used herein, the term “sequence identity” refers to the extent to which two optimally aligned polynucleotides or polypeptide sequences are invariant throughout a window of alignment of residues, e.g., nucleotides or amino acids. An “identity fraction” for aligned segments of a test sequence and a reference sequence is the number of identical residues which are shared by the two aligned sequences divided by the total number of residues in the reference sequence segment, i.e., the entire reference sequence or a smaller defined part of the reference sequence. “Percent identity” is the identity fraction times 100. Percentage identity can be calculated using the alignment program Clustal Omega, available at ebi.ac.uk/Tools/msa/clustalo using default parameters. See, Sievers et al., “Fast, scalable generation of high-quality protein multiple sequence alignments using Clustal Omega” (2011 Oct. 11) Molecular Systems Biology 7:539. For the purposes of calculating identity to the sequence, extensions, such as tags, are not included.

As used herein, a regulatory sequence (e.g., a promoter) is considered to be “operatively linked” when it is in a functional location and orientation in relation to a nucleic acid sequence it regulates to control transcriptional initiation and/or expression of that sequence.

As used herein, the term “self-complementary” when referring to an AAV vector refers to an AAV vector comprising a nucleic acid (i.e., a DNA) that forms a dimeric inverted repeat molecule that spontaneously anneals, resulting in earlier and more robust transgene expression compared with conventional single-strand (ss) AAV genomes. See, e.g., McCarty, Molecular Therapy 16(10):1648-1656 (2008). Unlike conventional ssAAV, self-complementary AAV (scAAV) can bypass second-strand synthesis, the rate-limiting step for gene expression. Moreover, double-stranded scAAV is less prone to DNA degradation after viral transduction, thereby increasing the number of copies of stable episomes.

As used herein, the terms “treat,” “treating” or “treatment of” (and grammatical variations thereof) mean that the severity of the subject's condition is reduced, at least partially improved or stabilized and/or that some alleviation, mitigation, decrease or stabilization in at least one clinical symptom is achieved and/or there is a delay in the progression of the disease or disorder.

As used herein, the terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the compositions and/or methods described herein. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the compositions and/or methods described herein.

In some embodiments, a nucleic acid sequence provided herein is a nucleic acid sense strand (e.g., 5′ to 3′ strand), or in the context of a viral sequences a plus (+) strand. In some embodiments, a nucleic acid sequence is a nucleic acid antisense strand (e.g., 3′ to 5′ strand), or in the context of viral sequences a minus (−) strand.

As used herein, a “therapeutically effective amount” is the amount of a vector, viral particles, a population of viral particles or a pharmaceutical composition provided herein that is effective to treat a disease or disorder in a subject or to ameliorate a sign or symptom thereof. The “therapeutically effective amount” may vary depending, for example, on the disease and/or symptoms of the disease, severity of the disease and/or symptoms of the disease or disorder, the age, weight, and/or health of the patient to be treated, and the judgment of the prescribing physician.

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

TABLE 1
Exemplary SCNA1 targeting guide sequences

| | | SEQ |
|---|---|---|
| Guide ID | Guide sequence | ID NO |
| SCN1A_P1_gRNA_1 | GGTGTTTGCAGTAAACCTAAC | 1 |
| SCN1A_P1_gRNA_2 | GTCCACCTTTAACTCATTGTG | 2 |
| SCN1A_P1_gRNA_3 | TTTGCAGTAAACCTAACAAGA | 3 |
| SCN1A_P1_gRNA_4 | TATTTCCCTCTACTCACCACA | 4 |
| SCN1A_P1_gRNA_5 | TCTGGTCATTCTTCCCACAAA | 5 |
| SCN1A_P1_gRNA_6 | GAAGGCGAGTTAAGAATAGCT | 6 |
| SCN1A_P1_gRNA_7 | AAAAGCCCTCGGTGACTGTCT | 7 |
| SCN1A_P1_gRNA_8 | AATGAGATTATTATTGGGGGT | 8 |
| SCN1A_P1_gRNA_9 | GTCATTCTTCCCACAAAAAGA | 9 |
| SCN1A_P1_gRNA_10 | ACCAGTTTAACCCACCACGTT | 10 |
| SCN1A_P1_gRNA_11 | GGATATCAAATTCAGAACGTG | 11 |
| SCN1A_P1_gRNA_12 | CAGCAGGCAGCCAGGCAGGCT | 12 |
| SCN1A_P1_gRNA_13 | TCTTGTTTGTGAGCAAGGTAA | 13 |
| SCN1A_P2_gRNA_1 | GGCCACCTGATTGTTTCCTGG | 14 |
| SCN1A_P2_gRNA_2 | TTCATATTATAGTGAACAGCT | 15 |
| SCN1A_P2_gRNA_3 | TCATGATATTATATAGCAATA | 16 |
| SCN1A_P2_gRNA_4 | AACTGTTACTTTATTCCTAAT | 17 |
| SCN1A_P2_gRNA_5 | TAGATAACAATGATTCATATT | 18 |
| SCN1A_P2_gRNA_6 | GTTGAGGAAGAAATCATAAAT | 19 |
| SCN1A_P2_gRNA_7 | GTAATTACAGATGAAGATAGT | 20 |
| SCN1A_E2_gRNA_1 | ACTGGCCATTAGGCTTTCTCT | 21 |
| SCN1A_E2_gRNA_2 | GCAAAACCTGCCATATACAAC | 22 |
| SCN1A_E2_gRNA_3 | TGAACATCGAGAAAGGGGAAG | 23 |
| SCN1A_E2_gRNA_4 | GCCACTGGACCACCCCTGTAT | 24 |
| SCN1A_E2_gRNA_5 | ATTTATCTAGGACTCATATAC | 25 |
| SCN1A_E2_gRNA_6 | CGCACCTGACAGCAATAATTT | 26 |
| SCN1A_E2_gRNA_7 | GGAAATTTGCTCCTTATTATT | 27 |
| SCN1A_E2_gRNA_8 | ACAGTGGATGAAGCCCTTTGT | 28 |
| SCN1A_E2_gRNA_9 | GTATTGTATGTCTCCCAGGAA | 29 |
| SCN1A_E2_gRNA_10 | GAAGCCCTTTGTTTGGATTAA | 30 |
| SCN1A_E2_gRNA_11 | AGGTGCGTCTGCCATACAACC | 31 |
| SCN1A_E2_gRNA_12 | CCATACAACCATGAGTCCTGA | 32 |
| (G1) Scn1a_Ms-P1_gRNA_2 | CAACTCCTGAACCATCACATC | 160 |
| (G2) Scn1a_Ms-P2_gRNA_9 | AAAGGAACCAGAAGGAAGCGG | 161 |

TABLE 2
Exemplary mouse enhancer sequences

| | Mouse | Size | | SEQ |
|---|---|---|---|---|
| Name | coordinates | (bp) | Mouse sequence 5′-3′ | ID NO |
| E1 | mm10_chr2: | 1279 | caaagtggacagaggggaggggaggggatgcgaggggaggggaggggaatgatcgcgaaagg | 33 |
| | 66256056- | | cttccaaaccttgtccttgtttttcaccatttctgaaatatatgctgagtgcaactatggga | |
| | 66257335 | | agaccattttcataatctataatactcctccttttaaaggactttcgttaaccgcttgcaaa | |
| | | | ggtgagtgccgggtagaggacattagctcgctaagtccctagaaatcacacttggagactaa | |
| | | | gcaggctttcccaggagaagtccaaagccaacataagcaggaggctgggggctggccgttaa | |
| | | | ccgcaaggcagtggttgagccctcgggatcatcccggcggggggcgcagcatctccgccaag | |
| | | | gccgcaggctctcaccatcagctgcccgagccaccctgtacctcgcagtccactcgccctgc | |
| | | | ccacgccccgcgccgcccgctcaccttcagcccctgggagtccatggccgccggctacccgg | |
| | | | agggtgcccaccgctgcccgccgcagggttaggggttcagacccacttcccgggccctcaaa | |
| | | | ccctaagggacgcggcgtgcagcacgaggggcgtggcccgatctccattggttgtcggcgtg | |
| | | | agggggcggagcttagttgtaggactaggaaggagggggccaccggagcaggcgaggaggga | |
| | | | accccgagggaggaccgcgagggcgactggggctggaatccgctgagcattgagtgctgccg | |
| | | | agttgtggggctagaggagggaggtccagcctggaaacggcgcgaggaggagggattgggtg | |
| | | | gagcaagagatatgagattaaagaataaagatgatgaagcagcaaataggagggagagccat | |
| | | | gccgcttttcatccctgcaaacaaaggccgactccattttctcagcattttttgtggaagcc | |
| | | | gatttgcgcaatgcggcttagtacttgaccagggaaaatgatttacctgacacgtgtagtaa | |
| | | | tcgtgtctgggccacaaggtggcgcagaaaaatcacagttcggcaaaaaccttgaagcctgg | |
| | | | cttgggcttgttctaaatcttttcaggcgctgctgtaattttgctattcgagtgcttattaa | |
| | | | actgctccgccagatttccacccccaaagtcttatttaaaaatatgtggttacctcttttag | |
| | | | atttctatttcttaagtgtttgctgtagtttggatctaaactgtccctcaaagacacacgtg | |
| | | | ctgaatgttccccagcccgtgtgctgttgggagtggtgga | |
| E2 | mm10_chr2: | 617 | aatctaacatggctgctatagcttactgactagaagttaagtgcacacttcctaaaagaagg | 34 |
| | 66364036- | | ctttgacacaagccacttcagttccctcctcattttcttgtccccattcctctctctgtaga | |
| | 66364653 | | attctgagatttcaattcagttttatacagaaaccacattactgtaagccctacaaagttat | |
| | | | ggcaatatagctatatggagtcaagtaatgtaggttatttttttcccaatggtgctggtgaa | |
| | | | ggtggcaattatgtagctatacttagcagactgaggaaattctgctagagtcagcatttgtc | |
| | | | tcttcattgctatgaaacagtaatggaaaaataaacaaaaacaaaaggcaaacactatgcat | |
| | | | aattccctcagatcatattaacatgtgatgttggagtaaattgttataaccccattttggaa | |
| | | | atacttaccttaattaactatgatttccttaaaataatgcagtatttacaatctatatgaaa | |
| | | | gcactatatgggacacatggtatgatggaacagtgcacccaagagacaccaagaacattcct | |
| | | | gtctgtggcagtcttttctctatacagaggcatttagtctcaattgctcagagttatttt | |
| E3 | mm10_chr2: | 831 | ataaaattttattttcctaaaatttacatttaaaagccatccaacttaccaaagtgatttca | 35 |
| | 66383190- | | aaccacaattacattttatctcaactaccagcattttatttagccaggatctacatgagaca | |
| | 66384021 | | catatcatgatgtgctatgtacatcttgttatacagtgttatattgataacaaatgtcatca | |
| | | | atataacattgaattaatcttccataatttatggggaaaaaaggagcagccttactgaaggg | |
| | | | caaagttatacaacagctttacagaagctgcatgcgagtgcagtaccgggacacgggcacgg | |
| | | | acggcggcactataaccattttccgtggtggtaatcttgctttcatctgacacagaaaagag | |
| | | | accgccgttttgaaaactcacagaactagcctcacggttttgtgagtccattgagcgctggc | |
| | | | tgcgaagaacggtgtttaactcgagaaatcattgaacaagttttagaaaataaagatgctta | |
| | | | tgacaatttcaaacttgaaggtctccaaagaaggactgagatattggtgagaggagtaaaga | |
| | | | gaatcctggtgcatttatttcatgcttccttctgttcgaagatcattttgaggtttataaaa | |
| | | | ggtggggtgatccaaaaatctccaggctgagagtcctggctgaggctgtgaactgggctgca | |
| | | | gagaaagggccacgcctccctcctctgctcgcattactcagcagcttttctgcatgtggctg | |
| | | | gctgcagacaatctaaacccttccgctgtcgctccccccttatactgttctgccaaaaggaa | |
| | | | ggcagagaggaaatcagctacggggc | |
| E4 | mm10_chr2: | 260 | tctgacagagcaagtcttgacctgcttaacattatgttatgctagtcattttaaaatgagtc | 36 |
| | 66387764- | | tttatttcccatagaaggtcagtttttttacattattatataatcttttgacagaataacaa | |
| | 66388024 | | ataacattctgaatgtctcatttctaaatacaaaacatcttagtataaaattatgcattgtt | |
| | | | ttaaatgcttggaagtaggtccacatgtagaaaacaaagtacgtatgataaaaaatatcaaa | |
| | | | attgtatattcag | |
| E5 | mm10_chr2: | 662 | aatgttttgatatttaggagaaaattgcaaaacaaaatgatgacagtgtttgaaagtgtttg | 37 |
| | 66392447- | | atcagtgccaagcatcactttatgtacttggcaaacatgacttgaggccttaagctgtgatt | |
| | 66393109 | | tgcaaatgtagattggaatcaagatctttatagatgaggaagcaaaaatcagaagacaaaat | |
| | | | aacattatcaacttgatctcatgtgcagccagggctgaactgcaaatgctgatttgccccag | |
| | | | tctgggctcctcaaatcgttccttggaatcctattagttggaactttatctctgctcgtggc | |
| | | | agggtgcctgggaccatgtttataaatatctgctgaatgaagaataagtgagtcaatcgaac | |
| | | | cagaactcactttggttagttaatttcattcgtggtatttatggagagcagaagaaagaatt | |
| | | | ccagagacacgatttgtcaaaactctctaaagaaaatgatgacactatatattgatgaaaat | |
| | | | gaatgttcttgttcttgctttatttgattttcttgtccccccactccccatctgctagggtc | |
| | | | tcattacagcatagttcttgaatatcccaggttgacctgaagttacaatatattcttgattt | |
| | | | agatggcagacattgggaatattttgactcttaaaatttaata | |
| E6 | mm10_chr2: | 605 | ttgtcactttgttactctacagtgttgcctggagttcgatacttcattactctatagtgggg | 38 |
| | 66401767- | | tgaagaagttcaccttcttattttcatttccttccctcaatgatttcttcagagctagctct | |
| | 66402372 | | taccagctagaaattcttcaaacgacactcgtgccttccttcacacaggttgaactatttgt | |
| | | | ctctaatgccctaaagtactggtgttcaatcttccaggcacttccaatgatctgaaatctga | |
| | | | cctgcttaggtcagctggctctgagattatggtattctagtcctcaaaccaacctgttggct | |
| | | | cgttggttttgtaccaaacacactgacttacatagctcaaaataccactggccttttaaaaa | |
| | | | tggcatatcacattccaggggaggatcaaaactgctggctggtgatatttgtcaagtctctc | |
| | | | aaagttgcactttccaggattttcaattcactgaattcttagacagacatgtttatgtgaaa | |
| | | | gaattctttatatattttttctcctctttgagtgggcaaatgaaaatcttgacctctgggtt | |
| | | | ccttattttatttgactctctgtagtatttaaatcttaaaattttcct | |
| E7 | mm10_chr2: | 2429 | gatactgtataattaattaggcctccaatcatgccttcccagcctccacggatggagaaacc | 39 |
| | 66407834- | | ctctccgccatgccttaaagaggaattgctgtaataaatgagtctcctgatagcaaatttct | |
| | 66410263 | | cagcaagggggaatcgcgtaaatggagacatagtattgacagcaaagtccaatgtgttattt | |
| | | | ttaccagaacgaactctccggttcaagcctttgaaagagacatttgaaaaccaaaaacaaac | |
| | | | aatgtaatggagcgaggaaaaaagccacagaagtgagtggcagggagtttaaaagagcagat | |
| | | | gccactgccaggtctatgggacataaccagccacttgtgctgggtcttggcagtttataatg | |
| | | | ctacctcatcttctccgcgaaattgttttcccgtaaatctctgtggccatccattcctgtct | |
| | | | acacattatgttcctaaaatagacaccatctaaaaatcacttcaaggagctttgtggaggaa | |
| | | | ggcctaaattgcaacactcctccagcgaagatagatgcagtgtttgatggcattaccagtcg | |
| | | | gtagccaggaaggggagtttgtgaggagtttttccaccacagttaatctgtttctggaagga | |
| | | | aagggaagtgtcagacttcccgaggaggcaaacgtgtgtggaagctctcatttgcatcaccc | |
| | | | ccggcctgtcaggtattgcagcaaaagggagaggtgagctaccctggctctccttgggcagg | |
| | | | agggacagaatcaggaagcatcaacctcagcatggaattttcctattcctgtttggcatcct | |
| | | | cctcttgggatgatttacagcgcgggttggagaaacacgctctgccactccactagcgcacc | |
| | | | agatagacagtgcagacctgcagatccatacccgaggagaagccacatttcctacgtgtgat | |
| | | | agcaacagcgtttggcaatttgcgactttgctactgcagcttagaaaatatttagtcacatg | |
| | | | cacatctgaacagaaagacacccaggcttgactcagtcatttccgtcagacacacgaaagaa | |
| | | | aaagcgtctctgctcacaagcttatttggactgctttgttgaaaggaggggcggcagacact | |
| | | | ttgtagatgtggcaagagggctttatatccagacctcaaacaggtaggagagaaggaagcca | |
| | | | ggagaggtaaggaaggggcgtggaaaagcctcacagccacctcgaagaaaacagtttttttg | |
| | | | ccctgttcagaaagcaagaggttccacagtggttttgtgtcaatggagcacatctgcagtat | |
| | | | cattgccgttggtgacctctgtctaattaaaagtaagtcagtccttcccacccggcattgtc | |
| | | | tgaaacccgggactctttatcactttgctaaagttcatttgcaagtgtagttaaggaagagt | |
| | | | caggggggaaacagcatctgtcccttctggtcctggggaggaggcactcctttccaagagtc | |
| | | | aagcctctgcccaaagaagctgcctcccctgcaatgctaggatccaggagcagccccgctgc | |
| | | | cttcttgcttcctctgtgaggtctaatttttgcatcatctttaggagcgatatgacctctat | |
| | | | tcacagccatcgaatccagttccaaagcaccaatgacagagggggcttcaagacaagacctt | |
| | | | gcctaggaggatgcaggcaagcaaaggcaagagctggcccgatgccaagttattttaggcca | |
| | | | aagaatctcatccttctatcaaaatgctgaactgcaaaacgaacctgatttcagttcatgga | |
| | | | aggttgagaggaggaggagggggaggggaggaggaggaagagaggaggaggggaggaggagg | |
| | | | gaggaggaggaggggaggaggagggggacagttggtccgaattcacatgcaaaaatagactt | |
| | | | cctgttctgccccaactcttatttccgtgggctcttctccccaaggatttaccaggtaagaa | |
| | | | ttcaccaccaaagaagatcacaatgagataatcagatggcttacctgataaaaaggaaaatt | |
| | | | atccatctgcagtgaggagcaacatctccccacgacgagtccgcaccttccgttgcaacgat | |
| | | | tcagattccttcttgcaaaaggtgaccaagtgcttcacaagggctgcagcctcataggggca | |
| | | | gaacacacgtacacaaacacacgcacacacacacacacatgcaccagagacctctgcagtat | |
| | | | cctctcggcttcatcctcgcctcactctatggtacctaatacaaatcagcaaatagcttgtt | |
| | | | ttaaaaaaaaaagaaagaaaaaaaagcggagacagcacctaacgttacagtgccatctagtg | |
| | | | gctacatcgtaaataggttctcacagcctggatttctgtgttctttctcaaccgcttccttc | |
| | | | tggttccttttt | |
| E8 | mm10_chr2: | 1643 | attgatctccaactttttaaatccctctgtcattttaaatgaggtgcactcggttgtgtcat | 40 |
| | 66439814- | | catctcggttttaattgtgtgaaaatttcctgccaatctcacaccgctgcggcaacctcacc | |
| | 66441457 | | ttgctacttgccctgcaagttctgcagtgtgccgttctgagtatgccgttttaactagttct | |
| | | | tgcagcaggacacaaagcgagatagtctgatagaaccagttctcctctggttttacctttac | |
| | | | tcttagatgagttaagggtcacatcaaaccagggctcagcccgccagatctcctaagcacag | |
| | | | cccctcctgacccaatgcagttaacccaacctcattcagcgctagtatcaaatgacactgga | |
| | | | gctgctgcagtatgcatcccgagactaagtaggcaggatttattatcagcagaagtccccta | |
| | | | actaccaggttattcaagctccgttcttgtcacaaacaggcgcggcggaagacacagtgcag | |
| | | | cagactcagagctcatttacaagacaagcgaattctcagttagagacaagggcagcgcggca | |
| | | | gcgaactgcagtaaatcttttcacgctcacagcaacatctaacaatgctctcctgcaacgcc | |
| | | | tcagatcaaacgaatcctacttggtttaaacatcaaatcaacaccataaaaaaggcttcatt | |
| | | | agcaaagttcaatttaggatgtttttaatcgtgtcttaattctagaaccagtgcgagacttt | |
| | | | ccatgcttattcaagcatgctgacagaattggaacctcttagaattgcctacctgcacctat | |
| | | | cagcctggctgacaggagcccgccaaaggattaaaaaaaaacaaaacccaaaacataaaatc | |
| | | | atgcaaaaaaatatttacccccgaaagatgtatgtagttaaagctcagcttcctgcagcctc | |
| | | | gatagcccctgaagtgttaatctgaagaaacagttccatgagtttccacaggccggtagtga | |
| | | | gtctcctacacttgacctagacagacttacataatgaagcatcagtgctggggagcttgcac | |
| | | | gatgtcatcaccagcaagagtaagaagtattggcagcagcaagcaggcgggcaggctgagat | |
| | | | cttgcatggaaatcatgaaccaggtcttgcttttcgtttttgaaacgttttggaaggagagt | |
| | | | tatgaatagcccagaaataggtctcattttgtgggtaggaagaatgaccagaagcatgaaag | |
| | | | ctaaatctcctggcaagtgcaggggacctctcttggagtgtgcagtaaacccgaggggacga | |
| | | | cttctcctgctgtcaactcctgaaccatcacatctggagtgaaggaaggggctggtgaagcc | |
| | | | ttgtaataaatgcaaaggatgctgctgagagctttggtctgcctttaactcattgtggtgag | |
| | | | tagaggggatgtggcagtatgcaatgagagttggttgtgtaggttgctttgcagagtaataa | |
| | | | ccaaaaaaaaaaaaatctgtgaagtgctcaatactttagacacattttaataaacaagatga | |
| | | | tagtaaaattactcttctccatcaaattgagactgtgctgggttaaactgttttaatgcatt | |
| | | | ttaactcctgatgttcatccaagtaataagag | |
| E9 | mm10_chr2: | 520 | atctcaagtgtatgtaacatgagctacagtcttaaaacctacaaacagtacatccagtctcc | 41 |
| | 66441748- | | taccatgattctgagtgtgatgatttcatatgagcacaagatgacatcatactatttagtta | |
| | 66442268 | | tatgtaaaatcatggtcttacatgggttgtggacaaaaccatctagttttggaggtgacaga | |
| | | | aatagagaggacgccatgcactacttaaaaataatcgcagccttcttttcttagctagggag | |
| | | | tttgctgctatgagccacattaagaccagggtgaggagatgagacgatacaggggcatgaaa | |
| | | | gaacacggtgatctactttctcctgttaattaacgagtaaggaaatagacattaaaagaagt | |
| | | | taaatgtgtctgagccaacgtaggtgaggtttcccccaaattcacctggtagttttgctact | |
| | | | gcagtatagtaaatacttgttttcatttgtttttttttttttgttttttttgtttttttgtt | |
| | | | tttttgtctttttgttttttttttt | |
| E10 | mm10_chr2: | 546 | tattgcaaaaggaaggaatgagacagtttatgcagagctaagggtttgtgcgttattatgat | 42 |
| | 66450594- | | taatcacaaggacagctgccaagcttccatcatgacaatattctctgggagaattcatcagg | |
| | 66451140 | | ttctactgtctattaatttctgttgatgtatcttatctggcatcttcaatgacagaggacac | |
| | | | ttgttagtttttttttttaagtgaaggttaaaagacaaagttcattaaagaaatgatttata | |
| | | | tatgacatttaagaactagcaatgtcattgcttcaagaaaattatgagaatttagtcttggt | |
| | | | aggagtttacaccatgtccttgaagtgtctaattatgtgacttgatagttttacttagtaca | |
| | | | tatcgattaggctgtatctattatttatcaagaaattatggaaggaggcaatgtggcatagg | |
| | | | catacacattctgattttaaaataatcctgcttttaccattaactccttctcagataattct | |
| | | | gaatacatatcttgtctatgaatctgtgtaatcatggaaaaagaaaaaatc | |
| E11 | mm10_chr1 | 503 | actgtttgagcgggcagagcaagtgcgaaaagattaaaaagtgttctcctcatcctttctgc | 43 |
| | 5: | | tcatatgaccagcgctgcagtgtcgcgcgccaggcgcacgcccgctcggcctggctccgcgc | |
| | 78204152- | | cagggggcgcccggctcagcgcacagctcagcagctcagtcccgccgcaccgccaagctgag | |
| | 78204655 | | cgggccacatgcccatggggtccgggcattcaggaggctcgcccaggaccccgccgccaggg | |
| | | | gccatctcctgatctaaagggccaccagcagagactggagcgttctgccgctgtcaagggca | |
| | | | tacgggaggcagagaggctccgatctgggcatctcggacaatgtcctctcgcgtggggaaaa | |
| | | | aatgatgctgaccagcctccaccccgatgtggcccctgggctatgcactctgtctctccacg | |
| | | | cctccagtaccctcttgtcttctgtcctggatgcaaatttatgccgcgaaaaatctgaatgt | |
| | | | taagaact | |
| E12 | mm10_chr1 | 201 | acgcctccagtaccctcttgtcttctgtcctggatgcaaatttatgccgcgaaaaatctgaa | 44 |
| | 5: | | tgttaagaacttgaaacctgagccacaggacccagggaggaggttgttggactatcagagct | |
| | 78204583- | | cctctgtgcagaggctctctgtctgggctccatccctccctcacctgaccacattctgaaac | |
| | 78204784 | | ttgatttggtttctgc | |
| E13 | mm10_chr1 | 532 | tctctctggtcatgtgttgtgttaaagatgggaggatcactgtgtgggacctgaggcaggtg | 45 |
| | 5:78205234- | | ggcagcgtctggggtgatgatgatactggggaccaagggacagcttcccagctgcccagtag | |
| | 78205766 | | ctcaagttgcctgacaacttgctgtcaacactgtctcccctagctgagccctggaaggctgc | |
| | | | ccttcctggaaaccttaggagtgcctggctccagctgccccctcctggtgagtcagcgtctt | |
| | | | tgggccacagcttgccagctggcttccaaggctcctaggctacagcccctgacttcatagct | |
| | | | ctgggcatgaggcccacttcagaccccccagctgtcctgcacccttcagctgctttagctcc | |
| | | | tatgtctcctgcctcagttgacttagtggcctcagcctctaccccttgttcccagctggact | |
| | | | gccagttccagtgtgccaggcttctgtgttgacagttcaatgtctgggctctctttatactg | |
| | | | tggccccagttacagcatctgtttctccaactggaga | |
| E14 | mm10 chr7: | 495 | tcggttctctctgtagtctgcaaagaacccacttcttgcccagtgagagattcgggagattt | 46 |
| | 79052127- | | ccactgcactgcctgaggccactgtgctttaatgagaagaacacccgcttcctctcaaggcc | |
| | 79052622 | | agcagcggtctgaatccccagcccccaaacacactgcagcttgcccagctagctgctcctgt | |
| | | | gaggggtgaccttgacttgaagttagccccaacttgctggacaactccaccactttctgagc | |
| | | | cccatggtatatgtgagcattgttgggaggtgagaggtagtgttgtaatatttaacttattg | |
| | | | gactcgttagagaaattaaaatcactgtatgctgcaactagcttatagaagggatccaaaga | |
| | | | ctcttagatttttctttttccgggaaacaaatttattaaagagtgttgtagaggccaggccg | |
| | | | tagcgaattcctgcttgagatgtccttggtctatgttgcgttttgaagacacccctccccgc | |
| E15 | mm10_chr7: | 317 | tggccccgggggtggggtttccctgtgcgctcgcccccacccctcgtgtgtgccctcccctc | 47 |
| | 79053118- | | ccccgcccgcccctatgtatgtgtcaccgcgcaccattcccgcccacctacctccccgcggt | |
| | 79053435 | | gccagaggggctcacagagctgaggacgcgtgcggaacggctcaaggtccctcactctctct | |
| | | | ccgcatcctcgccggctagcatctgacgcaggtgccgagggagttcgggcacctttctgtgt | |
| | | | cccttcccttcatcgaccctgctccgaaacctagtcagccagccaccactgcggccaccgcc | |
| | | | cgagggga | |
| E16 | mm10_chr7: | 501 | agtcacagaattggactcctgtgtcccaggaatataagagcacaattgatgtagaggttcct | 48 |
| | 79056553- | | ttaaggatgacgccctccttcctggagactaactgcctgttagaaaggtctggagaaaggtg | |
| | 79057054 | | ggggcaagctggagagagagcgcaggcaagaagagaatcggaccagagaggaaatttgtagg | |
| | | | gaaactggtaaaacggtttcttttttcaaaaagttgaatccagggcaagaacggaaatggtt | |
| | | | gagtttactttgaaaaactcagagcctccttgtatgcagctgtgtgtgtgagtgtgtgtgtg | |
| | | | tgtgtgtgtgtgtgtgtgtgtgtgtgtgtgtgtgtgtgtgtgttggaggttgggccctgtgg | |
| | | | gctggctagagtgtgtgaaggcagctctgctccctcaatggccttacagtgtatcctaaaga | |
| | | | agctttttgttaaactcctgaataggtggattggggggtgtgaggctggacatcacgaaata | |
| | | | gctatt | |
| E17 | mm10_chr7: | 473 | acggcctcgctttccttcccattgagaaaggtctctctgtccacgagtgagcgtgggctggc | 49 |
| | 79079999- | | aaagtcagtatcctcacataaaagcctcactgagtctctgagaggcttggaacccagacaga | |
| | 79080472 | | atggaattgctccctatttttctttacaactgagaaaacacacacaaacacaagagcatatt | |
| | | | tattgcgataattctatcccaaagtttttaagaagaagaaataaaattagtcttgctgccta | |
| | | | ctccgcaccagccccctccctttcattccttcacatttcctggtctaagccagggagaaaaa | |
| | | | cagagccagcccaaaccccacagagcaccattgcatcccagacaatgtccagcttgttcaga | |
| | | | gagcgagggaagaaacacaactccgaaacatacacaagacttccaagtgaagggtgttctga | |
| | | | gggaggaacaggctagctttgtgtctgggtcttgggtaaa | |
| E18 | mm10_chr5: | 673 | aagtggggagggcttctaaggttggttctggaagcccgtctgccaaatgtgttttcactttg | 50 |
| | 127243448- | | tatggctgatgaaagctacagttcgtccccggtgtcttcctaacccagcctggtgtttactc | |
| | 127244121 | | tgtaagatgtcgcgtgcgtgcgtgcgtgtgcaagcgggtgtgtgttcgcacctatgtgtaga | |
| | | | gacaggagatcaatcctcgctgtcttccgcagtccgtctccacggagggttttttgtttttt | |
| | | | tctgagacagagcttctcatctggcggggaagttccctcattaagttccttccagccctgcc | |
| | | | ctgtgcattactgcctcctaagcgctgggaagccagcctgcttcacctgcctcgcttttctg | |
| | | | ttttcctgagtctgggattcaggtcctcctgcatacacgcgcatgcgtgagtctacccctga | |
| | | | gccacctcccggctcctccacagcatgtttgacagaaacaaacaccttctcacccttgttta | |
| | | | aatttccactgtcttaaaaacagctatgccaccagggactttgactatacaagggtctctgc | |
| | | | gcatgtttctggaaccttggaaatctgtccagtcatcggccaccaaccaagtgtgtgttttt | |
| | | | gacggttagttctgaagctttgcaacacatcaggggttacagaaagcacagttg | |
| E19 | mm10_chr5: | 338 | ccgctgttttacatactaattgcatttctgtaaaggaaaaaaaaatcattgagataaataca | 51 |
| | 127257256- | | tagctgatagcatgtttaaaggtccttctgtatttcctcccccattatctgcaatggcacac | |
| | 127257594 | | ttgggcaaacactggcctggggcccaggtacagatggagcaggcagatctagtgtcagctgt | |
| | | | ttcctccccgcgactggacctccagattgcatcctctgtcagaactttgaaggaaggctttc | |
| | | | agtaaagatggtattctaatttcctttctgatgctgtgataaaaggcatctcaccaaaagca | |
| | | | acttagggaaggaaagagtttatttcagc | |
| E20 | mm10_chr5: | 501 | tcggtggtgtcctggccccacacagggaaaatgaaacaggagtctgacgactgtccattgag | 52 |
| | 127290515- | | agcagctaagcttgctgtgcctggactgattggacacatggaagacagtgttgggtcgtcca | |
| | 127291016 | | ccaaagattggcctggagccaccgtacacagagccgagacacattacccccccacaccccgc | |
| | | | tacccccctggcctggaaggagactgtcaggcgtgacaaaacaggaatctgagcctaagaat | |
| | | | attcacagattgcaggcccagaaggaagtaggaagttcatattctctttatagcctcgctcg | |
| | | | ctgggggaccggcatgtcttttattgttaatataaaaatcaatatagcaggaggcgagtctt | |
| | | | tgccataatgatgttggctccttgcagcaggcgcgtggaattagattgctcataggtgtgcc | |
| | | | tctgtcgcctccccgggctccagagagaagctgtgggctttgccaataagcacagtcacaga | |
| | | | gtatgc | |
| E21 | mm10_chr5: | 340 | ccggcactgcctccggtgctccgaaagaatgtaaagggaaggttgtacccatcaagcccctc | 53 |
| | 127300767- | | ttgctctaccctgtgccccccatgctgtcagccaagtctggcccccaaggatacccctcctt | |
| | 127301107 | | ccctggggtgtcctggagcttggaattggcttcattttctccagaaggaggaatcagctttt | |
| | | | cccagccgtctttgagattatctttattcttcaaggagctccagtggccagagcctctgtgc | |
| | | | atcctgccaagtgccctgggatcttggaaatctgcccaggggctcagtggaggcctccactc | |
| | | | ctccagacagcacacagttatgaaagaaaac | |
| E22 | mm10_chr5: | 442 | tcggggtttaaccgttcagagtaattttccattataattttgtgttctaaataaatagaacc | 54 |
| | 127305150- | | agcttcactctagaaacgcttgtctgagtgctgtacaatgcatgtttaagtggctctggtga | |
| | 127305592 | | aattactaatcatacccctgcagtggataaaatagaattgtttgtgagttttgctcattcga | |
| | | | tgggaggacgctaaatttataaaagtagactcatgcccttgatagtgaatttaaataggtgc | |
| | | | atatgtaaacagatacttactcatttagagacaccgtcatcagggagctttgtgtgagaatg | |
| | | | ttttctatgctccggctcctgctctgtggtaggggaactaagcagacatcccaggtccattt | |
| | | | cctaacccagtgtctctagttgagtaatcacacaaggcagacatcccagctgggactctata | |
| | | | tttaaaccc | |
| E23 | mm10_chr5: | 544 | ctcttcccactggtctgggggcactcctcttcctgagggctatggttctcagaccacataaa | 55 |
| | 127323924- | | attcaaatctcctggaacagcgcagctccctcctccccccacagcgagctccggatggtgtc | |
| | 127324468 | | tgagtttcatctttacaaggggcctttaaagccgcctgggcccccttcaaaatgcccagccc | |
| | | | cctttgtccagatggggactggggtggccaggctgcccggctgattgtgtgccgagcagtcc | |
| | | | tcccagggtaggctgtgatttatacgcacgggcttgtcaccgggtgaaaggaagggccactt | |
| | | | tttcattttgatcaaatgctaggtttgaaagccacccactgccgtaaactcagctggatccg | |
| | | | cggggcccgagatgaaacacattgcctgctttgttgctgagatggtgtttcggaaggctgtg | |
| | | | tgaatgctcttcccttggctcagctctcacacacacaagaagtgtagtgcagagactgactc | |
| | | | cctggctaggacctcagtcagggggagagattggagaaggtgctgttca | |
| E24 | mm10_chr5: | 556 | acgctgctgggctcagaggtggccaggagcacattggccttagggagttgtgaggagaggac | 56 |
| | 127331966- | | ttcagagctctgagttgggtttgtgctgtttttttctggcttcctgtttggaggaggctgtc | |
| | 127332522 | | tgcacagcgttggctccgcagctgttaattggcaggaagcgataatcacgcaagaatggctg | |
| | | | aaatcatcagttagggatcttgtttttttggaagtaagtgaaatcctgagatagcctgccct | |
| | | | aattatttcctggcttaaagaaaccaggtgcattgaagacatttttttttcctcttctgatt | |
| | | | tatggaggtcttagggtctgagttttgaaagtgctgagggaatgaagttctgggtgtcgtcc | |
| | | | taggaatacaaaacttttctatatatctcacgataaaaatctcttcgattagcttatctaaa | |
| | | | ttctgacaagttaaaatgtgtcctagatttttttttgttgtttctgtaggtaccagtttaaa | |
| | | | tatttcattacgtctattggggggggggtgtatgtgtgttctcatgcatgcacacatgtgc | |
| E25 | mm10_chr5: | 315 | tcggtattttgagaagtacagtgagtttcttaaacactcagctctgacacttccaaggactc | 57 |
| | 127355818- | | ttcctggaatgttgtaccttggcaaagcttccgagtgtgtctgaagctcgaccgggactgag | |
| | 127356133 | | tgggttaaactggagatgttctgccgtccctaatgagaaaggaatgaaggtgcccttgtcac | |
| | | | tggtgaccccggcgcagggcctctgggctagaaacagagtgttatttctttgaagagacatc | |
| | | | ttgacaacccaagttcatctggacgcagaaccaaaggcagctttgatctgcttgccagtgtc | |
| | | | cctgcc | |
| E26 | mm10_chr4: | 857 | tcggaatagctggctccaccaatccccagcagtgccaggggatggggcagggtcaatggggg | 58 |
| | 143348892- | | ccagtaactgacagctgttccgtgggttattttgggccaggggctgagcttctcggttccag | |
| | 143349749 | | ggtgttaggattaaaatagccacctgcagggcgatttgcctgtagcagtgtaggggcttttc | |
| | | | caaaaagcaaacattcaaagcccggcagaggagtagaatacggggttggtttgaggaatggc | |
| | | | agggaaggaattctatcactgtctaagaccaacctcacctctgaggagctgggctctaagcg | |
| | | | ttggcatctctctgaccagatcactttcctgggccatcaaaatgaagggttgatggggaaag | |
| | | | aagggttggtaactcctaaacacagcttctggccttcttcagtcctcaaagggagcggtgag | |
| | | | atgtccccacccttgccttttcacagatggttccacaggagttcagggggcgcttggaggct | |
| | | | ctggttgcaggtcaccaaactgccccagtacctcttgctttggccttgctcagagctacaga | |
| | | | agattcgtgttgggaggataatcagttctatctccaagctcaggaccagcatggaaaacgct | |
| | | | gactggccctggctatccttccagtcacagcgggaccttggcagccacccgcctagctcaca | |
| | | | ccctcctcctgcaggaggtggggcaaaagcaggtgggaggagggggtcgaaggggaagccca | |
| | | | accatcctggagcctagccaaagctcaacttcgcagccccaggcggggacttgacagcctgc | |
| | | | gggctttgttcaggacttcgagttagagtgacctgggaccctttggcagaga | |
| E27 | mm10_chr4: | 954 | ccggaggtatttctaccagacagatttgtctaaggagatttgctaggtccatttaggagagg | 59 |
| | 143361408- | | acctagaaaggtctaaactattgcgcaaactgtatttataagtttttccacctgccttccgt | |
| | 143362362 | | tcgtttattgctgttgtctcagtgtgtacgcaggatttgccttgtaagcagaattaggagag | |
| | | | cagaagcctatctttaacaacagaagtgcttaggagagcttgtggccaatcgcattgagcag | |
| | | | ctagctctcttgaggccagagcctgagaaaggccccctgggaggtctgcgcccagcctgttc | |
| | | | cttggcgaggggccgggccaggcttgagagattaatctcaatctaccatctgctgtgtcatt | |
| | | | gtctatttctaggatggcagtttgtcaccctcgccactcaggtgtcagctgtgggaagaagt | |
| | | | catcattcaggagatggatcctgggggtgtgcacgggaggaggaccccagccactcacggga | |
| | | | gtgaggcacccatcccaggagagaagctgggccagaagattgggctggctggtgtgacgaat | |
| | | | ggatagaaggaagggccactacagggtggaaaccagaaggacacttaagctgcccaccgggg | |
| | | | cgtcaccccaaggcgaggtgatcttcacagacaccttttctatagccctccaggatgcagaa | |
| | | | gcctccaagctgggggtttggcaggaaatctgggcagccatagataatgtgtggttgcaggg | |
| | | | caggaactgtgaggacagggacgaactcatctgtgatttccacttttcacaatccatcgcag | |
| | | | ggccactgagcacaggccagagaacccaaccctggctgagtagacaggaaggtatcaacgga | |
| | | | aggatgccagctgatcctcagagctgaagaagcaggctcagaaaaggacagggctgcagctc | |
| | | | agctgcaagagtgctccctcggcac | |
| E28 | mm10_chr1 | 423 | cagtgatgtcatcagccatttcaacctgctgagaatttaaaggcacaggcacccacttccca | 60 |
| | 1: | | tattgaaggtggcaagggctaagtggctacccgctaggaacacttcagctggctgctgatgc | |
| | 3504821- | | tagaacatccaggggtaacataagcacccatcagcttggctctcctgcatgctgaaggagac | |
| | 3505244 | | ccccacctgtctacctccagacagtaggcaggacccacccttgatccagggaggggcacagg | |
| | | | cacactcacaggtaatacagggacaggtgggtcagaggtcagcagcctcctttccagggcct | |
| | | | cagggacatctgccttgctctgtcacttttctaactttctgtttagctcaggaattgtcttg | |
| | | | gcccccacccttgccccatttttaagccatgggctagctcccccctccacct | |
| E29 | mm10_chr1 | 627 | atagtggcgctatgtgggagatgtctgggtggcagagagaagcatcagggaagatgatggca | 61 |
| | 1: | | agggagcactccttctcggagggagcactccttctcggagggtcctgtcataggactggctc | |
| | 3509025- | | ctcgctcccctttctgggacagatggatattaccttcattcctatgctgccactcaggaaat | |
| | 3509652 | | gggctggaggaggagtgacttttccaaggtcacacagctggggctgagctcagacatccctc | |
| | | | ctccctctcctctgctccccctcctcccaggcacccgatgtcagctgctctgaagtgagtgg | |
| | | | ctatttttattctggtgcgagggacctgaaacagcagagccatgtaggttagaagactctag | |
| | | | gacaccggcctcagcaggcaaggaaaagctcactgcaggcacaggaccctgctaagcttgcc | |
| | | | tgggggaactcttgttccttaaagactaccctgtcctgggcttctgggtctccaaacagttc | |
| | | | ttcattagaggtcagaatagagcacagaaccagaataagccgtgttcatagccctcatcctg | |
| | | | tctccagctcgaacactaagcttgaaattgactaagacttgatcccatgccaaggcaaagcc | |
| | | | tctactct | |
| E30 | mm10_chr | 765 | ccgttaacttcacttaagaaatcatgactgccaaagtggagtcttacaagattttgtcctag | 62 |
| | 3: | | catactccttcttagcacgctttctagagttggactgttaaatttgtgccagatacatcaat | |
| | 83503268- | | gaataccgcctgcctatctttttcttccaactgaatttacttatctaaaaaagaaatcgtct | |
| | 83504033 | | aaaaattcacctgctaataaatgtaaatgtccttaccttaaccagtcattaaggaataccag | |
| | | | ctaaatcagggtcacacatcaagggtctccacagacgtgagtgtccattttaaatggtacag | |
| | | | tagcattgtgggttcccaatcgtttaagtgccatgaccatccagttttgacaccgagtctct | |
| | | | tagagttacaagctttctaatttgggagcatgattaatccctcctatgtgataagttttaac | |
| | | | cttctaatatttctttggattgaaaaaagcaaatgagctgcggcaaagaatggcaaataact | |
| | | | acagtgcttaacacagtttaataacctgaaatgaagcaagtgtgtgctatgtttccattaaa | |
| | | | aagtttccagccacaattaattgaacaaaaacttgtcttgttccaagattattcttggaaat | |
| | | | gtaattttaaagcctgtgtgaaatgaggaaacttaactttttataccatatgaaagcaattt | |
| | | | cattttttaggaatgattttggatagacttccgattggatattttccattggaactaacagt | |
| | | | gtagaggcttggggtggggaga | |
| E31 | mm10_chr1 | 222 | tcgtgaaatccttaaaagccacatgaggcaggctttgaagctccaacttctcattttaaagt | 63 |
| | 3: | | aattccatagaagaataaaactgaacctaactcaaagtctctggtgtgaggaggtagaccca | |
| | 83507235- | | gatttgggttgtctctcaggcccatattcagttttctttgatttcagtaatatggacaatca | |
| | 83507457 | | aactgacagatgaaacgtatcacgtgaaaacttacac | |
| E32 | mm10_chr1 | 287 | cagagtactgcccttagatccttcttgaacagaatgctacattaggcattccatgagttccc | 64 |
| | 3: | | ccttcccaggaggcctgtgcccagtctagttgccatagatggccatcagacatttgcaatga | |
| | 83515122- | | catcaggagactatctgagcagccactgccctaaagaatctgctagcaggagactggggata | |
| | 83515409 | | tcagtgtcttcttggccgacaactgcattccaaaggtcaactggggtgaaaaggatgggctt | |
| | | | ggagcatctggtgcgttttcagccacaaggcagctaacaa | |
| E33 | mm10_chr1 | 911 | tcacgtggcacacgcagcgcccccccttctaccctcaacacacacacacacacacacacaca | 65 |
| | 3: | | cacacacacacacacacacacacacactagcagcctgggagctctagaaactagccggggaa | |
| | 83518268- | | agagtccccaccccctcagctccgtctgccggagaaaagtgggtgggaggaagttgcgaccc | |
| | 83519179 | | acggagaagagcagataggaggggagagccatgtcggaggggaggaccagcagtaattgggt | |
| | | | gacgttgcagccgttgtgcctctccagtccgcccagccagcgggtcagctttcaacacaaat | |
| | | | cagacagagggattgcttctgcctgcaatcaataactcagtcgagtgagctgctgaaaccag | |
| | | | aggcgtcccttgtctgctctttatggggccctgcggactgaatcccagcccctctgcttcac | |
| | | | aaacaaagaagtgggtaccaaatgccagaggaaaattttaaccccaaacttccatcgcatcc | |
| | | | cctggttacgatctgttaaaatgaaaagagcacggacctagtaaggtctctagaagaaaggg | |
| | | | ttaaagggagaaatctacgccttaaatttcaggtctcctgtgatatgaaatggagtagaacc | |
| | | | agtgtttcaataagcatttaacaatgggaagagaagatgtaaagtcccattaaatttagctg | |
| | | | tccccatagatgctttcgagtcagccgcacacttataagcaaattaccaatcaagtcttatt | |
| | | | tgcacttactgatagaagggaagaagaagaaaaaatgtatcatactttgtagaggagaattt | |
| | | | ggaaggggggtgttagataatttattacagactttatttcgtgaagagtaaggttaagaggt | |
| | | | cctaggagatatatcgtgatcccaacttaataatatacattaaa | |
| E34 | mm10_chr6: | 189 | cctgcaagagtggaacccgagggtcagtcccggctctccatctcttcctcgcactgccccgc | 66 |
| | 147263395- | | tccctcttttggaaccccgtggcttcccaacagccactccaaagtcgagggcatctcacaag | |
| | 147263584 | | ttaggaggaaaaaagaaaacaagaaaaagaaagcaaaaaagtgtctccgagcccctgagcac | |
| | | | ttac | |
| E35 | mm10_chr6: | 516 | cacttttccacatccatttaaaccttggaaatgctctgctttctccaaacatgacctgaaaa | 67 |
| | 147266874- | | cccagctaggagatgcttttcagagcccaagatgatttgagatacaaaggtcttaggaaaat | |
| | 147267390 | | gggttctctaggtcccacacttgtgtaccctcattcccatggctagcaggaaatatctgagg | |
| | | | agaaggaggaagggagggagggaaggagacagggtgggagggaaggacaaagggaggggaac | |
| | | | agagaacatcatttgggctgctgctcactagaagccttttgctgagtcaagttcttggctcc | |
| | | | cttgagcagacagctctggacttgcatgtgaccacaaataggattcaaagacaccccctgtt | |
| | | | caaaggacagtcacaccgtcatcacatctcttgccctgctgcagaaagttctagctgtggtc | |
| | | | cagttccatatgagctgcctagaaggtggcatccccgggtgagactgggaccttggagcatt | |
| | | | tacttctttatcccattgtcc | |

“bp” = base pairs

TABLE 3
Exemplary human enhancer sequences

| | Human | Size | | SEQ |
|---|---|---|---|---|
| Name | coordinates | (bp) | Human sequence 5′-3′ | ID NO |
| E1 | hg38_chr2: | 1766 | tctaatggacatacagtaacccttcataaatatttgctgaacgagtgattcagtgaacaaat | 68 |
| | 165953030- | | gaatagagaagaccaacatccgaaaagttattttattttcaagcctcatgtctttaactgtt | |
| | 165954796 | | ttatatcagcctttcttaagttgaccgtcattaatatttgctgaatgaatgagtcagtgata | |
| | | | aacagagaagaccatcaccctaaaataacgacccctccacttttaagtcttacgtctttaat | |
| | | | gggtttcatataatctttctgcgctctttttactgtccagtgtgggagctgacactagtttg | |
| | | | ccttaagtccttaaaaatcgcacccggaggcgcagtgtcataggtaacccaagctttcctag | |
| | | | taaacatgatacaaaagtaaacacaaccaacagcatggggaccagcaattcagaaacaccga | |
| | | | gcgggcgggctgcccagacctgggcttccccagcagggcccgcggagaccggccgtgagcag | |
| | | | aggctgcaggcccaccccgcaacccgagcagccggggcaccgcagggaaacagcggcctagc | |
| | | | gaagccacccgagctccctccgcgcccccgggccaaaaggccgcaaaggaactccgcccgcc | |
| | | | cgcccgctcacccgctcacccgctcacccgctcaccttcaattcctgcgagtccatggctgc | |
| | | | cccgaggccgggccgcggggctctggggattgtctcgccgcagcctaaaggaagacgcagaa | |
| | | | ttcagctcccctagcctcccggagcgctctagcgccccgggccccagcgggaggggcggggt | |
| | | | cgcgccgcgattggctgtcggagggagaggcgggcctgtgtggcggggatcgtgctgtaatg | |
| | | | gagcaggggcggcggggacccggaggtgagggctgcgagggccgcccgggagggtccgggct | |
| | | | gggaaaagggcctccgccggagagtgcagctggaaaaggaggtcacactgggaaacggctgt | |
| | | | ctgaggacagtgggtgggcgggccgaggaaatggaattcaggaataaaggaaacggagtatg | |
| | | | aagaaggggaagtctgtttcctgtcactggttgtaaaggaagacaccattttctgcacgttt | |
| | | | gtctggaggcggattcccgcagtgcggctctcagcaaggctctgccggcgcgggaaaaagcg | |
| | | | gtcaactttcacgtgggcaagttgttttacggccacaaggtggcgcagaaaaaaaaaatcac | |
| | | | acgttcttaacagaaatacggtgcgcttgggcccgtctttgcaggcgttgctgcaatctttg | |
| | | | ttagaatgtgtgttcaattagcccttttttaccagccccgataataagagggacaaataaat | |
| | | | taaacttccagaaaattagtgtcttgttttcaatgatactactgattttaaactgagaataa | |
| | | | aatgaatcccaatgcaaatttttatgtttgcaccccattaggcaactcaatcagtcacacat | |
| | | | agatttcttaagtccaggaaattaaatggaaatataatagactaagattttctatttctgct | |
| | | | taaataaatatttaaaatagtgcataaggtctgagatttaagtgatctttgcagaatctttc | |
| | | | acgtggattccaaattttgatcctagtgttaattatcttactttagttgacatgatacgtag | |
| | | | ttgccttttccagattttaagtttcttaaggagtttataaacattgactttttccccatgcc | |
| | | | aataggttatgtaaggacagtcttgagaaaa | |
| E2 | hg38_chr2: | 849 | agtgtgggcctcccagggctgtttagctagcaatgagagaggcactgcctatatccaagttg | 69 |
| | 166084035- | | tatatggcaggttttgcacaaagtggattactcgaagagaaagcctaatggccagtctattc | |
| | 166084884 | | atcttcccctttctcgatgttcatcttttctctcccagctctcctttattctcaattttctt | |
| | | | tttttttttttttttgctcagcctccatctcacttccgttgctgtcctctccccaccccttc | |
| | | | ccactctggactgtgcctctcctttgtagacacttcaagtccattctatttcattcaaaaac | |
| | | | catggtctagaagtaacttaatgtaaacccacaaagatggagacagaatgaatgccattctt | |
| | | | cttgctgctctctcagacaatgcaggtcatttttgcctatggtgctggtaaagccaggagtt | |
| | | | atgtagctataagtagcagccagaggaaatagtgcctgagtcagcaattgtctttttattgc | |
| | | | tgtggggcaataatgggagaaaaaatcaggcttggtacaattccctttgaaggaaaaagatg | |
| | | | ccaacactagcattttaacacaaaatgctggttgggggttgggaggaaggatgcttacattc | |
| | | | cttctttggaaatatctactttgataaccattttggtaaaataatgcagtgttttcagtgtg | |
| | | | caaatcctttcaggactcatggttgtatggcagacgcacctgacagcaataatttaagggta | |
| | | | ccctgagaatgactctgtggtctaaaaagaatgtgtgtttggaagtctgaggtaagaaatct | |
| | | | ggctggaagtggccaacctggaaatttgctccttattattaagg | |
| E3 | hg38_chr2: | 844 | atagtgcaaagtttaaatttcattttcctaagatttgttttaaaataacacgatttacccaa | 70 |
| | 166090876- | | gtgatttcaaaccacaattacattctgtttaaattactaatattttattgcatcacaatctg | |
| | 166091720 | | catgaaacagatgtcaggatataatgaactaacctgcattgtatttttatttttgtctcctg | |
| | | | tggcataacgatttcataggaaagagaactacacagctgactgactgatggggaaagttaca | |
| | | | caatggatagctttgcagcaacatactaatgcggtagggagatgctgcagagaggctagaaa | |
| | | | taaaatcatttctttccggagcagcactgcttgctgtcggctgagacaaaaaagagatttcc | |
| | | | tttttttcctttcttttttttgaaaactcacataacattaattctgttaagcactggataca | |
| | | | cggaaaggtgtttaccttagaaaatcatttagcaatttttagaaactagacatatagcaatt | |
| | | | ttaaatctttttaactatctaatgaccaaagcagagggtcctcacaagagggatttagatgc | |
| | | | tactgaattgaataaagaaaatatggatacatttattgtatgccttattcagtttgaggttc | |
| | | | attttgagtttagaaatagggatataaaaacatcaggggttaaatagcatgggtaaaggaca | |
| | | | tgaaccaagctgcagagaagaggctgactgcctgctatatttgcaggcattactcagcactt | |
| | | | ttcttaaaccgatacatcttgctggctgcataagcaagacaagacccttttccctatggctc | |
| | | | aggaaggcagagaagtcaacttcagccttgaaaaaggca | |
| E4 | hg38_chr2: | 267 | tgccagacagaacaagttttagtgtagttgatagtaagttgtgcccagaatattaaattgag | 71 |
| | 166094366- | | tcaaatttattttccacataaagtcacagttttatatgtcattatataatctcttggcagaa | |
| | 166094633 | | ataaggaataacattctgaatgttgcactccaaaattcaaagaatcttagtataaaaatatc | |
| | | | tagcattttagatgtttcaaagtagggccaaatgcagaaaataagttggatatgataaaaat | |
| | | | accagaaagttctattcagt | |
| E5 | hg38_chr2: | 894 | catgtaaaattaatatgatcttttagtcacttagaaaaaataccataaagaacactaatagt | 72 |
| | 166103693- | | gtttaaaagcatctacccagtgccaagaactgcattatgtattggtgaacataactttagac | |
| | 166104587 | | tttaccatacaacgtgaaaatatatattattatcactattttacagatgaagcaataaaagt | |
| | | | cagaaaaaatgtagctaattaaagtgatactgtgtatagctagagcagtgtatagctagagc | |
| | | | tgatttgtctgactctagccctagtttctttccattatatcaatttcctggaaatgtatctc | |
| | | | tgttcatggcatagtgcctgacactatgcttattaatatcttttgaataaaagaaccactga | |
| | | | gtgatttgaaataaaactaaatttagttagttaattttattggtggtatatagagatagtag | |
| | | | gaaaaataattgaaaagagacataaacagatttgccaatactttctaagaaaaattatggaa | |
| | | | ctagagtttagtcaaaatgaatgctttcattgttagaattcaactttaatctttgcagaata | |
| | | | caaacaaagacccattttctagaagaagtaacagggaagagagagtaagaaagagataatga | |
| | | | tgaacattgtctaatgttacagcataatctagtaaggtaagaacagaagagagttcattgac | |
| | | | ttaccaacatagttgtccctaatcacctctgtgaacctagagtgctacgatataataatgat | |
| | | | tgtggtggtttaaaaagtaaatggggctgggcatggtggctcacatctgtaatcccatcact | |
| | | | ttggaaggctgaggcaggtgtattgcttgagctcacaagctcgacaccagcctgggcaacat | |
| | | | ggcaaaaccccgtctctacaaaaaata | |
| E6 | hg38_chr2: | 665 | tccactttttgctattccacagagatttcaggaagaaaaatcacactcctattttctttttc | 73 |
| | 166118214- | | tttgcttactgatttctatttagtttcttttttttttttttttttttttttttttgagaaag | |
| | 166118879 | | cgtctcactctcttgcgcaggctggagtgcagtggctagattcttcttgagtatgctcaaac | |
| | | | ttcctttttggaatgtcttccaaaggcactcttgccttcatttgtacaagttgattgaccct | |
| | | | ttaaaggccttaaatattattgtgcgacctcacagactcctcaaatcacctgaaacctgaaa | |
| | | | tgctgaggcccaggtggcactgaaatgatggtattctagacctgacaccggactgttttctc | |
| | | | cttggttttgtcccaacacactgacatacatagcccaaaatactactggcctttttaagtgg | |
| | | | catatcacattccagggtaatatcaaaactgctgcctggtagcatttgtgaagtctcaaagt | |
| | | | aactctttccaggattttcaaatccactgaatttcttagattgaaatatgtatgtgacagaa | |
| | | | ttctcttagctttctttcctctatgaatatgtaattggaaactctgagatccggtttctcat | |
| | | | ctttattggattttttctttaatcttaaaattatgaatatttgctt | |
| E7 | hg38_chr2: | 5124 | tggcaaaaacgcaaaacgttgatggataacggtgatgacttacacaacaatgcgaatgcatt | 74 |
| | 165892760- | | taatgccactgaacagtacacttaaaaatggttaagatgatgaattttgtgttatatatgtt | |
| | 165897884 | | tcaccacaatacaaaatattctttaaaaaagacttttggaaatactgtatctacttaattac | |
| | | | aggatgtcaaactaatacaggctgatagtatcatttgtccccttgacacacaatcttgggtc | |
| | | | cagagattttgttcaccacaccttttagcatcactaaaaagggcacaataagaatatggttt | |
| | | | cagaaaaagacaattcaaatattggtcttgtcctttagctatgtgaattcaatcaaattact | |
| | | | caaattctttgagtccaatatacttattttcttaaaataggattataatattgactgtagga | |
| | | | gtgctacagaaataaaggcatgaaaaatatttataaattacaaatgttattaataatattta | |
| | | | tacttccaaaaatgttgacaagaaatagagtaactaccccataataaagccacagcatctgg | |
| | | | aagctatattggattaagcaagaactaaaggttaaaatttcggattaaattttttttgcatg | |
| | | | atactgctagtattatcaacattgggaaggcaatttcttgaatatttcttatatactattga | |
| | | | aatgtattcattattagttcaagttataattaccagtgacagattaaattacattcacttgt | |
| | | | ctttggttaaccatgacatttgacagaaggcaaatttctgcacttaagaaatgtattaaaaa | |
| | | | ctaaaatgtatattaccttctaaaaaacttagctggtccatctttattgatgaatagtagga | |
| | | | agatatcaaaatagttatagggtgatgagatgtggcaagcatgcagtgctatggtatggtat | |
| | | | tacaaagcacaggattcttaactttgcctggaggagttgggaaatttcacataggagttgac | |
| | | | ctttgagcagcctcaaggataggaggaagatcttactagacggacaaaggcattccaagtag | |
| | | | cagaaggcatgcgccaagagggaagcagagaacagtgtggggagtgttggtaactttgatat | |
| | | | tattaaagcggaggaagaaggataagaaatataaatggccaaataatttgcggccatattat | |
| | | | tattaaaataatgctatgattttagactttatcctgaagcactaacttaaattttaagcaaa | |
| | | | gggtaggttttgatttttagaactgatatgctagtcctatgatgacctggagcagccagaac | |
| | | | ctagaagctggaagatgagttgggaatctgcactagttcagatgagaggtgataagggtctt | |
| | | | cattagagcagtgggttaggatacgacagactggatgtgttagctagctatcaagcaaacag | |
| | | | agctgaggagacatgttaaccaattagtatgaaggaaggggaaagctcaaggcgatctggag | |
| | | | attctgagagagaaaaggggcaatctgtcgtgagagcagtaattagatctagaagaggaatt | |
| | | | tttcaactacttaaattaggtcaaatttgtatggtacatttctgaaataagctaaaatagag | |
| | | | ccttaatctaaagtacaagatgagttactgaggataaccaataatgtacacataaaatgaac | |
| | | | ggagatgcatgttttagagtaattccaacaaaatagatctgtggataagtatgtaaggtact | |
| | | | agtaagaataaagcatacaacacaagattaaaaactcttaagattaaaaatatcacatagac | |
| | | | aataaaaatttacttaaaattttgtggttgtttttgagaccaagtctcactctgtcaccgag | |
| | | | gctggagtgcagtggtgtgatcttggctcacggcaacctccacctcccaggttcaagcgatt | |
| | | | ctcctgctgtgtttttaattgacttggtgttttacagtcattcactgatccattcaaccaat | |
| | | | aaacatctatgttgccacatccatgtgtgtggcattttgtctgatattgggaatatggtgtg | |
| | | | atccctgaactcgaggagtctacagtgtaataaagaacacaaatatgcacataaatattttt | |
| | | | agtaaaatactataagtaataaagacgtatggacaaagtaaccgaggattagggttaccaac | |
| | | | tcatcccagtttgcctgtgactttctgttagcatgggaagtcctgcatccaggaaaaccctt | |
| | | | tgctccgaggcaaatctaggatggttggtcatgctacccagcaccacataagacatttttta | |
| | | | atgtaggtggtggtagtgggatgcagatttacttttttattttcccccaagagggatatatt | |
| | | | ttagattatgtgtttggaaagagaaaagggataaggaagctaaagttcattttaggcaaaag | |
| | | | gaaaaaacccaagcaaagacttggaagcatggttgtatgtcatttggtgttgctggagtaca | |
| | | | agatccttatattgcatttataaaaaattgcttttatatttgtttacaaacagtccaaagca | |
| | | | gccagtctactaagccaatttttttgggaaaaaggctgctgccaagcaacagaaacttacgt | |
| | | | gaaacaaaacccacaagacacatgaagacttcttcaaatcttagaaaactataatgtgtgag | |
| | | | attcttcaaatcttagaaaactataatacttttaatgacttaaaatattcacagtggaagaa | |
| | | | gtctgttttttaaagaaataaagttagatcattgtctcaaagggaaagactgtgaaatggga | |
| | | | acagcttgagatagaatgaatatattatgtatattacttttaaatggtagtttagagaagag | |
| | | | gaataagagaaaacagtgtggaacacaaggtagaaatggcagggaaaaaaacgatacaggcc | |
| | | | tgaagaaaataaaagtaggtttgggcaatgtgggtggcaagatgagcccatattttggaccc | |
| | | | agagtgagtggaagaggtgagatggtcaagtagttcacagcattcttttaaataacatctga | |
| | | | gtatactctggaatagacagggcaaaaaacaaatgaaattgcctgtggtagtccccatatta | |
| | | | aataaatttaatttatttaattaaaaaactcaaagagtaaaaataataaagagaaagtgttg | |
| | | | atattactgtaaaaaacagaccatattttctcctttcaatttttgtgctcgctggggatatt | |
| | | | ttatttttaaatacaaactgatgttctctaaattcaaacatctttttattaaaagcgttact | |
| | | | agaggtagcctgcagagcatatctagttctttgagttgccctgcttgaaggattgccacctc | |
| | | | tgtgtctctgaggctctgagcacagatgcctagggcatcagcctgagatgaaggtggtgggg | |
| | | | tttagaagaactgaaacacagctctaggacttcctctgccatttcaaactctttttagtaac | |
| | | | acaggtagtaacatacagtcatgtattagtcaaacagttcccacctctttctttttctcttt | |
| | | | cctctcctccctccatccctgcttccctttcttccttttctccctcactcttctttccatct | |
| | | | agtatttgttgagtactaactaggccagatattcttctaaattctggaaacacagcagtgaa | |
| | | | ggacaacgtttctggtcacttgatgcttccattctatgatttgtagttttttgcttttgttt | |
| | | | cacaagggctacacaaaacccaaaaatcctaaagccaaaccccaaaaactcaactgaacaga | |
| | | | aaacaggataaagggacagagagcaagaagtggtgctacttgatgtaggccagtgagggagg | |
| | | | ggcattctaaggaagaagcagcaccagagcaaaggcacaaaagaaaatgaggcagcagccag | |
| | | | atagacctgtgggagcagcatattccgggaagtagaaacagcaaacaaaaaggctcaaggct | |
| | | | gtaattggcttgggcttttgcctacctagtttctgttgtccctttcttctttactatcagaa | |
| | | | ttctgattttattctacagggtattagattcagctaaaagataacatttcccaccttctttt | |
| | | | gcagccacggaggtgatactactaagggtttttttgtttgtttgtttgttttgtttttaata | |
| | | | aaacgatgatttgctggttgggaagcccttttgcccagctctgcacccaccttccccacctg | |
| | | | ggccctggaacatcaatgcccagcactgcggtggtcatcttgaaccatgaggtgatgctcag | |
| | | | gcaagtcagggtaacaacgtagaacaaaaagagagaaggctctggtttcctgatgatactgt | |
| | | | ggaaccgccacaccagtccctacacagtgtacttttcatttcttttacagagagaaaactaa | |
| | | | aaaccgtgatttttagcctagaattttcaggggtatctctgccattttcaatgaaagtattt | |
| | | | ctaaattcttcataggctagggatggaaacacagatgagttatgacgacgctgcaataatct | |
| | | | atgtggaagatggcaatgccttagaccagggtggcactaacagaggtggtaaaaagtgatgg | |
| | | | cattctaggtatactttgaatgtagcactaacaaggatttgctgatagactggaggtgatat | |
| | | | atgagagaaagatgagacaaaggttaactgtgaggtctgggccggcacaacagtgagcagtg | |
| | | | atgccagtcactgaggtgagaggtgggggtggagcaaacttagaggcgagggaaagttcagg | |
| | | | tgttctattttggacatgttaagcttgagttactcctagacatctgagtgggaatgcaaaga | |
| | | | ggcagaggtgtacatgagtaagggctgcagataaatgtttaggacacatctgcacatacatg | |
| | | | ggatataaagccacgcacctggacaaagtcacctagggggtgaatatataaaaagaaaggga | |
| | | | gttcaggaactgaagctacgagtgttctaatatttaaacgtcatacagagaagagaactcca | |
| | | | aaaaaggaaactcaaacagcaggcaatggggcaaaagaagaactgagtgagtttaggatctc | |
| | | | agagacaaatgaagaaagtctttcattgtggaagggaataa | |
| E8 | hg38_chr2: | 1636 | tttgtcttcaactttttaaatatccatctattttttagattagatgccatctgttgtattat | 75 |
| | 166148156- | | catatctggttaaatcttattaaaattcctggccaatatatcactctgctgcggcaatctta | |
| | 166149792 | | ccctgctacctctctaggcttttctgcagcattcaataaagagcctgctattttaactaatt | |
| | | | gctgaggcaggacacagtgtgagagtctaaaacagaatcagcgctatattgtttctactttt | |
| | | | acccatagagtgaacaactgttcatatcaaacctggaatcatcccttcaagtctcctaaaca | |
| | | | cagcacagtttgagccaatgcagttaatccaccctccttcagtgctagtgtcgaatggcgct | |
| | | | tttgctgcagtattcaccttgaaactaagtaggcaggattcattatttgttgaagtcaccta | |
| | | | actgccagtttattcttatacgatcacaaacaatcacaacagaagacaaatacaggcatata | |
| | | | tataactcacttacaagggaagcaaatttgcagccagagacaagggcaacgtaacagccaag | |
| | | | aactgcagtaaatctctttgagctaatagccacctctaataatgctctcctacaacacctcc | |
| | | | aatcaaataaattgtattcagagtttaaatatcaaatcaacattcattccttgatggttaca | |
| | | | gatgatgtccgataagcaaatttgaatttatgatttatttactccttaagtgtctcaaagcc | |
| | | | aggattactggaagacttactatgcttattcaagcatgctgacagaactgtaatctcagtaa | |
| | | | tttctccctgcacctctcagcatgactgacaggcatctgccaagactgtagtacataaactg | |
| | | | ctgaaacatgcaaaaatatttacccccaaaagatgtagtaaaagctcagcttcctccagctt | |
| | | | ccataacccctgaagtgttaatctggaggaacagttccatgagtttccacaggccagcagtg | |
| | | | tgtctcctacacttgacctagacagccttacataacgaagcaccagtgctggggagctctct | |
| | | | gaatgtcatcaccagcaagagcaagaagtattggcagcagcaggcagccaggcaggctggga | |
| | | | gtttgcatggaaatcatgaagtctttcttgtcttcctcttttgaaatattttggaaggcgag | |
| | | | ttaagaatagctcagaaactggtctcattctttttgtgggaagaatgaccagaagcataaaa | |
| | | | gctaagtcttccagcaagtgcaagacacctcttttggtgtttgcagtaaacctaacaagaat | |
| | | | gaattgctatcagtaaagtcctgtaccatgacacctaaaaggaaggaaatggtaaagcaaag | |
| | | | taataactcaaagacagtcaccgagggcttttgtccacctttaactcattgtggtgagtaga | |
| | | | gggaaatatgtatatatgtaatgagattattattgggggtgtgagttgttttgcaggatggt | |
| | | | aactaaaggatttgtaaagagtgtttatgttccttaaagatcttgtttgtgagcaaggtaat | |
| | | | aggatatcaaattcagaacgtggtgggttaaactggttgtatttaatgtatttcaacttctg | |
| | | | aaaatcttatgcaactaataagaaa | |
| E9 | hg38_chr2: | 636 | tcccaaatgtgtgcaatgcaagttatgcttttaaaagctagaaataataaaaccagtcttct | 76 |
| | 166150066- | | attctgattttgagtatggtgatatagtattattaataaaagatgacattatattgtttaat | |
| | 166150702 | | tatatataaaattgtggtttgatatgagttgttggctaatatgtataattcctgaggtaaca | |
| | | | gaaatagagaaggaaccacacatcatttaaaaataatcttaatgttctgctcttagctggga | |
| | | | aacctatctgctaatgcatcacactaagtagagtgaggaaataagagaatttagatctatga | |
| | | | gggaacacagtgatctaattccaacccattacttaactcataaggaaactgaggtagaaaga | |
| | | | agttagatgatatgcctgacatagaggaagaggtgagtgaaaaatggttttcctgacactaa | |
| | | | cttgttattttgtcagctatactgcaatgaataattgtcttttgatactggagtaaaggctt | |
| | | | gatgtacagtgatttttttatatcatacaaatgacagaaaaaaaaagtggagtagtactaaa | |
| | | | tatctgcttttagcagtagtctgattttggaaaaacaagttctgtactgattggaatgagaa | |
| | | | actttcttcagttattt | |
| E10 | hg38_chr2: | 586 | tatcccagaaagaaggaaatggtcagtttatctggagttaagcatttgtgtattatcatgat | 77 |
| | 166160023- | | taatcacaggaacagttgccaagctttcattataaaaatattctccaggagaattcatcaag | |
| | 166160609 | | ttccattgcctattaatttctgtccatgcattttatttggcatcttcaatgacagaggacac | |
| | | | ttttaaaaaaaagaaatgaagacaaaagaaaaagttcattagagaaataatgtatgtgtgat | |
| | | | atttaaaaattaagccacatcatcatcctaagaaaactacgagactttagttttagtataaa | |
| | | | cttgcagtgtgttcttgagatttctaaatataaggcttaacattttttccttaatacacatc | |
| | | | gatttggcatctcatatgtattatttatcaggaaattataaaacaaagtaaaatgatgtttt | |
| | | | actaaaatgcacatcatttttagatatgggattttaaaacttgatttataatactactttta | |
| | | | ccatgaaatactcttttgttgtatgaccttgagtacatttcccatctgtgaatctgtgtaat | |
| | | | catgtacaaaaataaatgagacaaaacct | |
| E11 | hg38_chr22: | 628 | tcagcaagtctgtcatcgacatcctgcaactgtttgagcgggcagagcaagtgcgaaaagat | 78 |
| | 36816984- | | taaaaagtgcttttctcatcatttctgctcatatgaccagcgctgcagtgctgcgcgccggg | |
| | 36817612 | | cgcacgcccgccgggcctggcatggcgccaggggcccggactctgagcgcagcgggagcggc | |
| | | | tcagtccagccgcgccgctgagcagcgccggccgccggcaagaaggcgcgcggacctgctac | |
| | | | cactcctgcaccgccaggccaggggtccgcgggatcccaggggctgcggccagggcacgagg | |
| | | | gaaggggccacctctgggatttagggggcactggcgtcaccagctgggtctggaaagtccac | |
| | | | ctgccgtcaaggacacgcaggaggtgcgccgtctcagatctgggaaccttggcggatgtcct | |
| | | | gccgcgtgggggaagatcctgaaccttcagcggccagcctgcacctcaggacctcctaggcc | |
| | | | ctgctccctttctctctccactcctacctcagcctctgctctggtctgtcctggatgcaaat | |
| | | | ttatgctgcaaaatctgagcgctgaggtcctgaaacctgacccacccgacgcagggaggagg | |
| | | | tggcaggga | |
| E12 | hg38_chr22: | 236 | tccctttctctctccactcctacctcagcctctgctctggtctgtcctggatgcaaatttat | 79 |
| | 36817484- | | gctgcaaaatctgagcgctgaggtcctgaaacctgacccacccgacgcagggaggaggtggc | |
| | 36817720 | | agggacagggacagggacaggcaggagctgctggggcccacttcgggtgccccatcccacat | |
| | | | ctggccagggatgcatattctaaaacctgatttgatgttttacttttattt | |
| E13 | hg38_chr22: | 593 | cctgggctgcactaagtcccagtgtgaccttgggttgtgaccttctctgggcttctgtctcc | 80 |
| | 36818134- | | ttctgatgtgttgatgacgtcagtggtcccatgtagtgggacctggggactgcaacttaagg | |
| | 36818727 | | tattggcaggtaggcagggccttgggctgtggtggccctgggtggtggggaccagggagagc | |
| | | | agctgtccagctgcccagtaactcaagttccctgacatcgctgtcaacattgtctcctgcag | |
| | | | ctcagccctggatggctgcccttcctggaaaccttaggatacctctgctggctccagctgcc | |
| | | | ccctccctgtgagtcagctccttcaagccacagcccgccagatggcttccaaggcaccaagg | |
| | | | atgcagctcctgacctgatgcctctcagctccaggacttcccaggacccctcagctgccctg | |
| | | | gaccctgctgctactgccgtcacctctgcaccttgtccccagctgggctgctgactcagata | |
| | | | tgccaggctcctatgctatcatttcaactcccaggctcagctcactccaggagcctagttgg | |
| | | | agaatggatttccccagctgaaggacgcttcagcta | |
| E14 | hg38_chr15: | 637 | gcacttcagttccttttcatcaaggaactgattaaagtgtggtctattatctctgtagtggg | 81 |
| | 88802240- | | gagtcccgaaagattccacttcttcctcttcttgcccaatgagagggtcaggaagcttccac | |
| | 88802877 | | caccatcctgactgtggccaccacatcctggtgtgaagcaccaccagcttcctccacaagaa | |
| | | | ctctgaaggtcaccagccagcttgagtcctccgaaggtgctgtggctcaaccagcagcctgt | |
| | | | gtagcagagagcacaagacctgggactcgaactcagtcccaccttcaagagggatctccacc | |
| | | | actttctgagcctcagttttcacatctttggttaggggcaggtgggaagttgccatatttac | |
| | | | cttgttgggctctttggaaaaattaaatgaaatgttaatgtatgttacacaacgggctcata | |
| | | | ggagggattcaaaagctgctagttctttttctccttttcctgaaaacggtattaaagagtac | |
| | | | tgtagagacactggagaattcctgcttcatatgagatgtccttggtctctcccggggaattt | |
| | | | gaagacccagagactcgcagctccccgtgagcctccccgctgacgccttacctccccctctc | |
| | | | caccatcccctgccatcc | |
| E15 | hg38_chr15: | 388 | tgccttcccctccccctgtcggccgcccctcggtccctgggggtggggtttccctttgcgct | 82 |
| | 88803290- | | cgccccctcccgcccccacccctcacgggccctcccctcccccgcccgtccctatgtatgtg | |
| | 88803678 | | tcacagcgcgccatgcccgcccgcccgcccacctacctccccgccgctccagagggggctcg | |
| | | | cagagctgaggacgcgcgcagcgctgctcaaggtctctctctctcagcaccctcgccggccg | |
| | | | gcgtctgacgcgggtgccagggtctccgggcacctttcagtgtccattccctcagccagcca | |
| | | | ggactccgcaacccagcagttgccgctgcggccacagcccgaggggacctgcggacaggacg | |
| | | | ccggcaggaggaggggt | |
| E16 | hg38_chr15: | 672 | gccctgggaaaggggatcaggagcacatcttgggaacaggagccttcctctcctgctgtcaa | 83 |
| | 88807290- | | cggcctggcctcgtggccatgcttcgtgtcctgatggtagccgcactgccgccctgataact | |
| | 88807962 | | taaaggaagccccttcaatgggatgaagggcccgtttctgtgacagctactttgggagtggc | |
| | | | agctccccttcccccagatccaacaggacacagccatccctgcgagggagtctgggcccctg | |
| | | | gacgcagttgaagaagggccactgggaggccggcagaacaaggggagggctggagaaggagc | |
| | | | gggagtgcaggcgagaggaggaccagagagggggaatttgtagagaaaacggtaaaacggtt | |
| | | | tcttttttcaaaagttgaatccagggcaagaacggaaacggtggagtttacttttaaaaact | |
| | | | cagagcctccttataagtggtggagtgtgtgtgtgtgtgtgtgtgtgtgtgtgtgtgtgtgt | |
| | | | gcatgctggcagggcgggccctgcgggctggccaggcctgtgagaggtgacttctctccctc | |
| | | | tcaatggccttacagtgtatcctaaagaagctttttgttaaactcatgaataggtggatttg | |
| | | | gggtgtgtgatgctgggcatcacgatttggatatttggttacctttgaggtta | |
| E17 | hg38_chr15: | 594 | ttcatttcaggctccccgtccccttcctctgcctcacctatgctgtcgccctgtcagcacct | 84 |
| | 88833390- | | aattcagcttcccatggagaaaggcctccctgttgacagggccgtgctgggactcagggctg | |
| | 88833984 | | ccaaagtcagtcttctcgcataaaaggctcagtgagtcctggagacacttggaagccagaca | |
| | | | gaatggaatttctccctattttctttacagctgagaaaacacacacaaacacaagagcatat | |
| | | | ttattgcgatatttctatcccaaagtttgtctttaaaaaaaaaaaagaaataaaattagttt | |
| | | | ctctgcccactccacccctatccccctacccccactctcctccccacctcttttcattcctt | |
| | | | cccatttcctggtttaggccagggagagaaatcaagccgtccaagccccacagagcactcct | |
| | | | acacccccggacaatgtccagcttgttcagagagtgagggaagaaacacagctccgaaaaca | |
| | | | tacacacaacctcccaatgaagggtgttctgagggaagaacaggcgggccttgtgtctgaac | |
| | | | acgaatccctaaggctctgggaagagaggagccggaa | |
| E18 | hg38_chr12: | 1030 | ggtcttaaagtaggaaaacacatggtgtgatttgggttggtgaatagtcgacttgtagatct | 85 |
| | 128377753- | | gcggactcgtcaacattgctctgacatcagattttctgaagagcagtgtgggctccttcccc | |
| | 128378783 | | agggctggccgagttttgaggggaactgcaggttctgatgtttccaactctgtatctctgcc | |
| | | | ctcgtcatttccatggacaagttatttgtgctggtgtgagatccagaatcggtcctgctacg | |
| | | | tgacaatgcctggagcacggagccaggaacccaagctgcctcaccagcgtgttagggttgtt | |
| | | | actgtgcccgttttagaagatcacttggaggtgcaaaaatagagcagtttttttttgttttt | |
| | | | ttttttttgacatggagtctcgctctgtcgcccaggctggagtgcagtggcgcaatctcggc | |
| | | | tcactgcaagctccgcctccccggttcacgccattctcctgccttagcctcccgagtagctg | |
| | | | ggactacaggagcctgccaccatgcccggctaatttttttgtattttttagtagagacgggg | |
| | | | tttgaccatgttagccaggatggtctcgatctcctgacttcatgatccgcccgcctcggcct | |
| | | | cccaaagtgctgggattacaggcgtgagccaccacgcctggccaagagcagatttttttaaa | |
| | | | aaaattaagtacctctattcatttgcaccttcactacccagtgaggagatcaaaatttccta | |
| | | | gagcaaatgcattcgatgccactcacagatttcgacaggagagcacaatttcaggaacgcct | |
| | | | acatcaaagcactaattggcacttttacagtgtctttctccgcacgtgagccttgctggtgg | |
| | | | aaggagctgtcatagtaatgcgtattcctccatgcccagtgagtagggtgacggtcaattca | |
| | | | cagttcactaggcacaaaagatgacggggctctcctctgctcgggacagcaagaaggttgag | |
| | | | gtgatacggtttgtcggtgtccccacccaaatctcatct | |
| E19 | hg38_chr12: | 476 | catgaaagtcactgttttacatactaattgcattttccccaaaacatcaagctaaatacata | 86 |
| | 128289803- | | actgatagcatgtttaaaggtcctatgtttcacctcaaattatctcatatttcacatgaggc | |
| | 128290279 | | aaaccctgtcctgaggccctgatgaagatgggcaggcagatctgatatcagctgcttttctt | |
| | | | tccttgatggaacctctggattgcgtgccctatcctataatgtgaaaaaagggcttccagaa | |
| | | | aaggtggaggaattactttctgaaattctgaaaggctggatccaaaggtgcagaaaggaaca | |
| | | | ttatttcctaccatataaaacccagtagggcgtgtgatgctgggacactgtatgagtccatt | |
| | | | ctcatactgccataaagaagtacctgagactgggtattttataaaggaaagaggtttggttg | |
| | | | actcacagttctgcaggcttaatagaaagcataactgggaggc | |
| E20 | hg38_chr12: | 565 | aaaaatgaagaatttatatgccaactccaggagaaatatttcagtgaggtcctggcttggtg | 87 |
| | 128323153- | | caaggatatggaaccagagccaagaattctatcagttaaaagcagcttagtttcctgagcct | |
| | 128323718 | | ggactgatgggggacgtggaagacaaagtgtcaggtccatcagtggaagattggccttgagc | |
| | | | cactgtacacagaatggagagcccactggcctaaaaggagagattgtcaggcgtgacgaagc | |
| | | | aggaattttagccgaagaatattcacaaattacaggccaagagggaagtggggacgttcgtc | |
| | | | ttctcttcatagccttgctcgttgggggaccagctgtcctttattgttaatagaaaaatcaa | |
| | | | tatagcaagaggcgaatctttgctgtgataacattggctcctttcaccaggcgtgtggaatt | |
| | | | agattactgatagatgcacctctgtcgcctccccaggctccagatagaatctatgggctttg | |
| | | | ccaataagcacggtaacagagtgtggatcaggaaccagcgggtggccaatggcagtggagaa | |
| | | | aatgtaat | |
| E21 | hg38_chr12: | 471 | atagaccatataattctcacatgtcaaggttttaagccaaagccctcaggcaccacttctga | 88 |
| | 128332503- | | ttttcttgaaggatcaaaaataaaaggttgcaaccctcacagccgtaggctcctgcagcaac | |
| | 128332974 | | tctttggtgcacctgtcaccctgatacctgggaggaggctctgagtccatgctgtgggaagg | |
| | | | tgctggccttcatggatgggcctcccctggtgtgtccactgtggacctgagtgggtgtccca | |
| | | | gagccctggctctcccttcttttcctctagaaagggaatcggcatgttcccaatcatctctg | |
| | | | agattatctttattcttcaaggagttgcagtggctcttgccaagtgccctggggtcttggac | |
| | | | atctgcctagtggccctgtagagacctccaccctccagacagctcagaatttgctaaagaaa | |
| | | | atgtgaattttggagtccagggctagaaaatatgacat | |
| E22 | hg38_chr12: | 488 | aagaaaagttttattttgcctctgtagtattggggtttaagtgatcacggtaattttccatt | 89 |
| | 128336003- | | atcattttgtgttttaaataaatacaacaggctttattgtgaaaatatttgtctaatattgg | |
| | 128336491 | | gcagtaaatgtttaagtgattttggtttaattactattacagtcatactattacagtgcata | |
| | | | aaatagaattcttcttgagtttgttcattagatgggaagaggctgcatttttaaaaaatata | |
| | | | tgcatgcctataatactacatttaaatatgtgcgtatataaagagatgctttcttatttata | |
| | | | tacatggtcattatagagctttgtgagaaatagaattttctctgtgcaatctgtactctggg | |
| | | | aggggttatttgctgacactgtatgcccatttcctaacagaatgtctctagttaagtaatca | |
| | | | tatgatgaagacatcccagctgggactctatatttaagccaagttactatttcta | |
| E23 | hg38_chr12: | 578 | ctaaaaatgcctcctcgcctctgattttagccgtggttgttggagtaccggttccagcagga | 90 |
| | 128365603- | | gctgtgatttccattgagctctcaaaccaaataaaatgcaaatctccgaggatggctcctct | |
| | 128366181 | | ccctgcccccacagttgtgctccgaatagtgtctgagtttcatttttacaaggggcctttaa | |
| | | | aaactcctgggccccttgaaaactcccagccccctttgtccagatggggatggaggtggcca | |
| | | | ggctgccccgttgattgtgtgccgaggagccctccccgggaaggctgtgatttatacgcgca | |
| | | | ggcttgtcacggggtgaaaggaagggccactttttcattttgatccaatgttaggtttgaaa | |
| | | | gccacccactgctgtaaactcagctggatccgcgggccgtgattaaacacattgcccgcttt | |
| | | | gttgccgagatggtgtttcggaaggcgctgtgaatgcacttccctttgcggggctcacacag | |
| | | | acaagatgtgtgttgcaaggatgaggcgcctgctcggcctccagcccagggccgggaaggga | |
| | | | gaaggtgctgtgcgtcgctgc | |
| E24 | hg38_chr12: | 753 | ttgaggcaagagcgagggtggcatatccagggtggccactgggtctggagtgtcagtagcag | 91 |
| | 128375853- | | ggcagatttagaaggtgactttgcatacctaggcaaggccagctcatgcgggatgtcggagc | |
| | 128376606 | | ccatgggaagcaccttgcgtttgaggctgcctgcggtgggaagcttcagagtttcaagcggg | |
| | | | gctttgctatgggtttgttctgctttcccgttttcccctttggaggaggcttacagagatag | |
| | | | tgatgactttgcagctgttaatcatcaggaagctgtaatcactaagaatgtttgaaatcatc | |
| | | | agttaaggatttttagaaggaagtaaaccaaagaaatactgcagtagcctgccctaattatt | |
| | | | tcctgggcttaaagtaaccaggtgcattggagagattatttttcttcttctgatttatgaag | |
| | | | gtctcagggtccaaattttgaaactgctgatcgaatttgttcttggatgttgtcatagaaat | |
| | | | ctgaaactttcctacttgtctgagagtgaaatttctttgattattcactcaagggtttgata | |
| | | | ggtttaaaaaaaggccttcgggacatctcttgttataaagtgtcaactttagatatcaagag | |
| | | | aatcatgatatatttattactacaaaagagaaaataagcaactgaaaaactcatgaacttga | |
| | | | agcatgaagcaaaccccttaagttctaggggtttcaagatgtggatgccaacatgtgatgac | |
| | | | atttaaaaga | |
| E25 | hg38_chr12: | 377 | ggcttttggtttttacaaaatattacaagttgcctaaatagtccgtgtttaaggacatagag | 92 |
| | 128408553- | | ccagagctctttctggaatgtcatacctcggcagggccttttgtgcatgttttaagctgatt | |
| | 128408930 | | ctgaaattagggggttaaaatggaagcgccgagccatccctaaagagagggaggcgaatgtg | |
| | | | cccttgttgctggtgaccccagaacaaggcctctgggctgagaacaggagagaatgttattt | |
| | | | ctttgaaaagccatcttgacaatccaagtccgtttggctgcagcaccaaaggcagctttgat | |
| | | | ctgctcgccagtgtccctgccgggaaaaggattagggtccttccagaggacagcagagccag | |
| | | | gctgcc | |
| E26 | hg38_chr1: | 1489 | gcccaggctggagtgtggtggcaaaatctcagataactgaaacctctgcttcccaggctcaa | 93 |
| | 13388723- | | gccatcctcccacctctgtctgcagagtagctgagactataggcatgtgccacaatgctcag | |
| | 13390212 | | ataattacttaacattctagtagagtctagtagacatgggctatcactatgttgccctggct | |
| | | | ggtctggaactcctgggctcaagtgattgttctgccttggcttcccaaagtgttgggattac | |
| | | | ggctgtaagccgccatgcttggcttcgctttacaatttttttttttttttttttgagacaga | |
| | | | gtcttactctgccacccaggctggagtgtagtggctagattttggctcactgcaaactctgg | |
| | | | cccttgggttaagagattctcctgcctcagcttcccaagtagctgggattacaggcatggac | |
| | | | aaccatacctggctaatattttgtattagcagagacggtatttcaccgtgtcggccgggctg | |
| | | | gtctcgaactcccgacctcatgatccgcctacctcgggctcccaaagtgctgggattacagg | |
| | | | catgagccaccgtgcttggccaagaagacattttgttttctcaaaaaagtggagatctgagc | |
| | | | ttcaaagatccttggtaacacttcccagtgctatcagtgtagtggtgcagtggctaataatt | |
| | | | catggaccctataggagggatcttgcctgctctttagaggttgggacacactcttcttggta | |
| | | | ccagaagggcagaactatgcctctgtggccacttattgcagaatggaattggagtaaactga | |
| | | | gggccctttcacacatgctagagaactgactttggccctaggagaagtgggggttgcagggg | |
| | | | attggcctgagaaacttgccttttcactggattgtcctctagagtttttcactggagatttg | |
| | | | tcagaatgagcctccagtccccatccagactcctggagctggcaggccagagcctgctgagg | |
| | | | aaccagttcttgaccatcttcatcctggacgagctgcccagggaggtcttccctctgatgtt | |
| | | | catggaggcctccagcatgagacattttgaggccctgaagctgatggtgcaggcctggccct | |
| | | | tcctccgcctccctctgggatccctgatgaagacacctcatctggagaccttgcaagctgtg | |
| | | | ctgaagggacttgatacactgctggcccagaagcttcgccccaggtgaggtgactcaggtgg | |
| | | | cctggtgggaagggtccaggcatccagggaagggacagctggctcaggaggagtggtggggt | |
| | | | tggggagctagggtggctcagaggcttctgatggtgcccatgagagaccttgaccattgccc | |
| | | | agatcctctggaaaaggactgctcaccatacagggtccactgaggaaacaggaacctgcttc | |
| | | | ctcccagtggaaggtaaaggttctagaagtgagaaccaggcagaatccaagggggagcggga | |
| | | | tg | |
| E27 | hg38_chr1: | 1738 | ttgggttccaatggaactacagagagcaatgactactggtcctgaatgtgggtataaagttt | 94 |
| | 13469123- | | taccctaaaagtaccttgtatttttttccaaggccaaaatataacaactcatttgcaccctg | |
| | 13470861 | | gaaaggtatgtttatttaaaaaaaaagattgcatttgcaaacagtagagaacactgctcttt | |
| | | | tttatttaaaaaaatctttaccatggaaaaacaataaagtttgcgtgtgtgtttattggtct | |
| | | | ggggacttaaagaacaaagataccttgtggattgcagacaaatgaaacccacagaggtttgc | |
| | | | tttgggtaggtttcaccatacaggtgtttcaatagaccactttgcaaataataaattactta | |
| | | | acactcaaggccgctagaggccactaaaaaggagtttatggccaaggcacagggctggtggc | |
| | | | tggctcagtgagcggtggcaggatattaatgagactcagagcctggacgtgctctggatcca | |
| | | | gttaaatgtaatagagttggaaaaccacctgcccccagccactgacggcacctaggattcat | |
| | | | gcctgtaactttgaccatctgagcctgtagggacattggggaggagggggagggtgagagga | |
| | | | ggcagtggcaacagcagcatggatgttccatgcaaacccttctctgtcaccagggaaagcag | |
| | | | tctgagcacatgaatttcttagcctctcttccaggatgaagcctagtttgaaccagcactgc | |
| | | | caggttgaagtgttactgcatcctgcagccagagccagggcatgtggccacccccttggtcc | |
| | | | ctgctgtggtacccagagtcacttggaacatgtgtgaagccaggatgagggtgcatataccc | |
| | | | tccagatgctgatatctaaatatttacaagtcacaaatacagagaaactgttttttttttgt | |
| | | | tattgttgttgttgttttgagaaggagtctcgctctgtcgcccaggctggagtgcagtagcg | |
| | | | tgatctcggctcaaagttctgcctcccgggttcacgccattctcctgcctcagcctcccaag | |
| | | | tagctgggattataggcatgcaccaccacgcctagcaaattttgtatttttagtagagatgg | |
| | | | ggtttctccatgttggtcaggctggtctcaactcctgatctcaggtgatctgcccacctcag | |
| | | | cctcccaaagtgctgggattacaggtgtgagccaccgcacccggccaacaaaagtaccttct | |
| | | | taatgacttcgaagactaggtttaaatggtaaattattaaattcttggaaatctgccacaga | |
| | | | atatggcattgtggggacagctgagctgattgaaacctgctccctttctcttcccactccca | |
| | | | gctccatcctgcaccttaggggtctatgcacacctgtgtggacatcccaccctcacatccaa | |
| | | | cctctattcacattccccaccaccatcctgtgtggccactcagcctgctctaaagcagggat | |
| | | | gctgggaagatgcccacatccaagcttggaatcgtttttgccagaaattgggggccctaagt | |
| | | | acccaaaaaatgttctagaaggggacatgttctggatggccatggactccttgctccctggg | |
| | | | gaagagcacagctggaggaggactggagcaaggccccctaaagcactggacccaagataatg | |
| | | | cccctcttgcccaggtccaagggctgtactagtggtacccgctgtcatcacagcattcatta | |
| | | | ctg | |
| E28 | hg38_chr22: | 735 | cctcggaaccaaggttggctctggcacctgtagggccacgggcagctatgtcagcttcctcg | 95 |
| | 31124894- | | gaaggaccgaggctggctctggcatctccccgaccaatcctggctccactgtgtacccctga | |
| | 31125629 | | agggcagaaaacagctactgcccaccgcagctccagcctggccccaacatctgtgggccagc | |
| | | | tggtgatgtctgcctcagctggaccaaagcctcccccagcgaccacaggctcagttctggct | |
| | | | ccgacgtccctggggctggtgatgcctgcctcagcagggccaagatctcccccagtcaccct | |
| | | | ggggcccaatctggccccaacctccagagaccagaagcaggagccacctgcctccgtgggac | |
| | | | ccaagccaacactggcagcctctggcctgagcctggccctggcttctgaggagcagccccca | |
| | | | gaactcccctccaccccttccccggtgcccagtccagttctgtctccaactcaggaacaggc | |
| | | | cctggctccagcatccacggcatcaggcgcagcctctgtgggacagacatcagctagaaaga | |
| | | | gggatgccccagcccctagacctctccctgcttctgaggggcatctccagcctccagctcag | |
| | | | acatctggtcctacaggctccccaccctgcatccaaacctccccagaccctcggctctcccc | |
| | | | ctccttccgagcccggcctgaggccctccacagcagccctgaggatcctgtttt | |
| E29 | hg38_chr22: | 1287 | agcctggccaacatggtgaaacgctgcctctactaaaaatacaaaaattagccaggcgtgat | 96 |
| | 31132544- | | ggcggtacctgtggtctcagctactggggaggctgagacaggagaatcacttgaacccggga | |
| | 31133831 | | ggtggaggttgcagtgagccgagattgcaccactgcactccagcctgggtgacagagcgaga | |
| | | | ctccatctcagaaaaagaaaaaaaaaaaaaagagtctctgagtttacagatgagggccctgg | |
| | | | cattcagagaggctgaggaactcacccagcctgtcaacggcagaaccagagccaaatccagg | |
| | | | atttgctagcttcaaagctatgttctcactcactccctaaggaggctgtgggcagaaggaac | |
| | | | cctgggctgggaggcagcacagggcttggtatttatactagacctgttctgcctcagtttcc | |
| | | | cagtctgtaaagtggccctttgtctcaggcaatttgtgctaagacccaagagccttaagtgt | |
| | | | gtgggatactagagggtctcccctgatgtggccccctgcccctgccttgcctggacagtttg | |
| | | | ccttcagggacgacatgccactggtgcggctggaggtggcagatgagtgggtgcggcccgag | |
| | | | caggcggtggtgaggtaccgcatggaaacagtgttcgcccgcagctcctgggactggatcgg | |
| | | | cttataccgggtgagaggggcagtggtggtcagcgactcagggaagaaaggggcctggagga | |
| | | | gcagctgaacagcatggtggggtcactggcttgtccagatcttgatgccacactgggagact | |
| | | | gctgggatcagacattatagggtcacaacactgattccacaacactgatcccccaggtgggt | |
| | | | ttccgccattgcaaggactatgtggcttatgtctgggccaaacatgaagatgtggatgggaa | |
| | | | tacctaccaggtacttaaaaggagtgggagagtcagggcaagtccttgttgcctttgggacc | |
| | | | tcagaactcaccttgggggctctcaggtggcctccctgacccccaacttaggcttatacccc | |
| | | | tgggcctaccaggtaacattcagtgaggaatcactgcccaagggccatggagacttcatcct | |
| | | | gggctactatagtcacaaccacagcatcctcatcggcatcactgaacccttccaggtaagta | |
| | | | ggccagactgctgggctgggggtgcctaaagacttttgtcaaatgccacagcctctacattc | |
| | | | tgctccttgagttcagacaaataacctgacctcccaagatctgccaac | |
| E30 | hg38_chr5: | 1646 | acatcttaaacagtcttttaatgttatgaatttgatttttcaagaaatacatgtcatttatt | 97 |
| | 88655733- | | ttcaaaacgaaatgatagctatactttctagagtctatcaatagtatttaaaataagatact | |
| | 88657379 | | cataactttcaaatactgcttttactagtcatcactcgtcattaaatgtaactgtatattca | |
| | | | agagctttctaataatagcctttaattaaacgaaggactgttagagggtttctgttgccctt | |
| | | | tgaagttcttaattattacttgtatccagcattttatggtacacttaaggttaaattaaatc | |
| | | | atttaaatataccttgaagagaaatatgaagacttttgcccattttaattaaatctctgaat | |
| | | | ttcagtatttgaaaataataacatatgttttgatttttttttcatggccgaatggcaaaatg | |
| | | | ctcactatattaaacaacaaaaaaagaaatggtagctttttatgggactaatcgctaagcag | |
| | | | atgcatgtaaatgagctattttctatgcatggcttccaaaagtgctaattaaatagttggta | |
| | | | ttcaaggctatgctcgctcattgtttagtgacacacaaatccagcgatgtgtgccagcagac | |
| | | | attttaagttgaatgttttctcctctacggtctttgtcatgaaatggtggcaccatgatgag | |
| | | | aacactagtgtaagcaaaacattgaaatatgctttaataatgttttaaccatgtagtgacac | |
| | | | tagcctagttttctaatgaatttttaatttctgttttcttataagggtgatatgagttatcg | |
| | | | ctgatgcatattaaatcatatacatgagtcattttctctaaatttgcataaaatggctaaat | |
| | | | gctaatgcaccaaatggagcttactatatgtggtacagcaaatattcccttgaagattttct | |
| | | | gcaatcaatctcctgtatttcattagcaaccagataaggtgtggtctgcagaataaaaaaag | |
| | | | aaaagtgtgtagctcatgaacttatgaggcttcagatgatttctacgtggtgattagagtgg | |
| | | | attctgcaattagaatttatgtaggtaaaacacacatgtgcttcctttaaaggcacagtgca | |
| | | | acaaaagttctgaatacagccttgcaattgttaaacaatgaaaaggcaccattcaattattg | |
| | | | tgatttttttacatctataattaaatgaaggaaagccatactttaaatttagtatcatttga | |
| | | | ttggcataacccttactgaaattttacaatttccctactatgtttataaaagaacttttaaa | |
| | | | aataaccatgtgtgaaatattttgtttgctaactgttcccattttccttgtcaaataatggt | |
| | | | gaagaattttctggactaatgtttaacatttaaaaatgttttttctatcatcaaatactctt | |
| | | | actgaactgacattaggatcatatgctttataaaaaattgcattagggtaacagtattattg | |
| | | | ggcaaaccagagatgtttacttgaaggataaacttgctgcttactcactccactcatcaacc | |
| | | | cttttctcgtctcctacagttccaccatctggaatattttttaacccagtaaagaaaaaatt | |
| | | | ggggaaggggatggctatttaaaaataaatgcttt | |
| E31 | hg38_chr5: | 314 | tattatcctagtaaatccttaaaaaactttaagaggtgggtgattttataattcccatttta | 98 |
| | 88872683- | | cagatcctggtactggggctttctggtcattaaaacacctgcctaaaaccactaatcagtaa | |
| | 88872997 | | atgggaggctggcttttgaacccagttttggtcgttgttcttaatcattattctttattgtt | |
| | | | tatggacatgtttgtctaatagcataatatgtagaatcaaagaaatgatattaagtgtggaa | |
| | | | atggagtctccaaactctttatgcttgtttaaacgatcttctctctcgagagtgtatcttca | |
| | | | tcctt | |
| E32 | hg38_chr5: | 402 | tttcccttactcagctaacaaacatttaccaagtatctgctgtgtgctaacgcttaggtgtt | 99 |
| | 88745133- | | aaactgggcatacaaactgaatgagaaagagtttgcctccacagagctgagcgtcctagaga | |
| | 88745535 | | gatgtgcccagatgttgcaatcataatgcaatgagaaatgtaatgttggtacaggctactat | |
| | | | gtaagcacaggaaagaggtgcataacttgtctgttagagtcaggaaaggcttttctcaaatg | |
| | | | gctgaactgaattctgtgggatgacaaagagtgctcaatagcatgaagcagaagaaggaaag | |
| | | | gcatgctaggattgcataggtaagagtaagcggccgtgacattgccaagtggcggcacagtg | |
| | | | tagcaattaagagcacacactgaggccgggt | |
| E33 | hg38_chr5: | 1571 | ataaaatatcaggtaaatataatctctcatctctcatcttctctccatctccctatgtccct | 100 |
| | 88799783- | | ttccttctctctctctctctctctctcacacacacacacacacacacacacacacacacaca | |
| | 88801354 | | cacacacagagagagagacacacatgttcttcctctaaaaagaaaaaccaataatcctctac | |
| | | | tgagacagttgtgaatcaaaggtttcttctgcaggagttacatccatctctgaatttcctag | |
| | | | agagcagcaaagggccttgtgttttattccccttccacacttaatcactggactgtgggccc | |
| | | | agactgaatgagtagctcattagaatcactgagttcactgaggggatgagagattccttcct | |
| | | | ggctgggtgctaagtgatactcccataaggattttgtggttacaaaacgtgctggatatgga | |
| | | | ggtaacctgtctgggagtcctgtcactccaaggatcacttggaatgctctggaaaaacacat | |
| | | | gacctggctgaatgagttctgttgaattgtttagcctacaccttcatttcagcagcttatac | |
| | | | tgcattaatgaggttattgttcctttgccgtccaattgttcccaagctgattttttgcatat | |
| | | | atgttttacatccttaacaagaatgcctgtctcctgctgtttcagagtctcttccacagtgc | |
| | | | tgagcatgagtggagcttgctaaatcattgctaaatgaagcaatgggctgtaagcatgtcct | |
| | | | gtgggatctgcatcttcagatcatcctgaagtactcaacaaccacatcttcttccaggaaca | |
| | | | gagcccaacataaactggtagggtttgctgtcttagacagctaagagaacgaggagtggagc | |
| | | | tagtgaacaagcagtgaagggggcagttccttaatgccatccgaactgaatttcaacagtct | |
| | | | gacaagctagcgttttgggtaaatatcccagtatacttgtcacagagttaagtaaaatggac | |
| | | | ttccttcaaaggaagtgcttttaatacaataactgtttttgtttttttaaccaatggattaa | |
| | | | aaatttaacacatttactaaatctggcatatttatatattgtatctaaacagatattcaagc | |
| | | | tgcattataatataatcataaaaaaactgatctcagtctgtctgttaagcctttgtgagtct | |
| | | | ttgtgccattgttggagtagtgctaattatcaagcaaagacatgataattacgcagagcttt | |
| | | | tttgctaaaagaaggaatctttttcaacacccacgcactgcacaatttcctatgaccctgta | |
| | | | gcactactctggtatggcccagaaatttgtatttctgtgtaaaggctggaaattatattatt | |
| | | | tctatctctcccgataccttttcttcttgtgagtaaactgtttttagagggttaaggaagag | |
| | | | gggtaatggtccaaatgggaaatataaaactaatgacccttcatgaaacatattatgctcct | |
| | | | cattaaacttattcagttaaatgtattccattaaattaaaataaataggatttaaaatttta | |
| | | | cccaggagcagtaaacaatctt | |
| E34 | hg38_chr12: | 218 | tgctgaaccagtctccgctgcatcgtctccgctcgcgctcgggacctgcaacagaagggaat | 101 |
| | 27969472- | | gggacccgagtgtcagtctggactctccatctccccgcactactccgctccccctttttagc | |
| | 27969690 | | ccgctctcaaaaagcctcttcaacatcaagggcatctcccaagttgaaaagaaaaaaaattt | |
| | | | ctctggagcctctcagcacttacttatttagca | |
| E35 | hg38_chr12: | 667 | caagctgatgggatcctcccatgggaaaagtgggcctcacacccttcctcacccttccctac | 102 |
| | 27973822- | | ttcctgggcaatgttctgcttcccccaaactgaagcaggaggcccagagaggaggcggtttc | |
| | 27974489 | | ctgggaggaacccaaaccaatgtgagatgagaaggtctttaggaaatgggggtctctgagaa | |
| | | | ccggttcttaaaggtcaagcacttgagcacctcgcaaactcctgacaattgaaacatatctg | |
| | | | aagagtcttcttcagatatgtctctgtgtgtgtgtgtgtgtgtgtgtgtgtgtgtgtgtgag | |
| | | | agagagagagagagagagagagagagaatatgaatgtgcagtgtcccagtcctgatctcctg | |
| | | | gactggtgccagccagccagatgcctgcccttggctggccaagtttttggctcctgaaagta | |
| | | | ggcagctctggacttgtacgaggccacagagagagttccaagccccacctggctcaggcgac | |
| | | | aacctctcaacctgaagtcaatctccggtggcatcacagggccctcctggcagcagcccagt | |
| | | | tccccacatgaaccgaatggtcctttcttaaattttgagccgggggctgcctaaaaggggct | |
| | | | gcccccgcaagcattttacctccctaacaccattctctgcccgtgcca | |

“bp” = base pairs

TABLE 4
Exemplary nucleotide sequences encoding DNA-targeting module components

| | | | | SEQ |
|---|---|---|---|---|
| | | | | ID |
| Name | Species | Variant | Sequence | NO |
| dCas9 | S. aureus | Long | ccaaagaagaagcggaaggtcggtatccacggagtcccagcagccaagaggaactacatcctgggcc | 103 |
| | | | tggccatcggcatcaccagcgtgggctacggcatcatcgactacgagaccagggacgtgatcgacgc | |
| | | | cggcgtgaggctgttcaaggaggccaacgtggagaacaacgagggcaggaggagcaagaggggcgcc | |
| | | | aggaggctgaagaggaggaggaggcacaggatccagagggtgaagaagctgctgttcgactacaacc | |
| | | | tgctgaccgaccacagcgagctgagcggcatcaacccctacgaggccagggtgaagggcctgagcca | |
| | | | gaagctgagcgaggaggagttcagcgccgccctgctgcacctggccaagaggaggggcgtgcacaac | |
| | | | gtgaacgaggtggaggaggacaccggcaacgagctgagcaccaaggagcagatcagcaggaacagca | |
| | | | aggccctggaggagaagtacgtggccgagctgcagctggagaggctgaagaaggacggcgaggtgag | |
| | | | gggcagcatcaacaggttcaagaccagcgactacgtgaaggaggccaagcagctgctgaaggtgcag | |
| | | | aaggcctaccaccagctggaccagagcttcatcgacacctacatcgacctgctggagaccaggagga | |
| | | | cctactacgagggccccggcgagggcagccccttcggctggaaggacatcaaggagtggtacgagat | |
| | | | gctgaagaggaggaggaggcacaggatcctgagccccgtggtgaagaggagcttcatccagagcatc | |
| | | | aaggtgatcaacgccatcatcaagaagtacggcctgcccaacgacatcatcatcgagctggccaggg | |
| | | | agaagaacagcaaggacgcccagaagatgatcaacgagatgcagaagaggaacaggcagaccaacga | |
| | | | gaggatcgaggagatcatcaggaccaccggcaaggagaacgccaagtacctgatcgagaagatcaag | |
| | | | ctgcacgacatgcaggagggcaagtgcctgtacagcctggaggccatccccctggaggacctgctga | |
| | | | acaaccccttcaactacgaggtggaccacatcatccccaggagcgtgagcttcgacaacagcttcaa | |
| | | | caacaaggtgctggtgaagcaggaggaggccagcaagaagggcaacaggacccccttccagtacctg | |
| | | | agcagcagcgacagcaagatcagctacgagaccttcaagaagcacatcctgaacctggccaagggca | |
| | | | agggcaggatcagcaagaccaagaaggagtacctgctggaggagagggacatcaacaggttcagcgt | |
| | | | gcagaaggacttcatcaacaggaacctggtggacaccaggtacgccaccaggggcctgatgaacctg | |
| | | | ctgaggagctacttcagggtgaacaacctggacgtgaaggtgaagagcatcaacggcggcttcacca | |
| | | | gcttcctgaggaggaagtggaagttcaagaaggagaggaacaagggctacaagcaccacgccgagga | |
| | | | cgccctgatcatcgccaacgccgacttcatcttcaaggagtggaagaagctggacaaggccaagaag | |
| | | | gtgatggagaaccagatgttcgaggagaagcaggccgagagcatgcccgagatcgagaccgagcagg | |
| | | | agtacaaggagatcttcatcaccccccaccagatcaagcacatcaaggacttcaaggactacaagta | |
| | | | cagccacagggtggacaagaagcccaacaggaagctgatcaacgacaccctgtacagcaccaggaag | |
| | | | gacgacaagggcaacaccctgatcgtgaacaacctgaacggcctgtacgacaaggacaacgacaagc | |
| | | | tgaagaagctgatcaacaagagccccgagaagctgctgatgtaccaccacgacccccagacctacca | |
| | | | gaagctgaagctgatcatggagcagtacggcgacgagaagaaccccctgtacaagtactacgaggag | |
| | | | accggcaactacctgaccaagtacagcaagaaggacaacggccccgtgatcaagaagatcaagtact | |
| | | | acggcaacaagctgaacgcccacctggacatcaccgacgactaccccaacagcaggaacaaggtggt | |
| | | | gaagctgagcctgaagccctacaggttcgacgtgtacctggacaacggcgtgtacaagttcgtgacc | |
| | | | gtgaagaacctggacgtgatcaagaaggagaactactacgaggtgaacagcaagtgctacgaggagg | |
| | | | ccaagaagctgaagaagatcagcaaccaggccgagttcatcgccagcttctacaagaacgacctgat | |
| | | | caagatcaacggcgagctgtacagggtgatcggcgtgaacaacgacctgctgaacaggatcgaggtg | |
| | | | aacatgatcgacatcacctacagggagtacctggagaacatgaacgacaagaggcccccccacatca | |
| | | | tcaagaccatcgccagcaagacccagagcatcaagaagtacagcaccgacatcctgggcaacctgta | |
| | | | cgaggtgaagagcaagaagcacccccagatcatcaagaagggcaaaaggccggcggccacgaaaaag | |
| | | | gccggccaggcaaaaaagaaaaagggatccgaggccagcggttccggacgggctgacgcattggacg | |
| | | | attttgatctggatatgctgggaagtgacgccctcgatgattttgaccttgacatgcttggttcgga | |
| | | | tgcccttgatgactttgacctcgacatgctcggcagtgacgcccttgatgatttcgacctggacatg | |
| | | | ctg | |
| dCas9 | S. aureus | Short | ccaaagaagaagcggaaggtcggtatccacggagtcccagcagccaagaggaactacatcctgggcc | 104 |
| | | | tggccatcggcatcaccagcgtgggctacggcatcatcgactacgagaccagggacgtgatcgacgc | |
| | | | cggcgtgaggctgttcaaggaggccaacgtggagaacaacgagggcaggaggagcaagaggggcgcc | |
| | | | aggaggctgaagaggaggaggaggcacaggatccagagggtgaagaagctgctgttcgactacaacc | |
| | | | tgctgaccgaccacagcgagctgagcggcatcaacccctacgaggccagggtgaagggcctgagcca | |
| | | | gaagctgagcgaggaggagttcagcgccgccctgctgcacctggccaagaggaggggcgtgcacaac | |
| | | | gtgaacgaggtggaggaggacaccggcaacgagctgagcaccaaggagcagatcagcaggaacagca | |
| | | | aggccctggaggagaagtacgtggccgagctgcagctggagaggctgaagaaggacggcgaggtgag | |
| | | | gggcagcatcaacaggttcaagaccagcgactacgtgaaggaggccaagcagctgctgaaggtgcag | |
| | | | aaggcctaccaccagctggaccagagcttcatcgacacctacatcgacctgctggagaccaggagga | |
| | | | cctactacgagggccccggcgagggcagccccttcggctggaaggacatcaaggagtggtacgagat | |
| | | | gctgaagaggaggaggaggcacaggatcctgagccccgtggtgaagaggagcttcatccagagcatc | |
| | | | aaggtgatcaacgccatcatcaagaagtacggcctgcccaacgacatcatcatcgagctggccaggg | |
| | | | agaagaacagcaaggacgcccagaagatgatcaacgagatgcagaagaggaacaggcagaccaacga | |
| | | | gaggatcgaggagatcatcaggaccaccggcaaggagaacgccaagtacctgatcgagaagatcaag | |
| | | | ctgcacgacatgcaggagggcaagtgcctgtacagcctggaggccatccccctggaggacctgctga | |
| | | | acaaccccttcaactacgaggtggaccacatcatccccaggagcgtgagcttcgacaacagcttcaa | |
| | | | caacaaggtgctggtgaagcaggaggaggccagcaagaagggcaacaggacccccttccagtacctg | |
| | | | agcagcagcgacagcaagatcagctacgagaccttcaagaagcacatcctgaacctggccaagggca | |
| | | | agggcaggatcagcaagaccaagaaggagtacctgctggaggagagggacatcaacaggttcagcgt | |
| | | | gcagaaggacttcatcaacaggaacctggtggacaccaggtacgccaccaggggcctgatgaacctg | |
| | | | ctgaggagctacttcagggtgaacaacctggacgtgaaggtgaagagcatcaacggcggcttcacca | |
| | | | gcttcctgaggaggaagtggaagttcaagaaggagaggaacaagggctacaagcaccacgccgagga | |
| | | | cgccctgatcatcgccaacgccgacttcatcttcaaggagtggaagaagctggacaaggccaagaag | |
| | | | gtgatggagaaccagatgttcgaggagaagcaggccgagagcatgcccgagatcgagaccgagcagg | |
| | | | agtacaaggagatcttcatcaccccccaccagatcaagcacatcaaggacttcaaggactacaagta | |
| | | | cagccacagggtggacaagaagcccaacaggaagctgatcaacgacaccctgtacagcaccaggaag | |
| | | | gacgacaagggcaacaccctgatcgtgaacaacctgaacggcctgtacgacaaggacaacgacaagc | |
| | | | tgaagaagctgatcaacaagagccccgagaagctgctgatgtaccaccacgacccccagacctacca | |
| | | | gaagctgaagctgatcatggagcagtacggcgacgagaagaaccccctgtacaagtactacgaggag | |
| | | | accggcaactacctgaccaagtacagcaagaaggacaacggccccgtgatcaagaagatcaagtact | |
| | | | acggcaacaagctgaacgcccacctggacatcaccgacgactaccccaacagcaggaacaaggtggt | |
| | | | gaagctgagcctgaagccctacaggttcgacgtgtacctggacaacggcgtgtacaagttcgtgacc | |
| | | | gtgaagaacctggacgtgatcaagaaggagaactactacgaggtgaacagcaagtgctacgaggagg | |
| | | | ccaagaagctgaagaagatcagcaaccaggccgagttcatcgccagcttctacaagaacgacctgat | |
| | | | caagatcaacggcgagctgtacagggtgatcggcgtgaacaacgacctgctgaacaggatcgaggtg | |
| | | | aacatgatcgacatcacctacagggagtacctggagaacatgaacgacaagaggcccccccacatca | |
| | | | tcaagaccatcgccagcaagacccagagcatcaagaagtacagcaccgacatcctgggcaacctgta | |
| | | | cgaggtgaagagcaagaagcacccccagatcatcaagaagggcaaaaggccggcggccacgaaaaag | |
| | | | gccggccaggcaaaaaagaaaaagggatccgaggccagcggttccggacgggctgacgcattggacg | |
| | | | attttgatctggatatgctgggaagtgacgccctcgatgattttgaccttgacatgcttggttcgga | |
| | | | tgcccttgatgactttgacctcgacatgctcggcagtgacgcccttgatgatttcgacctggacatg | |
| | | | ctg | |
| dCas9 | S. aureus | Short / | ccaaagaagaagagaaaggttggcattcacggggtgccagccgccaaaaggaattatatcctcggat | 105 |
| | | codon | tggcaataggcataaccagtgtaggttatggaatcatagactatgaaacccgggacgttatagacgc | |
| | | optimized | gggagtacggttgttcaaagaggcaaatgttgaaaacaatgagggccggcggtccaaacggggtgca | |
| | | #1 | cggaggcttaaacgccgcaggagacatcggattcaaagggtcaagaaacttctttttgattataacc | |
| | | | tcctgaccgaccacagtgagctctccgggataaatccttacgaagccagagtcaagggacttagcca | |
| | | | aaagctgtcagaggaagagttttcagcggcactgcttcatcttgcaaaaaggcggggcgtccataat | |
| | | | gttaatgaggtagaagaagatactgggaacgagctcagtacgaaggaacaaatctcaagaaatagca | |
| | | | aggctcttgaggaaaaatacgtagcagaacttcagttggaaagacttaagaaagacggagaggttcg | |
| | | | gggaagtattaaccgctttaagacttcagattatgttaaagaggcaaagcaattgttgaaagttcag | |
| | | | aaagcttatcatcaattggatcaatcatttatagatacatacatagatctccttgaaaccagacgga | |
| | | | cgtactacgaaggtccaggagaagggtcacccttcggatggaaggacatcaaggaatggtacgaaat | |
| | | | gcttaaaaggaggcgaagacatagaattctcagtcctgtcgtgaagcgaagcttcatacagagcata | |
| | | | aaggtaataaacgctataatcaagaagtacggattgcctaacgatattataatagaattggcacgag | |
| | | | agaaaaacagtaaggatgcacagaagatgattaacgagatgcagaagcgaaatcgccaaacgaatga | |
| | | | gcgcatagaagagattataagaactacaggcaaggagaacgccaagtatcttatagaaaagataaaa | |
| | | | ctccatgacatgcaggaaggtaaatgtctttattctctcgaggctatcccgttggaggatctgctga | |
| | | | ataaccctttcaattacgaagtagaccatatcatacctcgatccgtttcattcgataattcctttaa | |
| | | | caataaagtgctcgttaaacaggaagaggcatccaagaaagggaatagaactccgtttcaatacctg | |
| | | | tctagttctgactctaaaataagttatgaaacctttaaaaagcatatactcaaccttgcgaagggca | |
| | | | aggggcggatatcaaagaccaaaaaggaatacctcctcgaggagcgggacattaatagattttctgt | |
| | | | gcagaaagattttataaaccgcaacttggtagacacgcgatatgctacacgaggtctgatgaacttg | |
| | | | ctccgatcttattttcgcgtcaataatctggatgtcaaggtgaagtctattaacggtggtttcacat | |
| | | | ccttcctgcgacgaaagtggaagtttaagaaagagcggaataagggctacaagcatcatgccgagga | |
| | | | cgctctgattatcgctaacgcggattttatctttaaagagtggaagaaactcgacaaagctaagaaa | |
| | | | gtgatggagaaccagatgtttgaggaaaagcaggccgaatcaatgccggaaatcgagacggagcagg | |
| | | | aatataaggagattttcatcactcctcatcagataaagcatattaaagacttcaaagactacaagta | |
| | | | tagccacagggtggacaagaagcccaacaggaaactgattaacgatacactctattccacccggaaa | |
| | | | gatgataaaggaaatacgctgatcgtaaataacctcaacggactttacgataaggataatgataaac | |
| | | | tcaaaaagcttatcaataagtctcccgagaagctgctcatgtaccaccatgatccccagacatacca | |
| | | | aaagctgaagctcatcatggaacagtatggagatgagaagaacccgttgtacaaatactatgaagag | |
| | | | acgggcaactaccttactaagtacagcaagaaggataacgggccggtgatcaaaaagattaagtatt | |
| | | | acgggaataaacttaatgcgcatcttgacatcactgacgactaccccaattctcggaataaggttgt | |
| | | | aaaactgtcccttaagccatatcggttcgacgtctatctcgacaacggtgtttacaagttcgtaaca | |
| | | | gtcaagaatcttgatgttataaaaaaagagaattattacgaagttaactcaaagtgctatgaggaag | |
| | | | cgaaaaaacttaaaaaaatatccaaccaggccgagtttatagcttccttttataaaaacgatctgat | |
| | | | aaaaatcaacggcgaattgtacagagtaatcggtgttaataatgatttgctgaataggatagaagtt | |
| | | | aatatgatagatattacttacagggaatatttggaaaacatgaatgacaagcggcccccgcacatca | |
| | | | tcaagacaatcgcatccaaaactcagtccatcaagaagtattctacggacattcttggaaatcttta | |
| | | | tgaggtaaagagcaaaaaacatccgcaaatcattaaaaagggaaagagacccgcagccactaagaag | |
| | | | gccgggcaggcgaagaagaaaaagggttctgaggctagcgggtctggccgagcggatgctttggatg | |
| | | | attttgacttggacatgcttggttctgacgctcttgatgattttgatctggatatgttgggaagcga | |
| | | | cgcgctggatgattttgatctggatatgttgggctcagacgcacttgatgacttcgaccttgatatg | |
| | | | ctg | |
| dCas9 | S. pyogenes | Long | atggacaagaagtactccattgggctcgctatcggcacaaacagcgtcggctgggccgtcattacgg | 106 |
| | | | acgagtacaaggtgccgagcaaaaaattcaaagttctgggcaataccgatcgccacagcataaagaa | |
| | | | gaacctcattggcgccctcctgttcgactccggggagacggccgaagccacgcggctcaaaagaaca | |
| | | | gcacggcgcagatatacccgcagaaagaatcggatctgctacctgcaggagatctttagtaatgaga | |
| | | | tggctaaggtggatgactctttcttccataggctggaggagtcctttttggtggaggaggataaaaa | |
| | | | gcacgagcgccacccaatctttggcaatatcgtggacgaggtggcgtaccatgaaaagtacccaacc | |
| | | | atatatcatctgaggaagaagcttgtagacagtactgataaggctgacttgcggttgatctatctcg | |
| | | | cgctggcgcatatgatcaaatttcggggacacttcctcatcgagggggacctgaacccagacaacag | |
| | | | cgatgtcgacaaactctttatccaactggttcagacttacaatcagcttttcgaagagaacccgatc | |
| | | | aacgcatccggagttgacgccaaagcaatcctgagcgctaggctgtccaaatcccggcggctcgaaa | |
| | | | acctcatcgcacagctccctggggagaagaagaacggcctgtttggtaatcttatcgccctgtcact | |
| | | | cgggctgacccccaactttaaatctaacttcgacctggccgaagatgccaagcttcaactgagcaaa | |
| | | | gacacctacgatgatgatctcgacaatctgctggcccagatcggcgaccagtacgcagacctttttt | |
| | | | tggcggcaaagaacctgtcagacgccattctgctgagtgatattctgcgagtgaacacggagatcac | |
| | | | caaagctccgctgagcgctagtatgatcaagcgctatgatgagcaccaccaagacttgactttgctg | |
| | | | aaggcccttgtcagacagcaactgcctgagaagtacaaggaaattttcttcgatcagtctaaaaatg | |
| | | | gctacgccggatacattgacggcggagcaagccaggaggaattttacaaatttattaagcccatctt | |
| | | | ggaaaaaatggacggcaccgaggagctgctggtaaagcttaacagagaagatctgttgcgcaaacag | |
| | | | cgcactttcgacaatggaagcatcccccaccagattcacctgggcgaactgcacgctatcctcaggc | |
| | | | ggcaagaggatttctacccctttttgaaagataacagggaaaagattgagaaaatcctcacatttcg | |
| | | | gataccctactatgtaggccccctcgcccggggaaattccagattcgcgtggatgactcgcaaatca | |
| | | | gaagagaccatcactccctggaacttcgaggaagtcgtggataagggggcctctgcccagtccttca | |
| | | | tcgaaaggatgactaactttgataaaaatctgcctaacgaaaaggtgcttcctaaacactctctgct | |
| | | | gtacgagtacttcacagtttataacgagctcaccaaggtcaaatacgtcacagaagggatgagaaag | |
| | | | ccagcattcctgtctggagagcagaagaaagctatcgtggacctcctcttcaagacgaaccggaaag | |
| | | | ttaccgtgaaacagctcaaagaagactatttcaaaaagattgaatgtttcgactctgttgaaatcag | |
| | | | cggagtggaggatcgcttcaacgcatccctgggaacgtatcacgatctcctgaaaatcattaaagac | |
| | | | aaggacttcctggacaatgaggagaacgaggacattcttgaggacattgtcctcacccttacgttgt | |
| | | | ttgaagatagggagatgattgaagaacgcttgaaaacttacgctcatctcttcgacgacaaagtcat | |
| | | | gaaacagctcaagaggcgccgatatacaggatgggggcggctgtcaagaaaactgatcaatgggatc | |
| | | | cgagacaagcagagtggaaagacaatcctggattttcttaagtccgatggatttgccaaccggaact | |
| | | | tcatgcagttgatccatgatgactctctcacctttaaggaggacatccagaaagcacaagtttctgg | |
| | | | ccagggggacagtcttcacgagcacatcgctaatcttgcaggtagcccagctatcaaaaagggaata | |
| | | | ctgcagaccgttaaggtcgtggatgaactcgtcaaagtaatgggaaggcataagcccgagaatatcg | |
| | | | ttatcgagatggcccgagagaaccaaactacccagaagggacagaagaacagtagggaaaggatgaa | |
| | | | gaggattgaagagggtataaaagaactggggtcccaaatccttaaggaacacccagttgaaaacacc | |
| | | | cagcttcagaatgagaagctctacctgtactacctgcagaacggcagggacatgtacgtggatcagg | |
| | | | aactggacatcaatcggctctccgactacgacgtggctgctatcgtgccccagtcttttctcaaaga | |
| | | | tgattctattgataataaagtgttgacaagatccgataaagctagagggaagagtgataacgtcccc | |
| | | | tcagaagaagttgtcaagaaaatgaaaaattattggcggcagctgctgaacgccaaactgatcacac | |
| | | | aacggaagttcgataatctgactaaggctgaacgaggtggcctgtctgagttggataaagccggctt | |
| | | | catcaaaaggcagcttgttgagacacgccagatcaccaagcacgtggcccaaattctcgattcacgc | |
| | | | atgaacaccaagtacgatgaaaatgacaaactgattcgagaggtgaaagttattactctgaagtcta | |
| | | | agctggtctcagatttcagaaaggactttcagttttataaggtgagagagatcaacaattaccacca | |
| | | | tgcgcatgatgcctacctgaatgcagtggtaggcactgcacttatcaaaaaatatcccaagcttgaa | |
| | | | tctgaatttgtttacggagactataaagtgtacgatgttaggaaaatgatcgcaaagtctgagcagg | |
| | | | aaataggcaaggccaccgctaagtacttcttttacagcaatattatgaattttttcaagaccgagat | |
| | | | tacactggccaatggagagattcggaagcgaccacttatcgaaacaaacggagaaacaggagaaatc | |
| | | | gtgtgggacaagggtagggatttcgcgacagtccggaaggtcctgtccatgccgcaggtgaacatcg | |
| | | | ttaaaaagaccgaagtacagaccggaggcttctccaaggaaagtatcctcccgaaaaggaacagcga | |
| | | | caagctgatcgcacgcaaaaaagattgggaccccaagaaatacggcggattcgattctcctacagtc | |
| | | | gcttacagtgtactggttgtggccaaagtggagaaagggaagtctaaaaaactcaaaagcgtcaagg | |
| | | | aactgctgggcatcacaatcatggagcgatcaagcttcgaaaaaaaccccatcgactttctcgaggc | |
| | | | gaaaggatataaagaggtcaaaaaagacctcatcattaagcttcccaagtactctctctttgagctt | |
| | | | gaaaacggccggaaacgaatgctcgctagtgcgggcgagctgcagaaaggtaacgagctggcactgc | |
| | | | cctctaaatacgttaatttcttgtatctggccagccactatgaaaagctcaaagggtctcccgaaga | |
| | | | taatgagcagaagcagctgttcgtggaacaacacaaacactaccttgatgagatcatcgagcaaata | |
| | | | agcgaattctccaaaagagtgatcctcgccgacgctaacctcgataaggtgctttctgcttacaata | |
| | | | agcacagggataagcccatcagggagcaggcagaaaacattatccacttgtttactctgaccaactt | |
| | | | gggcgcgcctgcagccttcaagtacttcgacaccaccatagacagaaagcggtacacctctacaaag | |
| | | | gaggtcctggacgccacactgattcatcagtcaattacggggctctatgaaacaagaatcgacctct | |
| | | | ctcagctcggtggagac | |

TABLE 5
Exemplary nucleotide sequences encoding transactivators

| Name | Sequence | SEQ ID NO |
|---|---|---|
| VP16(1) | gacgcgctggacgatttcgatctcgacatgctgggttct | 107 |
| VP16(2) | gacgcgctggacgatttcgatctcgacatgctgggttctgatgccctcgatgactttgacctggatatgttgggaagc | 108 |
| VP16(3) | gacgcgctggacgatttcgatctcgacatgctgggttctgatgccctcgatgactttgacctggatatgttgggaagcgacgca | 109 |
| | ttggatgactttgatctggacatgctcggctcc | |
| VP64 | gacgcgctggacgatttcgatctcgacatgctgggttctgatgccctcgatgactttgacctggatatgttgggaagcgacgca | 110 |
| | ttggatgactttgatctggacatgctcggctccgatgctctggacgatttcgatctcgatatgtta | |
| VPR | gacgcattggacgattttgatctggatatgctgggaagtgacgccctcgatgattttgaccttgacatgcttggttcggatgcc | 111 |
| | cttgatgactttgacctcgacatgctcggcagtgacgcccttgatgatttcgacctggacatgctgattaactctagaagttcc | |
| | ggatctccgaaaaagaaacgcaaagttggtagccagtacctgcccgacaccgacgaccggcaccggatcgaggaaaagcggaag | |
| | cggacctacgagacattcaagagcatcatgaagaagtcccccttcagcggccccaccgaccctagacctccacctagaagaatc | |
| | gccgtgcccagcagatccagcgccagcgtgccaaaacctgccccccagccttaccccttcaccagcagcctgagcaccatcaac | |
| | tacgacgagttccctaccatggtgttccccagcggccagatctctcaggcctctgctctggctccagcccctcctcaggtgctg | |
| | cctcaggctcctgctcctgcaccagctccagccatggtgtctgcactggctcaggcaccagcacccgtgcctgtgctggctcct | |
| | ggacctccacaggctgtggctccaccagcccctaaacctacacaggccggcgagggcacactgtctgaagctctgctgcagctg | |
| | cagttcgacgacgaggatctgggagccctgctgggaaacagcaccgatcctgccgtgttcaccgacctggccagcgtggacaac | |
| | agcgagttccagcagctgctgaaccagggcatccctgtggcccctcacaccaccgagcccatgctgatggaataccccgaggcc | |
| | atcacccggctcgtgacaggcgctcagaggcctcctgatccagctcctgcccctctgggagcaccaggcctgcctaatggactg | |
| | ctgtctggcgacgaggacttcagctctatcgccgatatggatttctcagccttgctgggctctggcagcggcagccgggattcc | |
| | agggaagggatgtttttgccgaagcctgaggccggctccgctattagtgacgtgtttgagggccgcgaggtgtgccagccaaaa | |
| | cgaatccggccatttcatcctccaggaagtccatgggccaaccgcccactccccgccagcctcgcaccaacaccaaccggtcca | |
| | gtacatgagccagtcgggtcactgaccccggcaccagtccctcagccactggatccagcgcccgcagtgactcccgaggccagt | |
| | cacctgttggaggatcccgatgaagagacgagccaggctgtcaaagcccttcgggagatggccgatactgtgattccccagaag | |
| | gaagaggctgcaatctgtggccaaatggacctttcccatccgcccccaaggggccatctggatgagctgacaaccacacttgag | |
| | tccatgaccgaggatctgaacctggactcacccctgaccccggaattgaacgagattctggataccttcctgaacgacgagtgc | |
| | ctcttgcatgccatgcatatcagcacaggactgtccatcttcgacacatctctgttt | |
| p65 | cctacacaggccggcgagggcacactgtctgaagctctgctgcagctgcagttcgacgacgaggatctgggagccctgctggga | 112 |
| | aacagcaccgatcctgccgtgttcaccgacctggccagcgtggacaacagcgagttccagcagctgctgaaccagggcatccct | |
| | gtggcccctcacaccaccgagcccatgctgatggaataccccgaggccatcacccggctcgtgacaggcgctcagaggcctcct | |
| | gatccagctcctgcccctctgggagcaccaggcctgcctaatggactgctgtctggcgacgaggacttcagctctatcgccgat | |
| | atggatttctcagccttgctg | |
| Rta | cgggattccagggaagggatgtttttgccgaagcctgaggccggctccgctattagtgacgtgtttgagggccgcgaggtgtgc | 113 |
| | cagccaaaacgaatccggccatttcatcctccaggaagtccatgggccaaccgcccactccccgccagcctcgcaccaacacca | |
| | accggtccagtacatgagccagtcgggtcactgaccccggcaccagtccctcagccactggatccagcgcccgcagtgactccc | |
| | gaggccagtcacctgttggaggatcccgatgaagagacgagccaggctgtcaaagcccttcgggagatggccgatactgtgatt | |
| | ccccagaaggaagaggctgcaatctgtggccaaatggacctttcccatccgcccccaaggggccatctggatgagctgacaacc | |
| | acacttgagtccatgaccgaggatctgaacctggactcacccctgaccccggaattgaacgagattctggataccttcctgaac | |
| | gacgagtgcctcttgcatgccatgcatatcagcacaggactgtccatcttcgacacatctctgttt | |

TABLE 6
Exemplary transgene sequences and amino acid sequences encoded by transgenes

| Name | Sequence | SEQ ID NO |
|---|---|---|
| NLS-SadCas9- | MAPKKKRKVGIHGVPAAKRNYILGLAIGITSVGYGIIDYETRDVIDAGVRLFKEANVENNEGRRSKRGARRL | 114 |
| NLS-VP64 | KRRRRHRIQRVKKLLFDYNLLTDHSELSGINPYEARVKGLSQKLSEEEFSAALLHLAKRRGVHNVNEVEEDT | |
| (amino acid | GNELSTKEQISRNSKALEEKYVAELQLERLKKDGEVRGSINRFKTSDYVKEAKQLLKVQKAYHQLDQSFIDT | |
| sequence) | YIDLLETRRTYYEGPGEGSPFGWKDIKEWYEMLMGHCTYFPEELRSVKYAYNADLYNALNDLNNLVITRDEN | |
| | EKLEYYEKFQIIENVFKQKKKPTLKQIAKEILVNEEDIKGYRVTSTGKPEFTNLKVYHDIKDITARKEIIEN | |
| | AELLDQIAKILTIYQSSEDIQEELTNLNSELTQEEIEQISNLKGYTGTHNLSLKAINLILDELWHTNDNQIA | |
| | IFNRLKLVPKKVDLSQQKEIPTTLVDDFILSPVVKRSFIQSIKVINAIIKKYGLPNDIIIELAREKNSKDAQ | |
| | KMINEMQKRNRQTNERIEEIIRTTGKENAKYLIEKIKLHDMQEGKCLYSLEAIPLEDLLNNPFNYEVDHIIP | |
| | RSVSFDNSFNNKVLVKQEEASKKGNRTPFQYLSSSDSKISYETFKKHILNLAKGKGRISKTKKEYLLEERDI | |
| | NRFSVQKDFINRNLVDTRYATRGLMNLLRSYFRVNNLDVKVKSINGGFTSFLRRKWKFKKERNKGYKHHAED | |
| | ALIIANADFIFKEWKKLDKAKKVMENQMFEEKQAESMPEIETEQEYKEIFITPHQIKHIKDFKDYKYSHRVD | |
| | KKPNRELINDTLYSTRKDDKGNTLIVNNLNGLYDKDNDKLKKLINKSPEKLLMYHHDPQTYQKLKLIMEQYG | |
| | DEKNPLYKYYEETGNYLTKYSKKDNGPVIKKIKYYGNKLNAHLDITDDYPNSRNKVVKLSLKPYRFDVYLDN | |
| | GVYKFVTVKNLDVIKKENYYEVNSKCYEEAKKLKKISNQAEFIASFYNNDLIKINGELYRVIGVNNDLLNRI | |
| | EVNMIDITYREYLENMNDKRPPRIIKTIASKTQSIKKYSTDILGNLYEVKSKKHPQIIKKGKRPAATKKAGQ | |
| | AKKKKGSEASGSGRADALDDFDLDMLGSDALDDFDLDMLGSDALDDFDLDMLGSDALDDFDLDML | |
| NLS-SadCas9- | ccaaagaagaagcgaaaagttggaatccatggggtcccggccgctaaaagaaactatatcctggggttggcaataggaa | 115 |
| NLS-VP64 | tcaccagcgtcggttacggtataatagattatgagacacgagatgtaatcgatgcgggagtacgcttgttcaaagaagc | |
| (nucleotide | caacgtagaaaataatgagggacggcggtctaaacgaggagctagacggcttaagcgacgccgccggcaccggatccaa | |
| sequence) | cgcgttaaaaagttgctttttgactataatctgcttacggatcactccgagctctctgggattaatccgtacgaagccc | |
| | gcgtcaaggggttgagccaaaaattgagtgaagaggaatttagcgctgcgctcttgcacctggcgaaaaggcggggtgt | |
| | acataacgtaaatgaggttgaggaggatactggtaacgaactcagcaccaaggagcaaataagtcggaacagcaaagca | |
| | ttggaggaaaaatacgtggcggagctgcaacttgagcggctcaagaaggatggggaagtgcgcgggagcataaaccgat | |
| | ttaagaccagcgattatgtgaaagaagctaagcaattgttgaaggttcagaaggcttaccatcagctcgaccaatcatt | |
| | cattgatacgtatatagacctgttggaaacacgacgcacctattatgaaggtccgggtgaaggaagtcccttcgggtgg | |
| | aaggacatcaaagaatggtacgaaatgcttatgggacattgtacttacttccctgaggaactgaggagcgtaaagtatg | |
| | cctataatgcagatttgtacaacgcgctgaacgacttgaataatctcgtgataactcgggacgagaacgagaaacttga | |
| | gtattacgagaagttccagatcatcgagaatgtctttaagcagaagaaaaaaccgaccttgaaacaaattgccaaggag | |
| | atactggtaaatgaagaagacattaagggttatcgagtaacgtcaactgggaaaccagagttcacaaacttgaaagtct | |
| | accacgatataaaagatataacggctcgaaaggagatcatcgaaaatgcagagttgttggatcagatagcgaagatatt | |
| | gaccatctatcaaagcagcgaggatatacaggaagagctgaccaatttgaattccgagctcacacaggaggaaatagag | |
| | cagatcagtaacttgaaaggttacacggggactcacaacctgagtttgaaggcgatcaacttgattctggatgagttgt | |
| | ggcataccaatgacaatcaaatcgccatatttaacaggcttaagcttgtccccaaaaaagtggacctctcacagcagaa | |
| | agaaattccaaccaccctcgtggacgacttcatattgagtcccgtggtgaagcgatcatttatccaatcaatcaaagta | |
| | atcaatgccatcattaaaaaatacggcttgccaaatgacataatcattgagttggcacgcgaaaaaaatagtaaggatg | |
| | ctcaaaagatgataaacgagatgcagaaacggaatcgccaaactaatgaacgcattgaggaaataattcgcactaccgg | |
| | gaaggaaaacgcaaagtatttgattgagaaaatcaaactgcacgacatgcaagagggcaaatgtctttacagcctggag | |
| | gcgatccctctcgaagacttgcttaacaacccgtttaactatgaggtcgatcatattatacctcgatctgtgagttttg | |
| | ataacagtttcaataacaaagttcttgtcaagcaagaggaggcctccaagaaaggtaatcgcaccccgttccagtattt | |
| | gtctagtagcgattccaaaatatcatatgaaacttttaagaagcatatcttgaaccttgctaaggggaaggggcgcatc | |
| | tccaagacaaagaaagagtatcttctggaagaacgcgatatcaacaggttttcagttcaaaaagacttcatcaacagaa | |
| | acctcgtggatacgcggtatgccacacgaggactcatgaatcttctcaggtcatactttcgggttaacaacctggacgt | |
| | aaaagttaaaagcatcaacggggggtttacgtccttcttgcgccgcaaatggaaattcaaaaaggagagaaacaagggc | |
| | tacaaacatcacgcggaagacgcactgattatcgcgaacgctgattttatttttaaagaatggaagaaacttgataagg | |
| | cgaaaaaagtaatggaaaatcagatgtttgaggagaagcaggcggagtctatgccagaaattgaaacggagcaagaata | |
| | caaagaaatctttataacgccacatcagataaagcatattaaggactttaaagattacaaatactcacaccgggtcgac | |
| | aaaaaacccaatagggagctgataaacgacactctgtactccacacggaaagacgacaaaggcaacacactcattgtta | |
| | acaacttgaatggcttgtacgataaagacaatgacaagctgaaaaaacttataaataaatcaccggagaaactgctgat | |
| | gtatcatcatgacccgcaaacgtatcaaaagctcaagctgataatggaacagtatggagatgagaaaaatccattgtac | |
| | aaatattacgaagaaactggtaactatcttacaaaatactcaaaaaaggataatgggccagttataaagaagataaagt | |
| | attacggcaacaagcttaatgctcatctggacatcacagatgactatcccaactcccggaataaagtcgtaaagctgag | |
| | ccttaaaccgtacagattcgatgtttatctggacaatggagtctataaattcgtaactgtgaaaaacttggatgtgata | |
| | aagaaagagaactactatgaggtaaattctaaatgttacgaagaagcgaaaaaattgaaaaaaatctcaaatcaagccg | |
| | agtttatcgcaagcttctacaacaatgatctcattaaaattaacggagaactctaccgggttataggagtaaataatga | |
| | cctcctgaaccgaatagaggtgaatatgatagatattacttatagagagtatttggagaacatgaacgataagaggcct | |
| | cctcggattattaagaccatcgcctcaaagactcagtcaatcaaaaaatattcaacggacattcttgggaatttgtacg | |
| | aggttaaaagtaaaaaacatcctcaaataatcaaaaaagggaagaggccggcggctacgaaaaaggctggacaggcgaa | |
| | gaagaaaaagggttcagaagcgagtgggagcggtcgcgcggacgctctcgatgatttcgacctggacatgcttgggtcc | |
| | gatgctctcgacgattttgatctggacatgctcggttctgacgcgcttgacgacttcgacctcgacatgttggggtcag | |
| | acgcgctcgacgatttcgacctcgatatgctg | |
| NLS-SadCas9- | ccaaagaagaagcgaaaagttggaatccatggggtcccggccgctaaaagaaactatatcctggggttggcaataggaa | 116 |
| NLS-VP64 | tcaccagcgtcggttacggtataatagattatgagacacgagatgtaatcgatgcgggagtacgcttgttcaaagaagc | |
| (optimized | caacgtagaaaataatgagggacggcggtctaaacgaggagctagacggcttaagcgacgccgccggcaccggatccaa | |
| nucleotide | cgcgttaaaaagttgctttttgactataatctgcttacggatcactccgagctctctgggattaatccgtacgaagccc | |
| sequence 1) | gcgtcaaggggttgagccaaaaattgagtgaagaggaatttagcgctgcgctcttgcacctggcgaaaaggcggggtgt | |
| | acataacgtaaatgaggttgaggaggatactggtaacgaactcagcaccaaggagcaaataagtcggaacagcaaagca | |
| | ttggaggaaaaatacgtggcggagctgcaacttgagcggctcaagaaggatggggaagtgcgcgggagcataaaccgat | |
| | ttaagaccagcgattatgtgaaagaagctaagcaattgttgaaggttcagaaggcttaccatcagctcgaccaatcatt | |
| | cattgatacgtatatagacctgttggaaacacgacgcacctattatgaaggtccgggtgaaggaagtcccttcgggtgg | |
| | aaggacatcaaagaatggtacgaaatgcttatgggacattgtacttacttccctgaggaactgaggagcgtaaagtatg | |
| | cctataatgcagatttgtacaacgcgctgaacgacttgaataatctcgtgataactcgggacgagaacgagaaacttga | |
| | gtattacgagaagttccagatcatcgagaatgtctttaagcagaagaaaaaaccgaccttgaaacaaattgccaaggag | |
| | atactggtaaatgaagaagacattaagggttatcgagtaacgtcaactgggaaaccagagttcacaaacttgaaagtct | |
| | accacgatataaaagatataacggctcgaaaggagatcatcgaaaatgcagagttgttggatcagatagcgaagatatt | |
| | gaccatctatcaaagcagcgaggatatacaggaagagctgaccaatttgaattccgagctcacacaggaggaaatagag | |
| | cagatcagtaacttgaaaggttacacggggactcacaacctgagtttgaaggcgatcaacttgattctggatgagttgt | |
| | ggcataccaatgacaatcaaatcgccatatttaacaggcttaagcttgtccccaaaaaagtggacctctcacagcagaa | |
| | agaaattccaaccaccctcgtggacgacttcatattgagtcccgtggtgaagcgatcatttatccaatcaatcaaagta | |
| | atcaatgccatcattaaaaaatacggcttgccaaatgacataatcattgagttggcacgcgaaaaaaatagtaaggatg | |
| | ctcaaaagatgataaacgagatgcagaaacggaatcgccaaactaatgaacgcattgaggaaataattcgcactaccgg | |
| | gaaggaaaacgcaaagtatttgattgagaaaatcaaactgcacgacatgcaagagggcaaatgtctttacagcctggag | |
| | gcgatccctctcgaagacttgcttaacaacccgtttaactatgaggtcgatcatattatacctcgatctgtgagttttg | |
| | ataacagtttcaataacaaagttcttgtcaagcaagaggaggcctccaagaaaggtaatcgcaccccgttccagtattt | |
| | gtctagtagcgattccaaaatatcatatgaaacttttaagaagcatatcttgaaccttgctaaggggaaggggcgcatc | |
| | tccaagacaaagaaagagtatcttctggaagaacgcgatatcaacaggttttcagttcaaaaagacttcatcaacagaa | |
| | acctcgtggatacgcggtatgccacacgaggactcatgaatcttctcaggtcatactttcgggttaacaacctggacgt | |
| | aaaagttaaaagcatcaacggggggtttacgtccttcttgcgccgcaaatggaaattcaaaaaggagagaaacaagggc | |
| | tacaaacatcacgcggaagacgcactgattatcgcgaacgctgattttatttttaaagaatggaagaaacttgataagg | |
| | cgaaaaaagtaatggaaaatcagatgtttgaggagaagcaggcggagtctatgccagaaattgaaacggagcaagaata | |
| | caaagaaatctttataacgccacatcagataaagcatattaaggactttaaagattacaaatactcacaccgggtcgac | |
| | aaaaaacccaatagggagctgataaacgacactctgtactccacacggaaagacgacaaaggcaacacactcattgtta | |
| | acaacttgaatggcttgtacgataaagacaatgacaagctgaaaaaacttataaataaatcaccggagaaactgctgat | |
| | gtatcatcatgacccgcaaacgtatcaaaagctcaagctgataatggaacagtatggagatgagaaaaatccattgtac | |
| | aaatattacgaagaaactggtaactatcttacaaaatactcaaaaaaggataatgggccagttataaagaagataaagt | |
| | attacggcaacaagcttaatgctcatctggacatcacagatgactatcccaactcccggaataaagtcgtaaagctgag | |
| | ccttaaaccgtacagattcgatgtttatctggacaatggagtctataaattcgtaactgtgaaaaacttggatgtgata | |
| | aagaaagagaactactatgaggtaaattctaaatgttacgaagaagcgaaaaaattgaaaaaaatctcaaatcaagccg | |
| | agtttatcgcaagcttctacaacaatgatctcattaaaattaacggagaactctaccgggttataggagtaaataatga | |
| | cctcctgaaccgaatagaggtgaatatgatagatattacttatagagagtatttggagaacatgaacgataagaggcct | |
| | cctcggattattaagaccatcgcctcaaagactcagtcaatcaaaaaatattcaacggacattcttgggaatttgtacg | |
| | aggttaaaagtaaaaaacatcctcaaataatcaaaaaagggaagaggccggcggctacgaaaaaggctggacaggcgaa | |
| | gaagaaaaagggttcagaagcgagtgggagcggtcgcgcggacgctctcgatgatttcgacctggacatgcttgggtcc | |
| | gatgctctcgacgattttgatctggacatgctcggttctgacgcgcttgacgacttcgacctcgacatgttggggtcag | |
| | acgcgctcgacgatttcgacctcgatatgctg | |
| NLS-SadCas9- | cccaagaagaagagaaaagtcggcatccacggcgtgcccgccgccaagagaaactacatcctgggcctggccatcggca | 117 |
| NLS-VP64 | tcacaagcgtgggctacggcatcatcgactacgagacaagagacgtgatcgacgccggcgtgagactgttcaaggaggc | |
| (optimized | caacgtggagaacaacgagggcagaagaagcaagagaggcgctagaagactgaagaggaggagaagacatagaattcag | |
| nucleotide | agagtgaagaagctgctgttcgactacaacctgctgaccgaccacagcgagctcagcggcatcaacccctacgaggcta | |
| sequence 2) | gagtgaagggcctgtctcagaagctgagcgaggaggagttcagcgccgccctgctgcacctggccaagagaagaggcgt | |
| | gcacaacgtgaacgaggtggaggaggacaccggcaacgagctgagcaccaaggagcagatctcgagaaatagcaaggcc | |
| | ctggaggagaagtacgtggccgagctgcagctggagagactgaagaaggacggcgaggtgagaggcagcattaacagat | |
| | tcaagacaagcgactacgtgaaggaggccaagcagctgctgaaggtgcagaaggcctaccatcagctggatcagagctt | |
| | catcgacacctacatcgacctgctggagacaagaagaacctactacgagggccccggcgagggcagccccttcggctgg | |
| | aaggatatcaaggagtggtacgagatgctgatgggccactgcacctacttccccgaggagctgagaagcgtgaagtacg | |
| | cctacaacgccgacctgtacaacgccctgaacgacctgaacaatctggtgatcacaagagacgagaacgagaagctgga | |
| | gtactacgagaagttccaaatcatcgagaacgtgtttaagcagaagaagaagcccaccctgaagcagatcgcgaaggag | |
| | atactggtgaacgaggaggacatcaaaggctatagagtgacaagcaccggcaagcccgagttcaccaacctgaaggtgt | |
| | accacgacatcaaggacatcaccgctagaaaggaaattatcgagaatgcggaactgctggatcagatcgcgaaaatact | |
| | gaccatctatcagagcagcgaggacatccaagaggagctgaccaacctgaactcagagctgacccaagaggagatcgag | |
| | caaatttctaacctgaagggctacaccggcacccacaacctgagcctgaaggccattaacctgatcctggacgagctgt | |
| | ggcacaccaacgacaatcagatagccatttttaacagactgaagctggtgcccaagaaggtggacctgtctcagcagaa | |
| | ggagatccccaccaccctggtggacgacttcatcctgagccccgtggtgaagagaagcttcatccaaagcatcaaggtc | |
| | attaacgccattattaaaaagtacggcctgcccaacgacatcatcatcgagctggctagagagaagaacagcaaggacg | |
| | ctcagaagatgatcaacgagatgcagaagagaaacagacagaccaacgagagaatcgaggagatcatcagaaccaccgg | |
| | caaggagaacgccaagtacctgatcgagaagatcaagctgcacgacatgcaagagggcaagtgcctgtacagcctggag | |
| | gccatccccctggaggacttgctcaacaaccccttcaactacgaggtggaccacatcatccctagaagcgtgagcttcg | |
| | acaacagcttcaacaacaaggtgctggtgaagcaagaggaggctagcaaaaagggtaacagaaccccctttcagtacct | |
| | gagcagcagcgacagcaagatcagctacgagaccttcaagaagcacatcctgaacctggccaagggcaagggcagaatc | |
| | agcaagaccaagaaggagtacctgctggaggagagagacattaaccggttcagtgtgcagaaggacttcatcaacagaa | |
| | acctggtggacacaagatacgccacaagaggcctgatgaacctgctgagaagctacttcagagtgaacaacctggacgt | |
| | gaaggtaaaatctattaatggcggcttcacaagcttcctgagaagaaagtggaagttcaagaaggaacgcaacaagggc | |
| | tacaagcaccacgccgaggacgccctgatcatcgccaacgccgacttcatcttcaaggagtggaagaagctggacaagg | |
| | ccaagaaggtgatggagaatcagatgttcgaggagaagcaagccgagagcatgcccgagatcgagaccgagcaagagta | |
| | caaggagatcttcatcaccccccatcagatcaagcacatcaaggacttcaaggactacaagtacagccacagagtggac | |
| | aagaagcccaacagagagctgatcaacgacaccctgtacagcacaagaaaggacgacaagggcaacaccctgatcgtta | |
| | acaacctgaatggactgtacgacaaggacaacgacaagctgaaaaaactgatcaacaagagccccgagaagctgctgat | |
| | gtaccaccacgaccctcagacctatcagaagctgaagctgatcatggagcagtacggcgacgagaagaaccccctgtac | |
| | aagtactacgaggagaccggcaactacctgaccaagtacagcaagaaggacaacggccccgtgattaagaagatcaaat | |
| | actacggcaacaagctgaacgcccacctggacatcaccgacgactaccccaacagcagaaacaaggtggtgaagctgtc | |
| | cctgaagccttacagattcgacgtgtacctggacaacggcgtgtacaagttcgtgaccgtgaagaacctggatgtaata | |
| | aaaaaggagaactattacgaggtgaacagcaagtgctacgaggaggccaagaagttaaagaaaatcagcaaccaagccg | |
| | agttcatcgctagcttctacaacaacgatctgatcaagatcaacggcgagctgtacagagtgatcggcgtgaataatga | |
| | cctgctgaatcggatcgaggtgaacatgatcgacatcacctacagagagtacctggagaacatgaacgacaagagaccc | |
| | cctagaatcatcaagaccatcgctagcaagacccaaagtatcaaaaaatacagcaccgacatcctgggcaacctgtacg | |
| | aggtgaagagcaagaaacaccctcagatcatcaagaagggaaaaagacccgccgccaccaagaaggccggccaagccaa | |
| | gaagaaaaagggcagcgaggctagcggcagcggcagagccgatgccctggacgacttcgatctggacatgctgggaagc | |
| | gacgccctagatgacttcgaccttgacatgctggggtctgatgcgctagatgatttcgatcttgatatgctcgggagcg | |
| | acgctctagacgactttgatttggatatgctg | |
| NLS- | MAPKKKRKVGIHGVPAAKRNYILGLAIGITSVGYGIIDYETRDVIDAGVRLFKEANVENNEGRRSKRGARRLKRRRRHR | 118 |
| miniSadCas9v2- | IQRVKKLLFDYNLLTDHSELSGINPYEARVKGLSQKLSEEEFSAALLHLAKRRGVHNVNEVEEDTGNELSTKEQISRNS | |
| NLS-VP64 | KALEEKYVAELQLERLKKDGEVRGSINRFKTSDYVKEAKQLLKVQKAYHQLDQSFIDTYIDLLETRRTYYEGPGEGSPF | |
| (amino acid | GWKDIKEWYEMLKRRRRHRILSPVVKRSFIQSIKVINAIIKKYGLPNDIIIELAREKNSKDAQKMINEMQKRNRQTNER | |
| sequence) | IEEIIRTTGKENAKYLIEKIKLHDMQEGKCLYSLEAIPLEDLLNNPFNYEVDHIIPRSVSFDNSFNNKVLVKQEEASKK | |
| | GNRTPFQYLSSSDSKISYETFKKHILNLAKGKGRISKTKKEYLLEERDINRFSVQKDFINRNLVDTRYATRGLMNLLRS | |
| | YFRVNNLDVKVKSINGGFTSFLRRKWKFKKERNKGYKHHAEDALIIANADFIFKEWKKLDKAKKVMENQMFEEKQAESM | |
| | PEIETEQEYKEIFITPHQIKHIKDFKDYKYSHRVDKKPNRKLINDTLYSTRKDDKGNTLIVNNLNGLYDKDNDKLKKLI | |
| | NKSPEKLLMYHHDPQTYQKLKLIMEQYGDEKNPLYKYYEETGNYLTKYSKKDNGPVIKKIKYYGNKLNAHLDITDDYPN | |
| | SRNKVVKLSLKPYRFDVYLDNGVYKFVTVKNLDVIKKENYYEVNSKCYEEAKKLKKISNQAEFIASFYKNDLIKINGEL | |
| | YRVIGVNNDLLNRIEVNMIDITYREYLENMNDKRPPHIIKTIASKTQSIKKYSTDILGNLYEVKSKKHPQIIKKGKRPA | |
| | ATKKAGQAKKKKGSEASGSGRADALDDFDLDMLGSDALDDFDLDMLGSDALDDFDLDMLGSDALDDFDLDML | |
| NLS- | ccaaagaagaagcggaaggtcggtatccacggagtcccagcagccaagaggaactacatcctgggcctggccatcggca | 119 |
| miniSadCas9v2- | tcaccagcgtgggctacggcatcatcgactacgagaccagggacgtgatcgacgccggcgtgaggctgttcaaggaggc | |
| NLS-VP64 | caacgtggagaacaacgagggcaggaggagcaagaggggcgccaggaggctgaagaggaggaggaggcacaggatccag | |
| (nucleotide | agggtgaagaagctgctgttcgactacaacctgctgaccgaccacagcgagctgagcggcatcaacccctacgaggcca | |
| sequence) | gggtgaagggcctgagccagaagctgagcgaggaggagttcagcgccgccctgctgcacctggccaagaggaggggcgt | |
| | gcacaacgtgaacgaggtggaggaggacaccggcaacgagctgagcaccaaggagcagatcagcaggaacagcaaggcc | |
| | ctggaggagaagtacgtggccgagctgcagctggagaggctgaagaaggacggcgaggtgaggggcagcatcaacaggt | |
| | tcaagaccagcgactacgtgaaggaggccaagcagctgctgaaggtgcagaaggcctaccaccagctggaccagagctt | |
| | catcgacacctacatcgacctgctggagaccaggaggacctactacgagggccccggcgagggcagccccttcggctgg | |
| | aaggacatcaaggagtggtacgagatgctgaagaggaggaggaggcacaggatcctgagccccgtggtgaagaggagct | |
| | tcatccagagcatcaaggtgatcaacgccatcatcaagaagtacggcctgcccaacgacatcatcatcgagctggccag | |
| | ggagaagaacagcaaggacgcccagaagatgatcaacgagatgcagaagaggaacaggcagaccaacgagaggatcgag | |
| | gagatcatcaggaccaccggcaaggagaacgccaagtacctgatcgagaagatcaagctgcacgacatgcaggagggca | |
| | agtgcctgtacagcctggaggccatccccctggaggacctgctgaacaaccccttcaactacgaggtggaccacatcat | |
| | ccccaggagcgtgagcttcgacaacagcttcaacaacaaggtgctggtgaagcaggaggaggccagcaagaagggcaac | |
| | aggacccccttccagtacctgagcagcagcgacagcaagatcagctacgagaccttcaagaagcacatcctgaacctgg | |
| | ccaagggcaagggcaggatcagcaagaccaagaaggagtacctgctggaggagagggacatcaacaggttcagcgtgca | |
| | gaaggacttcatcaacaggaacctggtggacaccaggtacgccaccaggggcctgatgaacctgctgaggagctacttc | |
| | agggtgaacaacctggacgtgaaggtgaagagcatcaacggcggcttcaccagcttcctgaggaggaagtggaagttca | |
| | agaaggagaggaacaagggctacaagcaccacgccgaggacgccctgatcatcgccaacgccgacttcatcttcaagga | |
| | gtggaagaagctggacaaggccaagaaggtgatggagaaccagatgttcgaggagaagcaggccgagagcatgcccgag | |
| | atcgagaccgagcaggagtacaaggagatcttcatcaccccccaccagatcaagcacatcaaggacttcaaggactaca | |
| | agtacagccacagggtggacaagaagcccaacaggaagctgatcaacgacaccctgtacagcaccaggaaggacgacaa | |
| | gggcaacaccctgatcgtgaacaacctgaacggcctgtacgacaaggacaacgacaagctgaagaagctgatcaacaag | |
| | agccccgagaagctgctgatgtaccaccacgacccccagacctaccagaagctgaagctgatcatggagcagtacggcg | |
| | acgagaagaaccccctgtacaagtactacgaggagaccggcaactacctgaccaagtacagcaagaaggacaacggccc | |
| | cgtgatcaagaagatcaagtactacggcaacaagctgaacgcccacctggacatcaccgacgactaccccaacagcagg | |
| | aacaaggtggtgaagctgagcctgaagccctacaggttcgacgtgtacctggacaacggcgtgtacaagttcgtgaccg | |
| | tgaagaacctggacgtgatcaagaaggagaactactacgaggtgaacagcaagtgctacgaggaggccaagaagctgaa | |
| | gaagatcagcaaccaggccgagttcatcgccagcttctacaagaacgacctgatcaagatcaacggcgagctgtacagg | |
| | gtgatcggcgtgaacaacgacctgctgaacaggatcgaggtgaacatgatcgacatcacctacagggagtacctggaga | |
| | acatgaacgacaagaggcccccccacatcatcaagaccatcgccagcaagacccagagcatcaagaagtacagcaccga | |
| | catcctgggcaacctgtacgaggtgaagagcaagaagcacccccagatcatcaagaagggcaaaaggccggcggccacg | |
| | aaaaaggccggccaggcaaaaaagaaaaagggatccgaggccagcggttccggacgggctgacgcattggacgattttg | |
| | atctggatatgctgggaagtgacgccctcgatgattttgaccttgacatgcttggttcggatgcccttgatgactttga | |
| | cctcgacatgctcggcagtgacgcccttgatgatttcgacctggacatgctg | |
| NLS- | ccaaagaagaagagaaaggttggcattcacggggtgccagccgccaaaaggaattatatcctcggattggcaataggca | 120 |
| miniSadCas9v2- | taaccagtgtaggttatggaatcatagactatgaaacccgggacgttatagacgcgggagtacggttgttcaaagaggc | |
| NLS-VP64 | aaatgttgaaaacaatgagggccggcggtccaaacggggtgcacggaggcttaaacgccgcaggagacatcggattcaa | |
| (optimized | agggtcaagaaacttctttttgattataacctcctgaccgaccacagtgagctctccgggataaatccttacgaagcca | |
| nucleotide | gagtcaagggacttagccaaaagctgtcagaggaagagttttcagcggcactgcttcatcttgcaaaaaggcggggcgt | |
| sequence 1) | ccataatgttaatgaggtagaagaagatactgggaacgagctcagtacgaaggaacaaatctcaagaaatagcaaggct | |
| | cttgaggaaaaatacgtagcagaacttcagttggaaagacttaagaaagacggagaggttcggggaagtattaaccgct | |
| | ttaagacttcagattatgttaaagaggcaaagcaattgttgaaagttcagaaagcttatcatcaattggatcaatcatt | |
| | tatagatacatacatagatctccttgaaaccagacggacgtactacgaaggtccaggagaagggtcacccttcggatgg | |
| | aaggacatcaaggaatggtacgaaatgcttaaaaggaggcgaagacatagaattctcagtcctgtcgtgaagcgaagct | |
| | tcatacagagcataaaggtaataaacgctataatcaagaagtacggattgcctaacgatattataatagaattggcacg | |
| | agagaaaaacagtaaggatgcacagaagatgattaacgagatgcagaagcgaaatcgccaaacgaatgagcgcatagaa | |
| | gagattataagaactacaggcaaggagaacgccaagtatcttatagaaaagataaaactccatgacatgcaggaaggta | |
| | aatgtctttattctctcgaggctatcccgttggaggatctgctgaataaccctttcaattacgaagtagaccatatcat | |
| | acctcgatccgtttcattcgataattcctttaacaataaagtgctcgttaaacaggaagaggcatccaagaaagggaat | |
| | agaactccgtttcaatacctgtctagttctgactctaaaataagttatgaaacctttaaaaagcatatactcaaccttg | |
| | cgaagggcaaggggcggatatcaaagaccaaaaaggaatacctcctcgaggagcgggacattaatagattttctgtgca | |
| | gaaagattttataaaccgcaacttggtagacacgcgatatgctacacgaggtctgatgaacttgctccgatcttatttt | |
| | cgcgtcaataatctggatgtcaaggtgaagtctattaacggtggtttcacatccttcctgcgacgaaagtggaagttta | |
| | agaaagagcggaataagggctacaagcatcatgccgaggacgctctgattatcgctaacgcggattttatctttaaaga | |
| | gtggaagaaactcgacaaagctaagaaagtgatggagaaccagatgtttgaggaaaagcaggccgaatcaatgccggaa | |
| | atcgagacggagcaggaatataaggagattttcatcactcctcatcagataaagcatattaaagacttcaaagactaca | |
| | agtatagccacagggtggacaagaagcccaacaggaaactgattaacgatacactctattccacccggaaagatgataa | |
| | aggaaatacgctgatcgtaaataacctcaacggactttacgataaggataatgataaactcaaaaagcttatcaataag | |
| | tctcccgagaagctgctcatgtaccaccatgatccccagacataccaaaagctgaagctcatcatggaacagtatggag | |
| | atgagaagaacccgttgtacaaatactatgaagagacgggcaactaccttactaagtacagcaagaaggataacgggcc | |
| | ggtgatcaaaaagattaagtattacgggaataaacttaatgcgcatcttgacatcactgacgactaccccaattctcgg | |
| | aataaggttgtaaaactgtcccttaagccatatcggttcgacgtctatctcgacaacggtgtttacaagttcgtaacag | |
| | tcaagaatcttgatgttataaaaaaagagaattattacgaagttaactcaaagtgctatgaggaagcgaaaaaacttaa | |
| | aaaaatatccaaccaggccgagtttatagcttccttttataaaaacgatctgataaaaatcaacggcgaattgtacaga | |
| | gtaatcggtgttaataatgatttgctgaataggatagaagttaatatgatagatattacttacagggaatatttggaaa | |
| | acatgaatgacaagcggcccccgcacatcatcaagacaatcgcatccaaaactcagtccatcaagaagtattctacgga | |
| | cattcttggaaatctttatgaggtaaagagcaaaaaacatccgcaaatcattaaaaagggaaagagacccgcagccact | |
| | aagaaggccgggcaggcgaagaagaaaaagggttctgaggctagcgggtctggccgagcggatgctttggatgattttg | |
| | acttggacatgcttggttctgacgctcttgatgattttgatctggatatgttgggaagcgacgcgctggatgattttga | |
| | tctggatatgttgggctcagacgcacttgatgacttcgaccttgatatgctg | |
| NLS- | ccaaagaaaaagagaaaagtgggcatccacggcgtgcccgccgccaagagaaactacatcctgggcctggccatcggca | 121 |
| miniSadCas9v2- | tcacaagcgtgggctacggcatcatcgactacgagacaagagacgtgatcgacgccggcgtgagactgttcaaggaggc | |
| NLS-VP64 | caacgtggagaacaacgagggcagaagaagcaagagaggcgctagaagattgaaacggagaagaagacaccggattcag | |
| (optimized | agagtgaagaagctgctgttcgactacaacctgctgaccgaccacagcgagctgagcggcatcaacccctacgaggcta | |
| nucleotide | gagtgaagggcctgtctcagaagctgagcgaggaggagttcagcgccgccctgctgcacctggccaagagaagaggcgt | |
| sequence 2) | gcacaacgtgaacgaggtggaggaggacaccggcaacgagctgagcaccaaggagcagatcagcagaaacagcaaggcc | |
| | ctggaggagaagtacgtggccgagctgcagctggagagactgaagaaggacggcgaggtgagaggcagcattaaccgct | |
| | tcaagacaagcgactacgtgaaggaggccaagcagctgctgaaggtgcagaaggcctaccatcagctggatcagagctt | |
| | catcgacacctacatcgacctgctggagacaagaagaacctactacgagggccccggcgagggcagccccttcggctgg | |
| | aaggacatcaaggagtggtacgagatgcttaaacgacgtcgtagacacagaatcttaagccccgtggtgaagagaagct | |
| | tcatccaaagcatcaaggtgatcaacgccatcattaaaaagtacggcctacccaacgacatcatcatcgagctggctag | |
| | agagaagaacagcaaggacgctcagaagatgatcaacgagatgcagaagagaaacagacagaccaacgagagaatcgag | |
| | gagatcatcagaaccaccggcaaggagaacgccaagtacctgatcgagaagatcaagctgcacgacatgcaagagggca | |
| | agtgcctgtacagcctggaggccatccccctggaggacctgctgaacaacccattcaactacgaggtggaccacatcat | |
| | ccctagaagcgtgagcttcgacaacagcttcaacaacaaggtgctggtgaagcaagaggaggctagcaagaagggcaac | |
| | agaaccccctttcagtacctgagcagcagcgacagcaagatcagctacgagaccttcaagaagcacatcctgaacctgg | |
| | ccaagggcaagggcagaatcagcaagaccaagaaggagtacctgctggaggagagagacatcaatagatttagcgtgca | |
| | gaaggacttcatcaacagaaacctggtggacacaagatacgccacaagaggcctgatgaacctgctgagaagctacttc | |
| | agagtgaacaacctcgatgtcaaggtaaaatccatcaatggcggcttcacaagcttcctgagaagaaagtggaagttca | |
| | aaaaggagcgtaacaagggctacaagcaccacgccgaggacgccctgatcatcgccaacgccgacttcatcttcaagga | |
| | gtggaagaagctggacaaggccaagaaggtgatggagaatcagatgttcgaggagaagcaagccgagagcatgcccgag | |
| | atcgagaccgagcaagagtacaaggagatcttcatcaccccccatcagatcaagcacatcaaggacttcaaggactaca | |
| | agtacagccacagagtggacaagaagcccaacagaaagctgataaacgatactctgtacagcacaagaaaggacgacaa | |
| | gggcaacaccctgatcgtgaacaatctgaacggcctgtacgacaaggacaacgacaaactgaagaaattaataaacaag | |
| | agccccgagaagctgctgatgtaccaccacgaccctcagacctatcagaagctgaagctgatcatggagcagtacggcg | |
| | acgagaagaaccccctgtacaagtactacgaggagaccggcaactacctgactaagtacagcaagaaggacaacggccc | |
| | cgtaataaagaagatcaaatactacggcaacaagctgaacgcccacctggacatcaccgacgactaccccaacagcaga | |
| | aacaaggtggtgaagctgagcctgaagccctacagattcgacgtgtacctggacaacggcgtgtacaagttcgtgaccg | |
| | tgaagaacctggacgtgatcaagaaggaaaactattacgaggtgaacagcaagtgctacgaggaggccaagaagctgaa | |
| | gaagatctcaaaccaagccgagttcatcgctagcttctacaagaacgacctgatcaagatcaacggcgagctgtacaga | |
| | gtgatcggcgtgaacaacgatctgctgaacagaattgaggtgaacatgatcgacatcacctacagagagtacctggaga | |
| | acatgaacgacaagagacccccccacatcatcaagaccatcgctagcaagacacagagcatcaagaaatacagcacgga | |
| | catcctgggcaacctgtacgaggtgaaaagcaagaaacaccctcagatcataaaaaagggcaaaagacccgccgccacc | |
| | aagaaggccggccaagccaagaagaagaagggcagcgaggctagcggcagcggcagagccgatgccctggacgacttcg | |
| | atctcgacatgctgggcagcgacgccctagatgacttcgaccttgatatgctgggtagcgatgcgctagatgatttcga | |
| | tcttgatatgctcgggagcgacgctctagacgatttcgacctggacatgctg | |
| NLS- | MAPKKKRKVGIHGVPAAKRNYILGLAIGITSVGYGIIDYETRDVIDAGVRLFKEANVENNEGRRSKRGARRLKRRRRHR | 122 |
| miniSadCas9v4- | IQRVKKLLFDYNLLTDHSELSGINPYEARVKGLSQKLSEEEFSAALLHLAKRRGVHNVNEVEEDTGNELSTKEQISRNS | |
| NLS-VP64 | KALEEKYVAELQLERLKKDGEVRGSINRFKTSDYVKEAKQLLKVQKAYHQLDQSFIDTYIDLLETRRTYYEGPGEGSPF | |
| (amino acid | GWKDIKEWYEMLMGHCTYFPEELRSVKYAYNADLYNALNDLNNLVITRDENEKLEYYEKFQIIENVFKQKKKPTLKQIA | |
| sequence) | KEILVNEEDIKGYRVTSTGKPEFTNLKVYHDIKDITARKEIIENAELLDQIAKILTIYQSSEDIQEELTNLNSELTQEE | |
| | IEQISNLKGYTGTHNLSLKAINLILDELWHTNDNQIAIFNRLKLVPKKVDLSQQKEIPTTLVDDFILSPVVKRSFIQSI | |
| | KVINAIIKKYGLPNDIIIELGSKRYATRGLMNLLRSYFRVNNLDVKVKSINGGFTSFLRRKWKFKKERNKGYKHHAEDA | |
| | LIIANADFIFKEWKKLDKAKKVMENQMFEEKQAESMPEIETEQEYKEIFITPHQIKHIKDFKDYKYSHRVDKKPNRELI | |
| | NDTLYSTRKDDKGNTLIVNNLNGLYDKDNDKLKKLINKSPEKLLMYHHDPQTYQKLKLIMEQYGDEKNPLYKYYEETGN | |
| | YLTKYSKKDNGPVIKKIKYYGNKLNAHLDITDDYPNSRNKVVKLSLKPYRFDVYLDNGVYKFVTVKNLDVIKKENYYEV | |
| | NSKCYEEAKKLKKISNQAEFIASFYNNDLIKINGELYRVIGVNNDLLNRIEVNMIDITYREYLENMNDKRPPRIIKTIA | |
| | SKTQSIKKYSTDILGNLYEVKSKKHPQIIKKGKRPAATKKAGQAKKKKGSEASGSGRADALDDFDLDMLGSDALDDFDL | |
| | DMLGSDALDDFDLDMLGSDALDDFDLDML* | |
| NLS- | ccaaagaagaagcggaaggtcggtatccacggagtcccagcagccaagcggaactacatcctgggcctggccatcggca | 123 |
| miniSadCas9v4- | tcaccagcgtgggctacggcatcatcgactacgagacacgggacgtgatcgatgccggcgtgcggctgttcaaagaggc | |
| NLS-VP64 | caacgtggaaaacaacgagggcaggcggagcaagagaggcgccagaaggctgaagcggcggaggcggcatagaatccag | |
| (nucleotide | agagtgaagaagctgctgttcgactacaacctgctgaccgaccacagcgagctgagcggcatcaacccctacgaggcca | |
| sequence) | gagtgaagggcctgagccagaagctgagcgaggaagagttctctgccgccctgctgcacctggccaagagaagaggcgt | |
| | gcacaacgtgaacgaggtggaagaggacaccggcaacgagctgtccaccaaagagcagatcagccggaacagcaaggcc | |
| | ctggaagagaaatacgtggccgaactgcagctggaacggctgaagaaagacggcgaagtgcggggcagcatcaacagat | |
| | tcaagaccagcgactacgtgaaagaagccaaacagctgctgaaggtgcagaaggcctaccaccagctggaccagagctt | |
| | catcgacacctacatcgacctgctggaaacccggcggacctactatgagggacctggcgagggcagccccttcggctgg | |
| | aaggacatcaaagaatggtacgagatgctgatgggccactgcacctacttccccgaggaactgcggagcgtgaagtacg | |
| | cctacaacgccgacctgtacaacgccctgaacgacctgaacaatctcgtgatcaccagggacgagaacgagaagctgga | |
| | atattacgagaagttccagatcatcgagaacgtgttcaagcagaagaagaagcccaccctgaagcagatcgccaaagaa | |
| | atcctcgtgaacgaagaggatattaagggctacagagtgaccagcaccggcaagcccgagttcaccaacctgaaggtgt | |
| | accacgacatcaaggacattaccgcccggaaagagattattgagaacgccgagctgctggatcagattgccaagatcct | |
| | gaccatctaccagagcagcgaggacatccaggaagaactgaccaatctgaactccgagctgacccaggaagagatcgag | |
| | cagatctctaatctgaagggctataccggcacccacaacctgagcctgaaggccatcaacctgatcctggacgagctgt | |
| | ggcacaccaacgacaaccagatcgctatcttcaaccggctgaagctggtgcccaagaaggtggacctgtcccagcagaa | |
| | agagatccccaccaccctggtggacgacttcatcctgagccccgtcgtgaagagaagcttcatccagagcatcaaagtg | |
| | atcaacgccatcatcaagaagtacggcctgcccaacgacatcattatcgagctgggcagcaagagatacgccaccagag | |
| | gcctgatgaacctgctgcggagctacttcagagtgaacaacctggacgtgaaagtgaagtccatcaatggcggcttcac | |
| | cagctttctgcggcggaagtggaagtttaagaaagagcggaacaaggggtacaagcaccacgccgaggacgccctgatc | |
| | attgccaacgccgatttcatcttcaaagagtggaagaaactggacaaggccaaaaaagtgatggaaaaccagatgttcg | |
| | aggaaaagcaggccgagagcatgcccgagatcgaaaccgagcaggagtacaaagagatcttcatcaccccccaccagat | |
| | caagcacattaaggacttcaaggactacaagtacagccaccgggtggacaagaagcctaatagagagctgattaacgac | |
| | accctgtactccacccggaaggacgacaagggcaacaccctgatcgtgaacaatctgaacggcctgtacgacaaggaca | |
| | atgacaagctgaaaaagctgatcaacaagagccccgaaaagctgctgatgtaccaccacgacccccagacctaccagaa | |
| | actgaagctgattatggaacagtacggcgacgagaagaatcccctgtacaagtactacgaggaaaccgggaactacctg | |
| | accaagtactccaaaaaggacaacggccccgtgatcaagaagattaagtattacggcaacaaactgaacgcccatctgg | |
| | acatcaccgacgactaccccaacagcagaaacaaggtcgtgaagctgtccctgaagccctacagattcgacgtgtacct | |
| | ggacaatggcgtgtacaagttcgtgaccgtgaagaatctggatgtgatcaaaaaagaaaactactacgaagtgaatagc | |
| | aagtgctatgaggaagctaagaagctgaagaagatcagcaaccaggccgagtttatcgcctccttctacaacaacgatc | |
| | tgatcaagatcaacggcgagctgtatagagtgatcggcgtgaacaacgacctgctgaaccggatcgaagtgaacatgat | |
| | cgacatcacctaccgcgagtacctggaaaacatgaacgacaagaggccccccaggatcattaagacaatcgcctccaag | |
| | acccagagcattaagaagtacagcacagacattctgggcaacctgtatgaagtgaaatctaagaagcaccctcagatca | |
| | tcaaaaagggcaaaaggccggcggccacgaaaaaggccggccaggcaaaaaagaaaaagggatccgaggccagcggttc | |
| | cggacgggctgacgcattggacgattttgatctggatatgctgggaagtgacgccctcgatgattttgaccttgacatg | |
| | cttggttcggatgcccttgatgactttgacctcgacatgctcggcagtgacgcccttgatgatttcgacctggacatgc | |
| | tg | |
| NLS- | ccaaagaaaaaaaggaaagtaggtattcacggggtacctgccgctaaacgaaactatattctcggactggcaataggga | 124 |
| miniSadCas9v4- | tcacaagtgttggatacggcattatagattatgaaactcgagacgtgattgatgcaggtgtacggttgtttaaagaggc | |
| NLS-VP64 | caacgttgaaaacaacgaggggcgccggagtaaacgcggagcgcgaaggctcaagaggcggagaagacaccgcattcag | |
| (optimized | agagtaaaaaaactgcttttcgactataatctcttgacagatcacagcgaactgtcaggtatcaacccctacgaggcga | |
| nucleotide | gggttaagggactcagtcaaaagctttcagaagaagaattttccgctgcccttcttcatttggcaaagcgaagaggtgt | |
| sequence 1) | gcataatgtgaatgaagttgaggaagatacgggcaacgagctgtccactaaggagcagatctcacggaatagtaaggcc | |
| | ctcgaggaaaagtacgtcgctgaactgcagcttgaacggctgaaaaaggatggggaggtccgcgggtctatcaaccgct | |
| | tcaaaacaagtgactacgttaaggaggccaagcaattgcttaaggttcaaaaagcgtaccatcaattggatcaaagctt | |
| | tattgatacgtatattgacctcctggaaacaaggcgaacttactacgaggggcccggcgaaggctccccctttggatgg | |
| | aaagacatcaaggaatggtacgaaatgttgatgggacattgcacctatttccccgaagagctcagatctgtaaaatacg | |
| | catacaatgctgatctgtacaacgctctcaacgatttgaacaatcttgtcattacgagggacgaaaatgaaaagcttga | |
| | gtactatgaaaaattccagattattgagaacgttttcaagcagaagaagaaaccaacgttgaaacaaattgcaaaagaa | |
| | atacttgtaaatgaagaggatataaaagggtatcgagttacaagcacggggaaaccagagtttaccaacttgaaagtat | |
| | accacgatataaaggatattacagcacggaaggagatcattgagaacgccgaactccttgaccaaatcgcgaagattct | |
| | gactatttatcaatccagtgaggacatccaggaggagttgacaaaccttaattccgagcttacccaagaggaaattgag | |
| | caaatatcaaaccttaaaggctataccggtacgcataacttgagtctcaaggccataaatcttattcttgatgaactct | |
| | ggcataccaatgacaatcagatcgcgatctttaaccggctcaagcttgtccctaaaaaagtggacctttcacaacagaa | |
| | agaaattcctaccacgctcgtcgatgactttattttgagcccagtggttaaacgaagttttattcaaagtatcaaggta | |
| | attaacgctataatcaaaaagtatggattgccaaacgatataatcattgaactgggttcaaaacggtacgcgacccgag | |
| | gtttgatgaacctcctgcggagctatttccgggttaacaatttggacgtgaaggtgaagagcatcaatggcggttttac | |
| | aagtttcctgagacgcaaatggaaattcaagaaggagcggaataaaggttataagcaccatgctgaagacgccttgata | |
| | attgcgaatgccgacttcatttttaaagaatggaagaaacttgacaaagctaagaaggtgatggaaaatcagatgtttg | |
| | aagagaagcaagcggaatctatgcccgaaatcgagaccgaacaagaatataaagagatattcattacacctcatcagat | |
| | caagcacataaaggatttcaaggattacaaatatagccatcgggtggataaaaaacccaatcgcgaattgataaatgac | |
| | acgttgtactcaactaggaaagatgacaagggaaacactcttatagtaaacaacctgaatggactctacgataaagata | |
| | atgacaagcttaaaaaactcattaataaatctccagagaagttgcttatgtatcaccacgacccccagacgtaccagaa | |
| | actgaaattgattatggaacaatatggcgatgagaaaaacccgctgtacaagtactatgaggaaaccgggaactatctc | |
| | actaaatattccaaaaaagacaacggaccggtaatcaaaaaaatcaaatactacggtaacaaactcaacgcacatttgg | |
| | acatcacggacgactacccgaactccagaaacaaagtagttaagctgtctctgaagccgtatagatttgacgtttacct | |
| | ggacaacggcgtttataagttcgtcacagtcaagaatttggacgtcatcaagaaagaaaactactacgaagtgaattcc | |
| | aagtgttacgaggaagcaaaaaagctcaagaagatttctaatcaagctgaatttatcgccagtttctacaataatgatc | |
| | tgataaaaattaacggcgaactctacagagtgatcggggttaacaacgacctcctgaatagaatagaggtgaacatgat | |
| | agatattacttatagggagtacctcgaaaatatgaatgacaaacgacctccgagaataatcaagacgattgctagtaaa | |
| | acccagagcattaaaaagtactccacggatatactgggtaatctctacgaggttaagtcaaaaaaacaccctcaaataa | |
| | ttaagaagggtaaaagacccgctgccacgaagaaagcggggcaggcgaaaaaaaaaaaaggcagtgaagcctcaggtag | |
| | cggcagagctgacgcacttgatgatttcgatcttgacatgctgggatcagatgcgttggatgatttcgatcttgacatg | |
| | cttggttctgatgccctggatgactttgacttggacatgcttggctcagacgcccttgatgacttcgatctggatatgc | |
| | tg | |
| NLS- | cccaaaaaaaaaagaaaggtgggcatccacggcgtgcccgccgccaagagaaactacatcctgggcctggccatcggca | 125 |
| miniSadCas9v4- | tcacaagcgtgggctacggcatcatcgactacgagacaagagacgtgatcgacgccggcgtgagactgttcaaggaggc | |
| NLS-VP64 | caacgtggagaacaacgagggcagaagaagcaagagaggcgctagaagactgaagcggagacgtagacatcgtattcag | |
| (optimized | agagtgaagaagctgctgttcgactacaacctgctgaccgaccactcggagctgagcggaatcaacccctacgaggcta | |
| nucleotide | gagtgaagggcctgtctcagaagctgagcgaggaggagttcagcgccgccctgctgcacctggccaagagaagaggcgt | |
| sequence 2) | gcacaacgtgaacgaggtggaggaggacaccggcaacgagctgagcaccaaggaacagatcagcagaaacagcaaggcc | |
| | ctggaggagaagtacgtggccgagctgcagctggagagactgaagaaggacggcgaggtgagaggcagcatcaacagat | |
| | tcaagacaagcgactacgtgaaggaggccaagcagctgctgaaggtgcagaaggcctaccatcagctggatcagagctt | |
| | catcgacacctacatcgacctgctggagacaagaagaacctactacgagggccccggcgagggcagccccttcggctgg | |
| | aaggacatcaaggagtggtacgagatgctgatgggccactgcacctacttccccgaggagctgagaagcgtgaagtacg | |
| | cctacaacgccgacctgtacaacgccctgaacgacctgaacaacctcgtgatcacaagagacgagaacgagaagctgga | |
| | gtattatgagaagtttcagatcatcgagaacgtgtttaagcagaaaaagaaacccaccctgaagcagattgccaaggag | |
| | atcctggtgaacgaggaggacatcaaaggctacagagtgacaagcaccggcaagcccgagttcaccaacctgaaggtgt | |
| | accacgacatcaaggacatcaccgctagaaaggaaattatcgagaatgcggaactgctggatcagatagccaaaatcct | |
| | taccatctatcagagcagcgaggacatccaagaggagctgaccaacctgaactcagaactgacccaagaggagatcgaa | |
| | cagatttccaatctgaaaggctacaccggcacccacaacctgagccttaaagcaataaacctgatcctggacgagctgt | |
| | ggcacaccaacgacaatcagatcgcaatcttcaacagactgaagctggtgcccaagaaggtggacctgtctcagcagaa | |
| | ggagatccccaccaccctggtggacgacttcatcctgagccccgtggtgaagagaagcttcattcagagcatcaaagtg | |
| | ataaacgccattatcaaaaagtacggcctgcccaacgacatcatcatcgagctgggcagcaagagatacgccacaagag | |
| | gcctgatgaacctgctgagaagctacttcagagtgaataacctagacgttaaggtaaagagcatcaatggcggcttcac | |
| | aagcttcctgagaagaaagtggaagtttaagaaagagagaaataagggctacaagcaccacgccgaggacgccctgatc | |
| | atcgccaacgccgacttcatcttcaaggagtggaagaagctggacaaggccaagaaggtgatggagaatcagatgttcg | |
| | aggagaagcaagccgagagcatgcccgagatcgagaccgagcaagagtacaaggagatcttcatcaccccccatcagat | |
| | caagcacatcaaggacttcaaggactacaagtacagccacagagtggacaagaagcccaacagagagctgatcaacgac | |
| | accctgtacagcacaagaaaggacgacaagggcaacaccctgatcgtgaataacctgaatggcctgtacgacaaggaca | |
| | acgacaagctgaagaagttgatcaacaagagccccgagaagctgctgatgtaccaccacgaccctcagacctatcagaa | |
| | gctgaagctgatcatggagcagtacggcgacgagaagaaccccctgtacaagtactacgaggagaccggcaactacctg | |
| | accaagtacagcaagaaggacaacggccccgtgattaaaaagatcaagtactacggcaacaagctgaacgcccacctgg | |
| | acatcaccgacgactaccccaacagcagaaacaaggtggtgaagctgagcttaaagccttatagattcgacgtgtacct | |
| | ggacaacggcgtgtacaagttcgtgaccgtgaagaacttggacgtgataaaaaaagaaaactactacgaggtgaacagc | |
| | aagtgctacgaggaggccaagaagctgaaaaaaataagcaaccaagccgagttcatcgctagcttctacaacaatgacc | |
| | tgatcaagatcaacggcgagctgtacagagtgatcggcgtgaacaacgacttactgaacagaatcgaggtgaacatgat | |
| | cgacatcacctacagagagtacctggagaacatgaacgacaagagaccccctagaatcatcaagaccatcgctagcaag | |
| | acacagagcattaagaagtatagcaccgacatcctgggcaacctgtacgaggtgaaatcgaagaagcaccctcagatta | |
| | tcaagaagggtaagagacccgccgccaccaagaaggccggccaagccaagaaaaaaaagggcagcgaggctagcggcag | |
| | cggcagagccgatgccctggatgacttcgatttggacatgttgggttctgacgccctagatgacttcgacctagatatg | |
| | ctggggagcgatgcgctagatgatttcgatcttgatatgctcgggagcgacgctctagacgacttcgatttggatatgc | |
| | tg | |
| NLS- | MAPKKKRKVGIHGVPAAKRNYILGLAIGITSVGYGIIDYETRDVIDAGVRLFKEANVENNEGRRSKRGARRLKRRRRHR | 126 |
| miniSadCas9v5- | IQRVKKLLFDYNLLTDHSELSGINPYEARVKGLSQKLSEEEFSAALLHLAKRRGVHNVNEVEEDTGNELSTKEQISRNS | |
| NLS-VP64 | KALEEKYVAELQLERLKKDGEVRGSINRFKTSDYVKEAKQLLKVQKAYHQLDQSFIDTYIDLLETRRTYYEGPGEGSPF | |
| (amino acid | GWKDIKEWYEMLKRRRRHRILSPVVKRSFIQSIKVINAIIKKYGLPNDIIIELGSKRYATRGLMNLLRSYFRVNNLDVK | |
| sequence) | VKSINGGFTSFLRRKWKFKKERNKGYKHHAEDALIIANADFIFKEWKKLDKAKKVMENQMFEEKQAESMPEIETEQEYK | |
| | EIFITPHQIKHIKDFKDYKYSHRVDKKPNRKLINDTLYSTRKDDKGNTLIVNNLNGLYDKDNDKLKKLINKSPEKLLMY | |
| | HHDPQTYQKLKLIMEQYGDEKNPLYKYYEETGNYLTKYSKKDNGPVIKKIKYYGNKLNAHLDITDDYPNSRNKVVKLSL | |
| | KPYRFDVYLDNGVYKFVTVKNLDVIKKENYYEVNSKCYEEAKKLKKISNQAEFIASFYKNDLIKINGELYRVIGVNNDL | |
| | LNRIEVNMIDITYREYLENMNDKRPPHIIKTIASKTQSIKKYSTDILGNLYEVKSKKHPQIIKKGKRPAATKKAGQAKK | |
| | KKGSEASGSGRADALDDFDLDMLGSDALDDFDLDMLGSDALDDFDLDMLGSDALDDFDLDML | |
| NLS- | ccaaagaagaagcggaaggtcggtatccacggagtcccagcagccaagaggaactacatcctgggcctggccatcggca | 127 |
| miniSadCas9v5- | tcaccagcgtgggctacggcatcatcgactacgagaccagggacgtgatcgacgccggcgtgaggctgttcaaggaggc | |
| NLS-VP64 | caacgtggagaacaacgagggcaggaggagcaagaggggcgccaggaggctgaagaggaggaggaggcacaggatccag | |
| (nucleotide | agggtgaagaagctgctgttcgactacaacctgctgaccgaccacagcgagctgagcggcatcaacccctacgaggcca | |
| sequence) | gggtgaagggcctgagccagaagctgagcgaggaggagttcagcgccgccctgctgcacctggccaagaggaggggcgt | |
| | gcacaacgtgaacgaggtggaggaggacaccggcaacgagctgagcaccaaggagcagatcagcaggaacagcaaggcc | |
| | ctggaggagaagtacgtggccgagctgcagctggagaggctgaagaaggacggcgaggtgaggggcagcatcaacaggt | |
| | tcaagaccagcgactacgtgaaggaggccaagcagctgctgaaggtgcagaaggcctaccaccagctggaccagagctt | |
| | catcgacacctacatcgacctgctggagaccaggaggacctactacgagggccccggcgagggcagccccttcggctgg | |
| | aaggacatcaaggagtggtacgagatgctgaagaggaggaggaggcacaggatcctgagccccgtggtgaagaggagct | |
| | tcatccagagcatcaaggtgatcaacgccatcatcaagaagtacggcctgcccaacgacatcatcatcgagctgggcag | |
| | caagaggtacgccaccaggggcctgatgaacctgctgaggagctacttcagggtgaacaacctggacgtgaaggtgaag | |
| | agcatcaacggcggcttcaccagcttcctgaggaggaagtggaagttcaagaaggagaggaacaagggctacaagcacc | |
| | acgccgaggacgccctgatcatcgccaacgccgacttcatcttcaaggagtggaagaagctggacaaggccaagaaggt | |
| | gatggagaaccagatgttcgaggagaagcaggccgagagcatgcccgagatcgagaccgagcaggagtacaaggagatc | |
| | ttcatcaccccccaccagatcaagcacatcaaggacttcaaggactacaagtacagccacagggtggacaagaagccca | |
| | acaggaagctgatcaacgacaccctgtacagcaccaggaaggacgacaagggcaacaccctgatcgtgaacaacctgaa | |
| | cggcctgtacgacaaggacaacgacaagctgaagaagctgatcaacaagagccccgagaagctgctgatgtaccaccac | |
| | gacccccagacctaccagaagctgaagctgatcatggagcagtacggcgacgagaagaaccccctgtacaagtactacg | |
| | aggagaccggcaactacctgaccaagtacagcaagaaggacaacggccccgtgatcaagaagatcaagtactacggcaa | |
| | caagctgaacgcccacctggacatcaccgacgactaccccaacagcaggaacaaggtggtgaagctgagcctgaagccc | |
| | tacaggttcgacgtgtacctggacaacggcgtgtacaagttcgtgaccgtgaagaacctggacgtgatcaagaaggaga | |
| | actactacgaggtgaacagcaagtgctacgaggaggccaagaagctgaagaagatcagcaaccaggccgagttcatcgc | |
| | cagcttctacaagaacgacctgatcaagatcaacggcgagctgtacagggtgatcggcgtgaacaacgacctgctgaac | |
| | aggatcgaggtgaacatgatcgacatcacctacagggagtacctggagaacatgaacgacaagaggcccccccacatca | |
| | tcaagaccatcgccagcaagacccagagcatcaagaagtacagcaccgacatcctgggcaacctgtacgaggtgaagag | |
| | caagaagcacccccagatcatcaagaagggcaaaaggccggcggccacgaaaaaggccggccaggcaaaaaagaaaaag | |
| | ggatccgaggccagcggttccggacgggctgacgcattggacgattttgatctggatatgctgggaagtgacgccctcg | |
| | atgattttgaccttgacatgcttggttcggatgcccttgatgactttgacctcgacatgctcggcagtgacgcccttga | |
| | tgatttcgacctggacatgctg | |
| NLS- | ccaaagaagaagcgcaaagtgggaatacacggggtaccagccgcgaaaagaaattacatcctggggcttgccataggga | 128 |
| miniSadCas9v5- | taacctctgtgggatacgggattattgactatgaaacacgcgatgtcatagatgctggtgtacggcttttcaaagaagc | |
| NLS-VP64 | aaatgtagaaaataatgagggccgaagaagtaaacggggagccagacggttgaagagaagacggaggcatcgcatacaa | |
| (optimized | agagttaagaagctcttgtttgattacaatttgcttacggatcactcagaattgtccggaataaatccgtatgaggcgc | |
| nucleotide | gggttaagggactgtcccaaaaactgtctgaagaggagtttagtgcagccctccttcatctcgctaaacgccgcggcgt | |
| sequence 1) | ccataatgtcaatgaggtagaagaggacacaggtaacgagctcagtacgaaagagcaaatctctcggaactctaaggcc | |
| | ttggaggagaaatatgttgccgaacttcaattggaaaggctgaaaaaagatggagaggtgcggggatctattaatcggt | |
| | tcaaaacgagcgattacgttaaggaggctaaacagttgctgaaggtccagaaagcttaccatcagttggatcaaagttt | |
| | catcgatacctacattgatctgttggagacccgaaggacatactacgaaggaccgggagaaggctctcccttcggatgg | |
| | aaagatattaaggagtggtatgagatgttgaagaggcggcggcgccataggatcttgtcaccggtcgttaaaaggtctt | |
| | tcatccaatcaatcaaagttatcaacgctatcataaaaaagtacggattgcctaatgacattataatagaactcggttc | |
| | aaaacggtacgccaccagaggtctgatgaacttgctcaggtcatacttccgggttaacaatctggacgtcaaagtcaaa | |
| | agcatcaacggaggattcacatcctttttgcgaagaaaatggaaattcaaaaaggagcgaaacaagggctataagcatc | |
| | atgctgaagacgcccttataatcgcgaatgcagactttatcttcaaagaatggaagaagttggataaagcaaagaaggt | |
| | tatggagaatcaaatgtttgaggaaaaacaagctgagagcatgccagaaatcgaaacagagcaagaatataaagagatc | |
| | ttcatcaccccgcaccaaataaagcacataaaggatttcaaggattataagtactcccacagggtagataaaaagccga | |
| | accgaaaattgataaacgatacattgtactctacgcgcaaggacgataaagggaacacacttatagtaaacaatctcaa | |
| | cggtctttacgacaaggacaacgataagttgaaaaagctcatcaataaatctcccgagaagcttctgatgtaccaccac | |
| | gacccgcagacctatcaaaagttgaaattgattatggagcaatacggggacgagaaaaaccccctgtacaagtactacg | |
| | aggaaacagggaactaccttacgaagtacagtaagaaagataatgggcctgtgattaaaaaaataaaatactatggaaa | |
| | caaactcaatgcgcatttggacataacagacgactaccccaattcaaggaacaaagtcgtcaaattgtctcttaagcca | |
| | tatcggttcgacgtgtacctggacaacggagtctacaaattcgttaccgttaagaaccttgacgtaatcaaaaaagaaa | |
| | actattacgaggttaactcaaagtgttatgaagaggccaagaagcttaaaaagatatcaaatcaagcagaatttatcgc | |
| | aagcttctataagaatgacttgataaagatcaatggggagttgtatcgggttattggagtgaacaatgatctgcttaat | |
| | agaatagaggtcaacatgattgatattacctatcgggaatacttggagaacatgaatgacaaaagacccccacacatca | |
| | taaagacaattgcatcaaagacgcagtcaattaaaaaatattccactgacattctcggcaatctgtatgaagtcaaatc | |
| | taaaaagcacccacaaatcatcaaaaagggcaagcggcctgccgcgaccaaaaaagctggccaagccaaaaagaaaaag | |
| | ggctccgaagcttctggtagcggccgagcggacgccttggatgacttcgatttggatatgctgggtagtgacgcgctgg | |
| | acgactttgacctcgatatgcttggcagtgacgccctcgacgacttcgacctggacatgctgggttcagatgctctgga | |
| | cgattttgacctcgacatgctg | |
| NLS- | cccaagaaaaaaagaaaagtgggcatccacggcgtgcccgccgccaagagaaactacatcctgggcctggccatcggca | 129 |
| miniSadCas9v5- | tcacaagcgtgggctacggcatcatcgactacgagacaagagacgtgatcgacgccggcgtgagactgttcaaggaggc | |
| NLS-VP64 | caacgtggagaacaacgagggcagaagaagcaagagaggcgctagaagattgaaacgccggaggagacatcggattcag | |
| (optimized | agagtgaagaagctgctgttcgactacaacctgctgaccgaccacagcgagctgagcggcatcaacccctacgaggcta | |
| nucleotide | gagtgaagggcctgtctcagaagctgagcgaggaggagttcagcgccgccctgctgcacctggccaagagaagaggcgt | |
| sequence 2) | gcacaacgtgaacgaggtggaggaggacaccggcaacgagctgagcaccaaggagcagatcagcagaaacagcaaggcc | |
| | ctggaggagaagtacgtggccgagctgcagctggagagactgaagaaggacggcgaggtgagaggcagcatcaacagat | |
| | tcaagacaagcgactacgtgaaggaggccaagcagctgctgaaggtgcagaaggcctaccatcagctggatcagagctt | |
| | catcgacacctacatcgacctgctggagacaagaagaacctactacgagggccccggcgagggcagccccttcggctgg | |
| | aaggacatcaaggagtggtacgagatgcttaagcgacgccgtcggcacagaatcttaagccccgtggtgaagagaagct | |
| | tcatccaaagcataaaggtgatcaacgccattataaagaagtacgggctgcccaacgacatcatcatcgagctgggcag | |
| | caagagatacgccacaagaggcctgatgaacctgctgagaagctacttcagagttaacaacctagacgtgaaagttaag | |
| | agcataaacggcggcttcacaagcttcctgagaagaaagtggaagttcaagaaggagagaaataagggctacaagcacc | |
| | acgccgaggacgccctgatcatcgccaacgccgacttcatcttcaaggagtggaagaagctggacaaggccaagaaggt | |
| | gatggagaatcagatgttcgaggagaagcaagccgagagcatgcccgagatcgagaccgagcaagagtacaaggagatc | |
| | ttcatcaccccccatcagatcaagcacatcaaggacttcaaggactacaagtacagccacagagtggacaagaagccca | |
| | acagaaagctcatcaatgacaccctgtacagcacaagaaaggacgacaagggcaacaccctgatcgtgaacaacctgaa | |
| | cggcctgtacgacaaggacaacgacaagctcaagaaactgatcaacaagagccccgagaagctgctgatgtaccaccac | |
| | gaccctcagacctatcagaagctgaagctgatcatggagcagtacggcgacgagaagaaccccctgtacaagtactacg | |
| | aggagaccggcaactacctgaccaagtacagcaagaaggacaacggccccgtgatcaagaagatcaaatactacggcaa | |
| | caagctgaacgcccacctggacatcaccgacgactaccccaacagcagaaacaaggtggtgaagctgagcctgaagccc | |
| | tacagattcgacgtgtacctggacaacggcgtgtacaagttcgtgaccgtgaagaacctggatgttatcaaaaaggaga | |
| | attactacgaggtgaacagcaagtgctacgaggaggccaagaaactgaagaaaatctcgaaccaagccgagttcatcgc | |
| | tagcttctacaagaacgacctgatcaagatcaacggcgagctgtacagagtgatcggcgtgaacaacgacctgctgaac | |
| | agaatcgaggtgaacatgatcgacatcacctacagagagtacctggagaacatgaacgacaagagacccccccacatca | |
| | tcaagaccatcgctagcaagacacagtcgatcaaaaagtacagcaccgacatcctgggcaacctgtacgaggtaaaaag | |
| | caagaagcaccctcagatcatcaagaaaggcaaaagacccgccgccaccaagaaggccggccaagccaagaagaagaag | |
| | ggcagcgaggctagcggcagcggcagagccgatgccctggacgacttcgatttggacatgctgggtagcgacgccctag | |
| | atgacttcgacctggatatgctggggagcgatgcgctagatgatttcgatcttgatatgctcgggagcgacgctctaga | |
| | cgatttcgacttagatatgctg | |

“NLS” = nuclear localization signal

TABLE 7
Exemplary AAV expression cassettes

| Name | Sequence | SEQ ID NO |
|---|---|---|
| pAAV-SCN1A_SE1 | cctgcaggcagctgcgcgctcgctcgctcactgaggccgcccgggcgtcgggcgacctttggtcgcccg | 137 |
| | gcctcagtgagcgagcgagcgcgcagagagggagtggccaactccatcactaggggttcctgcggccgc | |
| | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctagcctgcaagtcgaggagcgcagccttccagaagcagagcgc | |
| | ggcgccttaagctgcagaagttggtcgtgaggcactgggcacgtaagtatcaaggttacaagacaggtt | |
| | taaggagaccaatagaaactgggcttgtcgagacagagaagactcttgcgtttctgataggcacctatt | |
| | ggtcttactgacatccactttgcctttctctccacagcgagctcgctagcgcgatcgctgccaccatgg | |
| | ccccaaagaagaagcggaaggtcggtatccacggagtcccagcagccaagcggaactacatcctgggcc | |
| | tggccatcggcatcaccagcgtgggctacggcatcatcgactacgagacacgggacgtgatcgatgccg | |
| | gcgtgcggctgttcaaagaggccaacgtggaaaacaacgagggcaggcggagcaagagaggcgccagaa | |
| | ggctgaagcggcggaggcggcatagaatccagagagtgaagaagctgctgttcgactacaacctgctga | |
| | ccgaccacagcgagctgagcggcatcaacccctacgaggccagagtgaagggcctgagccagaagctga | |
| | gcgaggaagagttctctgccgccctgctgcacctggccaagagaagaggcgtgcacaacgtgaacgagg | |
| | tggaagaggacaccggcaacgagctgtccaccaaagagcagatcagccggaacagcaaggccctggaag | |
| | agaaatacgtggccgaactgcagctggaacggctgaagaaagacggcgaagtgcggggcagcatcaaca | |
| | gattcaagaccagcgactacgtgaaagaagccaaacagctgctgaaggtgcagaaggcctaccaccagc | |
| | tggaccagagcttcatcgacacctacatcgacctgctggaaacccggcggacctactatgagggacctg | |
| | gcgagggcagccccttcggctggaaggacatcaaagaatggtacgagatgctgatgggccactgcacct | |
| | acttccccgaggaactgcggagcgtgaagtacgcctacaacgccgacctgtacaacgccctgaacgacc | |
| | tgaacaatctcgtgatcaccagggacgagaacgagaagctggaatattacgagaagttccagatcatcg | |
| | agaacgtgttcaagcagaagaagaagcccaccctgaagcagatcgccaaagaaatcctcgtgaacgaag | |
| | aggatattaagggctacagagtgaccagcaccggcaagcccgagttcaccaacctgaaggtgtaccacg | |
| | acatcaaggacattaccgcccggaaagagattattgagaacgccgagctgctggatcagattgccaaga | |
| | tcctgaccatctaccagagcagcgaggacatccaggaagaactgaccaatctgaactccgagctgaccc | |
| | aggaagagatcgagcagatctctaatctgaagggctataccggcacccacaacctgagcctgaaggcca | |
| | tcaacctgatcctggacgagctgtggcacaccaacgacaaccagatcgctatcttcaaccggctgaagc | |
| | tggtgcccaagaaggtggacctgtcccagcagaaagagatccccaccaccctggtggacgacttcatcc | |
| | tgagccccgtcgtgaagagaagcttcatccagagcatcaaagtgatcaacgccatcatcaagaagtacg | |
| | gcctgcccaacgacatcattatcgagctggcccgcgagaagaactccaaggacgcccagaaaatgatca | |
| | acgagatgcagaagcggaaccggcagaccaacgagcggatcgaggaaatcatccggaccaccggcaaag | |
| | agaacgccaagtacctgatcgagaagatcaagctgcacgacatgcaggaaggcaagtgcctgtacagcc | |
| | tggaagccatccctctggaagatctgctgaacaaccccttcaactatgaggtggaccacatcatcccca | |
| | gaagcgtgtccttcgacaacagcttcaacaacaaggtgctcgtgaagcaggaagaagccagcaagaagg | |
| | gcaaccggaccccattccagtacctgagcagcagcgacagcaagatcagctacgaaaccttcaagaagc | |
| | acatcctgaatctggccaagggcaagggcagaatcagcaagaccaagaaagagtatctgctggaagaac | |
| | gggacatcaacaggttctccgtgcagaaagacttcatcaaccggaacctggtggataccagatacgcca | |
| | ccagaggcctgatgaacctgctgcggagctacttcagagtgaacaacctggacgtgaaagtgaagtcca | |
| | tcaatggcggcttcaccagctttctgcggcggaagtggaagtttaagaaagagcggaacaaggggtaca | |
| | agcaccacgccgaggacgccctgatcattgccaacgccgatttcatcttcaaagagtggaagaaactgg | |
| | acaaggccaaaaaagtgatggaaaaccagatgttcgaggaaaagcaggccgagagcatgcccgagatcg | |
| | aaaccgagcaggagtacaaagagatcttcatcaccccccaccagatcaagcacattaaggacttcaagg | |
| | actacaagtacagccaccgggtggacaagaagcctaatagagagctgattaacgacaccctgtactcca | |
| | cccggaaggacgacaagggcaacaccctgatcgtgaacaatctgaacggcctgtacgacaaggacaatg | |
| | acaagctgaaaaagctgatcaacaagagccccgaaaagctgctgatgtaccaccacgacccccagacct | |
| | accagaaactgaagctgattatggaacagtacggcgacgagaagaatcccctgtacaagtactacgagg | |
| | aaaccgggaactacctgaccaagtactccaaaaaggacaacggccccgtgatcaagaagattaagtatt | |
| | acggcaacaaactgaacgcccatctggacatcaccgacgactaccccaacagcagaaacaaggtcgtga | |
| | agctgtccctgaagccctacagattcgacgtgtacctggacaatggcgtgtacaagttcgtgaccgtga | |
| | agaatctggatgtgatcaaaaaagaaaactactacgaagtgaatagcaagtgctatgaggaagctaaga | |
| | agctgaagaagatcagcaaccaggccgagtttatcgcctccttctacaacaacgatctgatcaagatca | |
| | acggcgagctgtatagagtgatcggcgtgaacaacgacctgctgaaccggatcgaagtgaacatgatcg | |
| | acatcacctaccgcgagtacctggaaaacatgaacgacaagaggccccccaggatcattaagacaatcg | |
| | cctccaagacccagagcattaagaagtacagcacagacattctgggcaacctgtatgaagtgaaatcta | |
| | agaagcaccctcagatcatcaaaaagggcaaaaggccggcggccacgaaaaaggccggccaggcaaaaa | |
| | agaaaaagggatccgaggccagcggttccggacgggctgacgcattggacgattttgatctggatatgc | |
| | tgggaagtgacgccctcgatgattttgaccttgacatgcttggttcggatgcccttgatgactttgacc | |
| | tcgacatgctcggcagtgacgcccttgatgatttcgacctggacatgctgtgaatcgaattccgctcga | |
| | gataatcaacctctggattacaaaatttgtgaaagattgactggtattcttaactatgttgctcctttt | |
| | acgctatgtggatacgctgctttaatgcctttgtatcatgctattgcttcccgtatggctttcattttc | |
| | tcctccttgtataaatcctggttagttcttgccacggcggaactcatcgccgcctgccttgcccgctgc | |
| | tggacaggggctcggctgttgggcactgacaattccgtggtgttctggatcaacttgtttattgcagct | |
| | tataatggttacaaataaagcaatagcatcacaaatttcacaaataaagcatttttttcactgcgcggc | |
| | cgcaggaacccctagtgatggagttggccactccctctctgcgcgctcgctcgctcactgaggccgggc | |
| | gaccaaaggtcgcccgacgcccgggctttgcccgggcggcctcagtgagcgagcgagcgcgcag | |
| pAAV- | cctgcaggcagctgcgcgctcgctcgctcactgaggccgcccgggcgtcgggcgacctttggtcgcccg | 138 |
| SCN1A_SE1_mini- | gcctcagtgagcgagcgagcgcgcagagagggagtggccaactccatcactaggggttcctgcggccgc | |
| SadCas9-v2 | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctagcctgcaagtcgaggagcgcagccttccagaagcagagcgc | |
| | ggcgccttaagctgcagaagttggtcgtgaggcactgggcacgtaagtatcaaggttacaagacaggtt | |
| | taaggagaccaatagaaactgggcttgtcgagacagagaagactcttgcgtttctgataggcacctatt | |
| | ggtcttactgacatccactttgcctttctctccacagcgagctcgctagcgcgatcgctgccaccatgg | |
| | ccccaaagaagaagcggaaggtcggtatccacggagtcccagcagccaagaggaactacatcctgggcc | |
| | tggccatcggcatcaccagcgtgggctacggcatcatcgactacgagaccagggacgtgatcgacgccg | |
| | gcgtgaggctgttcaaggaggccaacgtggagaacaacgagggcaggaggagcaagaggggcgccagga | |
| | ggctgaagaggaggaggaggcacaggatccagagggtgaagaagctgctgttcgactacaacctgctga | |
| | ccgaccacagcgagctgagcggcatcaacccctacgaggccagggtgaagggcctgagccagaagctga | |
| | gcgaggaggagttcagcgccgccctgctgcacctggccaagaggaggggcgtgcacaacgtgaacgagg | |
| | tggaggaggacaccggcaacgagctgagcaccaaggagcagatcagcaggaacagcaaggccctggagg | |
| | agaagtacgtggccgagctgcagctggagaggctgaagaaggacggcgaggtgaggggcagcatcaaca | |
| | ggttcaagaccagcgactacgtgaaggaggccaagcagctgctgaaggtgcagaaggcctaccaccagc | |
| | tggaccagagcttcatcgacacctacatcgacctgctggagaccaggaggacctactacgagggccccg | |
| | gcgagggcagccccttcggctggaaggacatcaaggagtggtacgagatgctgaagaggaggaggaggc | |
| | acaggatcctgagccccgtggtgaagaggagcttcatccagagcatcaaggtgatcaacgccatcatca | |
| | agaagtacggcctgcccaacgacatcatcatcgagctggccagggagaagaacagcaaggacgcccaga | |
| | agatgatcaacgagatgcagaagaggaacaggcagaccaacgagaggatcgaggagatcatcaggacca | |
| | ccggcaaggagaacgccaagtacctgatcgagaagatcaagctgcacgacatgcaggagggcaagtgcc | |
| | tgtacagcctggaggccatccccctggaggacctgctgaacaaccccttcaactacgaggtggaccaca | |
| | tcatccccaggagcgtgagcttcgacaacagcttcaacaacaaggtgctggtgaagcaggaggaggcca | |
| | gcaagaagggcaacaggacccccttccagtacctgagcagcagcgacagcaagatcagctacgagacct | |
| | tcaagaagcacatcctgaacctggccaagggcaagggcaggatcagcaagaccaagaaggagtacctgc | |
| | tggaggagagggacatcaacaggttcagcgtgcagaaggacttcatcaacaggaacctggtggacacca | |
| | ggtacgccaccaggggcctgatgaacctgctgaggagctacttcagggtgaacaacctggacgtgaagg | |
| | tgaagagcatcaacggcggcttcaccagcttcctgaggaggaagtggaagttcaagaaggagaggaaca | |
| | agggctacaagcaccacgccgaggacgccctgatcatcgccaacgccgacttcatcttcaaggagtgga | |
| | agaagctggacaaggccaagaaggtgatggagaaccagatgttcgaggagaagcaggccgagagcatgc | |
| | ccgagatcgagaccgagcaggagtacaaggagatcttcatcaccccccaccagatcaagcacatcaagg | |
| | acttcaaggactacaagtacagccacagggtggacaagaagcccaacaggaagctgatcaacgacaccc | |
| | tgtacagcaccaggaaggacgacaagggcaacaccctgatcgtgaacaacctgaacggcctgtacgaca | |
| | aggacaacgacaagctgaagaagctgatcaacaagagccccgagaagctgctgatgtaccaccacgacc | |
| | cccagacctaccagaagctgaagctgatcatggagcagtacggcgacgagaagaaccccctgtacaagt | |
| | actacgaggagaccggcaactacctgaccaagtacagcaagaaggacaacggccccgtgatcaagaaga | |
| | tcaagtactacggcaacaagctgaacgcccacctggacatcaccgacgactaccccaacagcaggaaca | |
| | aggtggtgaagctgagcctgaagccctacaggttcgacgtgtacctggacaacggcgtgtacaagttcg | |
| | tgaccgtgaagaacctggacgtgatcaagaaggagaactactacgaggtgaacagcaagtgctacgagg | |
| | aggccaagaagctgaagaagatcagcaaccaggccgagttcatcgccagcttctacaagaacgacctga | |
| | tcaagatcaacggcgagctgtacagggtgatcggcgtgaacaacgacctgctgaacaggatcgaggtga | |
| | acatgatcgacatcacctacagggagtacctggagaacatgaacgacaagaggcccccccacatcatca | |
| | agaccatcgccagcaagacccagagcatcaagaagtacagcaccgacatcctgggcaacctgtacgagg | |
| | tgaagagcaagaagcacccccagatcatcaagaagggcaaaaggccggcggccacgaaaaaggccggcc | |
| | aggcaaaaaagaaaaagggatccgaggccagcggttccggacgggctgacgcattggacgattttgatc | |
| | tggatatgctgggaagtgacgccctcgatgattttgaccttgacatgcttggttcggatgcccttgatg | |
| | actttgacctcgacatgctcggcagtgacgcccttgatgatttcgacctggacatgctgtgaatcgaat | |
| | tccgctcgagataatcaacctctggattacaaaatttgtgaaagattgactggtattcttaactatgtt | |
| | gctccttttacgctatgtggatacgctgctttaatgcctttgtatcatgctattgcttcccgtatggct | |
| | ttcattttctcctccttgtataaatcctggttagttcttgccacggcggaactcatcgccgcctgcctt | |
| | gcccgctgctggacaggggctcggctgttgggcactgacaattccgtggtgttctggatcaacttgttt | |
| | attgcagcttataatggttacaaataaagcaatagcatcacaaatttcacaaataaagcatttttttca | |
| | ctgcgcggccgcaggaacccctagtgatggagttggccactccctctctgcgcgctcgctcgctcactg | |
| | aggccgggcgaccaaaggtcgcccgacgcccgggctttgcccgggcggcctcagtgagcgagcgagcgc | |
| | gcag | |
| pAAV- | cctgcaggcagctgcgcgctcgctcgctcactgaggccgcccgggcgtcgggcgacctttggtcgcccg | 139 |
| SCN1A_SE1_mini- | gcctcagtgagcgagcgagcgcgcagagagggagtggccaactccatcactaggggttcctgcggccgc | |
| SadCas9-v4 | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctagcctgcaagtcgaggagcgcagccttccagaagcagagcgc | |
| | ggcgccttaagctgcagaagttggtcgtgaggcactgggcacgtaagtatcaaggttacaagacaggtt | |
| | taaggagaccaatagaaactgggcttgtcgagacagagaagactcttgcgtttctgataggcacctatt | |
| | ggtcttactgacatccactttgcctttctctccacagcgagctcgctagcgcgatcgctgccaccatgg | |
| | ccccaaagaagaagcggaaggtcggtatccacggagtcccagcagccaagcggaactacatcctgggcc | |
| | tggccatcggcatcaccagcgtgggctacggcatcatcgactacgagacacgggacgtgatcgatgccg | |
| | gcgtgcggctgttcaaagaggccaacgtggaaaacaacgagggcaggcggagcaagagaggcgccagaa | |
| | ggctgaagcggcggaggcggcatagaatccagagagtgaagaagctgctgttcgactacaacctgctga | |
| | ccgaccacagcgagctgagcggcatcaacccctacgaggccagagtgaagggcctgagccagaagctga | |
| | gcgaggaagagttctctgccgccctgctgcacctggccaagagaagaggcgtgcacaacgtgaacgagg | |
| | tggaagaggacaccggcaacgagctgtccaccaaagagcagatcagccggaacagcaaggccctggaag | |
| | agaaatacgtggccgaactgcagctggaacggctgaagaaagacggcgaagtgcggggcagcatcaaca | |
| | gattcaagaccagcgactacgtgaaagaagccaaacagctgctgaaggtgcagaaggcctaccaccagc | |
| | tggaccagagcttcatcgacacctacatcgacctgctggaaacccggcggacctactatgagggacctg | |
| | gcgagggcagccccttcggctggaaggacatcaaagaatggtacgagatgctgatgggccactgcacct | |
| | acttccccgaggaactgcggagcgtgaagtacgcctacaacgccgacctgtacaacgccctgaacgacc | |
| | tgaacaatctcgtgatcaccagggacgagaacgagaagctggaatattacgagaagttccagatcatcg | |
| | agaacgtgttcaagcagaagaagaagcccaccctgaagcagatcgccaaagaaatcctcgtgaacgaag | |
| | aggatattaagggctacagagtgaccagcaccggcaagcccgagttcaccaacctgaaggtgtaccacg | |
| | acatcaaggacattaccgcccggaaagagattattgagaacgccgagctgctggatcagattgccaaga | |
| | tcctgaccatctaccagagcagcgaggacatccaggaagaactgaccaatctgaactccgagctgaccc | |
| | aggaagagatcgagcagatctctaatctgaagggctataccggcacccacaacctgagcctgaaggcca | |
| | tcaacctgatcctggacgagctgtggcacaccaacgacaaccagatcgctatcttcaaccggctgaagc | |
| | tggtgcccaagaaggtggacctgtcccagcagaaagagatccccaccaccctggtggacgacttcatcc | |
| | tgagccccgtcgtgaagagaagcttcatccagagcatcaaagtgatcaacgccatcatcaagaagtacg | |
| | gcctgcccaacgacatcattatcgagctgggcagcaagagatacgccaccagaggcctgatgaacctgc | |
| | tgcggagctacttcagagtgaacaacctggacgtgaaagtgaagtccatcaatggcggcttcaccagct | |
| | ttctgcggcggaagtggaagtttaagaaagagcggaacaaggggtacaagcaccacgccgaggacgccc | |
| | tgatcattgccaacgccgatttcatcttcaaagagtggaagaaactggacaaggccaaaaaagtgatgg | |
| | aaaaccagatgttcgaggaaaagcaggccgagagcatgcccgagatcgaaaccgagcaggagtacaaag | |
| | agatcttcatcaccccccaccagatcaagcacattaaggacttcaaggactacaagtacagccaccggg | |
| | tggacaagaagcctaatagagagctgattaacgacaccctgtactccacccggaaggacgacaagggca | |
| | acaccctgatcgtgaacaatctgaacggcctgtacgacaaggacaatgacaagctgaaaaagctgatca | |
| | acaagagccccgaaaagctgctgatgtaccaccacgacccccagacctaccagaaactgaagctgatta | |
| | tggaacagtacggcgacgagaagaatcccctgtacaagtactacgaggaaaccgggaactacctgacca | |
| | agtactccaaaaaggacaacggccccgtgatcaagaagattaagtattacggcaacaaactgaacgccc | |
| | atctggacatcaccgacgactaccccaacagcagaaacaaggtcgtgaagctgtccctgaagccctaca | |
| | gattcgacgtgtacctggacaatggcgtgtacaagttcgtgaccgtgaagaatctggatgtgatcaaaa | |
| | aagaaaactactacgaagtgaatagcaagtgctatgaggaagctaagaagctgaagaagatcagcaacc | |
| | aggccgagtttatcgcctccttctacaacaacgatctgatcaagatcaacggcgagctgtatagagtga | |
| | tcggcgtgaacaacgacctgctgaaccggatcgaagtgaacatgatcgacatcacctaccgcgagtacc | |
| | tggaaaacatgaacgacaagaggccccccaggatcattaagacaatcgcctccaagacccagagcatta | |
| | agaagtacagcacagacattctgggcaacctgtatgaagtgaaatctaagaagcaccctcagatcatca | |
| | aaaagggcaaaaggccggcggccacgaaaaaggccggccaggcaaaaaagaaaaagggatccgaggcca | |
| | gcggttccggacgggctgacgcattggacgattttgatctggatatgctgggaagtgacgccctcgatg | |
| | attttgaccttgacatgcttggttcggatgcccttgatgactttgacctcgacatgctcggcagtgacg | |
| | cccttgatgatttcgacctggacatgctgtgaatcgaattccgctcgagataatcaacctctggattac | |
| | aaaatttgtgaaagattgactggtattcttaactatgttgctccttttacgctatgtggatacgctgct | |
| | ttaatgcctttgtatcatgctattgcttcccgtatggctttcattttctcctccttgtataaatcctgg | |
| | ttagttcttgccacggcggaactcatcgccgcctgccttgcccgctgctggacaggggctcggctgttg | |
| | ggcactgacaattccgtggtgttctggatcaacttgtttattgcagcttataatggttacaaataaagc | |
| | aatagcatcacaaatttcacaaataaagcatttttttcactgcgcggccgcaggaacccctagtgatgg | |
| | agttggccactccctctctgcgcgctcgctcgctcactgaggccgggcgaccaaaggtcgcccgacgcc | |
| | cgggctttgcccgggcggcctcagtgagcgagcgagcgcgcag | |
| pAAV- | cctgcaggcagctgcgcgctcgctcgctcactgaggccgcccgggcgtcgggcgacctttggtcgcccg | 140 |
| SCN1A_SE1_mini- | gcctcagtgagcgagcgagcgcgcagagagggagtggccaactccatcactaggggttcctgcggccgc | |
| SadCas9-v5 | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctagcctgcaagtcgaggagcgcagccttccagaagcagagcgc | |
| | ggcgccttaagctgcagaagttggtcgtgaggcactgggcacgtaagtatcaaggttacaagacaggtt | |
| | taaggagaccaatagaaactgggcttgtcgagacagagaagactcttgcgtttctgataggcacctatt | |
| | ggtcttactgacatccactttgcctttctctccacagcgagctcgctagcgcgatcgctgccaccatgg | |
| | ccccaaagaagaagcggaaggtcggtatccacggagtcccagcagccaagaggaactacatcctgggcc | |
| | tggccatcggcatcaccagcgtgggctacggcatcatcgactacgagaccagggacgtgatcgacgccg | |
| | gcgtgaggctgttcaaggaggccaacgtggagaacaacgagggcaggaggagcaagaggggcgccagga | |
| | ggctgaagaggaggaggaggcacaggatccagagggtgaagaagctgctgttcgactacaacctgctga | |
| | ccgaccacagcgagctgagcggcatcaacccctacgaggccagggtgaagggcctgagccagaagctga | |
| | gcgaggaggagttcagcgccgccctgctgcacctggccaagaggaggggcgtgcacaacgtgaacgagg | |
| | tggaggaggacaccggcaacgagctgagcaccaaggagcagatcagcaggaacagcaaggccctggagg | |
| | agaagtacgtggccgagctgcagctggagaggctgaagaaggacggcgaggtgaggggcagcatcaaca | |
| | ggttcaagaccagcgactacgtgaaggaggccaagcagctgctgaaggtgcagaaggcctaccaccagc | |
| | tggaccagagcttcatcgacacctacatcgacctgctggagaccaggaggacctactacgagggccccg | |
| | gcgagggcagccccttcggctggaaggacatcaaggagtggtacgagatgctgaagaggaggaggaggc | |
| | acaggatcctgagccccgtggtgaagaggagcttcatccagagcatcaaggtgatcaacgccatcatca | |
| | agaagtacggcctgcccaacgacatcatcatcgagctgggcagcaagaggtacgccaccaggggcctga | |
| | tgaacctgctgaggagctacttcagggtgaacaacctggacgtgaaggtgaagagcatcaacggcggct | |
| | tcaccagcttcctgaggaggaagtggaagttcaagaaggagaggaacaagggctacaagcaccacgccg | |
| | aggacgccctgatcatcgccaacgccgacttcatcttcaaggagtggaagaagctggacaaggccaaga | |
| | aggtgatggagaaccagatgttcgaggagaagcaggccgagagcatgcccgagatcgagaccgagcagg | |
| | agtacaaggagatcttcatcaccccccaccagatcaagcacatcaaggacttcaaggactacaagtaca | |
| | gccacagggtggacaagaagcccaacaggaagctgatcaacgacaccctgtacagcaccaggaaggacg | |
| | acaagggcaacaccctgatcgtgaacaacctgaacggcctgtacgacaaggacaacgacaagctgaaga | |
| | agctgatcaacaagagccccgagaagctgctgatgtaccaccacgacccccagacctaccagaagctga | |
| | agctgatcatggagcagtacggcgacgagaagaaccccctgtacaagtactacgaggagaccggcaact | |
| | acctgaccaagtacagcaagaaggacaacggccccgtgatcaagaagatcaagtactacggcaacaagc | |
| | tgaacgcccacctggacatcaccgacgactaccccaacagcaggaacaaggtggtgaagctgagcctga | |
| | agccctacaggttcgacgtgtacctggacaacggcgtgtacaagttcgtgaccgtgaagaacctggacg | |
| | tgatcaagaaggagaactactacgaggtgaacagcaagtgctacgaggaggccaagaagctgaagaaga | |
| | tcagcaaccaggccgagttcatcgccagcttctacaagaacgacctgatcaagatcaacggcgagctgt | |
| | acagggtgatcggcgtgaacaacgacctgctgaacaggatcgaggtgaacatgatcgacatcacctaca | |
| | gggagtacctggagaacatgaacgacaagaggcccccccacatcatcaagaccatcgccagcaagaccc | |
| | agagcatcaagaagtacagcaccgacatcctgggcaacctgtacgaggtgaagagcaagaagcaccccc | |
| | agatcatcaagaagggcaaaaggccggcggccacgaaaaaggccggccaggcaaaaaagaaaaagggat | |
| | ccgaggccagcggttccggacgggctgacgcattggacgattttgatctggatatgctgggaagtgacg | |
| | ccctcgatgattttgaccttgacatgcttggttcggatgcccttgatgactttgacctcgacatgctcg | |
| | gcagtgacgcccttgatgatttcgacctggacatgctgtgaatcgaattccgctcgagataatcaacct | |
| | ctggattacaaaatttgtgaaagattgactggtattcttaactatgttgctccttttacgctatgtgga | |
| | tacgctgctttaatgcctttgtatcatgctattgcttcccgtatggctttcattttctcctccttgtat | |
| | aaatcctggttagttcttgccacggcggaactcatcgccgcctgccttgcccgctgctggacaggggct | |
| | cggctgttgggcactgacaattccgtggtgttctggatcaacttgtttattgcagcttataatggttac | |
| | aaataaagcaatagcatcacaaatttcacaaataaagcatttttttcactgcgcggccgcaggaacccc | |
| | tagtgatggagttggccactccctctctgcgcgctcgctcgctcactgaggccgggcgaccaaaggtcg | |
| | cccgacgcccgggctttgcccgggcggcctcagtgagcgagcgagcgcgcag | |
| pAAV-SCN1A_SE2 | cctgcaggcagctgcgcgctcgctcgctcactgaggccgcccgggcgtcgggcgacctttggtcgcccg | 141 |
| | gcctcagtgagcgagcgagcgcgcagagagggagtggccaactccatcactaggggttcctgcggccgc | |
| | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctagcctgcaagtcgaggagcgcagccttccagaagcagagcgc | |
| | ggcgccttaagctgcagaagttggtcgtgaggcactgggcacgtaagtatcaaggttacaagacaggtt | |
| | taaggagaccaatagaaactgggcttgtcgagacagagaagactcttgcgtttctgataggcacctatt | |
| | ggtcttactgacatccactttgcctttctctccacagcgagctcgctagcgcgatcgctgccaccatgg | |
| | ccccaaagaagaagcggaaggtcggtatccacggagtcccagcagccaagcggaactacatcctgggcc | |
| | tggccatcggcatcaccagcgtgggctacggcatcatcgactacgagacacgggacgtgatcgatgccg | |
| | gcgtgcggctgttcaaagaggccaacgtggaaaacaacgagggcaggcggagcaagagaggcgccagaa | |
| | ggctgaagcggcggaggcggcatagaatccagagagtgaagaagctgctgttcgactacaacctgctga | |
| | ccgaccacagcgagctgagcggcatcaacccctacgaggccagagtgaagggcctgagccagaagctga | |
| | gcgaggaagagttctctgccgccctgctgcacctggccaagagaagaggcgtgcacaacgtgaacgagg | |
| | tggaagaggacaccggcaacgagctgtccaccaaagagcagatcagccggaacagcaaggccctggaag | |
| | agaaatacgtggccgaactgcagctggaacggctgaagaaagacggcgaagtgcggggcagcatcaaca | |
| | gattcaagaccagcgactacgtgaaagaagccaaacagctgctgaaggtgcagaaggcctaccaccagc | |
| | tggaccagagcttcatcgacacctacatcgacctgctggaaacccggcggacctactatgagggacctg | |
| | gcgagggcagccccttcggctggaaggacatcaaagaatggtacgagatgctgatgggccactgcacct | |
| | acttccccgaggaactgcggagcgtgaagtacgcctacaacgccgacctgtacaacgccctgaacgacc | |
| | tgaacaatctcgtgatcaccagggacgagaacgagaagctggaatattacgagaagttccagatcatcg | |
| | agaacgtgttcaagcagaagaagaagcccaccctgaagcagatcgccaaagaaatcctcgtgaacgaag | |
| | aggatattaagggctacagagtgaccagcaccggcaagcccgagttcaccaacctgaaggtgtaccacg | |
| | acatcaaggacattaccgcccggaaagagattattgagaacgccgagctgctggatcagattgccaaga | |
| | tcctgaccatctaccagagcagcgaggacatccaggaagaactgaccaatctgaactccgagctgaccc | |
| | aggaagagatcgagcagatctctaatctgaagggctataccggcacccacaacctgagcctgaaggcca | |
| | tcaacctgatcctggacgagctgtggcacaccaacgacaaccagatcgctatcttcaaccggctgaagc | |
| | tggtgcccaagaaggtggacctgtcccagcagaaagagatccccaccaccctggtggacgacttcatcc | |
| | tgagccccgtcgtgaagagaagcttcatccagagcatcaaagtgatcaacgccatcatcaagaagtacg | |
| | gcctgcccaacgacatcattatcgagctggcccgcgagaagaactccaaggacgcccagaaaatgatca | |
| | acgagatgcagaagcggaaccggcagaccaacgagcggatcgaggaaatcatccggaccaccggcaaag | |
| | agaacgccaagtacctgatcgagaagatcaagctgcacgacatgcaggaaggcaagtgcctgtacagcc | |
| | tggaagccatccctctggaagatctgctgaacaaccccttcaactatgaggtggaccacatcatcccca | |
| | gaagcgtgtccttcgacaacagcttcaacaacaaggtgctcgtgaagcaggaagaagccagcaagaagg | |
| | gcaaccggaccccattccagtacctgagcagcagcgacagcaagatcagctacgaaaccttcaagaagc | |
| | acatcctgaatctggccaagggcaagggcagaatcagcaagaccaagaaagagtatctgctggaagaac | |
| | gggacatcaacaggttctccgtgcagaaagacttcatcaaccggaacctggtggataccagatacgcca | |
| | ccagaggcctgatgaacctgctgcggagctacttcagagtgaacaacctggacgtgaaagtgaagtcca | |
| | tcaatggcggcttcaccagctttctgcggcggaagtggaagtttaagaaagagcggaacaaggggtaca | |
| | agcaccacgccgaggacgccctgatcattgccaacgccgatttcatcttcaaagagtggaagaaactgg | |
| | acaaggccaaaaaagtgatggaaaaccagatgttcgaggaaaagcaggccgagagcatgcccgagatcg | |
| | aaaccgagcaggagtacaaagagatcttcatcaccccccaccagatcaagcacattaaggacttcaagg | |
| | actacaagtacagccaccgggtggacaagaagcctaatagagagctgattaacgacaccctgtactcca | |
| | cccggaaggacgacaagggcaacaccctgatcgtgaacaatctgaacggcctgtacgacaaggacaatg | |
| | acaagctgaaaaagctgatcaacaagagccccgaaaagctgctgatgtaccaccacgacccccagacct | |
| | accagaaactgaagctgattatggaacagtacggcgacgagaagaatcccctgtacaagtactacgagg | |
| | aaaccgggaactacctgaccaagtactccaaaaaggacaacggccccgtgatcaagaagattaagtatt | |
| | acggcaacaaactgaacgcccatctggacatcaccgacgactaccccaacagcagaaacaaggtcgtga | |
| | agctgtccctgaagccctacagattcgacgtgtacctggacaatggcgtgtacaagttcgtgaccgtga | |
| | agaatctggatgtgatcaaaaaagaaaactactacgaagtgaatagcaagtgctatgaggaagctaaga | |
| | agctgaagaagatcagcaaccaggccgagtttatcgcctccttctacaacaacgatctgatcaagatca | |
| | acggcgagctgtatagagtgatcggcgtgaacaacgacctgctgaaccggatcgaagtgaacatgatcg | |
| | acatcacctaccgcgagtacctggaaaacatgaacgacaagaggccccccaggatcattaagacaatcg | |
| | cctccaagacccagagcattaagaagtacagcacagacattctgggcaacctgtatgaagtgaaatcta | |
| | agaagcaccctcagatcatcaaaaagggcaaaaggccggcggccacgaaaaaggccggccaggcaaaaa | |
| | agaaaaagggatccgaggccagcggttccggacgggctgacgcattggacgattttgatctggatatgc | |
| | tgggaagtgacgccctcgatgattttgaccttgacatgcttggttcggatgcccttgatgactttgacc | |
| | tcgacatgctcggcagtgacgcccttgatgatttcgacctggacatgctgtgaatcgaattccgctcga | |
| | gtggatcaacttgtttattgcagcttataatggttacaaataaagcaatagcatcacaaatttcacaaa | |
| | taaagcatttttttcactgcgcggccgcaggaacccctagtgatggagttggccactccctctctgcgc | |
| | gctcgctcgctcactgaggccgggcgaccaaaggtcgcccgacgcccgggctttgcccgggcggcctca | |
| | gtgagcgagcgagcgcgcag | |
| pAAV- | cctgcaggcagctgcgcgctcgctcgctcactgaggccgcccgggcgtcgggcgacctttggtcgcccg | 142 |
| SCN1A_SE2_mini- | gcctcagtgagcgagcgagcgcgcagagagggagtggccaactccatcactaggggttcctgcggccgc | |
| SadCas9-v2 | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctagcctgcaagtcgaggagcgcagccttccagaagcagagcgc | |
| | ggcgccttaagctgcagaagttggtcgtgaggcactgggcacgtaagtatcaaggttacaagacaggtt | |
| | taaggagaccaatagaaactgggcttgtcgagacagagaagactcttgcgtttctgataggcacctatt | |
| | ggtcttactgacatccactttgcctttctctccacagcgagctcgctagcgcgatcgctgccaccatgg | |
| | ccccaaagaagaagcggaaggtcggtatccacggagtcccagcagccaagaggaactacatcctgggcc | |
| | tggccatcggcatcaccagcgtgggctacggcatcatcgactacgagaccagggacgtgatcgacgccg | |
| | gcgtgaggctgttcaaggaggccaacgtggagaacaacgagggcaggaggagcaagaggggcgccagga | |
| | ggctgaagaggaggaggaggcacaggatccagagggtgaagaagctgctgttcgactacaacctgctga | |
| | ccgaccacagcgagctgagcggcatcaacccctacgaggccagggtgaagggcctgagccagaagctga | |
| | gcgaggaggagttcagcgccgccctgctgcacctggccaagaggaggggcgtgcacaacgtgaacgagg | |
| | tggaggaggacaccggcaacgagctgagcaccaaggagcagatcagcaggaacagcaaggccctggagg | |
| | agaagtacgtggccgagctgcagctggagaggctgaagaaggacggcgaggtgaggggcagcatcaaca | |
| | ggttcaagaccagcgactacgtgaaggaggccaagcagctgctgaaggtgcagaaggcctaccaccagc | |
| | tggaccagagcttcatcgacacctacatcgacctgctggagaccaggaggacctactacgagggccccg | |
| | gcgagggcagccccttcggctggaaggacatcaaggagtggtacgagatgctgaagaggaggaggaggc | |
| | acaggatcctgagccccgtggtgaagaggagcttcatccagagcatcaaggtgatcaacgccatcatca | |
| | agaagtacggcctgcccaacgacatcatcatcgagctggccagggagaagaacagcaaggacgcccaga | |
| | agatgatcaacgagatgcagaagaggaacaggcagaccaacgagaggatcgaggagatcatcaggacca | |
| | ccggcaaggagaacgccaagtacctgatcgagaagatcaagctgcacgacatgcaggagggcaagtgcc | |
| | tgtacagcctggaggccatccccctggaggacctgctgaacaaccccttcaactacgaggtggaccaca | |
| | tcatccccaggagcgtgagcttcgacaacagcttcaacaacaaggtgctggtgaagcaggaggaggcca | |
| | gcaagaagggcaacaggacccccttccagtacctgagcagcagcgacagcaagatcagctacgagacct | |
| | tcaagaagcacatcctgaacctggccaagggcaagggcaggatcagcaagaccaagaaggagtacctgc | |
| | tggaggagagggacatcaacaggttcagcgtgcagaaggacttcatcaacaggaacctggtggacacca | |
| | ggtacgccaccaggggcctgatgaacctgctgaggagctacttcagggtgaacaacctggacgtgaagg | |
| | tgaagagcatcaacggcggcttcaccagcttcctgaggaggaagtggaagttcaagaaggagaggaaca | |
| | agggctacaagcaccacgccgaggacgccctgatcategccaacgccgacttcatcttcaaggagtgga | |
| | agaagctggacaaggccaagaaggtgatggagaaccagatgttcgaggagaagcaggccgagagcatgc | |
| | ccgagatcgagaccgagcaggagtacaaggagatcttcatcaccccccaccagatcaagcacatcaagg | |
| | acttcaaggactacaagtacagccacagggtggacaagaagcccaacaggaagctgatcaacgacaccc | |
| | tgtacagcaccaggaaggacgacaagggcaacaccctgatcgtgaacaacctgaacggcctgtacgaca | |
| | aggacaacgacaagctgaagaagctgatcaacaagagccccgagaagctgctgatgtaccaccacgacc | |
| | cccagacctaccagaagctgaagctgatcatggagcagtacggcgacgagaagaaccccctgtacaagt | |
| | actacgaggagaccggcaactacctgaccaagtacagcaagaaggacaacggccccgtgatcaagaaga | |
| | tcaagtactacggcaacaagctgaacgcccacctggacatcaccgacgactaccccaacagcaggaaca | |
| | aggtggtgaagctgagcctgaagccctacaggttcgacgtgtacctggacaacggcgtgtacaagttcg | |
| | tgaccgtgaagaacctggacgtgatcaagaaggagaactactacgaggtgaacagcaagtgctacgagg | |
| | aggccaagaagctgaagaagatcagcaaccaggccgagttcatcgccagcttctacaagaacgacctga | |
| | tcaagatcaacggcgagctgtacagggtgatcggcgtgaacaacgacctgctgaacaggatcgaggtga | |
| | acatgatcgacatcacctacagggagtacctggagaacatgaacgacaagaggcccccccacatcatca | |
| | agaccatcgccagcaagacccagagcatcaagaagtacagcaccgacatcctgggcaacctgtacgagg | |
| | tgaagagcaagaagcacccccagatcatcaagaagggcaaaaggccggcggccacgaaaaaggccggcc | |
| | aggcaaaaaagaaaaagggatccgaggccagcggttccggacgggctgacgcattggacgattttgatc | |
| | tggatatgctgggaagtgacgccctcgatgattttgaccttgacatgcttggttcggatgcccttgatg | |
| | actttgacctcgacatgctcggcagtgacgcccttgatgatttcgacctggacatgctgtgaatcgaat | |
| | tccgctcgagtggatcaacttgtttattgcagcttataatggttacaaataaagcaatagcatcacaaa | |
| | tttcacaaataaagcatttttttcactgcgcggccgcaggaacccctagtgatggagttggccactccc | |
| | tctctgcgcgctcgctcgctcactgaggccgggcgaccaaaggtcgcccgacgcccgggctttgcccgg | |
| | gcggcctcagtgagcgagcgagcgcgcag | |
| pAAV- | cctgcaggcagctgcgcgctcgctcgctcactgaggccgcccgggcgtcgggcgacctttggtcgcccg | 143 |
| SCN1A_SE2_mini- | gcctcagtgagcgagcgagcgcgcagagagggagtggccaactccatcactaggggttcctgcggccgc | |
| SadCas9-v4 | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctagcctgcaagtcgaggagcgcagccttccagaagcagagcgc | |
| | ggcgccttaagctgcagaagttggtcgtgaggcactgggcacgtaagtatcaaggttacaagacaggtt | |
| | taaggagaccaatagaaactgggcttgtcgagacagagaagactcttgcgtttctgataggcacctatt | |
| | ggtcttactgacatccactttgcctttctctccacagcgagctcgctagcgcgatcgctgccaccatgg | |
| | ccccaaagaagaagcggaaggtcggtatccacggagtcccagcagccaagcggaactacatcctgggcc | |
| | tggccatcggcatcaccagcgtgggctacggcatcatcgactacgagacacgggacgtgatcgatgccg | |
| | gcgtgcggctgttcaaagaggccaacgtggaaaacaacgagggcaggcggagcaagagaggcgccagaa | |
| | ggctgaagcggcggaggcggcatagaatccagagagtgaagaagctgctgttcgactacaacctgctga | |
| | ccgaccacagcgagctgagcggcatcaacccctacgaggccagagtgaagggcctgagccagaagctga | |
| | gcgaggaagagttctctgccgccctgctgcacctggccaagagaagaggcgtgcacaacgtgaacgagg | |
| | tggaagaggacaccggcaacgagctgtccaccaaagagcagatcagccggaacagcaaggccctggaag | |
| | agaaatacgtggccgaactgcagctggaacggctgaagaaagacggcgaagtgcggggcagcatcaaca | |
| | gattcaagaccagcgactacgtgaaagaagccaaacagctgctgaaggtgcagaaggcctaccaccagc | |
| | tggaccagagcttcatcgacacctacatcgacctgctggaaacccggcggacctactatgagggacctg | |
| | gcgagggcagccccttcggctggaaggacatcaaagaatggtacgagatgctgatgggccactgcacct | |
| | acttccccgaggaactgcggagcgtgaagtacgcctacaacgccgacctgtacaacgccctgaacgacc | |
| | tgaacaatctcgtgatcaccagggacgagaacgagaagctggaatattacgagaagttccagatcatcg | |
| | agaacgtgttcaagcagaagaagaagcccaccctgaagcagatcgccaaagaaatcctcgtgaacgaag | |
| | aggatattaagggctacagagtgaccagcaccggcaagcccgagttcaccaacctgaaggtgtaccacg | |
| | acatcaaggacattaccgcccggaaagagattattgagaacgccgagctgctggatcagattgccaaga | |
| | tcctgaccatctaccagagcagcgaggacatccaggaagaactgaccaatctgaactccgagctgaccc | |
| | aggaagagatcgagcagatctctaatctgaagggctataccggcacccacaacctgagcctgaaggcca | |
| | tcaacctgatcctggacgagctgtggcacaccaacgacaaccagatcgctatcttcaaccggctgaagc | |
| | tggtgcccaagaaggtggacctgtcccagcagaaagagatccccaccaccctggtggacgacttcatcc | |
| | tgagccccgtcgtgaagagaagcttcatccagagcatcaaagtgatcaacgccatcatcaagaagtacg | |
| | gcctgcccaacgacatcattatcgagctgggcagcaagagatacgccaccagaggcctgatgaacctgc | |
| | tgcggagctacttcagagtgaacaacctggacgtgaaagtgaagtccatcaatggcggcttcaccagct | |
| | ttctgcggcggaagtggaagtttaagaaagagcggaacaaggggtacaagcaccacgccgaggacgccc | |
| | tgatcattgccaacgccgatttcatcttcaaagagtggaagaaactggacaaggccaaaaaagtgatgg | |
| | aaaaccagatgttcgaggaaaagcaggccgagagcatgcccgagatcgaaaccgagcaggagtacaaag | |
| | agatcttcatcaccccccaccagatcaagcacattaaggacttcaaggactacaagtacagccaccggg | |
| | tggacaagaagcctaatagagagctgattaacgacaccctgtactccacccggaaggacgacaagggca | |
| | acaccctgatcgtgaacaatctgaacggcctgtacgacaaggacaatgacaagctgaaaaagctgatca | |
| | acaagagccccgaaaagctgctgatgtaccaccacgacccccagacctaccagaaactgaagctgatta | |
| | tggaacagtacggcgacgagaagaatcccctgtacaagtactacgaggaaaccgggaactacctgacca | |
| | agtactccaaaaaggacaacggccccgtgatcaagaagattaagtattacggcaacaaactgaacgccc | |
| | atctggacatcaccgacgactaccccaacagcagaaacaaggtcgtgaagctgtccctgaagccctaca | |
| | gattcgacgtgtacctggacaatggcgtgtacaagttcgtgaccgtgaagaatctggatgtgatcaaaa | |
| | aagaaaactactacgaagtgaatagcaagtgctatgaggaagctaagaagctgaagaagatcagcaacc | |
| | aggccgagtttatcgcctccttctacaacaacgatctgatcaagatcaacggcgagctgtatagagtga | |
| | tcggcgtgaacaacgacctgctgaaccggatcgaagtgaacatgatcgacatcacctaccgcgagtacc | |
| | tggaaaacatgaacgacaagaggccccccaggatcattaagacaatcgcctccaagacccagagcatta | |
| | agaagtacagcacagacattctgggcaacctgtatgaagtgaaatctaagaagcaccctcagatcatca | |
| | aaaagggcaaaaggccggcggccacgaaaaaggccggccaggcaaaaaagaaaaagggatccgaggcca | |
| | gcggttccggacgggctgacgcattggacgattttgatctggatatgctgggaagtgacgccctcgatg | |
| | attttgaccttgacatgcttggttcggatgcccttgatgactttgacctcgacatgctcggcagtgacg | |
| | cccttgatgatttcgacctggacatgctgtgaatcgaattccgctcgagtggatcaacttgtttattgc | |
| | agcttataatggttacaaataaagcaatagcatcacaaatttcacaaataaagcatttttttcactgcg | |
| | cggccgcaggaacccctagtgatggagttggccactccctctctgcgcgctcgctcgctcactgaggcc | |
| | gggcgaccaaaggtcgcccgacgcccgggctttgcccgggcggcctcagtgagcgagcgagcgcgcag | |
| pAAV- | cctgcaggcagctgcgcgctcgctcgctcactgaggccgcccgggcgtcgggcgacctttggtcgcccg | 144 |
| SCN1A_SE2_mini- | gcctcagtgagcgagcgagcgcgcagagagggagtggccaactccatcactaggggttcctgcggccgc | |
| SadCas9-v5 | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctagcctgcaagtcgaggagcgcagccttccagaagcagagcgc | |
| | ggcgccttaagctgcagaagttggtcgtgaggcactgggcacgtaagtatcaaggttacaagacaggtt | |
| | taaggagaccaatagaaactgggcttgtcgagacagagaagactcttgcgtttctgataggcacctatt | |
| | ggtcttactgacatccactttgcctttctctccacagcgagctcgctagcgcgatcgctgccaccatgg | |
| | ccccaaagaagaagcggaaggtcggtatccacggagtcccagcagccaagaggaactacatcctgggcc | |
| | tggccatcggcatcaccagcgtgggctacggcatcatcgactacgagaccagggacgtgatcgacgccg | |
| | gcgtgaggctgttcaaggaggccaacgtggagaacaacgagggcaggaggagcaagaggggcgccagga | |
| | ggctgaagaggaggaggaggcacaggatccagagggtgaagaagctgctgttcgactacaacctgctga | |
| | ccgaccacagcgagctgagcggcatcaacccctacgaggccagggtgaagggcctgagccagaagctga | |
| | gcgaggaggagttcagcgccgccctgctgcacctggccaagaggaggggcgtgcacaacgtgaacgagg | |
| | tggaggaggacaccggcaacgagctgagcaccaaggagcagatcagcaggaacagcaaggccctggagg | |
| | agaagtacgtggccgagctgcagctggagaggctgaagaaggacggcgaggtgaggggcagcatcaaca | |
| | ggttcaagaccagcgactacgtgaaggaggccaagcagctgctgaaggtgcagaaggcctaccaccagc | |
| | tggaccagagcttcatcgacacctacatcgacctgctggagaccaggaggacctactacgagggccccg | |
| | gcgagggcagccccttcggctggaaggacatcaaggagtggtacgagatgctgaagaggaggaggaggc | |
| | acaggatcctgagccccgtggtgaagaggagcttcatccagagcatcaaggtgatcaacgccatcatca | |
| | agaagtacggcctgcccaacgacatcatcatcgagctgggcagcaagaggtacgccaccaggggcctga | |
| | tgaacctgctgaggagctacttcagggtgaacaacctggacgtgaaggtgaagagcatcaacggcggct | |
| | tcaccagcttcctgaggaggaagtggaagttcaagaaggagaggaacaagggctacaagcaccacgccg | |
| | aggacgccctgatcatcgccaacgccgacttcatcttcaaggagtggaagaagctggacaaggccaaga | |
| | aggtgatggagaaccagatgttcgaggagaagcaggccgagagcatgcccgagatcgagaccgagcagg | |
| | agtacaaggagatcttcatcaccccccaccagatcaagcacatcaaggacttcaaggactacaagtaca | |
| | gccacagggtggacaagaagcccaacaggaagctgatcaacgacaccctgtacagcaccaggaaggacg | |
| | acaagggcaacaccctgatcgtgaacaacctgaacggcctgtacgacaaggacaacgacaagctgaaga | |
| | agctgatcaacaagagccccgagaagctgctgatgtaccaccacgacccccagacctaccagaagctga | |
| | agctgatcatggagcagtacggcgacgagaagaaccccctgtacaagtactacgaggagaccggcaact | |
| | acctgaccaagtacagcaagaaggacaacggccccgtgatcaagaagatcaagtactacggcaacaagc | |
| | tgaacgcccacctggacatcaccgacgactaccccaacagcaggaacaaggtggtgaagctgagcctga | |
| | agccctacaggttcgacgtgtacctggacaacggcgtgtacaagttcgtgaccgtgaagaacctggacg | |
| | tgatcaagaaggagaactactacgaggtgaacagcaagtgctacgaggaggccaagaagctgaagaaga | |
| | tcagcaaccaggccgagttcatcgccagcttctacaagaacgacctgatcaagatcaacggcgagctgt | |
| | acagggtgatcggcgtgaacaacgacctgctgaacaggatcgaggtgaacatgatcgacatcacctaca | |
| | gggagtacctggagaacatgaacgacaagaggcccccccacatcatcaagaccatcgccagcaagaccc | |
| | agagcatcaagaagtacagcaccgacatcctgggcaacctgtacgaggtgaagagcaagaagcaccccc | |
| | agatcatcaagaagggcaaaaggccggcggccacgaaaaaggccggccaggcaaaaaagaaaaagggat | |
| | ccgaggccagcggttccggacgggctgacgcattggacgattttgatctggatatgctgggaagtgacg | |
| | ccctcgatgattttgaccttgacatgcttggttcggatgcccttgatgactttgacctcgacatgctcg | |
| | gcagtgacgcccttgatgatttcgacctggacatgctgtgaatcgaattccgctcgagtggatcaactt | |
| | gtttattgcagcttataatggttacaaataaagcaatagcatcacaaatttcacaaataaagcattttt | |
| | ttcactgcgcggccgcaggaacccctagtgatggagttggccactccctctctgcgcgctcgctcgctc | |
| | actgaggccgggcgaccaaaggtcgcccgacgcccgggctttgcccgggcggcctcagtgagcgagcga | |
| | gcgcgcag | |
| pAAV-SCN1A_SE3 | cctgcaggcagctgcgcgctcgctcgctcactgaggccgcccgggcgtcgggcgacctttggtcgcccg | 145 |
| | gcctcagtgagcgagcgagcgcgcagagagggagtggccaactccatcactaggggttcctgcggccgc | |
| | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctgagctcgctagcgcgatcgctgccaccatggccccaaagaag | |
| | aagcggaaggtcggtatccacggagtcccagcagccaagcggaactacatcctgggcctggccatcggc | |
| | atcaccagcgtgggctacggcatcatcgactacgagacacgggacgtgatcgatgccggcgtgcggctg | |
| | ttcaaagaggccaacgtggaaaacaacgagggcaggcggagcaagagaggcgccagaaggctgaagcgg | |
| | cggaggcggcatagaatccagagagtgaagaagctgctgttcgactacaacctgctgaccgaccacagc | |
| | gagctgagcggcatcaacccctacgaggccagagtgaagggcctgagccagaagctgagcgaggaagag | |
| | ttctctgccgccctgctgcacctggccaagagaagaggcgtgcacaacgtgaacgaggtggaagaggac | |
| | accggcaacgagctgtccaccaaagagcagatcagccggaacagcaaggccctggaagagaaatacgtg | |
| | gccgaactgcagctggaacggctgaagaaagacggcgaagtgcggggcagcatcaacagattcaagacc | |
| | agcgactacgtgaaagaagccaaacagctgctgaaggtgcagaaggcctaccaccagctggaccagagc | |
| | ttcatcgacacctacatcgacctgctggaaacccggcggacctactatgagggacctggcgagggcagc | |
| | cccttcggctggaaggacatcaaagaatggtacgagatgctgatgggccactgcacctacttccccgag | |
| | gaactgcggagcgtgaagtacgcctacaacgccgacctgtacaacgccctgaacgacctgaacaatctc | |
| | gtgatcaccagggacgagaacgagaagctggaatattacgagaagttccagatcatcgagaacgtgttc | |
| | aagcagaagaagaagcccaccctgaagcagatcgccaaagaaatcctcgtgaacgaagaggatattaag | |
| | ggctacagagtgaccagcaccggcaagcccgagttcaccaacctgaaggtgtaccacgacatcaaggac | |
| | attaccgcccggaaagagattattgagaacgccgagctgctggatcagattgccaagatcctgaccatc | |
| | taccagagcagcgaggacatccaggaagaactgaccaatctgaactccgagctgacccaggaagagatc | |
| | gagcagatctctaatctgaagggctataccggcacccacaacctgagcctgaaggccatcaacctgatc | |
| | ctggacgagctgtggcacaccaacgacaaccagatcgctatcttcaaccggctgaagctggtgcccaag | |
| | aaggtggacctgtcccagcagaaagagatccccaccaccctggtggacgacttcatcctgagccccgtc | |
| | gtgaagagaagcttcatccagagcatcaaagtgatcaacgccatcatcaagaagtacggcctgcccaac | |
| | gacatcattatcgagctggcccgcgagaagaactccaaggacgcccagaaaatgatcaacgagatgcag | |
| | aagcggaaccggcagaccaacgagcggatcgaggaaatcatccggaccaccggcaaagagaacgccaag | |
| | tacctgatcgagaagatcaagctgcacgacatgcaggaaggcaagtgcctgtacagcctggaagccatc | |
| | cctctggaagatctgctgaacaaccccttcaactatgaggtggaccacatcatccccagaagcgtgtcc | |
| | ttcgacaacagcttcaacaacaaggtgctcgtgaagcaggaagaagccagcaagaagggcaaccggacc | |
| | ccattccagtacctgagcagcagcgacagcaagatcagctacgaaaccttcaagaagcacatcctgaat | |
| | ctggccaagggcaagggcagaatcagcaagaccaagaaagagtatctgctggaagaacgggacatcaac | |
| | aggttctccgtgcagaaagacttcatcaaccggaacctggtggataccagatacgccaccagaggcctg | |
| | atgaacctgctgcggagctacttcagagtgaacaacctggacgtgaaagtgaagtccatcaatggcggc | |
| | ttcaccagctttctgcggcggaagtggaagtttaagaaagagcggaacaaggggtacaagcaccacgcc | |
| | gaggacgccctgatcattgccaacgccgatttcatcttcaaagagtggaagaaactggacaaggccaaa | |
| | aaagtgatggaaaaccagatgttcgaggaaaagcaggccgagagcatgcccgagatcgaaaccgagcag | |
| | gagtacaaagagatcttcatcaccccccaccagatcaagcacattaaggacttcaaggactacaagtac | |
| | agccaccgggtggacaagaagcctaatagagagctgattaacgacaccctgtactccacccggaaggac | |
| | gacaagggcaacaccctgatcgtgaacaatctgaacggcctgtacgacaaggacaatgacaagctgaaa | |
| | aagctgatcaacaagagccccgaaaagctgctgatgtaccaccacgacccccagacctaccagaaactg | |
| | aagctgattatggaacagtacggcgacgagaagaatcccctgtacaagtactacgaggaaaccgggaac | |
| | tacctgaccaagtactccaaaaaggacaacggccccgtgatcaagaagattaagtattacggcaacaaa | |
| | ctgaacgcccatctggacatcaccgacgactaccccaacagcagaaacaaggtcgtgaagctgtccctg | |
| | aagccctacagattcgacgtgtacctggacaatggcgtgtacaagttcgtgaccgtgaagaatctggat | |
| | gtgatcaaaaaagaaaactactacgaagtgaatagcaagtgctatgaggaagctaagaagctgaagaag | |
| | atcagcaaccaggccgagtttatcgcctccttctacaacaacgatctgatcaagatcaacggcgagctg | |
| | tatagagtgatcggcgtgaacaacgacctgctgaaccggatcgaagtgaacatgatcgacatcacctac | |
| | cgcgagtacctggaaaacatgaacgacaagaggccccccaggatcattaagacaatcgcctccaagacc | |
| | cagagcattaagaagtacagcacagacattctgggcaacctgtatgaagtgaaatctaagaagcaccct | |
| | cagatcatcaaaaagggcaaaaggccggcggccacgaaaaaggccggccaggcaaaaaagaaaaaggga | |
| | tccgaggccagcggttccggacgggctgacgcattggacgattttgatctggatatgctgggaagtgac | |
| | gccctcgatgattttgaccttgacatgcttggttcggatgcccttgatgactttgacctcgacatgctc | |
| | ggcagtgacgcccttgatgatttcgacctggacatgctgtgaatcgaattccgctcgagataatcaacc | |
| | tctggattacaaaatttgtgaaagattgactggtattcttaactatgttgctccttttacgctatgtgg | |
| | atacgctgctttaatgcctttgtatcatgctattgcttcccgtatggctttcattttctcctccttgta | |
| | taaatcctggttagttcttgccacggcggaactcatcgccgcctgccttgcccgctgctggacaggggc | |
| | tcggctgttgggcactgacaattccgtggtgttctggatcaacttgtttattgcagcttataatggtta | |
| | caaataaagcaatagcatcacaaatttcacaaataaagcatttttttcactgcgcggccgcaggaaccc | |
| | ctagtgatggagttggccactccctctctgcgcgctcgctcgctcactgaggccgggcgaccaaaggtc | |
| | gcccgacgcccgggctttgcccgggcggcctcagtgagcgagcgagcgcgcag | |
| pAAV- | cctgcaggcagctgcgcgctcgctcgctcactgaggccgcccgggcgtcgggcgacctttggtcgcccg | 146 |
| SCN1A_SE3_mini- | gcctcagtgagcgagcgagcgcgcagagagggagtggccaactccatcactaggggttcctgcggccgc | |
| SadCas9-v2 | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctgagctcgctagcgcgatcgctgccaccatggccccaaagaag | |
| | aagcggaaggtcggtatccacggagtcccagcagccaagaggaactacatcctgggcctggccatcggc | |
| | atcaccagcgtgggctacggcatcatcgactacgagaccagggacgtgatcgacgccggcgtgaggctg | |
| | ttcaaggaggccaacgtggagaacaacgagggcaggaggagcaagaggggcgccaggaggctgaagagg | |
| | aggaggaggcacaggatccagagggtgaagaagctgctgttcgactacaacctgctgaccgaccacagc | |
| | gagctgagcggcatcaacccctacgaggccagggtgaagggcctgagccagaagctgagcgaggaggag | |
| | ttcagcgccgccctgctgcacctggccaagaggaggggcgtgcacaacgtgaacgaggtggaggaggac | |
| | accggcaacgagctgagcaccaaggagcagatcagcaggaacagcaaggccctggaggagaagtacgtg | |
| | gccgagctgcagctggagaggctgaagaaggacggcgaggtgaggggcagcatcaacaggttcaagacc | |
| | agcgactacgtgaaggaggccaagcagctgctgaaggtgcagaaggcctaccaccagctggaccagagc | |
| | ttcatcgacacctacatcgacctgctggagaccaggaggacctactacgagggccccggcgagggcagc | |
| | cccttcggctggaaggacatcaaggagtggtacgagatgctgaagaggaggaggaggcacaggatcctg | |
| | agccccgtggtgaagaggagcttcatccagagcatcaaggtgatcaacgccatcatcaagaagtacggc | |
| | ctgcccaacgacatcatcatcgagctggccagggagaagaacagcaaggacgcccagaagatgatcaac | |
| | gagatgcagaagaggaacaggcagaccaacgagaggatcgaggagatcatcaggaccaccggcaaggag | |
| | aacgccaagtacctgatcgagaagatcaagctgcacgacatgcaggagggcaagtgcctgtacagcctg | |
| | gaggccatccccctggaggacctgctgaacaaccccttcaactacgaggtggaccacatcatccccagg | |
| | agcgtgagcttcgacaacagcttcaacaacaaggtgctggtgaagcaggaggaggccagcaagaagggc | |
| | aacaggacccccttccagtacctgagcagcagcgacagcaagatcagctacgagaccttcaagaagcac | |
| | atcctgaacctggccaagggcaagggcaggatcagcaagaccaagaaggagtacctgctggaggagagg | |
| | gacatcaacaggttcagcgtgcagaaggacttcatcaacaggaacctggtggacaccaggtacgccacc | |
| | aggggcctgatgaacctgctgaggagctacttcagggtgaacaacctggacgtgaaggtgaagagcatc | |
| | aacggcggcttcaccagcttcctgaggaggaagtggaagttcaagaaggagaggaacaagggctacaag | |
| | caccacgccgaggacgccctgatcatcgccaacgccgacttcatcttcaaggagtggaagaagctggac | |
| | aaggccaagaaggtgatggagaaccagatgttcgaggagaagcaggccgagagcatgcccgagatcgag | |
| | accgagcaggagtacaaggagatcttcatcaccccccaccagatcaagcacatcaaggacttcaaggac | |
| | tacaagtacagccacagggtggacaagaagcccaacaggaagctgatcaacgacaccctgtacagcacc | |
| | aggaaggacgacaagggcaacaccctgatcgtgaacaacctgaacggcctgtacgacaaggacaacgac | |
| | aagctgaagaagctgatcaacaagagccccgagaagctgctgatgtaccaccacgacccccagacctac | |
| | cagaagctgaagctgatcatggagcagtacggcgacgagaagaaccccctgtacaagtactacgaggag | |
| | accggcaactacctgaccaagtacagcaagaaggacaacggccccgtgatcaagaagatcaagtactac | |
| | ggcaacaagctgaacgcccacctggacatcaccgacgactaccccaacagcaggaacaaggtggtgaag | |
| | ctgagcctgaagccctacaggttcgacgtgtacctggacaacggcgtgtacaagttcgtgaccgtgaag | |
| | aacctggacgtgatcaagaaggagaactactacgaggtgaacagcaagtgctacgaggaggccaagaag | |
| | ctgaagaagatcagcaaccaggccgagttcatcgccagcttctacaagaacgacctgatcaagatcaac | |
| | ggcgagctgtacagggtgatcggcgtgaacaacgacctgctgaacaggatcgaggtgaacatgatcgac | |
| | atcacctacagggagtacctggagaacatgaacgacaagaggcccccccacatcatcaagaccatcgcc | |
| | agcaagacccagagcatcaagaagtacagcaccgacatcctgggcaacctgtacgaggtgaagagcaag | |
| | aagcacccccagatcatcaagaagggcaaaaggccggcggccacgaaaaaggccggccaggcaaaaaag | |
| | aaaaagggatccgaggccagcggttccggacgggctgacgcattggacgattttgatctggatatgctg | |
| | ggaagtgacgccctcgatgattttgaccttgacatgcttggttcggatgcccttgatgactttgacctc | |
| | gacatgctcggcagtgacgcccttgatgatttcgacctggacatgctgtgaatcgaattccgctcgaga | |
| | taatcaacctctggattacaaaatttgtgaaagattgactggtattcttaactatgttgctccttttac | |
| | gctatgtggatacgctgctttaatgcctttgtatcatgctattgcttcccgtatggctttcattttctc | |
| | ctccttgtataaatcctggttagttcttgccacggcggaactcatcgccgcctgccttgcccgctgctg | |
| | gacaggggctcggctgttgggcactgacaattccgtggtgttctggatcaacttgtttattgcagctta | |
| | taatggttacaaataaagcaatagcatcacaaatttcacaaataaagcatttttttcactgcgcggccg | |
| | caggaacccctagtgatggagttggccactccctctctgcgcgctcgctcgctcactgaggccgggcga | |
| | ccaaaggtcgcccgacgcccgggctttgcccgggcggcctcagtgagcgagcgagcgcgcag | |
| pAAV- | cctgcaggcagctgcgcgctcgctcgctcactgaggccgcccgggcgtcgggcgacctttggtcgcccg | 147 |
| SCN1A_SE3_mini- | gcctcagtgagcgagcgagcgcgcagagagggagtggccaactccatcactaggggttcctgcggccgc | |
| SadCas9-v4 | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctgagctcgctagcgcgatcgctgccaccatggccccaaagaag | |
| | aagcggaaggtcggtatccacggagtcccagcagccaagcggaactacatcctgggcctggccatcggc | |
| | atcaccagcgtgggctacggcatcatcgactacgagacacgggacgtgatcgatgccggcgtgcggctg | |
| | ttcaaagaggccaacgtggaaaacaacgagggcaggcggagcaagagaggcgccagaaggctgaagcgg | |
| | cggaggcggcatagaatccagagagtgaagaagctgctgttcgactacaacctgctgaccgaccacagc | |
| | gagctgagcggcatcaacccctacgaggccagagtgaagggcctgagccagaagctgagcgaggaagag | |
| | ttctctgccgccctgctgcacctggccaagagaagaggcgtgcacaacgtgaacgaggtggaagaggac | |
| | accggcaacgagctgtccaccaaagagcagatcagccggaacagcaaggccctggaagagaaatacgtg | |
| | gccgaactgcagctggaacggctgaagaaagacggcgaagtgcggggcagcatcaacagattcaagacc | |
| | agcgactacgtgaaagaagccaaacagctgctgaaggtgcagaaggcctaccaccagctggaccagagc | |
| | ttcatcgacacctacatcgacctgctggaaacccggcggacctactatgagggacctggcgagggcagc | |
| | cccttcggctggaaggacatcaaagaatggtacgagatgctgatgggccactgcacctacttccccgag | |
| | gaactgcggagcgtgaagtacgcctacaacgccgacctgtacaacgccctgaacgacctgaacaatctc | |
| | gtgatcaccagggacgagaacgagaagctggaatattacgagaagttccagatcatcgagaacgtgttc | |
| | aagcagaagaagaagcccaccctgaagcagatcgccaaagaaatcctcgtgaacgaagaggatattaag | |
| | ggctacagagtgaccagcaccggcaagcccgagttcaccaacctgaaggtgtaccacgacatcaaggac | |
| | attaccgcccggaaagagattattgagaacgccgagctgctggatcagattgccaagatcctgaccatc | |
| | taccagagcagcgaggacatccaggaagaactgaccaatctgaactccgagctgacccaggaagagatc | |
| | gagcagatctctaatctgaagggctataccggcacccacaacctgagcctgaaggccatcaacctgatc | |
| | ctggacgagctgtggcacaccaacgacaaccagatcgctatcttcaaccggctgaagctggtgcccaag | |
| | aaggtggacctgtcccagcagaaagagatccccaccaccctggtggacgacttcatcctgagccccgtc | |
| | gtgaagagaagcttcatccagagcatcaaagtgatcaacgccatcatcaagaagtacggcctgcccaac | |
| | gacatcattatcgagctgggcagcaagagatacgccaccagaggcctgatgaacctgctgcggagctac | |
| | ttcagagtgaacaacctggacgtgaaagtgaagtccatcaatggcggcttcaccagctttctgcggcgg | |
| | aagtggaagtttaagaaagagcggaacaaggggtacaagcaccacgccgaggacgccctgatcattgcc | |
| | aacgccgatttcatcttcaaagagtggaagaaactggacaaggccaaaaaagtgatggaaaaccagatg | |
| | ttcgaggaaaagcaggccgagagcatgcccgagatcgaaaccgagcaggagtacaaagagatcttcatc | |
| | accccccaccagatcaagcacattaaggacttcaaggactacaagtacagccaccgggtggacaagaag | |
| | cctaatagagagctgattaacgacaccctgtactccacccggaaggacgacaagggcaacaccctgatc | |
| | gtgaacaatctgaacggcctgtacgacaaggacaatgacaagctgaaaaagctgatcaacaagagcccc | |
| | gaaaagctgctgatgtaccaccacgacccccagacctaccagaaactgaagctgattatggaacagtac | |
| | ggcgacgagaagaatcccctgtacaagtactacgaggaaaccgggaactacctgaccaagtactccaaa | |
| | aaggacaacggccccgtgatcaagaagattaagtattacggcaacaaactgaacgcccatctggacatc | |
| | accgacgactaccccaacagcagaaacaaggtcgtgaagctgtccctgaagccctacagattcgacgtg | |
| | tacctggacaatggcgtgtacaagttcgtgaccgtgaagaatctggatgtgatcaaaaaagaaaactac | |
| | tacgaagtgaatagcaagtgctatgaggaagctaagaagctgaagaagatcagcaaccaggccgagttt | |
| | atcgcctccttctacaacaacgatctgatcaagatcaacggcgagctgtatagagtgatcggcgtgaac | |
| | aacgacctgctgaaccggatcgaagtgaacatgatcgacatcacctaccgcgagtacctggaaaacatg | |
| | aacgacaagaggccccccaggatcattaagacaatcgcctccaagacccagagcattaagaagtacagc | |
| | acagacattctgggcaacctgtatgaagtgaaatctaagaagcaccctcagatcatcaaaaagggcaaa | |
| | aggccggcggccacgaaaaaggccggccaggcaaaaaagaaaaagggatccgaggccagcggttccgga | |
| | cgggctgacgcattggacgattttgatctggatatgctgggaagtgacgccctcgatgattttgacctt | |
| | gacatgcttggttcggatgcccttgatgactttgacctcgacatgctcggcagtgacgcccttgatgat | |
| | ttcgacctggacatgctgtgaatcgaattccgctcgagataatcaacctctggattacaaaatttgtga | |
| | aagattgactggtattcttaactatgttgctccttttacgctatgtggatacgctgctttaatgccttt | |
| | gtatcatgctattgcttcccgtatggctttcattttctcctccttgtataaatcctggttagttcttgc | |
| | cacggcggaactcatcgccgcctgccttgcccgctgctggacaggggctcggctgttgggcactgacaa | |
| | ttccgtggtgttctggatcaacttgtttattgcagcttataatggttacaaataaagcaatagcatcac | |
| | aaatttcacaaataaagcatttttttcactgcgcggccgcaggaacccctagtgatggagttggccact | |
| | ccctctctgcgcgctcgctcgctcactgaggccgggcgaccaaaggtcgcccgacgcccgggctttgcc | |
| | cgggcggcctcagtgagcgagcgagcgcgcag | |
| pAAV- | cctgcaggcagctgcgcgctcgctcgctcactgaggccgcccgggcgtcgggcgacctttggtcgcccg | 148 |
| SCN1A_SE3_mini- | gcctcagtgagcgagcgagcgcgcagagagggagtggccaactccatcactaggggttcctgcggccgc | |
| SadCas9-v5 | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctgagctcgctagcgcgatcgctgccaccatggccccaaagaag | |
| | aagcggaaggtcggtatccacggagtcccagcagccaagaggaactacatcctgggcctggccatcggc | |
| | atcaccagcgtgggctacggcatcatcgactacgagaccagggacgtgatcgacgccggcgtgaggctg | |
| | ttcaaggaggccaacgtggagaacaacgagggcaggaggagcaagaggggcgccaggaggctgaagagg | |
| | aggaggaggcacaggatccagagggtgaagaagctgctgttcgactacaacctgctgaccgaccacagc | |
| | gagctgagcggcatcaacccctacgaggccagggtgaagggcctgagccagaagctgagcgaggaggag | |
| | ttcagcgccgccctgctgcacctggccaagaggaggggcgtgcacaacgtgaacgaggtggaggaggac | |
| | accggcaacgagctgagcaccaaggagcagatcagcaggaacagcaaggccctggaggagaagtacgtg | |
| | gccgagctgcagctggagaggctgaagaaggacggcgaggtgaggggcagcatcaacaggttcaagacc | |
| | agcgactacgtgaaggaggccaagcagctgctgaaggtgcagaaggcctaccaccagctggaccagagc | |
| | ttcatcgacacctacatcgacctgctggagaccaggaggacctactacgagggccccggcgagggcagc | |
| | cccttcggctggaaggacatcaaggagtggtacgagatgctgaagaggaggaggaggcacaggatcctg | |
| | agccccgtggtgaagaggagcttcatccagagcatcaaggtgatcaacgccatcatcaagaagtacggc | |
| | ctgcccaacgacatcatcatcgagctgggcagcaagaggtacgccaccaggggcctgatgaacctgctg | |
| | aggagctacttcagggtgaacaacctggacgtgaaggtgaagagcatcaacggcggcttcaccagcttc | |
| | ctgaggaggaagtggaagttcaagaaggagaggaacaagggctacaagcaccacgccgaggacgccctg | |
| | atcatcgccaacgccgacttcatcttcaaggagtggaagaagctggacaaggccaagaaggtgatggag | |
| | aaccagatgttcgaggagaagcaggccgagagcatgcccgagatcgagaccgagcaggagtacaaggag | |
| | atcttcatcaccccccaccagatcaagcacatcaaggacttcaaggactacaagtacagccacagggtg | |
| | gacaagaagcccaacaggaagctgatcaacgacaccctgtacagcaccaggaaggacgacaagggcaac | |
| | accctgatcgtgaacaacctgaacggcctgtacgacaaggacaacgacaagctgaagaagctgatcaac | |
| | aagagccccgagaagctgctgatgtaccaccacgacccccagacctaccagaagctgaagctgatcatg | |
| | gagcagtacggcgacgagaagaaccccctgtacaagtactacgaggagaccggcaactacctgaccaag | |
| | tacagcaagaaggacaacggccccgtgatcaagaagatcaagtactacggcaacaagctgaacgcccac | |
| | ctggacatcaccgacgactaccccaacagcaggaacaaggtggtgaagctgagcctgaagccctacagg | |
| | ttcgacgtgtacctggacaacggcgtgtacaagttcgtgaccgtgaagaacctggacgtgatcaagaag | |
| | gagaactactacgaggtgaacagcaagtgctacgaggaggccaagaagctgaagaagatcagcaaccag | |
| | gccgagttcatcgccagcttctacaagaacgacctgatcaagatcaacggcgagctgtacagggtgatc | |
| | ggcgtgaacaacgacctgctgaacaggatcgaggtgaacatgatcgacatcacctacagggagtacctg | |
| | gagaacatgaacgacaagaggcccccccacatcatcaagaccatcgccagcaagacccagagcatcaag | |
| | aagtacagcaccgacatcctgggcaacctgtacgaggtgaagagcaagaagcacccccagatcatcaag | |
| | aagggcaaaaggccggcggccacgaaaaaggccggccaggcaaaaaagaaaaagggatccgaggccagc | |
| | ggttccggacgggctgacgcattggacgattttgatctggatatgctgggaagtgacgccctcgatgat | |
| | tttgaccttgacatgcttggttcggatgcccttgatgactttgacctcgacatgctcggcagtgacgcc | |
| | cttgatgatttcgacctggacatgctgtgaatcgaattccgctcgagataatcaacctctggattacaa | |
| | aatttgtgaaagattgactggtattcttaactatgttgctccttttacgctatgtggatacgctgcttt | |
| | aatgcctttgtatcatgctattgcttcccgtatggctttcattttctcctccttgtataaatcctggtt | |
| | agttcttgccacggcggaactcatcgccgcctgccttgcccgctgctggacaggggctcggctgttggg | |
| | cactgacaattccgtggtgttctggatcaacttgtttattgcagcttataatggttacaaataaagcaa | |
| | tagcatcacaaatttcacaaataaagcatttttttcactgcgcggccgcaggaacccctagtgatggag | |
| | ttggccactccctctctgcgcgctcgctcgctcactgaggccgggcgaccaaaggtcgcccgacgcccg | |
| | ggctttgcccgggcggcctcagtgagcgagcgagcgcgcag | |
| pAAV-SCN1A_SE4 | cctgcaggcagctgcgcgctcgctcgctcactgaggccgcccgggcgtcgggcgacctttggtcgcccg | 149 |
| | gcctcagtgagcgagcgagcgcgcagagagggagtggccaactccatcactaggggttcctgcggccgc | |
| | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctgagctcgctagcgcgatcgctgccaccatggccccaaagaag | |
| | aagcggaaggtcggtatccacggagtcccagcagccaagcggaactacatcctgggcctggccatcggc | |
| | atcaccagcgtgggctacggcatcatcgactacgagacacgggacgtgategatgccggcgtgcggctg | |
| | ttcaaagaggccaacgtggaaaacaacgagggcaggcggagcaagagaggcgccagaaggctgaagcgg | |
| | cggaggcggcatagaatccagagagtgaagaagctgctgttcgactacaacctgctgaccgaccacagc | |
| | gagctgagcggcatcaacccctacgaggccagagtgaagggcctgagccagaagctgagcgaggaagag | |
| | ttctctgccgccctgctgcacctggccaagagaagaggcgtgcacaacgtgaacgaggtggaagaggac | |
| | accggcaacgagctgtccaccaaagagcagatcagccggaacagcaaggccctggaagagaaatacgtg | |
| | gccgaactgcagctggaacggctgaagaaagacggcgaagtgcggggcagcatcaacagattcaagacc | |
| | agcgactacgtgaaagaagccaaacagctgctgaaggtgcagaaggcctaccaccagctggaccagagc | |
| | ttcatcgacacctacatcgacctgctggaaacccggcggacctactatgagggacctggcgagggcagc | |
| | cccttcggctggaaggacatcaaagaatggtacgagatgctgatgggccactgcacctacttccccgag | |
| | gaactgcggagcgtgaagtacgcctacaacgccgacctgtacaacgccctgaacgacctgaacaatctc | |
| | gtgatcaccagggacgagaacgagaagctggaatattacgagaagttccagatcatcgagaacgtgttc | |
| | aagcagaagaagaagcccaccctgaagcagatcgccaaagaaatcctcgtgaacgaagaggatattaag | |
| | ggctacagagtgaccagcaccggcaagcccgagttcaccaacctgaaggtgtaccacgacatcaaggac | |
| | attaccgcccggaaagagattattgagaacgccgagctgctggatcagattgccaagatcctgaccatc | |
| | taccagagcagcgaggacatccaggaagaactgaccaatctgaactccgagctgacccaggaagagatc | |
| | gagcagatctctaatctgaagggctataccggcacccacaacctgagcctgaaggccatcaacctgatc | |
| | ctggacgagctgtggcacaccaacgacaaccagatcgctatcttcaaccggctgaagctggtgcccaag | |
| | aaggtggacctgtcccagcagaaagagatccccaccaccctggtggacgacttcatcctgagccccgtc | |
| | gtgaagagaagcttcatccagagcatcaaagtgatcaacgccatcatcaagaagtacggcctgcccaac | |
| | gacatcattatcgagctggcccgcgagaagaactccaaggacgcccagaaaatgatcaacgagatgcag | |
| | aagcggaaccggcagaccaacgagcggatcgaggaaatcatccggaccaccggcaaagagaacgccaag | |
| | tacctgatcgagaagatcaagctgcacgacatgcaggaaggcaagtgcctgtacagcctggaagccatc | |
| | cctctggaagatctgctgaacaaccccttcaactatgaggtggaccacatcatccccagaagcgtgtcc | |
| | ttcgacaacagcttcaacaacaaggtgctcgtgaagcaggaagaagccagcaagaagggcaaccggacc | |
| | ccattccagtacctgagcagcagcgacagcaagatcagctacgaaaccttcaagaagcacatcctgaat | |
| | ctggccaagggcaagggcagaatcagcaagaccaagaaagagtatctgctggaagaacgggacatcaac | |
| | aggttctccgtgcagaaagacttcatcaaccggaacctggtggataccagatacgccaccagaggcctg | |
| | atgaacctgctgcggagctacttcagagtgaacaacctggacgtgaaagtgaagtccatcaatggcggc | |
| | ttcaccagctttctgcggcggaagtggaagtttaagaaagagcggaacaaggggtacaagcaccacgcc | |
| | gaggacgccctgatcattgccaacgccgatttcatcttcaaagagtggaagaaactggacaaggccaaa | |
| | aaagtgatggaaaaccagatgttcgaggaaaagcaggccgagagcatgcccgagatcgaaaccgagcag | |
| | gagtacaaagagatcttcatcaccccccaccagatcaagcacattaaggacttcaaggactacaagtac | |
| | agccaccgggtggacaagaagcctaatagagagctgattaacgacaccctgtactccacccggaaggac | |
| | gacaagggcaacaccctgatcgtgaacaatctgaacggcctgtacgacaaggacaatgacaagctgaaa | |
| | aagctgatcaacaagagccccgaaaagctgctgatgtaccaccacgacccccagacctaccagaaactg | |
| | aagctgattatggaacagtacggcgacgagaagaatcccctgtacaagtactacgaggaaaccgggaac | |
| | tacctgaccaagtactccaaaaaggacaacggccccgtgatcaagaagattaagtattacggcaacaaa | |
| | ctgaacgcccatctggacatcaccgacgactaccccaacagcagaaacaaggtcgtgaagctgtccctg | |
| | aagccctacagattcgacgtgtacctggacaatggcgtgtacaagttcgtgaccgtgaagaatctggat | |
| | gtgatcaaaaaagaaaactactacgaagtgaatagcaagtgctatgaggaagctaagaagctgaagaag | |
| | atcagcaaccaggccgagtttatcgcctccttctacaacaacgatctgatcaagatcaacggcgagctg | |
| | tatagagtgatcggcgtgaacaacgacctgctgaaccggatcgaagtgaacatgatcgacatcacctac | |
| | cgcgagtacctggaaaacatgaacgacaagaggccccccaggatcattaagacaatcgcctccaagacc | |
| | cagagcattaagaagtacagcacagacattctgggcaacctgtatgaagtgaaatctaagaagcaccct | |
| | cagatcatcaaaaagggcaaaaggccggcggccacgaaaaaggccggccaggcaaaaaagaaaaaggga | |
| | tccgaggccagcggttccggacgggctgacgcattggacgattttgatctggatatgctgggaagtgac | |
| | gccctcgatgattttgaccttgacatgcttggttcggatgcccttgatgactttgacctcgacatgctc | |
| | ggcagtgacgcccttgatgatttcgacctggacatgctgtgaatcgaattccgctcgagtggatcaact | |
| | tgtttattgcagcttataatggttacaaataaagcaatagcatcacaaatttcacaaataaagcatttt | |
| | tttcactgcgcggccgcaggaacccctagtgatggagttggccactccctctctgcgcgctcgctcgct | |
| | cactgaggccgggcgaccaaaggtcgcccgacgcccgggctttgcccgggcggcctcagtgagcgagcg | |
| | agcgcgcag | |
| pAAV- | cctgcaggcagctgcgcgctcgctcgctcactgaggccgcccgggcgtcgggcgacctttggtcgcccg | 150 |
| SCN1A_SE4_mini- | gcctcagtgagcgagcgagcgcgcagagagggagtggccaactccatcactaggggttcctgcggccgc | |
| SadCas9-v2 | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctgagctcgctagcgcgatcgctgccaccatggccccaaagaag | |
| | aagcggaaggtcggtatccacggagtcccagcagccaagaggaactacatcctgggcctggccatcggc | |
| | atcaccagcgtgggctacggcatcatcgactacgagaccagggacgtgatcgacgccggcgtgaggctg | |
| | ttcaaggaggccaacgtggagaacaacgagggcaggaggagcaagaggggcgccaggaggctgaagagg | |
| | aggaggaggcacaggatccagagggtgaagaagctgctgttcgactacaacctgctgaccgaccacagc | |
| | gagctgagcggcatcaacccctacgaggccagggtgaagggcctgagccagaagctgagcgaggaggag | |
| | ttcagcgccgccctgctgcacctggccaagaggaggggcgtgcacaacgtgaacgaggtggaggaggac | |
| | accggcaacgagctgagcaccaaggagcagatcagcaggaacagcaaggccctggaggagaagtacgtg | |
| | gccgagctgcagctggagaggctgaagaaggacggcgaggtgaggggcagcatcaacaggttcaagacc | |
| | agcgactacgtgaaggaggccaagcagctgctgaaggtgcagaaggcctaccaccagctggaccagagc | |
| | ttcatcgacacctacatcgacctgctggagaccaggaggacctactacgagggccccggcgagggcagc | |
| | cccttcggctggaaggacatcaaggagtggtacgagatgctgaagaggaggaggaggcacaggatcctg | |
| | agccccgtggtgaagaggagcttcatccagagcatcaaggtgatcaacgccatcatcaagaagtacggc | |
| | ctgcccaacgacatcatcatcgagctggccagggagaagaacagcaaggacgcccagaagatgatcaac | |
| | gagatgcagaagaggaacaggcagaccaacgagaggatcgaggagatcatcaggaccaccggcaaggag | |
| | aacgccaagtacctgatcgagaagatcaagctgcacgacatgcaggagggcaagtgcctgtacagcctg | |
| | gaggccatccccctggaggacctgctgaacaaccccttcaactacgaggtggaccacatcatccccagg | |
| | agcgtgagcttcgacaacagcttcaacaacaaggtgctggtgaagcaggaggaggccagcaagaagggc | |
| | aacaggacccccttccagtacctgagcagcagcgacagcaagatcagctacgagaccttcaagaagcac | |
| | atcctgaacctggccaagggcaagggcaggatcagcaagaccaagaaggagtacctgctggaggagagg | |
| | gacatcaacaggttcagcgtgcagaaggacttcatcaacaggaacctggtggacaccaggtacgccacc | |
| | aggggcctgatgaacctgctgaggagctacttcagggtgaacaacctggacgtgaaggtgaagagcatc | |
| | aacggcggcttcaccagcttcctgaggaggaagtggaagttcaagaaggagaggaacaagggctacaag | |
| | caccacgccgaggacgccctgatcatcgccaacgccgacttcatcttcaaggagtggaagaagctggac | |
| | aaggccaagaaggtgatggagaaccagatgttcgaggagaagcaggccgagagcatgcccgagatcgag | |
| | accgagcaggagtacaaggagatcttcatcaccccccaccagatcaagcacatcaaggacttcaaggac | |
| | tacaagtacagccacagggtggacaagaagcccaacaggaagctgatcaacgacaccctgtacagcacc | |
| | aggaaggacgacaagggcaacaccctgatcgtgaacaacctgaacggcctgtacgacaaggacaacgac | |
| | aagctgaagaagctgatcaacaagagccccgagaagctgctgatgtaccaccacgacccccagacctac | |
| | cagaagctgaagctgatcatggagcagtacggcgacgagaagaaccccctgtacaagtactacgaggag | |
| | accggcaactacctgaccaagtacagcaagaaggacaacggccccgtgatcaagaagatcaagtactac | |
| | ggcaacaagctgaacgcccacctggacatcaccgacgactaccccaacagcaggaacaaggtggtgaag | |
| | ctgagcctgaagccctacaggttcgacgtgtacctggacaacggcgtgtacaagttcgtgaccgtgaag | |
| | aacctggacgtgatcaagaaggagaactactacgaggtgaacagcaagtgctacgaggaggccaagaag | |
| | ctgaagaagatcagcaaccaggccgagttcatcgccagcttctacaagaacgacctgatcaagatcaac | |
| | ggcgagctgtacagggtgatcggcgtgaacaacgacctgctgaacaggatcgaggtgaacatgatcgac | |
| | atcacctacagggagtacctggagaacatgaacgacaagaggcccccccacatcatcaagaccatcgcc | |
| | agcaagacccagagcatcaagaagtacagcaccgacatcctgggcaacctgtacgaggtgaagagcaag | |
| | aagcacccccagatcatcaagaagggcaaaaggccggcggccacgaaaaaggccggccaggcaaaaaag | |
| | aaaaagggatccgaggccagcggttccggacgggctgacgcattggacgattttgatctggatatgctg | |
| | ggaagtgacgccctcgatgattttgaccttgacatgcttggttcggatgcccttgatgactttgacctc | |
| | gacatgctcggcagtgacgcccttgatgatttcgacctggacatgctgtgaatcgaattccgctcgagt | |
| | ggatcaacttgtttattgcagcttataatggttacaaataaagcaatagcatcacaaatttcacaaata | |
| | aagcatttttttcactgcgcggccgcaggaacccctagtgatggagttggccactccctctctgcgcgc | |
| | tcgctcgctcactgaggccgggcgaccaaaggtcgcccgacgcccgggctttgcccgggcggcctcagt | |
| | gagcgagcgagcgcgcag | |
| pAAV- | cctgcaggcagctgcgcgctcgctcgctcactgaggccgcccgggcgtcgggcgacctttggtcgcccg | 151 |
| SCN1A_SE4_mini- | gcctcagtgagcgagcgagcgcgcagagagggagtggccaactccatcactaggggttcctgcggccgc | |
| SadCas9-v4 | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctgagctcgctagcgcgatcgctgccaccatggccccaaagaag | |
| | aagcggaaggtcggtatccacggagtcccagcagccaagcggaactacatcctgggcctggccatcggc | |
| | atcaccagcgtgggctacggcatcatcgactacgagacacgggacgtgatcgatgccggcgtgcggctg | |
| | ttcaaagaggccaacgtggaaaacaacgagggcaggcggagcaagagaggcgccagaaggctgaagcgg | |
| | cggaggcggcatagaatccagagagtgaagaagctgctgttcgactacaacctgctgaccgaccacagc | |
| | gagctgagcggcatcaacccctacgaggccagagtgaagggcctgagccagaagctgagcgaggaagag | |
| | ttctctgccgccctgctgcacctggccaagagaagaggcgtgcacaacgtgaacgaggtggaagaggac | |
| | accggcaacgagctgtccaccaaagagcagatcagccggaacagcaaggccctggaagagaaatacgtg | |
| | gccgaactgcagctggaacggctgaagaaagacggcgaagtgcggggcagcatcaacagattcaagacc | |
| | agcgactacgtgaaagaagccaaacagctgctgaaggtgcagaaggcctaccaccagctggaccagagc | |
| | ttcatcgacacctacatcgacctgctggaaacccggcggacctactatgagggacctggcgagggcagc | |
| | cccttcggctggaaggacatcaaagaatggtacgagatgctgatgggccactgcacctacttccccgag | |
| | gaactgcggagcgtgaagtacgcctacaacgccgacctgtacaacgccctgaacgacctgaacaatctc | |
| | gtgatcaccagggacgagaacgagaagctggaatattacgagaagttccagatcatcgagaacgtgttc | |
| | aagcagaagaagaagcccaccctgaagcagatcgccaaagaaatcctcgtgaacgaagaggatattaag | |
| | ggctacagagtgaccagcaccggcaagcccgagttcaccaacctgaaggtgtaccacgacatcaaggac | |
| | attaccgcccggaaagagattattgagaacgccgagctgctggatcagattgccaagatcctgaccatc | |
| | taccagagcagcgaggacatccaggaagaactgaccaatctgaactccgagctgacccaggaagagatc | |
| | gagcagatctctaatctgaagggctataccggcacccacaacctgagcctgaaggccatcaacctgatc | |
| | ctggacgagctgtggcacaccaacgacaaccagatcgctatcttcaaccggctgaagctggtgcccaag | |
| | aaggtggacctgtcccagcagaaagagatccccaccaccctggtggacgacttcatcctgagccccgtc | |
| | gtgaagagaagcttcatccagagcatcaaagtgatcaacgccatcatcaagaagtacggcctgcccaac | |
| | gacatcattatcgagctgggcagcaagagatacgccaccagaggcctgatgaacctgctgcggagctac | |
| | ttcagagtgaacaacctggacgtgaaagtgaagtccatcaatggcggcttcaccagctttctgcggcgg | |
| | aagtggaagtttaagaaagagcggaacaaggggtacaagcaccacgccgaggacgccctgatcattgcc | |
| | aacgccgatttcatcttcaaagagtggaagaaactggacaaggccaaaaaagtgatggaaaaccagatg | |
| | ttcgaggaaaagcaggccgagagcatgcccgagatcgaaaccgagcaggagtacaaagagatcttcatc | |
| | accccccaccagatcaagcacattaaggacttcaaggactacaagtacagccaccgggtggacaagaag | |
| | cctaatagagagctgattaacgacaccctgtactccacccggaaggacgacaagggcaacaccctgatc | |
| | gtgaacaatctgaacggcctgtacgacaaggacaatgacaagctgaaaaagctgatcaacaagagcccc | |
| | gaaaagctgctgatgtaccaccacgacccccagacctaccagaaactgaagctgattatggaacagtac | |
| | ggcgacgagaagaatcccctgtacaagtactacgaggaaaccgggaactacctgaccaagtactccaaa | |
| | aaggacaacggccccgtgatcaagaagattaagtattacggcaacaaactgaacgcccatctggacatc | |
| | accgacgactaccccaacagcagaaacaaggtcgtgaagctgtccctgaagccctacagattcgacgtg | |
| | tacctggacaatggcgtgtacaagttcgtgaccgtgaagaatctggatgtgatcaaaaaagaaaactac | |
| | tacgaagtgaatagcaagtgctatgaggaagctaagaagctgaagaagatcagcaaccaggccgagttt | |
| | atcgcctccttctacaacaacgatctgatcaagatcaacggcgagctgtatagagtgatcggcgtgaac | |
| | aacgacctgctgaaccggatcgaagtgaacatgatcgacatcacctaccgcgagtacctggaaaacatg | |
| | aacgacaagaggccccccaggatcattaagacaatcgcctccaagacccagagcattaagaagtacagc | |
| | acagacattctgggcaacctgtatgaagtgaaatctaagaagcaccctcagatcatcaaaaagggcaaa | |
| | aggccggcggccacgaaaaaggccggccaggcaaaaaagaaaaagggatccgaggccagcggttccgga | |
| | cgggctgacgcattggacgattttgatctggatatgctgggaagtgacgccctcgatgattttgacctt | |
| | gacatgcttggttcggatgcccttgatgactttgacctcgacatgctcggcagtgacgcccttgatgat | |
| | ttcgacctggacatgctgtgaatcgaattccgctcgagtggatcaacttgtttattgcagcttataatg | |
| | gttacaaataaagcaatagcatcacaaatttcacaaataaagcatttttttcactgcgcggccgcagga | |
| | acccctagtgatggagttggccactccctctctgcgcgctcgctcgctcactgaggccgggcgaccaaa | |
| | ggtcgcccgacgcccgggctttgcccgggcggcctcagtgagcgagcgagcgcgcag | |
| pAAV- | cctgcaggcagctgcgcgctcgctcgctcactgaggccgcccgggcgtcgggcgacctttggtcgcccg | 152 |
| SCN1A_SE4_mini- | gcctcagtgagcgagcgagcgcgcagagagggagtggccaactccatcactaggggttcctgcggccgc | |
| SadCas9-v5 | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactagaatctaacatggctgctatagcttactgactagaagtt | |
| | aagtgcacacttcctaaaagaaggctttgacacaagccacttcagttccctcctcattttcttgtcccc | |
| | attcctctctctgtagaattctgagatttcaattcagttttatacagaaaccacattactgtaagccct | |
| | acaaagttatggcaatatagctatatggagtcaagtaatgtaggttatttttttcccaatggtgctggt | |
| | gaaggtggcaattatgtagctatacttagcagactgaggaaattctgctagagtcagcatttgtctctt | |
| | cattgctatgaaacagtaatggaaaaataaacaaaaacaaaaggcaaacactatgcataattccctcag | |
| | atcatattaacatgtgatgttggagtaaattgttataaccccattttggaaatacttaccttaattaac | |
| | tatgatttccttaaaataatgcagtatttacaatctatatgaaagcactatatgggacacatggtatga | |
| | tggaacagtgcacccaagagacaccaagaacattcctgtctgtggcagtcttttctctatacagaggca | |
| | tttagtctcaattgctcagagttattttaaacttcctgcagcccgggctgggcataaaagtcagggcag | |
| | agccatctattgcttacatttgcttctgagctcgctagcgcgatcgctgccaccatggccccaaagaag | |
| | aagcggaaggtcggtatccacggagtcccagcagccaagaggaactacatcctgggcctggccatcggc | |
| | atcaccagcgtgggctacggcatcatcgactacgagaccagggacgtgatcgacgccggcgtgaggctg | |
| | ttcaaggaggccaacgtggagaacaacgagggcaggaggagcaagaggggcgccaggaggctgaagagg | |
| | aggaggaggcacaggatccagagggtgaagaagctgctgttcgactacaacctgctgaccgaccacagc | |
| | gagctgagcggcatcaacccctacgaggccagggtgaagggcctgagccagaagctgagcgaggaggag | |
| | ttcagcgccgccctgctgcacctggccaagaggaggggcgtgcacaacgtgaacgaggtggaggaggac | |
| | accggcaacgagctgagcaccaaggagcagatcagcaggaacagcaaggccctggaggagaagtacgtg | |
| | gccgagctgcagctggagaggctgaagaaggacggcgaggtgaggggcagcatcaacaggttcaagacc | |
| | agcgactacgtgaaggaggccaagcagctgctgaaggtgcagaaggcctaccaccagctggaccagagc | |
| | ttcatcgacacctacatcgacctgctggagaccaggaggacctactacgagggccccggcgagggcagc | |
| | cccttcggctggaaggacatcaaggagtggtacgagatgctgaagaggaggaggaggcacaggatcctg | |
| | agccccgtggtgaagaggagcttcatccagagcatcaaggtgatcaacgccatcatcaagaagtacggc | |
| | ctgcccaacgacatcatcatcgagctgggcagcaagaggtacgccaccaggggcctgatgaacctgctg | |
| | aggagctacttcagggtgaacaacctggacgtgaaggtgaagagcatcaacggcggcttcaccagcttc | |
| | ctgaggaggaagtggaagttcaagaaggagaggaacaagggctacaagcaccacgccgaggacgccctg | |
| | atcatcgccaacgccgacttcatcttcaaggagtggaagaagctggacaaggccaagaaggtgatggag | |
| | aaccagatgttcgaggagaagcaggccgagagcatgcccgagatcgagaccgagcaggagtacaaggag | |
| | atcttcatcaccccccaccagatcaagcacatcaaggacttcaaggactacaagtacagccacagggtg | |
| | gacaagaagcccaacaggaagctgatcaacgacaccctgtacagcaccaggaaggacgacaagggcaac | |
| | accctgatcgtgaacaacctgaacggcctgtacgacaaggacaacgacaagctgaagaagctgatcaac | |
| | aagagccccgagaagctgctgatgtaccaccacgacccccagacctaccagaagctgaagctgatcatg | |
| | gagcagtacggcgacgagaagaaccccctgtacaagtactacgaggagaccggcaactacctgaccaag | |
| | tacagcaagaaggacaacggccccgtgatcaagaagatcaagtactacggcaacaagctgaacgcccac | |
| | ctggacatcaccgacgactaccccaacagcaggaacaaggtggtgaagctgagcctgaagccctacagg | |
| | ttcgacgtgtacctggacaacggcgtgtacaagttcgtgaccgtgaagaacctggacgtgatcaagaag | |
| | gagaactactacgaggtgaacagcaagtgctacgaggaggccaagaagctgaagaagatcagcaaccag | |
| | gccgagttcatcgccagcttctacaagaacgacctgatcaagatcaacggcgagctgtacagggtgatc | |
| | ggcgtgaacaacgacctgctgaacaggatcgaggtgaacatgatcgacatcacctacagggagtacctg | |
| | gagaacatgaacgacaagaggcccccccacatcatcaagaccatcgccagcaagacccagagcatcaag | |
| | aagtacagcaccgacatcctgggcaacctgtacgaggtgaagagcaagaagcacccccagatcatcaag | |
| | aagggcaaaaggccggcggccacgaaaaaggccggccaggcaaaaaagaaaaagggatccgaggccagc | |
| | ggttccggacgggctgacgcattggacgattttgatctggatatgctgggaagtgacgccctcgatgat | |
| | tttgaccttgacatgcttggttcggatgcccttgatgactttgacctcgacatgctcggcagtgacgcc | |
| | cttgatgatttcgacctggacatgctgtgaatcgaattccgctcgagtggatcaacttgtttattgcag | |
| | cttataatggttacaaataaagcaatagcatcacaaatttcacaaataaagcatttttttcactgcgcg | |
| | gccgcaggaacccctagtgatggagttggccactccctctctgcgcgctcgctcgctcactgaggccgg | |
| | gcgaccaaaggtcgcccgacgcccgggctttgcccgggcggcctcagtgagcgagcgagcgcgcag | |
| pAAV-SCN1A_SE5 | cctgcaggcagctgcgcgctcgctcgctcactgaggccgcccgggcgtcgggcgacctttggtcgcccg | 153 |
| | gcctcagtgagcgagcgagcgcgcagagagggagtggccaactccatcactaggggttcctgcggccgc | |
| | gtcgactagagagggcctatttcccatgattccttcatatttgcatatacgatacaaggctgttagaga | |
| | gataattggaattaatttgactgtaaacacaaagatattagtacaaaatacgtgacgtagaaagtaata | |
| | atttcttgggtagtttgcagttttaaaattatgttttaaaatggactatcatatgcttaccgtaacttg | |
| | aaagtatttcgatttcttggctttatatatcttgtggaaaggacgagcgctcgcaggcaggctgggagt | |
| | ttgcagttatagtactctggaaacagaatctactataacaaggcaaaatgccgtgtttatctcgtcaac | |
| | ttgttggcgagattttttttctagatactaggacattgattattgactagttattaatagtaatcaatt | |
| | acggggtcattagttcatagcccatatatggagttccgcgttacataacttacggtaaatggcccgcct | |
| | ggctgaccgcccaacgacccccgcccattgacgtcaataatgacgtatgttoccatagtaacgccaata | |
| | gggactttccattgacgtcaatgggtggagtatttacggtaaactgcccacttggcagtacatcaagtg | |
| | tatcatatgccaagtacgccccctattgacgtcaatgacggtaaatggcccgcctggcattatgcccag | |
| | tacatgaccttatgggactttcctacttggcagtacatctacgtattagtcatcgctattaccatggtg | |
| | atgcggttttggcagtacatcaatgggcgtggatagcggtttgactcacggggatttccaagtctccac | |
| | cccattgacgtcaatgggagtttgttttggcaccaaaatcaacgggactttccaaaatgtcgtaacaac | |
| | tccgccccattgacgcaaatgggcggtaggcgtgtacggtgggaggtctatataagcagagctctcgct | |
| | cgctagcgcgatcgctgccaccatggccccaaagaagaagcggaaggtcggtatccacggagtcccagc | |
| | agccaagcggaactacatcctgggcctggccatcggcatcaccagcgtgggctacggcatcatcgacta | |
| | cgagacacgggacgtgatcgatgccggcgtgcggctgttcaaagaggccaacgtggaaaacaacgaggg | |
| | caggcggagcaagagaggcgccagaaggctgaagcggcggaggcggcatagaatccagagagtgaagaa | |
| | gctgctgttcgactacaacctgctgaccgaccacagcgagctgagcggcatcaacccctacgaggccag | |
| | agtgaagggcctgagccagaagctgagcgaggaagagttctctgccgccctgctgcacctggccaagag | |
| | aagaggcgtgcacaacgtgaacgaggtggaagaggacaccggcaacgagctgtccaccaaagagcagat | |
| | cagccggaacagcaaggccctggaagagaaatacgtggccgaactgcagctggaacggctgaagaaaga | |
| | cggcgaagtgcggggcagcatcaacagattcaagaccagcgactacgtgaaagaagccaaacagctgct | |
| | gaaggtgcagaaggcctaccaccagctggaccagagcttcatcgacacctacatcgacctgctggaaac | |
| | ccggcggacctactatgagggacctggcgagggcagccccttcggctggaaggacatcaaagaatggta | |
| | cgagatgctgatgggccactgcacctacttccccgaggaactgcggagcgtgaagtacgcctacaacgc | |
| | cgacctgtacaacgccctgaacgacctgaacaatctcgtgatcaccagggacgagaacgagaagctgga | |
| | atattacgagaagttccagatcatcgagaacgtgttcaagcagaagaagaagcccaccctgaagcagat | |
| | cgccaaagaaatcctcgtgaacgaagaggatattaagggctacagagtgaccagcaccggcaagcccga | |
| | gttcaccaacctgaaggtgtaccacgacatcaaggacattaccgcccggaaagagattattgagaacgc | |
| | cgagctgctggatcagattgccaagatcctgaccatctaccagagcagcgaggacatccaggaagaact | |
| | gaccaatctgaactccgagctgacccaggaagagatcgagcagatctctaatctgaagggctataccgg | |
| | cacccacaacctgagcctgaaggccatcaacctgatcctggacgagctgtggcacaccaacgacaacca | |
| | gatcgctatcttcaaccggctgaagctggtgcccaagaaggtggacctgtcccagcagaaagagatccc | |
| | caccaccctggtggacgacttcatcctgagccccgtcgtgaagagaagcttcatccagagcatcaaagt | |
| | gatcaacgccatcatcaagaagtacggcctgcccaacgacatcattatcgagctggcccgcgagaagaa | |
| | ctccaaggacgcccagaaaatgatcaacgagatgcagaagcggaaccggcagaccaacgagcggatcga | |
| | ggaaatcatccggaccaccggcaaagagaacgccaagtacctgatcgagaagatcaagctgcacgacat | |
| | gcaggaaggcaagtgcctgtacagcctggaagccatccctctggaagatctgctgaacaaccccttcaa | |
| | ctatgaggtggaccacatcatccccagaagcgtgtccttcgacaacagcttcaacaacaaggtgctcgt | |
| | gaagcaggaagaagccagcaagaagggcaaccggaccccattccagtacctgagcagcagcgacagcaa | |
| | gatcagctacgaaaccttcaagaagcacatcctgaatctggccaagggcaagggcagaatcagcaagac | |
| | caagaaagagtatctgctggaagaacgggacatcaacaggttctccgtgcagaaagacttcatcaaccg | |
| | gaacctggtggataccagatacgccaccagaggcctgatgaacctgctgcggagctacttcagagtgaa | |
| | caacctggacgtgaaagtgaagtccatcaatggcggcttcaccagctttctgcggcggaagtggaagtt | |
| | taagaaagagcggaacaaggggtacaagcaccacgccgaggacgccctgatcattgccaacgccgattt | |
| | catcttcaaagagtggaagaaactggacaaggccaaaaaagtgatggaaaaccagatgttcgaggaaaa | |
| | gcaggccgagagcatgcccgagatcgaaaccgagcaggagtacaaagagatcttcatcaccccccacca | |
| | gatcaagcacattaaggacttcaaggactacaagtacagccaccgggtggacaagaagcctaatagaga | |
| | gctgattaacgacaccctgtactccacccggaaggacgacaagggcaacaccctgatcgtgaacaatct | |
| | gaacggcctgtacgacaaggacaatgacaagctgaaaaagctgatcaacaagagccccgaaaagctgct | |
| | gatgtaccaccacgacccccagacctaccagaaactgaagctgattatggaacagtacggcgacgagaa | |
| | gaatcccctgtacaagtactacgaggaaaccgggaactacctgaccaagtactccaaaaaggacaacgg | |
| | ccccgtgatcaagaagattaagtattacggcaacaaactgaacgcccatctggacatcaccgacgacta | |
| | ccccaacagcagaaacaaggtcgtgaagctgtccctgaagccctacagattcgacgtgtacctggacaa | |
| | tggcgtgtacaagttcgtgaccgtgaagaatctggatgtgatcaaaaaagaaaactactacgaagtgaa | |
| | tagcaagtgctatgaggaagctaagaagctgaagaagatcagcaaccaggccgagtttatcgcctcctt | |
| | ctacaacaacgatctgatcaagatcaacggcgagctgtatagagtgatcggcgtgaacaacgacctgct | |
| | gaaccggatcgaagtgaacatgatcgacatcacctaccgcgagtacctggaaaacatgaacgacaagag | |
| | gccccccaggatcattaagacaatcgcctccaagacccagagcattaagaagtacagcacagacattct | |
| | gggcaacctgtatgaagtgaaatctaagaagcaccctcagatcatcaaaaagggcaaaaggccggcggc | |
| | cacgaaaaaggccggccaggcaaaaaagaaaaagggatccgaggccagcggttccggacgggctgacgc | |
| | attggacgattttgatctggatatgctgggaagtgacgccctcgatgattttgaccttgacatgcttgg | |
| | ttcggatgcccttgatgactttgacctcgacatgctcggcagtgacgcccttgatgatttcgacctgga | |
| | catgctgtgaatcgaattccgctcgagctgatcaacttgtttattgcagcttataatggttacaaataa | |
| | agcaatagcatcacaaatttcacaaataaagcatttttttcactgcgcggccgcaggaacccctagtga | |
| | tggagttggccactccctctctgcgcgctcgctcgctcactgaggccgggcgaccaaaggtcgcccgac | |
| | gcccgggctttgcccgggcggcctcagtgagcgagcgagcgcgcag | |

TABLE 8
Exemplary human SCNA1 and Nav1.1 alpha subunit sequences

| Name | Sequence | SEQ ID NO |
|---|---|---|
| Nav1.1 alpha subunit | MEQTVLVPPGPDSFNFFTRESLAAIERRIAEEKAKNPKPDKKDDDENGPKPNSDLEAGKN | 154 |
| | LPFIYGDIPPEMVSEPLEDLDPYYINKKTFIVLNKGKAIFRFSATSALYILTPFNPLRKI | |
| | AIKILVHSLFSMLIMCTILTNCVFMTMSNPPDWTKNVEYTFTGIYTFESLIKIIARGFCL | |
| | EDFTFLRDPWNWLDFTVITFAYVTEFVDLGNVSALRTFRVLRALKTISVIPGLKTIVGAL | |
| | IQSVKKLSDVMILTVFCLSVFALIGLQLFMGNLRNKCIQWPPTNASLEEHSIEKNITVNY | |
| | NGTLINETVFEFDWKSYIQDSRYHYFLEGFLDALLCGNSSDAGQCPEGYMCVKAGRNPNY | |
| | GYTSFDTFSWAFLSLFRLMTQDFWENLYQLTLRAAGKTYMIFFVLVIFLGSFYLINLILA | |
| | VVAMAYEEQNQATLEEAEQKEAEFQQMIEQLKKQQEAAQQAATATASEHSREPSAAGRLS | |
| | DSSSEASKLSSKSAKERRNRRKKRKQKEQSGGEEKDEDEFQKSESEDSIRRKGFRFSIEG | |
| | NRLTYEKRYSSPHQSLLSIRGSLFSPRRNSRTSLFSFRGRAKDVGSENDFADDEHSTFED | |
| | NESRRDSLFVPRRHGERRNSNLSQTSRSSRMLAVFPANGKMHSTVDCNGVVSLVGGPSVP | |
| | TSPVGQLLPEVIIDKPATDDNGTTTETEMRKRRSSSFHVSMDFLEDPSQRQRAMSIASIL | |
| | TNTVEELEESRQKCPPCWYKFSNIFLIWDCSPYWLKVKHVVNLVVMDPFVDLAITICIVL | |
| | NTLFMAMEHYPMTDHFNNVLTVGNLVFTGIFTAEMFLKIIAMDPYYYFQEGWNIFDGFIV | |
| | TLSLVELGLANVEGLSVLRSFRLLRVFKLAKSWPTLNMLIKIIGNSVGALGNLTLVLAII | |
| | VFIFAVVGMQLFGKSYKDCVCKIASDCQLPRWHMNDFFHSFLIVFRVLCGEWIETMWDCM | |
| | EVAGQAMCLTVFMMVMVIGNLVVLNLFLALLLSSFSADNLAATDDDNEMNNLQIAVDRMH | |
| | KGVAYVKRKIYEFIQQSFIRKQKILDEIKPLDDLNNKKDSCMSNHTAEIGKDLDYLKDVN | |
| | GTTSGIGTGSSVEKYIIDESDYMSFINNPSLTVTVPIAVGESDFENLNTEDFSSESDLEE | |
| | SKEKLNESSSSSEGSTVDIGAPVEEQPVVEPEETLEPEACFTEGCVQRFKCCQINVEEGR | |
| | GKQWWNLRRTCFRIVEHNWFETFIVEMILLSSGALAFEDIYIDQRKTIKTMLEYADKVFT | |
| | YIFILEMLLKWVAYGYQTYFTNAWCWLDFLIVDVSLVSLTANALGYSELGAIKSLRTLRA | |
| | LRPLRALSRFEGMRVVVNALLGAIPSIMNVLLVCLIFWLIFSIMGVNLFAGKFYHCINTT | |
| | TGDRFDIEDVNNHTDCLKLIERNETARWKNVKVNFDNVGFGYLSLLQVATFKGWMDIMYA | |
| | AVDSRNVELQPKYEESLYMYLYFVIFIIFGSFFTLNLFIGVIIDNFNQQKKKFGGQDIFM | |
| | TEEQKKYYNAMKKLGSKKPQKPIPRPGNKFQGMVFDFVTRQVFDISIMILICLNMVTMMV | |
| | ETDDQSEYVTTILSRINLVFIVLFTGECVLKLISLRHYYFTIGWNIFDFVVVILSIVGMF | |
| | LAELIEKYFVSPTLFRVIRLARIGRILRLIKGAKGIRTLLFALMMSLPALFNIGLLLFLV | |
| | MFIYAIFGMSNFAYVKREVGIDDMFNFETFGNSMICLFQITTSAGWDGLLAPILNSKPPD | |
| | CDPNKVNPGSSVKGDCGNPSVGIFFFVSYIIISFLVVVNMYIAVILENFSVATEESAEPL | |
| | SEDDFEMFYEVWEKFDPDATQFMEFEKLSQFAAALEPPLNLPQPNKLQLIAMDLPMVSGD | |
| | RIHCLDILFAFTKRVLGESGEMDALRIQMEERFMASNPSKVSYQPITTTLKRKQEEVSAV | |
| | IIQRAYRRHLLKRTVKQASFTYNKNKIKGGANLLIKEDMIIDRINENSITEKTDLTMSTA | |
| | ACPPSYDRVTKPIVEKHEQEGKDEKAKGK | |
| SCNA1 sequence | atggagcaaacagtgcttgtaccaccaggacctgacagcttcaacttcttcaccagagaa | 155 |
| | tctcttgcggctattgaaagacgcattgcagaagaaaaggcaaagaatcccaaaccagac | |
| | aaaaaagatgacgacgaaaatggcccaaagccaaatagtgacttggaagctggaaagaac | |
| | cttccatttatttatggagacattcctccagagatggtgtcagagcccctggaggacctg | |
| | gacccctactatatcaataagaaaacttttatagtattgaataaagggaaggccatcttc | |
| | cggttcagtgccacctctgccctgtacattttaactcccttcaatcctcttaggaaaata | |
| | gctattaagattttggtacattcattattcagcatgctaattatgtgcactattttgaca | |
| | aactgtgtgtttatgacaatgagtaaccctcctgattggacaaagaatgtagaatacacc | |
| | ttcacaggaatatatacttttgaatcacttataaaaattattgcaaggggattctgttta | |
| | gaagattttactttccttcgggatccatggaactggctcgatttcactgtcattacattt | |
| | gcgtacgtcacagagtttgtggacctgggcaatgtctcggcattgagaacattcagagtt | |
| | ctccgagcattgaagacgatttcagtcattccaggcctgaaaaccattgtgggagccctg | |
| | atccagtctgtgaagaagctctcagatgtaatgatcctgactgtgttctgtctgagcgta | |
| | tttgctctaattgggctgcagctgttcatgggcaacctgaggaataaatgtatacaatgg | |
| | cctcccaccaatgcttccttggaggaacatagtatagaaaagaatataactgtgaattat | |
| | aatggtacacttataaatgaaactgtctttgagtttgactggaagtcatatattcaagat | |
| | tcaagatatcattatttcctggagggttttttagatgcactactatgtggaaatagctct | |
| | gatgcaggccaatgtccagagggatatatgtgtgtgaaagctggtagaaatcccaattat | |
| | ggctacacaagctttgataccttcagttgggcttttttgtccttgtttcgactaatgact | |
| | caggacttctgggaaaatctttatcaactgacattacgtgctgctgggaaaacgtacatg | |
| | atattttttgtattggtcattttcttgggctcattctacctaataaatttgatcctggct | |
| | gtggtggccatggcctacgaggaacagaatcaggccaccttggaagaagcagaacagaaa | |
| | gaggccgaatttcagcagatgattgaacagcttaaaaagcaacaggaggcagctcagcag | |
| | gcagcaacggcaactgcctcagaacattccagagagcccagtgcagcaggcaggctctca | |
| | gacagctcatctgaagcctctaagttgagttccaagagtgctaaggaaagaagaaatcgg | |
| | aggaagaaaagaaaacagaaagagcagtctggtggggaagagaaagatgaggatgaattc | |
| | caaaaatctgaatctgaggacagcatcaggaggaaaggttttcgcttctccattgaaggg | |
| | aaccgattgacatatgaaaagaggtactcctccccacaccagtctttgttgagcatccgt | |
| | ggctccctattttcaccaaggcgaaatagcagaacaagccttttcagctttagagggcga | |
| | gcaaaggatgtgggatctgagaacgacttcgcagatgatgagcacagcacctttgaggat | |
| | aacgagagccgtagagattccttgtttgtgccccgacgacacggagagagacgcaacagc | |
| | aacctgagtcagaccagtaggtcatcccggatgctggcagtgtttccagcgaatgggaag | |
| | atgcacagcactgtggattgcaatggtgtggtttccttggttggtggaccttcagttcct | |
| | acatcgcctgttggacagcttctgccagaggtgataatagataagccagctactgatgac | |
| | aatggaacaaccactgaaactgaaatgagaaagagaaggtcaagttctttccacgtttcc | |
| | atggactttctagaagatccttcccaaaggcaacgagcaatgagtatagccagcattcta | |
| | acaaatacagtagaagaacttgaagaatccaggcagaaatgcccaccctgttggtataaa | |
| | ttttccaacatattcttaatctgggactgttctccatattggttaaaagtgaaacatgtt | |
| | gtcaacctggttgtgatggacccatttgttgacctggccatcaccatctgtattgtctta | |
| | aatactcttttcatggccatggagcactatccaatgacggaccatttcaataatgtgctt | |
| | acagtaggaaacttggttttcactgggatctttacagcagaaatgtttctgaaaattatt | |
| | gccatggatccttactattatttccaagaaggctggaatatctttgacggttttattgtg | |
| | acgcttagcctggtagaacttggactcgccaatgtggaaggattatctgttctccgttca | |
| | tttcgattgctgcgagttttcaagttggcaaaatcttggccaacgttaaatatgctaata | |
| | aagatcatcggcaattccgtgggggctctgggaaatttaaccctcgtcttggccatcatc | |
| | gtcttcatttttgccgtggtcggcatgcagctctttggtaaaagctacaaagattgtgtc | |
| | tgcaagatcgccagtgattgtcaactcccacgctggcacatgaatgacttcttccactcc | |
| | ttcctgattgtgttccgcgtgctgtgtggggagtggatagagaccatgtgggactgtatg | |
| | gaggttgctggtcaagccatgtgccttactgtcttcatgatggtcatggtgattggaaac | |
| | ctagtggtcctgaatctctttctggccttgcttctgagctcatttagtgcagacaacctt | |
| | gcagccactgatgatgataatgaaatgaataatctccaaattgctgtggataggatgcac | |
| | aaaggagtagcttatgtgaaaagaaaaatatatgaatttattcaacagtccttcattagg | |
| | aaacaaaagattttagatgaaattaaaccacttgatgatctaaacaacaagaaagacagt | |
| | tgtatgtccaatcatacagcagaaattgggaaagatcttgactatcttaaagatgtaaat | |
| | ggaactacaagtggtataggaactggcagcagtgttgaaaaatacattattgatgaaagt | |
| | gattacatgtcattcataaacaaccccagtcttactgtgactgtaccaattgctgtagga | |
| | gaatctgactttgaaaatttaaacacggaagactttagtagtgaatcggatctggaagaa | |
| | agcaaagagaaactgaatgaaagcagtagctcatcagaaggtagcactgtggacatcggc | |
| | gcacctgtagaagaacagcccgtagtggaacctgaagaaactcttgaaccagaagcttgt | |
| | ttcactgaaggctgtgtacaaagattcaagtgttgtcaaatcaatgtggaagaaggcaga | |
| | ggaaaacaatggtggaacctgagaaggacgtgtttccgaatagttgaacataactggttt | |
| | gagaccttcattgttttcatgattctccttagtagtggtgctctggcatttgaagatata | |
| | tatattgatcagcgaaagacgattaagacgatgttggaatatgctgacaaggttttcact | |
| | tacattttcattctggaaatgcttctaaaatgggtggcatatggctatcaaacatatttc | |
| | accaatgcctggtgttggctggacttcttaattgttgatgtttcattggtcagtttaaca | |
| | gcaaatgccttgggttactcagaacttggagccatcaaatctctcaggacactaagagct | |
| | ctgagacctctaagagccttatctcgatttgaagggatgagggtggttgtgaatgccctt | |
| | ttaggagcaattccatccatcatgaatgtgcttctggtttgtcttatattctggctaatt | |
| | ttcagcatcatgggcgtaaatttgtttgctggcaaattctaccactgtattaacaccaca | |
| | actggtgacaggtttgacatcgaagacgtgaataatcatactgattgcctaaaactaata | |
| | gaaagaaatgagactgctcgatggaaaaatgtgaaagtaaactttgataatgtaggattt | |
| | gggtatctctctttgcttcaagttgccacattcaaaggatggatggatataatgtatgca | |
| | gcagttgattccagaaatgtggaactccagcctaagtatgaagaaagtctgtacatgtat | |
| | ctttactttgttattttcatcatctttgggtccttcttcaccttgaacctgtttattggt | |
| | gtcatcatagataatttcaaccagcagaaaaagaagtttggaggtcaagacatctttatg | |
| | acagaagaacagaagaaatactataatgcaatgaaaaaattaggatcgaaaaaaccgcaa | |
| | aagcctatacctcgaccaggaaacaaatttcaaggaatggtctttgacttcgtaaccaga | |
| | caagtttttgacataagcatcatgattctcatctgtcttaacatggtcacaatgatggtg | |
| | gaaacagatgaccagagtgaatatgtgactaccattttgtcacgcatcaatctggtgttc | |
| | attgtgctatttactggagagtgtgtactgaaactcatctctctacgccattattatttt | |
| | accattggatggaatatttttgattttgtggttgtcattctctccattgtaggtatgttt | |
| | cttgccgagctgatagaaaagtatttcgtgtcccctaccctgttccgagtgatccgtctt | |
| | gctaggattggccgaatcctacgtctgatcaaaggagcaaaggggatccgcacgctgctc | |
| | tttgctttgatgatgtcccttcctgcgttgtttaacatcggcctcctactcttcctagtc | |
| | atgttcatctacgccatctttgggatgtccaactttgcctatgttaagagggaagttggg | |
| | atcgatgacatgttcaactttgagacctttggcaacagcatgatctgcctattccaaatt | |
| | acaacctctgctggctgggatggattgctagcacccattctcaacagtaagccacccgac | |
| | tgtgaccctaataaagttaaccctggaagctcagttaagggagactgtgggaacccatct | |
| | gttggaattttcttttttgtcagttacatcatcatatccttcctggttgtggtgaacatg | |
| | tacatcgcggtcatcctggagaacttcagtgttgctactgaagaaagtgcagagcctctg | |
| | agtgaggatgactttgagatgttctatgaggtttgggagaagtttgatcccgatgcaact | |
| | cagttcatggaatttgaaaaattatctcagtttgcagctgcgcttgaaccgcctctcaat | |
| | ctgccacaaccaaacaaactccagctcattgccatggatttgcccatggtgagtggtgac | |
| | cggatccactgtcttgatatcttatttgcttttacaaagcgggttctaggagagagtgga | |
| | gagatggatgctctacgaatacagatggaagagcgattcatggcttccaatccttccaag | |
| | gtctcctatcagccaatcactactactttaaaacgaaaacaagaggaagtatctgctgtc | |
| | attattcagcgtgcttacagacgccaccttttaaagcgaactgtaaaacaagcttccttt | |
| | acgtacaataaaaacaaaatcaaaggtggggctaatcttcttataaaagaagacatgata | |
| | attgacagaataaatgaaaactctattacagaaaaaactgatctgaccatgtccactgca | |
| | gcttgtccaccttcctatgaccgggtgacaaagccaattgtggaaaaacatgagcaagaa | |
| | ggcaaagatgaaaaagccaaagggaaataa | |

TABLE 9
Exemplary amino acid sequences of dCas9

| Name | Sequence | SEQ ID NO |
|---|---|---|
| Sa dCas9 | KRNYILGLAIGITSVGYGIIDYETRDVIDAGVRLFKEANVENNEGRRSKRGARRLKRRRRHRIQRVKKLLFDYNL | 156 |
| | LTDHSELSGINPYEARVKGLSQKLSEEEFSAALLHLAKRRGVHNVNEVEEDTGNELSTKEQISRNSKALEEKYVA | |
| | ELQLERLKKDGEVRGSINRFKTSDYVKEAKQLLKVQKAYHOLDQSFIDTYIDLLETRRTYYEGPGEGSPFGWKDI | |
| | KEWYEMLMGHCTYFPEELRSVKYAYNADLYNALNDLNNLVITRDENEKLEYYEKFQIIENVFKQKKKPTLKQIAK | |
| | EILVNEEDIKGYRVTSTGKPEFTNLKVYHDIKDITARKEIIENAELLDQIAKILTIYQSSEDIQEELTNLNSELT | |
| | QEEIEQISNLKGYTGTHNLSLKAINLILDELWHTNDNQIAIFNRLKLVPKKVDLSQQKEIPTTLVDDFILSPVVK | |
| | RSFIQSIKVINAIIKKYGLPNDIIIELAREKNSKDAQKMINEMQKRNRQTNERIEEIIRTTGKENAKYLIEKIKL | |
| | HDMQEGKCLYSLEAIPLEDLLNNPFNYEVDHIIPRSVSFDNSFNNKVLVKQEEASKKGNRTPFQYLSSSDSKISY | |
| | ETFKKHILNLAKGKGRISKTKKEYLLEERDINRFSVQKDFINRNLVDTRYATRGLMNLLRSYFRVNNLDVKVKSI | |
| | NGGFTSFLRRKWKFKKERNKGYKHHAEDALIIANADFIFKEWKKLDKAKKVMENQMFEEKQAESMPEIETEQEYK | |
| | EIFITPHQIKHIKDFKDYKYSHRVDKKPNRELINDTLYSTRKDDKGNTLIVNNLNGLYDKDNDKLKKLINKSPEK | |
| | LLMYHHDPQTYQKLKLIMEQYGDEKNPLYKYYEETGNYLTKYSKKDNGPVIKKIKYYGNKLNAHLDITDDYPNSR | |
| | NKVVKLSLKPYRFDVYLDNGVYKFVTVKNLDVIKKENYYEVNSKCYEEAKKLKKISNQAEFIASFYNNDLIKING | |
| | ELYRVIGVNNDLLNRIEVNMIDITYREYLENMNDKRPPRIIKTIASKTQSIKKYSTDILGNLYEVKSKKHPQIIK | |
| | KG | |
| mini- | KRNYILGLAIGITSVGYGIIDYETRDVIDAGVRLFKEANVENNEGRRSKRGARRLKRRRRHRIQRVKKLLFDYNL | 157 |
| Sadcas9-v2- | LTDHSELSGINPYEARVKGLSQKLSEEEFSAALLHLAKRRGVHNVNEVEEDTGNELSTKEQISRNSKALEEKYVA | |
| del234-444 | ELQLERLKKDGEVRGSINRFKTSDYVKEAKQLLKVQKAYHOLDQSFIDTYIDLLETRRTYYEGPGEGSPFGWKDI | |
| | KEWYEMLKRRRRHRILSPVVKRSFIQSIKVINAIIKKYGLPNDIIIELAREKNSKDAQKMINEMQKRNRQTNERI | |
| | EEIIRTTGKENAKYLIEKIKLHDMQEGKCLYSLEAIPLEDLLNNPFNYEVDHIIPRSVSFDNSFNNKVLVKQEEA | |
| | SKKGNRTPFQYLSSSDSKISYETFKKHILNLAKGKGRISKTKKEYLLEERDINRFSVQKDFINRNLVDTRYATRG | |
| | LMNLLRSYFRVNNLDVKVKSINGGFTSFLRRKWKFKKERNKGYKHHAEDALIIANADFIFKEWKKLDKAKKVMEN | |
| | QMFEEKQAESMPEIETEQEYKEIFITPHQIKHIKDFKDYKYSHRVDKKPNRKLINDTLYSTRKDDKGNTLIVNNL | |
| | NGLYDKDNDKLKKLINKSPEKLLMYHHDPQTYQKLKLIMEQYGDEKNPLYKYYEETGNYLTKYSKKDNGPVIKKI | |
| | KYYGNKLNAHLDITDDYPNSRNKVVKLSLKPYRFDVYLDNGVYKFVTVKNLDVIKKENYYEVNSKCYEEAKKLKK | |
| | ISNQAEFIASFYKNDLIKINGELYRVIGVNNDLLNRIEVNMIDITYREYLENMNDKRPPHIIKTIASKTQSIKKY | |
| | STDILGNLYEVKSKKHPQIIKKG | |
| mini- | KRNYILGLAIGITSVGYGIIDYETRDVIDAGVRLFKEANVENNEGRRSKRGARRLKRRRRHRIQRVKKLLFDYNL | 158 |
| Sadcas9- | LTDHSELSGINPYEARVKGLSQKLSEEEFSAALLHLAKRRGVHNVNEVEEDTGNELSTKEQISRNSKALEEKYVA | |
| vp64-v4- | ELQLERLKKDGEVRGSINRFKTSDYVKEAKQLLKVQKAYHOLDQSFIDTYIDLLETRRTYYEGPGEGSPFGWKDI | |
| del479-649 | KEWYEMLMGHCTYFPEELRSVKYAYNADLYNALNDLNNLVITRDENEKLEYYEKFQIIENVFKQKKKPTLKQIAK | |
| | EILVNEEDIKGYRVTSTGKPEFTNLKVYHDIKDITARKEIIENAELLDQIAKILTIYQSSEDIQEELTNLNSELT | |
| | QEEIEQISNLKGYTGTHNLSLKAINLILDELWHTNDNQIAIFNRLKLVPKKVDLSQQKEIPTTLVDDFILSPVVK | |
| | RSFIQSIKVINAIIKKYGLPNDIIIELGSKRYATRGLMNLLRSYFRVNNLDVKVKSINGGFTSFLRRKWKFKKER | |
| | NKGYKHHAEDALIIANADFIFKEWKKLDKAKKVMENQMFEEKQAESMPEIETEQEYKEIFITPHQIKHIKDFKDY | |
| | KYSHRVDKKPNRELINDTLYSTRKDDKGNTLIVNNLNGLYDKDNDKLKKLINKSPEKLLMYHHDPQTYQKLKLIM | |
| | EQYGDEKNPLYKYYEETGNYLTKYSKKDNGPVIKKIKYYGNKLNAHLDITDDYPNSRNKVVKLSLKPYRFDVYLD | |
| | NGVYKFVTVKNLDVIKKENYYEVNSKCYEEAKKLKKISNQAEFIASFYNNDLIKINGELYRVIGVNNDLLNRIEV | |
| | NMIDITYREYLENMNDKRPPRIIKTIASKTQSIKKYSTDILGNLYEVKSKKHPQIIKKG | |
| mini- | KRNYILGLAIGITSVGYGIIDYETRDVIDAGVRLFKEANVENNEGRRSKRGARRLKRRRRHRIQRVKKLLFDYNL | 159 |
| Sadcas9-v5- | LTDHSELSGINPYEARVKGLSQKLSEEEFSAALLHLAKRRGVHNVNEVEEDTGNELSTKEQISRNSKALEEKYVA | |
| del234- | ELQLERLKKDGEVRGSINRFKTSDYVKEAKQLLKVQKAYHOLDQSFIDTYIDLLETRRTYYEGPGEGSPFGWKDI | |
| 444, 479- | KEWYEMLKRRRRHRILSPVVKRSFIQSIKVINAIIKKYGLPNDIIIELGSKRYATRGLMNLLRSYFRVNNLDVKV | |
| 649 | KSINGGFTSFLRRKWKFKKERNKGYKHHAEDALIIANADFIFKEWKKLDKAKKVMENQMFEEKQAESMPEIETEQ | |
| | EYKEIFITPHQIKHIKDFKDYKYSHRVDKKPNRKLINDTLYSTRKDDKGNTLIVNNLNGLYDKDNDKLKKLINKS | |
| | PEKLLMYHHDPQTYQKLKLIMEQYGDEKNPLYKYYEETGNYLTKYSKKDNGPVIKKIKYYGNKLNAHLDITDDYP | |
| | NSRNKVVKLSLKPYRFDVYLDNGVYKFVTVKNLDVIKKENYYEVNSKCYEEAKKLKKISNQAEFIASFYKNDLIK | |
| | INGELYRVIGVNNDLLNRIEVNMIDITYREYLENMNDKRPPHIIKTIASKTQSIKKYSTDILGNLYEVKSKKHPQ | |
| | IIKKG | |

The disclosure will be further clarified by the following examples, which are intended to be purely exemplary of the disclosure and in no way limiting.

### EXAMPLES

### Example 1. In Vitro Modulation of SCNA1 Expression in Cell Lines

A vector provided herein was tested for capability to normalize SCN1A expression levels in relevant cells in vitro.

Kelly cells (a human neuroblastoma cell line) were lipo-transfected with plasmids containing the indicated gRNA and the dCas9-VP64 transgene (encoding SEQ ID NO: 114) driven by the E2 enhancer (SEQ ID NO: 34). After 48 h of incubation, RNA extraction and cDNA synthesis was performed, followed by qPCR using SCN1A-specific primers. The values were normalized to the level of SCN1A obtained in control conditions (i.e., with non-targeting control gRNA). Error bars represent standard deviation derived from at least four biological replicates.

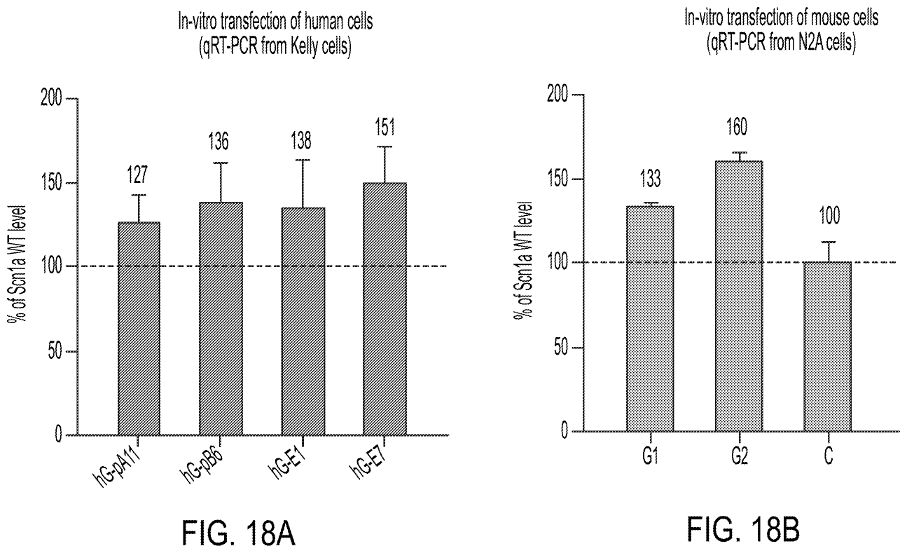

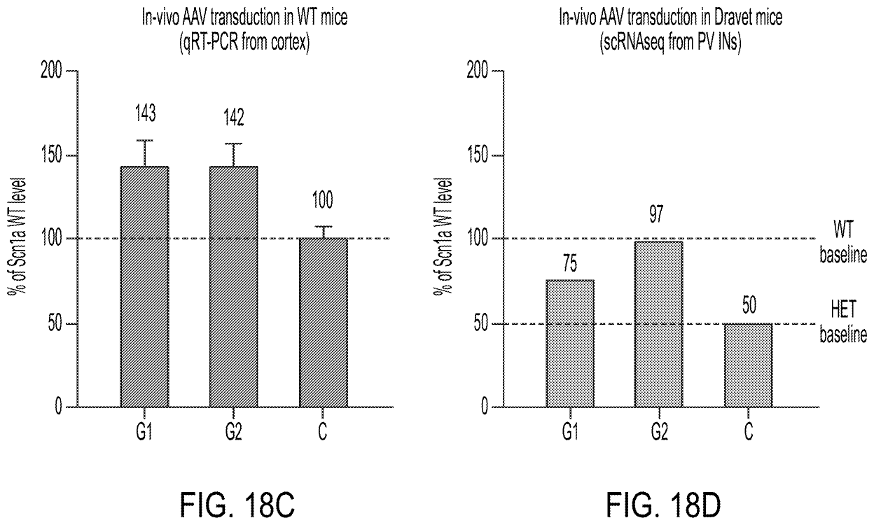

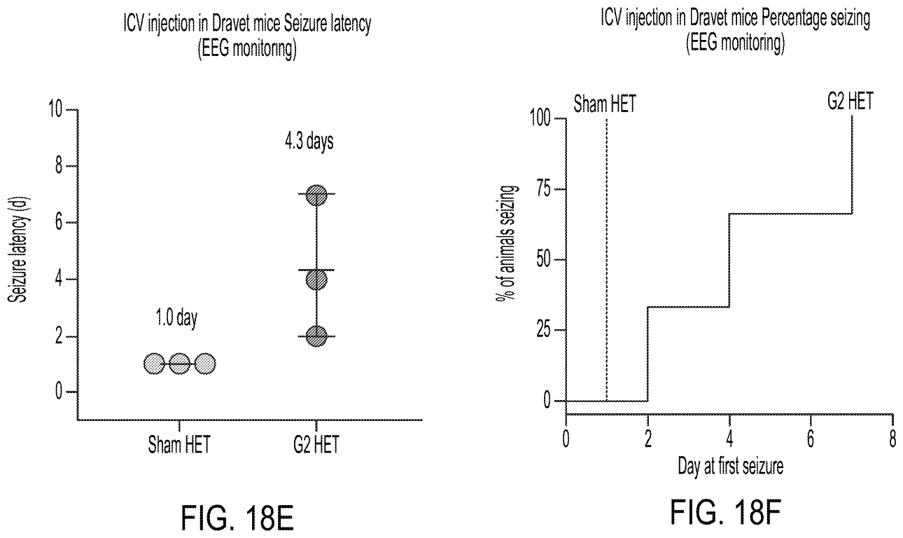

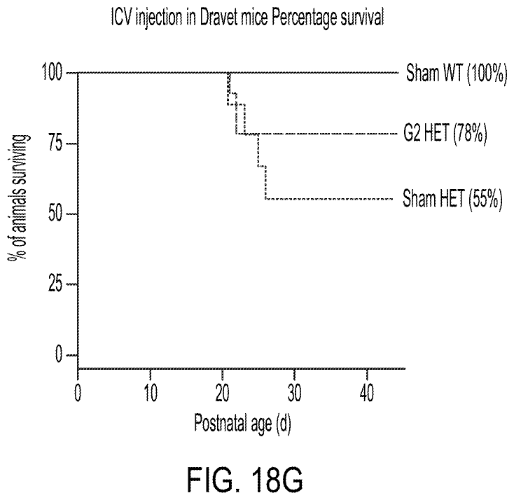

As shown in FIG. 18A, the top four performing human gRNAs, used in the vector containing E2 driving dCas9-VP64, upregulated SCN1A expression in vitro in Kelly cells. Non-targeting gRNA was used as control. The human gRNAs used were hG-pA11 (SEQ ID NO: 11), hG-pB6 (SEQ ID NO: 19), hG-E1 (SEQ ID NO: 21), and hG-E7 (SEQ ID NO: 27).

Mouse Neuro 2A (N2A) cells were lipo-transfected with plasmids containing the indicated gRNA and the dCas9-VP64 transgene driven by the E2 enhancer (SEQ ID NO: 34). After 48 h of incubation, RNA extraction and cDNA synthesis was performed, followed by qPCR using SCN1A-specific primers. The values were normalized to the level of SCN1A obtained in control conditions (i.e., with non-targeting control gRNA). Error bars represent standard deviation derived from at least two biological replicates.

As shown in FIG. 18B, the top two performing gRNAs (G1 (SEQ ID NO: 160) and G2 (SEQ ID NO: 161)), used in the vector containing E2 driving dCas9-VP64, upregulated SCN1A expression in vitro in N2A cells. Non-targeting gRNA was used as control (C).

### Example 2. In Vivo Modulation of SCNA1 Expression in Wild-Type Mice and Dravet Mice

Wild-type (WT) mouse pups were treated via intracerebroventricular (ICV) injection with an AAV9 (comprising AAV expression cassette of SEQ ID NO: 141) driving the expression of the dCas9-VP64 transgene under the control of the E2 enhancer in addition to the indicated guide RNA. After 4 weeks of incubation, RNA extraction and cDNA synthesis was performed from the cortical tissue followed by qPCR using SCN1A-specific primers. The values were normalized to the level of SCN1A obtained in control conditions (i.e., with non-targeting control gRNA). Error bars represent standard deviation derived from at least two biological replicates.

As shown in FIG. 18C, the top two performing gRNAs (G1 (SEQ ID NO: 160) and G2 (SEQ ID NO: 161)), used in the vector containing E2 driving dCas9-VP64, upregulated the expression level of SCN1A in the cortex of WT mice above WT level. Non-targeting gRNA was used as control (C).

Dravet mouse pups (Scn1at^{m1Kea} Dravet mouse model; Miller et al., (2014) Genes, Brain and Behavior, 13: 163-172) were treated via ICV injection with an AAV (comprising AAV expression cassette of SEQ ID NO: 141) driving the expression of the dCas9-VP64 transgene under the control of the E2 enhancer in addition to the indicated guide RNA. The genotype of the animals (WT: wild type for SCN1A; or HET: heterozygous for SCN1A) was defined by PCR. After 4 weeks of incubation, the cortexes of the mice were extracted and subject to single-nucleus isolation (total of ˜8,000 cells including ˜200 parvalbumin-expressing interneurons), library synthesis and sequencing. SCN1A in the subset of parvalbumin-expressing cells was extracted across conditions and the expression level values were normalized to control conditions (i.e., with non-targeting control gRNA).

As shown in FIG. 18D, the top two performing gRNAs (G1 (SEQ ID NO: 160) and G2 (SEQ ID NO: 161)), used in the vector containing E2 driving dCas9-VP64, normalized the level of SCN1A in the parvalbumin-expressing cells of Dravet mice, above baseline level of expression in heterozygous and closer to WT level. Non-targeting gRNA was used as control (C). Thus, administration of the vector resulted in a normalization of SCN1A gene expression in a mouse model for Dravet syndrome.

### Example 3. Effects on Seizure and Survival Phenotype in Dravet Model Mice

Dravet mouse pups (Scn1at^{m1Kea} Dravet mouse model) were treated via ICV injection with an AAV9 (comprising AAV expression cassette of SEQ ID NO: 141) driving the expression of the dCas9-VP64 transgene under the control of the E2 enhancer in addition to the indicated guide RNA (G2; SEQ ID NO: 161). The genotype of the animals (WT: wild type for SCN1A; or HET: heterozygous for SCN1A) was defined by PCR. Survival of the animals was monitored for the duration of the experiment and after 4 weeks of incubation. Electroencephalogram (EEG) activity was monitored constantly for a period of 10 days. Each experimental group contained at least three animals.

Injection of the AAV vector in Dravet mice followed by EEG monitoring of cortical activity resulted in an amelioration of the seizure phenotype as measured by seizure latency (FIG. 18E) and time to first seizure (FIG. 18F). Treated mice also showed an increase in survival (78% compared to a baseline survival of 55% in untreated control animals of this cohort (FIG. 18G)). Thus, administration of the vector resulted in amelioration of the seizure and survival phenotype in a mouse model for Dravet syndrome.

### Numbered Embodiments

Notwithstanding the appended claims, the disclosure sets forth the following numbered embodiments:

Embodiment 1. A vector comprising:
(a) a transgene polynucleotide sequence encoding a sequence-specific DNA-targeting module (DTM) fused to a transactivator;
(b) an enhancer polynucleotide sequence that specifically restricts expression of the transgene to sodium voltage-gated channel alpha subunit 1 (SCN1A)-expressing cells in the brain; and
(c) a promoter polynucleotide sequence.

Embodiment 2. The vector of embodiment 1, wherein the SCN1A-expressing cells are GABAergic interneuron cells.

Embodiment 3. The vector of embodiment 1, wherein the SCN1A-expressing cells are parvalbumin (PV)-expressing interneurons.

Embodiment 4. The vector of any one of embodiments 1-3, wherein the DTM comprises a nuclease-deficient CRISPR-associated protein.

Embodiment 5. The vector of embodiment 4, wherein the vector further comprises a polynucleotide sequence encoding a guide RNA (gRNA).

Embodiment 6. The vector of embodiment 5, wherein the gRNA targets the SCN1A gene.

Embodiment 7. The vector of embodiment 5 or 6, wherein the gRNA specifically hybridizes to a regulatory region of the SCN1A gene.

Embodiment 8. The vector of embodiment 7, wherein the regulatory region of the SCN1A gene is a promoter or an enhancer.

Embodiment 9. The vector of any one of embodiments 5-8, wherein the gRNA is encoded by or specifically hybridizes to the nucleotide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 160 or 161.

Embodiment 10. The vector of any one of embodiments 5-9, wherein the gRNA is operatively linked to a promoter recognized by RNA polymerase III.

Embodiment 11. The vector of any one of embodiments 5-9, wherein the gRNA is operatively linked to a human U6 promoter.

Embodiment 12. The vector of any one of embodiments 5-11, wherein the vector further comprises a polynucleotide sequence encoding a second gRNA targeting the SCN1A gene.

Embodiment 13. The vector of any one of embodiments 5-12, wherein the nuclease-deficient CRISPR-associated protein is a nuclease-deficient Cas1, Cas1B, Cas2, Cas3, Cas4, Cas5, Cas6, Cas7, Cas8, Cas9, Cas1O, Cas11, Cas12, Cas13, CasX, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, or another Cas ortholog.

Embodiment 14. The vector of any one of embodiments 5-12, wherein the nuclease-deficient CRISPR-associated protein is dCas9.

Embodiment 15. The vector of embodiment 14, wherein the dCas9 is Staphylococcus aureus dCas9, Streptococcus pyogenes dCas9 or Campylobacter jejuni dCas9 or a dCas9 from an orthologous bacterial species.

Embodiment 16. The vector of embodiment 14, wherein the dCas9 comprises the amino acid sequence of SEQ ID NO: 156, 157, 158 or 159.

Embodiment 17. The vector of embodiment 14, wherein the dCas9 comprises an amino acid sequence at least about 90%, about 91%, about 92%, about 93%, about 94%, about 95%, about 96%, about 97%, about 98% or about 99% identical to the amino acid sequence of SEQ ID NO: 156, 157, 158 or 159.

Embodiment 18. The vector of embodiment 14, wherein the dCas9 is encoded by the nucleotide sequence of SEQ ID NO: 103, 104, 105 or 106.

Embodiment 19. The vector of embodiment 14, wherein the dCas9 is encoded by a nucleotide sequence at least about 90%, about 91%, about 92%, about 93%, about 94%, about 95%, about 96%, about 97%, about 98% or about 99% identical to the nucleotide sequence of SEQ ID NO: 103, 104, 105 or 106.

Embodiment 20. The vector of embodiment 14, wherein the dCas9 comprises a fragment of the amino acid sequence of SEQ ID NO: 156, 157, 158 or 159, wherein the fragment is a protein that is capable of forming a complex with the gRNA and targeting the SCN1A gene.

Embodiment 21. The vector of any one of embodiments 1-3, wherein the DTM comprises
(a) a zinc finger transcription factor or a portion of a zinc finger transcription factor; or
(b) a DNA binding domain of a zinc finger transcription factor.

Embodiment 22. The vector of embodiment 21, wherein the zinc finger transcription factor is a C2H2 zinc finger transcription factor.

Embodiment 23. The vector of embodiment 21, wherein the C2H2 zinc finger transcription factor comprises the DNA binding domain of a Zif268 zinc finger transcription factor or another humanized C2H2 zinc finger transcription factor, or a sequence derived from said DNA binding domain.

Embodiment 24. The vector of embodiment 23, wherein the DNA binding domain of a Zif268 zinc finger transcription factor targets the SCN1A gene.

Embodiment 25. The vector of any one of embodiments 1-3, wherein the DTM comprises
(a) a transcription activator-like protein effector (TALE) protein or a portion of a TALE protein; or
(b) a DNA binding domain of a TALE protein.

Embodiment 26. The vector of embodiment 25, wherein the DNA binding domain of a TALE protein targets the SCN1A gene.

Embodiment 27. The vector of any one of embodiments 1-26, wherein the promoter is a minimal promoter.

Embodiment 28. The vector of any one of embodiments 1-26, wherein the promoter is recognized by RNA polymerase II.

Embodiment 29. The vector of any one of embodiments 1-26, wherein the promoter is a human U6 promoter, a mouse U6 promoter or a human H1 promoter.

Embodiment 30. The vector of any one of embodiments 1-29, wherein the enhancer polynucleotide sequence comprises the nucleotide sequence of any one of SEQ ID NOs: 33-102.

Embodiment 31. The vector of any one of embodiments 1-29, wherein the enhancer polynucleotide sequence comprises the nucleotide sequence of SEQ ID NO: 69 or 34.

Embodiment 32. The vector of any one of embodiments 1-31, wherein the transactivator is VP16, VP32, VP48, VP64, VPR, a MS2-SAM system, p65, Rta, the CITE-D domains of p300 or a SunTag.

Embodiment 33. The vector of any one of embodiments 1-31, wherein the transactivator is encoded by the nucleotide sequence of SEQ ID NO: 107, 108, 109, 110, 111, 112 or 113 or a nucleotide sequence at least about 90%, about 91%, about 92%, about 93%, about 94%, about 95%, about 96%, about 97%, about 98% or about 99% identical to the nucleotide sequence of SEQ ID NO: 107, 108, 109, 110, 111, 112 or 113.

Embodiment 34. The vector of any one of embodiments 1-33, comprising in 5′-3′ order:
(a) the promoter polynucleotide sequence;
(b) the enhancer polynucleotide sequence; and
(c) the transgene polynucleotide sequence.

Embodiment 35. The vector of any one of embodiments 1-33, comprising in 5′-3′ order:
(a) the enhancer polynucleotide sequence;
(b) the promoter polynucleotide sequence; and
(c) the transgene polynucleotide sequence.

Embodiment 36. The vector of any one of embodiments 1-35, wherein
(a) the transgene polynucleotide sequence encodes the amino acid sequence of SEQ ID NO: 114, 118, 122 or 126; or an amino acid sequence at least about 90%, about 91%, about 92%, about 93%, about 94%, about 95%, about 96%, about 97%, about 98% or about 99% identical to the amino acid sequence of SEQ ID NO: 114, 118, 122 or 126; or
(b) the transgene polynucleotide sequence comprises or consists of the nucleotide sequence of SEQ ID NO: 115, 116, 117, 119, 120, 121, 123, 124, 125, 127, 128 or 129, or a nucleotide sequence at least about 90%, about 91%, about 92%, about 93%, about 94%, about 95%, about 96%, about 97%, about 98% or about 99% identical to the nucleotide sequence of SEQ ID NO: 115, 116, 117, 119, 120, 121, 123, 124, 125, 127, 128 or 129.

Embodiment 37. The vector of any one of embodiments 1-36, further comprising an artificial intron.

Embodiment 38. The vector of any one of embodiments 1-37, further comprising a Woodchuck Hepatitis Virus Posttranscriptional Regulatory Element (WPRE), a hepatitis B virus posttranscriptional regulatory element (HBVPRE), a RNA transport element (RTE), a WPRE3 or a ws13 regulatory element.

Embodiment 39. The vector of any one of embodiments 1-38, further comprising a polyadenylation signal sequence.

Embodiment 40. The vector of embodiment 39, wherein the polyadenylation signal sequence is a SV40 polyadenylation signal sequence.

Embodiment 41. The vector of any one of embodiments 1-40, wherein the vector is a viral vector.

Embodiment 42. The vector of embodiment 41, wherein the viral vector is an adeno-associated virus (AAV) vector.

Embodiment 43. The vector of embodiment 42, wherein the AAV vector comprises a first AAV inverted terminal repeat (ITR) located upstream of the promoter polynucleotide sequence and a second AAV ITR located downstream of the transgene polynucleotide sequence.

Embodiment 44. The vector of embodiment 43, wherein the first AAV ITR is an AAV2 ITR and the second AAV ITR is an AAV2 ITR.

Embodiment 45. The vector of any one of embodiments 42-44, comprising in 5′-3′ order:
(a) a 5′ ITR;
(b) a RNA polymerase III promoter;
(c) a polynucleotide sequence encoding a gRNA;
(d) the enhancer polynucleotide sequence;
(e) a minimal promoter;
(f) an artificial intron;
(g) the transgene polynucleotide sequence;
(h) a WPRE;
(i) a polyadenylation signal sequence; and
(j) a 3′ ITR.

Embodiment 46. The vector of any one of embodiments 42-44, comprising in 5′-3′ order:
(a) a 5′ ITR;
(b) a RNA polymerase III promoter;
(c) a polynucleotide sequence encoding a gRNA;
(d) a minimal promoter;
(e) the enhancer polynucleotide sequence;
(f) an artificial intron;
(g) the transgene polynucleotide sequence;
(h) a WPRE;
(i) a polyadenylation signal sequence; and
(j) a 3′ ITR.

Embodiment 47. The vector of embodiment 44, wherein the vector comprises the nucleotide sequence of SEQ ID NO: 137, 138, 139, 140, 141, 142, 143, 144, 145, 146, 147, 148, 149, 150, 151, 152 or 153, or a nucleotide sequence at least about 90%, about 91%, about 92%, about 93%, about 94%, about 95%, about 96%, about 97%, about 98% or about 99% identical to the nucleotide sequence of SEQ ID NO: 137, 138, 139, 140, 141, 142, 143, 144, 145, 146, 147, 148, 149, 150, 151, 152 or 153.

Embodiment 48. The vector of any one of embodiments 1-40, wherein the vector is suitable for delivery via a non-viral delivery system.

Embodiment 49. The vector of embodiment 48, wherein the non-viral delivery system is a lipid nanoparticle or an exosome.

Embodiment 50. A viral particle comprising the vector of any one of embodiments 41-47.

Embodiment 51. The viral particle of embodiment 50, wherein the viral particle is a recombinant AAV (rAAV) particle.

Embodiment 52. The viral particle of embodiment 51, wherein the rAAV particle is an AAV9, AAV-PHP.eB, AAV-DJ or AAV2 serotype particle.

Embodiment 53. A population of viral particles comprising a plurality of viral particles of any one of embodiments 50-52.

Embodiment 54. A pharmaceutical composition comprising the vector of any one of embodiments 1-49, the viral particle of any one of embodiments 50-52 or the population of embodiment 53, and a pharmaceutically acceptable carrier, vehicle or diluent.

Embodiment 55. A cell comprising the vector of any one of embodiments 1-49 or the viral particle of any one of embodiments 50-52.

Embodiment 56. The cell of embodiment 55, wherein the cell is a mammalian cell or an insect cell.

Embodiment 57. A method of producing a rAAV particle, the method comprising:
(i) culturing the cell of embodiment 55 or 56 under conditions allowing for packaging the rAAV particle; and
(ii) harvesting the cultured host cell or culture medium for collection of the rAAV particle.

Embodiment 58. The method of embodiment 57, wherein the rAAV particle comprises an AAV9, AAV-PHP.eB, AAV-DJ or AAV2 capsid protein.

Embodiment 59. A method for treating Dravet syndrome (DS) in a subject having or suspected of having DS, the method comprising administering to the subject a therapeutically effective amount of the vector of any one of embodiments 1-49, the viral particle of any one of embodiments 50-52, the population of embodiment 53 or the pharmaceutical composition of embodiment 54.

Embodiment 60. A method for treating or reducing the risk, severity, frequency or length of epilepsy and/or seizures in a subject who has or is at risk of having epilepsy, seizures, or Dravet syndrome (DS), the method comprising administering to the subject a therapeutically effective amount of the vector of any one of embodiments 1-49, the viral particle of any one of embodiments 50-52, the population of embodiment 53 or the pharmaceutical composition of embodiment 54.

Embodiment 61. A method for preventing or reducing the risk of Sudden Unexpected Death in Epilepsy (SUDEP) in a subject who has or is at risk of having epilepsy, seizures, or Dravet syndrome (DS), the method comprising administering to the subject a therapeutically effective amount of the vector of any one of embodiments 1-49, the viral particle of any one of embodiments 50-52, the population of embodiment 53 or the pharmaceutical composition of embodiment 54.

Embodiment 62. The method of any one of embodiments 59-61, wherein the subject is between about 2 years old and about 18 years old.

Embodiment 63. The method of any one of embodiments 59-61, wherein the subject is older than 18 years.

Embodiment 64. The method of any one of embodiments 59-63, wherein the vector, viral particle, population or pharmaceutical composition is administered to the subject via intracerebroventricular injection, intrathecal injection, intracarotid artery injection, or intraparenchymal injection.

Embodiment 65. The method of any one of embodiments 59-64, wherein the vector, viral particle, population or pharmaceutical composition is administered to the subject in a single dose.

Embodiment 66. The method of embodiment 65, wherein the single dose comprises from about 10E+9 to about 10E+14 viral particles.

Embodiment 67. A method for increasing levels of SCN1A expression in SCN1A-expressing cells in the brain, the method comprising contacting the cells with the vector of any one of embodiments 1-49, the viral particle of any one of embodiments 50-52, the population of embodiment 53 or the pharmaceutical composition of embodiment 54.

Embodiment 68. The method of embodiment 67, wherein the SCN1A-expressing cells comprise a loss-of-function mutation in one copy of the SCN1A gene.

Embodiment 69. The method of embodiment 67 or 68, wherein the SCN1A-expressing cells are GABAergic interneuron cells.

Embodiment 70. The method of embodiment 69, wherein the GABAergic interneuron cells express parvalbumin (PV).

Embodiment 71. The vector of any one of embodiments 1-49, the viral particle of any one of embodiments 50-52, the population of embodiment 53 or the pharmaceutical composition of embodiment 54, for use as a medicament.

1. A vector comprising:
(a) a transgene polynucleotide sequence encoding a sequence-specific DNA-targeting module (DTM) fused to a transactivator;
(b) an enhancer polynucleotide sequence that specifically restricts expression of the transgene to sodium voltage-gated channel alpha subunit 1 (SCN1A)-expressing cells in the brain; and
(c) a promoter polynucleotide sequence.

2. The vector of claim 1, wherein the SCN1A-expressing cells are GABAergic interneuron cells.

3. The vector of claim 1, wherein the SCN1A-expressing cells are parvalbumin (PV)-expressing interneurons.

4. The vector of any one of claims 1-3, wherein the DTM comprises a nuclease-deficient CRISPR-associated protein.

5. The vector of claim 4, wherein the vector further comprises a polynucleotide sequence encoding a guide RNA (gRNA).

6. The vector of claim 5, wherein the gRNA targets the SCN1A gene.

7. The vector of claim 5 or 6, wherein the gRNA specifically hybridizes to a regulatory region of the SCN1A gene.

8. The vector of claim 7, wherein the regulatory region of the SCN1A gene is a promoter or an enhancer.

9. The vector of any one of claims 5-8, wherein the gRNA is encoded by or specifically hybridizes to the nucleotide sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 160 or 161.

10. The vector of any one of claims 5-8, wherein the gRNA is encoded by or specifically hybridizes to the nucleotide sequence of SEQ ID NO: 11, 19, 21 or 27.

11. The vector of any one of claims 5-10, wherein the gRNA is operatively linked to a promoter recognized by RNA polymerase III.

12. The vector of any one of claims 5-10, wherein the gRNA is operatively linked to a human U6 promoter.

13. The vector of any one of claims 5-12, wherein the vector further comprises a polynucleotide sequence encoding a second gRNA targeting the SCN1A gene.

14. The vector of any one of claims 5-13, wherein the nuclease-deficient CRISPR-associated protein is a nuclease-deficient Cas1, Cas1B, Cas2, Cas3, Cas4, Cas5, Cas6, Cas7, Cas8, Cas9, Cas10, Cas11, Cas12, Cas13, CasX, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, or another Cas ortholog.

15. The vector of any one of claims 5-13, wherein the nuclease-deficient CRISPR-associated protein is dCas9.

16. The vector of claim 15, wherein the dCas9 is Staphylococcus aureus dCas9, Streptococcus pyogenes dCas9 or Campylobacter jejuni dCas9 or a dCas9 from an orthologous bacterial species.

17. The vector of claim 15, wherein the dCas9 comprises the amino acid sequence of SEQ ID NO: 156.

18. The vector of claim 15, wherein the dCas9 comprises an amino acid sequence at least about 90%, about 91%, about 92%, about 93%, about 94%, about 95%, about 96%, about 97%, about 98% or about 99% identical to the amino acid sequence of SEQ ID NO: 156.

19. The vector of claim 15, wherein the dCas9 is encoded by the nucleotide sequence of SEQ ID NO: 103, 104, 105 or 106.

20. The vector of claim 15, wherein the dCas9 is encoded by a nucleotide sequence at least about 90%, about 91%, about 92%, about 93%, about 94%, about 95%, about 96%, about 97%, about 98% or about 99% identical to the nucleotide sequence of SEQ ID NO: 103, 104, 105 or 106.

21. The vector of claim 15, wherein the dCas9 comprises a fragment of the amino acid sequence of SEQ ID NO: 156, wherein the fragment is a protein that is capable of forming a complex with the gRNA and targeting the SCN1A gene.

22. The vector of any one of claims 1-21, wherein the promoter is a minimal promoter.

23. The vector of any one of claims 1-21, wherein the promoter is recognized by RNA polymerase II.

24. The vector of any one of claims 1-21, wherein the promoter is a human U6 promoter, a mouse U6 promoter or a human H1 promoter.

25. The vector of any one of claims 1-24, wherein the enhancer polynucleotide sequence comprises the nucleotide sequence of any one of SEQ ID NOs: 33-102.

26. The vector of any one of claims 1-24, wherein the enhancer polynucleotide sequence comprises the nucleotide sequence of SEQ ID NO: 69 or 34.

27. The vector of any one of claims 1-26, wherein the transactivator is VP16, VP32, VP48, VP64, VPR, a MS2-SAM system, p65, Rta, the CITE-D domains of p300 or a SunTag.

28. The vector of any one of claims 1-26, wherein the transactivator is encoded by the nucleotide sequence of SEQ ID NO: 107, 108, 109, 110, 111, 112 or 113 or a nucleotide sequence at least about 90%, about 91%, about 92%, about 93%, about 94%, about 95%, about 96%, about 97%, about 98% or about 99% identical to the nucleotide sequence of SEQ ID NO: 107, 108, 109, 110, 111, 112 or 113.

29. The vector of any one of claims 1-28, comprising in 5′-3′ order:
(a) the promoter polynucleotide sequence;
(b) the enhancer polynucleotide sequence; and
(c) the transgene polynucleotide sequence.

30. The vector of any one of claims 1-28, comprising in 5′-3′ order:
(a) the enhancer polynucleotide sequence;
(b) the promoter polynucleotide sequence; and
(c) the transgene polynucleotide sequence.

31. The vector of any one of claims 1-30, wherein
(a) the transgene polynucleotide sequence encodes the amino acid sequence of SEQ ID NO: 114; or an amino acid sequence at least about 90%, about 91%, about 92%, about 93%, about 94%, about 95%, about 96%, about 97%, about 98% or about 99% identical to the amino acid sequence of SEQ ID NO: 114; or
(b) the transgene polynucleotide sequence comprises or consists of the nucleotide sequence of SEQ ID NO: 115, 116 or 117, or a nucleotide sequence at least about 90%, about 91%, about 92%, about 93%, about 94%, about 95%, about 96%, about 97%, about 98% or about 99% identical to the nucleotide sequence of SEQ ID NO: 115, 116 or 117.

32. The vector of any one of claims 1-31, further comprising an artificial intron.

33. The vector of any one of claims 1-32, further comprising a Woodchuck Hepatitis Virus Posttranscriptional Regulatory Element (WPRE), a hepatitis B virus posttranscriptional regulatory element (HBVPRE), a RNA transport element (RTE), a WPRE3 or a ws13 regulatory element.

34. The vector of any one of claims 1-33, further comprising a polyadenylation signal sequence.

35. The vector of claim 34, wherein the polyadenylation signal sequence is a SV40 polyadenylation signal sequence.

36. The vector of any one of claims 1-35, wherein the vector is a viral vector.

37. The vector of claim 36, wherein the viral vector is an adeno-associated virus (AAV) vector.

38. The vector of claim 37, wherein the AAV vector comprises a first AAV inverted terminal repeat (ITR) located upstream of the promoter polynucleotide sequence and a second AAV ITR located downstream of the transgene polynucleotide sequence.

39. The vector of claim 38, wherein the first AAV ITR is an AAV2 ITR and the second AAV ITR is an AAV2 ITR.

40. The vector of any one of claims 37-39, comprising in 5′-3′ order:
(a) a 5′ ITR;
(b) a RNA polymerase III promoter;
(c) a polynucleotide sequence encoding a gRNA;
(d) the enhancer polynucleotide sequence;
(e) a minimal promoter;
(f) an artificial intron;
(g) the transgene polynucleotide sequence;
(h) a WPRE;
(i) a polyadenylation signal sequence; and
(j) a 3′ ITR.

41. The vector of any one of claims 37-39, comprising in 5′-3′ order:
(a) a 5′ ITR;
(b) a RNA polymerase III promoter;
(c) a polynucleotide sequence encoding a gRNA;
(d) a minimal promoter;
(e) the enhancer polynucleotide sequence;
(f) an artificial intron;
(g) the transgene polynucleotide sequence;
(h) a WPRE;
(i) a polyadenylation signal sequence; and
(j) a 3′ ITR.

42. The vector of claim 39, wherein the vector comprises the nucleotide sequence of SEQ ID NO: 137, 141, 145, 149 or 153, or a nucleotide sequence at least about 90%, about 91%, about 92%, about 93%, about 94%, about 95%, about 96%, about 97%, about 98% or about 99% identical to the nucleotide sequence of SEQ ID NO: 137, 141, 145, 149 or 153.

43. The vector of any one of claims 1-35, wherein the vector is suitable for delivery via a non-viral delivery system.

44. The vector of claim 43, wherein the non-viral delivery system is a lipid nanoparticle or an exosome.

45. A viral particle comprising the vector of any one of claims 36-42.

46. The viral particle of claim 45, wherein the viral particle is a recombinant AAV (rAAV) particle.

47. The viral particle of claim 46, wherein the rAAV particle is an AAV9, AAV-PHP.eB, AAV-DJ or AAV2 serotype particle.

48. A population of viral particles comprising a plurality of viral particles of any one of claims 45-47.

49. A pharmaceutical composition comprising the vector of any one of claims 1-44, the viral particle of any one of claims 45-47or the population of claim 48, and a pharmaceutically acceptable carrier, vehicle or diluent.

50. A cell comprising the vector of any one of claims 1-44 or the viral particle of any one of claims 45-47.

51. The cell of claim 50, wherein the cell is a mammalian cell or an insect cell.

52. A method of producing a rAAV particle, the method comprising:
(i) culturing the cell of claim 50 or 51 under conditions allowing for packaging the rAAV particle; and
(ii) harvesting the cultured host cell or culture medium for collection of the rAAV particle.

53. The method of claim 52, wherein the rAAV particle comprises an AAV9, AAV-PHP.eB, AAV-DJ or AAV2 capsid protein.

54. A method for treating Dravet syndrome (DS) in a subject having or suspected of having DS, the method comprising administering to the subject a therapeutically effective amount of the vector of any one of claims 1-44, the viral particle of any one of claims 45-47, the population of claim 48 or the pharmaceutical composition of claim 49.

55. A method for treating or reducing the risk, severity, frequency or length of epilepsy and/or seizures in a subject who has or is at risk of having epilepsy, seizures, or Dravet syndrome (DS), the method comprising administering to the subject a therapeutically effective amount of the vector of any one of claims 1-44, the viral particle of any one of claims 45-47, the population of claim 48 or the pharmaceutical composition of claim 49.

56. A method for preventing or reducing the risk of Sudden Unexpected Death in Epilepsy (SUDEP) in a subject who has or is at risk of having epilepsy, seizures, or Dravet syndrome (DS), the method comprising administering to the subject a therapeutically effective amount of the vector of any one of claims 1-44, the viral particle of any one of claims 45-47, the population of claim 48 or the pharmaceutical composition of claim 49.

57. The method of any one of claims 54-56, wherein the subject is between about 2 years old and about 18 years old.

58. The method of any one of claims 54-56, wherein the subject is older than 18 years.

59. The method of any one of claims 54-58, wherein the vector, viral particle, population or pharmaceutical composition is administered to the subject via intracerebroventricular injection, intrathecal injection, intracarotid artery injection, or intraparenchymal injection.

60. The method of any one of claims 54-59, wherein the vector, viral particle, population or pharmaceutical composition is administered to the subject in a single dose.

61. The method of claim 60, wherein the single dose comprises from about 10E+9 to about 10E+14 viral particles.

62. A method for increasing levels of SCN1A expression in SCN1A-expressing cells in the brain, the method comprising contacting the cells with the vector of any one of claims 1-44, the viral particle of any one of claims 45-47, the population of claim 48 or the pharmaceutical composition of claim 49.

63. The method of claim 62, wherein the SCN1A-expressing cells comprise a loss-of-function mutation in one copy of the SCN1A gene.

64. The method of claim 62 or 63, wherein the SCN1A-expressing cells are GABAergic interneuron cells.

65. The method of claim 64, wherein the GABAergic interneuron cells express parvalbumin (PV).

66. The vector of any one of claims 1-44, the viral particle of any one of claims 45-47, the population of claim 48 or the pharmaceutical composition of claim 49, for use as a medicament.
